(19) United States
(12) Patent Application Publication
(10) Pub. No.: US 2024/0092802 A1
(43) Pub. Date: Mar. 21, 2024

(54) MU-OPIOID RECEPTOR AGONISTS AND USES THEREFOR

(71) Applicant: Kures, Inc., New York, NY (US)

(72) Inventor: Andrew Kruegel, Secaucus, NJ (US)

(21) Appl. No.: 18/030,496

(22) PCT Filed: Oct. 6, 2021
(86) PCT No.: PCT/IB2021/059178

§ 371 Date: Apr. 6, 2023

Related U.S. Application Data
(60) Provisional application No. 63/088,413, filed on Oct. 6, 2020.

(51) Int. Cl. C07D 513/04; C07D 281/02; C07D 417/12; A61P 25/28

(52) U.S. Cl. CPC C07D 513/04; C07D 281/02; C07D 417/12; A61P 25/28

(57) ABSTRACT

The present disclosure relates to a compound having the structure: Formula (I) or a pharmaceutically acceptable salt or ester thereof, for treating or preventing a neurological disorder, including Huntington's disease, Rett syndrome, and CDKL5 disorder.

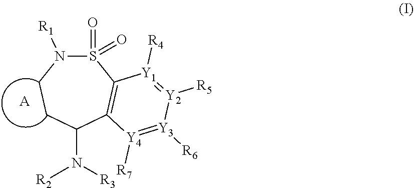

All references, including patents and patent applications, cited in this specification are hereby incorporated by reference. No admission is made that any reference constitutes prior art. Nor does discussion of any reference constitute an admission that such reference forms part of the common general knowledge in the art, in any country.

### BACKGROUND OF THE INVENTION

Mu-opioid receptor (MOR) has been recognized as an important molecular target for several decades. However, the vast majority of MOR agonists used clinically today are structurally related to or derived from morphine (and other opioid alkaloids). These compounds suffer from many serious problems, including development of tolerance (increased dosing is required to achieve the same analgesic effects), high addiction liability, and other side effects (e.g., respiratory depression, nausea, and constipation) (Williams, J. T. et al. 2013). Therefore, there is a continuing interest in the development of new MOR agonists with improved therapeutic profile (Corbett, A. D. et al. 2006).

MOR agonists have been widely used for pain treatment. There is also historical and growing interest in the use of MOR agonists as medicaments for depression. Prior to the adoption of tricyclic antidepressants and electroshock therapy as favored treatments for depression, opiates were among the only options available, with the “opium cure” being an accepted treatment modality in the early 20th century (Berrocoso, E. et al. 2009). More recently, studies in both rodents (Besson, A. et al. 1996) and humans (Bodkin, J. A. et al. 1995) have suggested that MOR activation may lead to antidepressant and/or anxiolytic effects.

On the molecular level, MORs are extensively expressed in the hippocampus and have been shown to exert a variety of indirect modulatory effects on glutamatergic neurons in this brain region (Xie, C. W. et al. 1997; Svoboda, K. R. et al. 1999). Normalization and modulation of glutamate signaling has been strongly associated with the actions of antidepressants (Paul, I. A. and Skolnick, P. 2003) and indeed, the NMDA antagonist ketamine, shows rapid and efficacious antidepressant activity in human clinical trials (Zarate, C. A. Jr et al. 2006). Further, agonists of the related delta-opioid receptor (DOR) have been demonstrated to show robust antidepressant efficacy (Jutkiewicz, E. M. 2006).

In addition to pain, anxiety, behavioral disorders, and mood disorders, there are other serious neurological disorders that afflict patients. Disorders such as Huntington's disease, Rett syndrome, and CDKL5 disorder, amongst others, have significant deleterious effects, with limited or no treatment options available. Therefore, there is an ongoing need for therapeutics to address these neurological disorders and their symptoms.

### SUMMARY OF THE INVENTION

The present disclosure provides a compound having the structure:

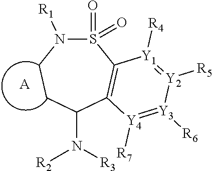

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkylaryl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
wherein when A is phenyl, R₁ is —CH₃, R₃, R₄, R₆, and R₇ are each —H, and R₅ is Cl, then R₂ is other than —(CH₂)₄C(O)NH₂, —(CH₂)₄CO₂H, —(CH₂)₅CO₂H, —(CH₂)₆CO₂H, —(CH₂)₇CO₂H, —(CH₂)₁₀CO₂H, —(CH₂)₆CO₂CH₂CH₃, —(CH₂)₆CH₃, —(CH₂)₂OH, —(CH₂)₄OH, —(CH₂)₇OH,
wherein when A is phenyl, R₁ is —CH₃, R₃, R₄, R₅, R₆, and R₇ are each —H, then R₂ is other than —(CH₂)CO₂CH₂CH₃, —(CH₂)₂CO₂CH₂CH₃, —(CH₂) CO₂H, —(CH₂)₃CO₂H, —(CH₂)₄CO₂H or —(CH₂)₆CO₂H,
wherein when R₁ is —CH₃, R₂ is —(CH₂)₅CO₂H, R₃ is —H, R₄ and R₇ are each H, R₅ is —Cl and R₆ is —H or R₅ and R₆ are each —H, then A is other than 2-chlorophenyl or 3-chlorophenyl,
wherein when R₁ is —CH₃, R₂ is —(CH₂)₅CO₂H, R₂ is —H or —CH₃, R₄ and R₇ are each —H, R₅ is Cl and R₆ is —H or R₆ is —Cl and R₅ is —H, then A is other than phenyl,
wherein when R₁ is —CH₃, R₂ is —(CH₂)₃CO₂H, R₃ is —CH₃, and R₄, R₅, R₆ and R₇ are each —H, then A is other than phenyl,
wherein when A is phenyl, R₁ is —CH₃, R₃, R₄, R₆, and R₇ are each —H, and R₅ is —SO₂CH₃, then R₂ is other than —(CH₂)₃OCH₃,
wherein when A is phenyl, R₁ is —CH₃, R₃, R₄, R₆, and R₇ are each —H, and R₅ is —F, then R₂ is other than —(CH₂)₆CO₂H,
   or a pharmaceutically acceptable salt or ester thereof,
   wherein the compound is for treating or preventing a neurological disorder in a subject, wherein the neurological disorder is selected from the group consisting of Huntington's disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; and Lennox-Gastaut syndrome.

The present disclosure also provides a compound having the structure:

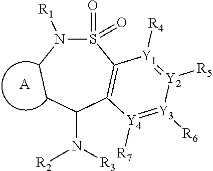

wherein
A is an aryl or heteroaryl, with or without substitution;
R₁ is —H or -(alkyl);
R₂ is -(alkyl), -(alkenyl), -(alkynyl)-(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-OCH₃, -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
wherein when A is phenyl, R₁ is —CH₃, R₃, R₄, R₆, and R₇ are each —H, and R₅ is Cl, then R₂ is other than —(CH₂)₄CO₂H, —(CH₂)₆CO₂H, —(CH₂)₆CO₂CH₂CH₃, or —(CH₂)₆CH₃,
wherein when A is phenyl, R₁ is —CH₃, R₃, R₄, R₆, and R₇ are each —H, and R₅ is —SO₂CH₃, then R₂ is other than —(CH₂)₃OCH₃,
   or a pharmaceutically acceptable salt thereof,
   wherein the compound is for treating or preventing a neurological disorder in a subject, wherein the neurological disorder is selected from the group consisting of Huntington's disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; and Lennox-Gastaut syndrome.

The present disclosure further provides a compound having the structure:

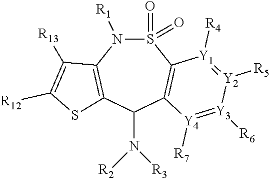

wherein
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-OCH₃, -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrocxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl);
R₁₂ and R₁₃ are each independently —H, —Cl, —Br, —F, —I, —CN, —CF₃, —OCF₃, -(alkyl), -(aryl), -(heteroaryl)-(alkenyl), -(alkynyl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
      or a pharmaceutically acceptable salt thereof,
      wherein the compound is for treating or preventing a neurological disorder in a subject, wherein the neurological disorder is selected from the group consisting of Huntington's disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; and Lennox-Gastaut syndrome.

The present disclosure yet further provides a compound having the structure:

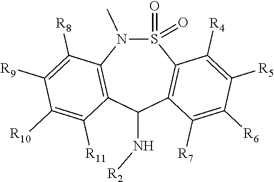

wherein
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, (alkyl)-pyrrolidine, -(alkyl)-N-methylpyrrolidine, -(alkyl)-(1,3-dioxane) or -(alkyl)-(4,5-dihydrooxazole);
R₅ is —Br, or —I;
R₄, R₆ and R/are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl) or —SO₂-(heteroaryl),
   or a pharmaceutically acceptable salt thereof,
   wherein the compound is for treating or preventing a neurological disorder in a subject, wherein the neurological disorder is selected from the group consisting of Huntington's disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; and Lennox-Gastaut syndrome.

The present disclosure also provides a compound having the structure:

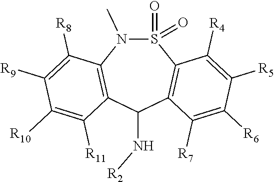

wherein
R₂ is -(alkyl)-O-(alkyl) or -(alkyl)-O-(alkyl)-O-(alkyl);
R₅ is —Cl, —Br, —F, or —I;
R₄, R₆ and R₇ are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl) or —SO₂-(heteroaryl);
R₈, R₉, R₁₀ and R₁₁ are each independently —H, —Cl, —Br, —F, —I, —CN, —CF₃, —OCF₃, -(alkyl), -(aryl), -(heteroaryl)-(alkenyl), -(alkynyl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl),
   or a pharmaceutically acceptable salt thereof,
   wherein the compound is for treating or preventing a neurological disorder in a subject, wherein the neurological disorder is selected from the group consisting of Huntington's disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; and Lennox-Gastaut syndrome.

The present disclosure provides a method of treating or preventing a neurological disorder in a subject, wherein the neurological disorder is selected from the group consisting of Huntington's disease; a Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; and Lennox-Gastaut syndrome, wherein the method comprises administering to the subject a compound having the structure:

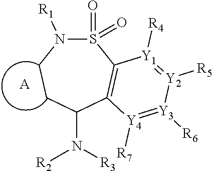

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkylaryl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
      or a pharmaceutically acceptable salt or ester thereof, so as to thereby treat or prevent the neurological disorder in the subject.

The present disclosure provides a method of treating or preventing a neurological disorder in a subject, wherein the neurological disorder is Huntington's disease, wherein the method comprises administering to the subject a compound having the structure:

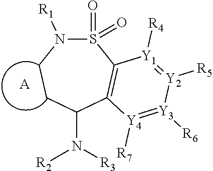

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkylaryl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
      or a pharmaceutically acceptable salt thereof, so as to thereby treat or prevent the neurological disorder in the subject.

The present disclosure provides a method of treating a subject afflicted with a neurological disorder, wherein the neurological disorder is selected from the group consisting of Huntington's disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; and Lennox-Gastaut syndrome, comprising administering to the subject an effective amount of an NMDA receptor antagonist, an NMDA receptor partial agonist, a neurokinin 1 receptor antagonist, a neurokinin 2 receptor antagonist or a neurokinin 3 receptor antagonist and an effective amount of a compound having the structure:

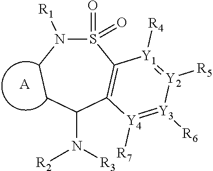

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkylaryl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
      or a pharmaceutically acceptable salt thereof, so as to thereby treat the neurological disorder in the subject.

The present disclosure provides a method of treating a subject afflicted with a neurological disorder, wherein the neurological disorder is Huntington's disease, comprising administering to the subject an effective amount of an NMDA receptor antagonist, an NMDA receptor partial agonist, a neurokinin 1 receptor antagonist, a neurokinin 2 receptor antagonist or a neurokinin 3 receptor antagonist and an effective amount of a compound having the structure:

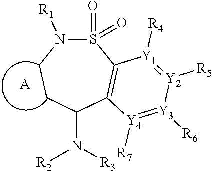

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkylaryl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
      or a pharmaceutically acceptable salt thereof, so as to thereby treat the neurological disorder in the subject.

The present disclosure provides a compound having the structure

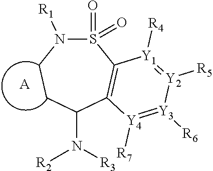

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrocxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkylaryl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
or a salt or ester thereof, for use as an add-on therapy or in combination with an NMDA receptor antagonist, an NMDA receptor partial agonist, a neurokinin 1 receptor antagonist, a neurokinin 2 receptor antagonist or a neurokinin 3 receptor antagonist in treating a subject afflicted with a neurological disorder, wherein the neurological disorder is selected from the group consisting of Huntington's disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; and Lennox-Gastaut syndrome.

The present disclosure provides a compound having the structure

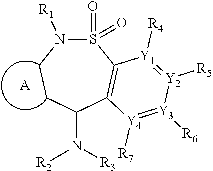

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkylaryl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
or a salt or ester thereof, for use as an add-on therapy or in combination with an NMDA receptor antagonist, an NMDA receptor partial agonist, a neurokinin 1 receptor antagonist, a neurokinin 2 receptor antagonist or a neurokinin 3 receptor antagonist in treating a subject afflicted with a neurological disorder, wherein the neurological disorder is Huntington's disease.

The present disclosure provides a pharmaceutical composition comprising an amount of a compound having the structure

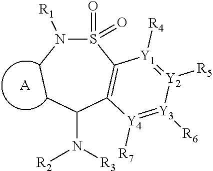

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrocxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkylaryl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
or a salt or ester thereof, and an amount of an NMDA receptor antagonist, an NMDA receptor partial agonist, a neurokinin 1 receptor antagonist, a neurokinin 2 receptor antagonist or a neurokinin 3 receptor antagonist for use in treating a subject afflicted with a neurological disorder, wherein the neurological disorder is selected from the group consisting of Huntington's disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; and Lennox-Gastaut syndrome.

The present disclosure provides a pharmaceutical composition comprising an amount of a compound having the structure

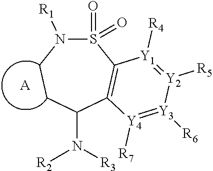

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrocxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkylaryl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
or a salt or ester thereof, and an amount of an NMDA receptor antagonist, an NMDA receptor partial agonist, a neurokinin 1 receptor antagonist, a neurokinin 2 receptor antagonist or a neurokinin 3 receptor antagonist for use in treating a subject afflicted with a neurological disorder, wherein the neurological disorder is Huntington's disease.

The present disclosure provides a compound having the structure:

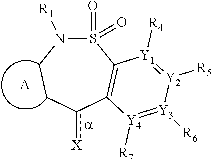

wherein
α is a bond, which may be present or absent;
X is O, OH, OTf, Cl, or Br,
   wherein when a is present, then X is O, and when a is absent, then X is OH, OTf, Cl, or Br; A is an aryl or heteroaryl, with or without substitution;
R₁ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
      or a pharmaceutically acceptable salt thereof.

In some embodiments, the compound has the structure of:

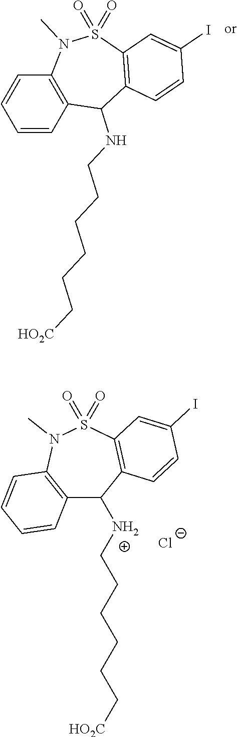 [SMILES: CN1c2ccccc2C(NCCCCCCC(=O)O)c2ccc(I)cc2S1(=O)=O ; "or" ; CN1c2ccccc2C([NH2+]CCCCCCC(=O)O)c2ccc(I)cc2S1(=O)=O ; [Cl-]]

It should also be appreciated that compounds as defined herein, or compositions as defined herein can be used for the manufacture of a medicament for treating a subject afflicted with a neurological disorder, wherein the neurological disorder is selected from the group consisting of Huntington's disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; and Lennox-Gastaut syndrome.

The foregoing brief summary broadly describes certain features and technical advantages. Further features and technical advantages will be described in the detailed description and examples that follow.

Other features and advantages will be better understood from the detailed description read in connection with the accompanying figures and examples. However, the figures and examples provided herein are intended to help illustrate the invention or assist with developing an understanding of the invention, and are not intended to limit the invention's scope.

### BRIEF DESCRIPTION OF THE FIGURES

FIG. 1A: EC₅₀ (human MOR) of DAMGO, tianeptine and compound 23.

FIG. 1B: EC₅₀ (mouse MOR) of DAMGO, tianeptine and compound 23.

FIG. 2A: DSBA data for compound 84. Odds ratios that the behavioral phenotypes induced by drug treatments are similar to the inverse of the behavioral phenotype exhibited by Q175 heterozygous mice at 6 months of age. An odds ratio of 1 suggests a random relationship. *p<0.05; **p<0.01.

FIG. 2B: DBSA data for compound 7e. Odds ratios that the behavioral phenotypes induced by drug treatments are similar to the inverse of the behavioral phenotype exhibited by Q175 heterozygous mice at 6 months of age. An odds ratio of 1 suggests a random relationship. *p<0.05; **p<0.01.

FIG. 3A: DSBA data for compound 84. Odds ratios that the behavioral phenotypes induced by drug treatments are similar to the inverse of the behavioral phenotype exhibited by Q175 heterozygous mice at 10 months of age. An odds ratio of 1 suggests a random relationship. *p<0.05; **p<0.01.

FIG. 3B: DSBA data for compound 7e. Odds ratios that the behavioral phenotypes induced by drug treatments are similar to the inverse of the behavioral phenotype exhibited by Q175 heterozygous mice at 10 months of age. An odds ratio of 1 suggests a random relationship. *p<0.05; **p<0.01.

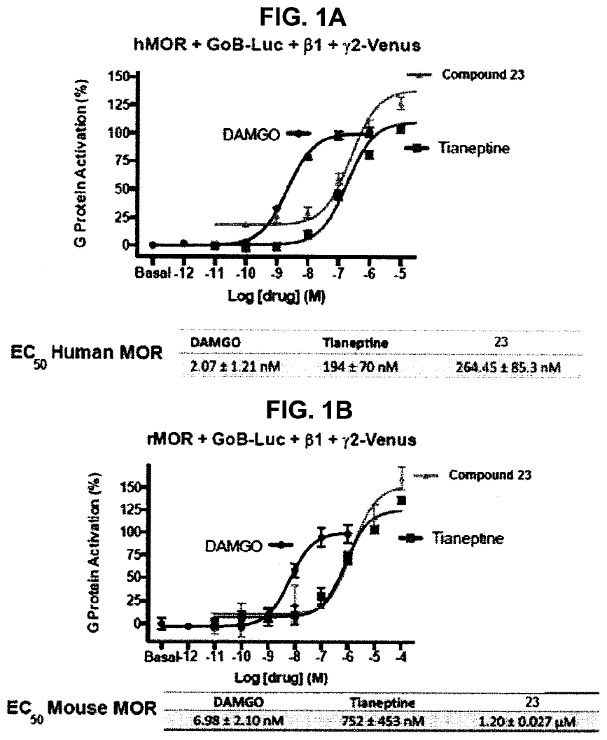

### DETAILED DESCRIPTION OF THE INVENTION

The following description sets forth numerous exemplary configurations, parameters, and the like. It should be recognized, however, that such description is not intended as a limitation on the scope of the present disclosure, but is instead provided as a description of exemplary embodiments.

In each instance herein, in descriptions, embodiments, and examples of the present disclosure, the terms “comprising”, “including”, etc., are to be read expansively, without limitation. Thus, unless the context clearly requires otherwise, throughout the description and the claims, the words “comprise”, “comprising”, and the like are to be construed in an inclusive sense as opposed to an exclusive sense, that is to say in the sense of “including but not limited to”.

The term “consisting essentially of”, as used herein, may refer to the presence of a component in a composition. For example, a concentrate may be at least 80% by weight of the composition, or at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.8%, or at least 99.9% by weight of the composition (% w/w). For liquids, a concentrate may be at least 80% by volume of the composition volume, or at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.8%, or at least 99.9% by volume of the composition volume (% v/v).

In the present description, the articles “a” and “an” are used to refer to one or to more than one (i.e., to at least one) of the grammatical object of the article. By way of example, “an element” can be taken to mean one element or more than one element.

Throughout this description, the term “approximately” is used to indicate that a value includes the standard deviation of error for the method being employed to determine the value, for example, dosage levels, as described in detail herein. In particular, the term “approximately” encompasses a 10% to 15% deviation (positive and negative) in the stated value or range, particularly 10% deviation (positive and negative) in the stated value or range.

“Neurological disorder” refers to various conditions of the neurological system including neurodegenerative and neurodevelopmental conditions. Specifically included are Huntington's disease; Rett syndrome; Rett syndrome variants; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; and Lennox-Gastaut syndrome.

“Symptoms” of a neurological disorder refer to various physical effects exhibited in subjects. Physical symptoms can include one or more of: involuntary movements (e.g., chorea); abnormal muscle rigidity or contraction (e.g., dystonia); abnormal hand movements (e.g., stereotypies); poor muscle tone (e.g., hypotonia), unusual eye movements; unusual facial expressions; tremors; impaired gait, posture, or balance; frequent falls; loss of balance; difficulty with swallowing; teeth grinding (e.g., bruxism); fatigue; and insomnia. Cognitive symptoms are also noted, and may include one or more of: difficulties with concentration or organization; communication difficulties; perseveration; lack of impulse control; lack of self-awareness; slowed thought processing; and learning difficulties. Serious symptoms are noted, including breathing problems; irregular heartbeat; and seizures.

“Treating” as used herein refers to reducing, ameliorating, or resolving a disorder, for example a neurological disorder, such as Huntington's disease, Rett syndrome, or CDKL5 disorder. A treatment is expected to result in the reduction, amelioration, or elimination of one or more symptoms of the disorder.

“Preventing” as used herein refers to stopping or delaying the onset of a disorder, for example a neurological disorder, such as Huntington's disease, Rett syndrome, or CDKL5 disorder. A preventative measure is expected to result in the inhibition or delay in onset of one or more symptoms of the disorder, the lessening of symptoms if such do arise, and/or the inhibition or delay of the progression of the disorder.

It should be understood that the term “treating or preventing” does not exclude the possibility of obtaining both treatment and prevention (e.g., at the same time or at different times) of a disorder in any given subject.

Of particular interest in the present disclosure is the use of a compound of this disclosure in the treatment or prevention of one or more neurological disorders in a subject. This includes neurodegenerative diseases and neurodevelopmental disorders, for example, Huntington's disease; Rett syndrome which includes Rett syndrome variants, such as the Rett syndrome Rolando variant (congenital variant), the Rett syndrome Zappella variant, the Rett syndrome Hanefeld variant (early epilepsy variant); Angelman syndrome; Prader-Willi syndrome; neonatal onset encephalopathy; X-linked recessive mental retardation (MRX); fetal alcohol spectrum disorder; Hirschsprung disease; CDKL5 disorder; West syndrome (infantile spasms, ISSX); FOXG1 syndrome; and Lennox-Gastaut syndrome.

Notably, the Huntingtin gene product (Htt), is known to interact with the gene product associated with Rett syndrome (MeCP2). The MeCP2 gene product is also associated with Angelman syndrome, Prader-Willi syndrome; neonatal onset encephalopathy, X-linked recessive mental retardation, fetal alcohol spectrum disorder, and Hirschsprung disease. CDKL5 and FOXG1 mutations may be associated with Rett syndrome, and CDKL5 and FOXG1 mutations lead to symptoms that overlap with Rett syndrome symptoms. Accordingly, CDKL5 and FOXG1 disorders have been referred to as Rett syndrome related disorders.

The present disclosure provides a compound having the structure:

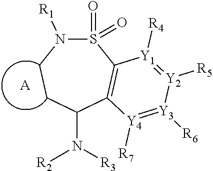

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkylaryl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
wherein when A is phenyl, R₁ is —CH₃, R₃, R₄, R₆, and R₇ are each —H, and R₅ is Cl, then R₂ is other than —(CH₂)₄C(O)NH₂, —(CH₂)₄CO₂H,
(CH₂)₅CO₂H, —(CH₂)₆CO₂H, —(CH₂)₇CO₂H, —(CH₂)₁₀CO₂H, —(CH₂)₆CO₂CH₂CH₃, —(CH₂)₆CH₃, —(CH₂)₂OH, —(CH₂)₄OH, —(CH₂)₇OH,
wherein when A is phenyl, R₁ is —CH₃, R₃, R₄, R₅, R₆, and R₇ are each —H, then R₂ is other than —(CH₂) CO₂CH₂CH₃, —(CH₂)₂CO₂CH₂CH₃, —(CH₂) CO₂H, —(CH₂)₃CO₂H, or —(CH₂)₄CO₂H,
wherein when R₁ is —CH₃, R₂ is —(CH₂)₅CO₂H, R₃ is —H, R₄ and R₇ are each H, R₅ is —Cl and R₆ is —H or R₅ and R₆ are each —H, then A is other than 2-chlorophenyl or 3-chlorophenyl,
wherein when R₁ is —CH₃, R₂ is —(CH₂)₃CO₂H, R₃ is —CH₃, and R₄, R₅, R₆ and R₇ are each —H, then A is other than phenyl,
wherein when R₁ is —CH₃, R₂ is —(CH₂)₅CO₂H, R₃ is —H or —CH₃, R₄ and R₇ are each —H, R₅ is Cl and R₆ is —H or R₆ is —Cl and R₅ is —H, then A is other than phenyl,
wherein when A is phenyl, R₁ is —CH₃, R₃, R₄, R₆, and R₇ are each —H, and R₅ is —SO₂CH₃, then R₂ is other than —(CH₂)₃OCH₃,
wherein when A is phenyl, R₁ is —CH₃, R₃, R₄, R₆, and R₇ are each —H, and R₅ is —F, then R₂ is other than —(CH₂)₆CO₂H,
   or a pharmaceutically acceptable salt or ester thereof,
   wherein the compound is for treating or preventing a neurological disorder in a subject, wherein the neurological disorder is selected from the group consisting of Huntington's disease, Rett syndrome, and CDKL5 disorder, and the other neurological disorders as described herein.

In some embodiments,
R₂ is -(alkyl), -(alkenyl), -(alkynyl), -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-OCH₃, -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole).

In some embodiments,
R₂ is -(alkyl), -(alkenyl), -(alkynyl), -(alkyl)-OH, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-OCH₃, -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole).

In some embodiments,
R₂ is -(alkyl), -(alkenyl), -(alkynyl), -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-OCH₃, -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole).

In some embodiments,
R₂ is -(alkyl), -(alkenyl), -(alkynyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-OCH₃, -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole).

The present disclosure also provides a compound having the structure:

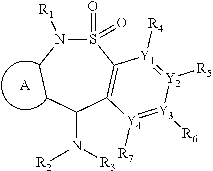

wherein
A is an aryl or heteroaryl, with or without substitution;
R₁ is —H or -(alkyl);
R₂ is -(alkyl), -(alkenyl), -(alkynyl)-(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-OCH₃, -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
wherein when A is phenyl, R₁ is —CH₃, R₃, R₄, R₆, and R₇ are each —H, and R₅ is Cl, then R₂ is other than —(CH₂)₄CO₂H, —(CH₂)₆CO₂H,
(CH₂)₆CO₂CH₂CH₃, or —(CH₂)₆CH₃,
wherein when A is phenyl, R₁ is —CH₃, R₃, R₄, R₆, and R₇ are each —H, and R₅ is —SO₂CH₃, then R₂ is other than —(CH₂)₃OCH₃,
   or a pharmaceutically acceptable salt thereof,
   wherein the compound is for treating or preventing a neurological disorder in a subject, wherein the neurological disorder is selected from the group consisting of Huntington's disease, Rett syndrome, and CDKL5 disorder, and the other neurological disorders as described herein.

In some embodiments, a compound having the structure:

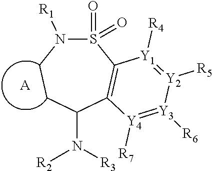

wherein
A is an aryl or heteroaryl, with or without substitution;
R₁ is —H or -(alkyl);
R₂ is -(alkyl), -(alkenyl), -(alkynyl), -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-OCH₃, -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
wherein when A is phenyl, R₁ is —CH₃, R₃, R₄, R₆, and R₇ are each —H, and R₅ is Cl, then R₂ is other than —(CH₂)₆CH₃,
wherein when A is phenyl, R₁ is —CH₃, R₃, R₄, R₆, and R₇ are each —H, and R₅ is —SO₂CH₃, then R₂ is other than —(CH₂)₃OCH₃,
   or a pharmaceutically acceptable salt thereof,
   wherein the compound is for treating or preventing a neurological disorder in a subject, wherein the neurological disorder is selected from the group consisting of Huntington's disease, Rett syndrome, and CDKL5 disorder, and the other neurological disorders as described herein.

In some embodiments, the compound wherein
A is

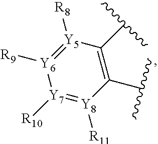

wherein R₈, R₉, R₁₀ and R₁₁ are each absent or present, and when present, are each independently —H, —Cl, —Br, —F, —I, —CN, —CF₃, —OCF₃, -(alkyl), -(aryl), -(heteroaryl)-(alkenyl), -(alkynyl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl);
Y₅, Y₆, Y₇ and Y₈ are each independently N or C,
   wherein when Y₅ is N, then R₈ is absent, and when Y₅ is C, then R₈ is present; when Y₆ is N, then R₉ is absent, and when Y₆ is C, then R₉ is present; when Y₇ is N, then R₁₀ is absent, and when Y₇ is C, then R₁₀ is present; when Y₈ is N, then R₁₁ is absent, and when Y₈ is C, then R₁₁ is present.

In some embodiments, the compound wherein
Y₅, Y₆, Y₇ and Y₈ are each C; and
R₈, R₉, R₁₀ and R₁₁ are each independently —H, —Cl, —Br, —F, —I, —CN, —CF₃, —OCF₃, —OH, —OAc, —(C₁-C₆ alkyl), —O—(C₁-C₆ alkyl), —S—(C₁-C₆ alkyl), —SO₂—(C₁-C₆ alkyl), —S(O)—(C₁-C₆ alkyl), —O-(aryl) or —S-(aryl), or -(aryl).

In some embodiments, the compound wherein
Y₅, Y₆, Y₇ and Y₈ are each C; and
R₈, R₉, R₁₀ and R₁₁ are each independently —H, —CH₃, —Cl, —Br, —F, —I, —OCH₃, —OH, —OAc, —SCH₃, —SO₂CH₃, —S(O) CH₃, -(phenyl), or —O-(phenyl).

In some embodiments, the compound wherein
Y₅, Y₆, Y₇ and Y₈ are each C; and
R₈, R₉, R₁₀ and R₁₁ are each —H.

In some embodiments, the compound wherein
Y₅, Y₆, Y₇ and Y₈ are each C;
R₇, R₈, and R₁₁ are each —H; and R₁₀ is —Br.

In some embodiments, the compound wherein
Y₅, Y₆, Y₇ and Y₈ are each C
R₉, R₁₀, and R₁₁ are each —H; and R₈ is —OCH₃.

In some embodiments, the compound wherein
Y₅, Y₆, Y₇ and Y₈ are each C
R₉, R₁₀, and R₁₁ are each —H; and R₈ is —OH.

In some embodiments, the compound wherein
Y₅, Y₆, Y₇ and Y₈ are each C
R₉, R₁₀, and R₁₁ are each —H; and R is —F, —Cl, —Br, or —I.

In some embodiments, the compound wherein
Y₁, Y₂, Y₃ and Y₄ are each C; and
R₄, R₅, R₆ and R₇ are each independently —H, —Cl, —Br, —F, —I, —CN, —CF₃, —OCF₃, —OH, —OAc, —(C₁-C₆ alkyl), —O—(C₁-C₆ alkyl), —S—(C₁-C₆ alkyl), —SO₂—(C₁-C₆ alkyl), —S(O)—(C₁-C₆ alkyl), —O-(aryl) or —S-(aryl), or -(aryl).

In some embodiments, the compound wherein
Y₁, Y₂, Y₃ and Y₄ are each C; and
R₄, R₅, R₆ and R₇ are each independently —H, —CH₃, —Cl, —Br, —F, —I, —OCH₃, —OH, —OAc, —SCH₃, —SO₂CH₃, —S(O) CH₃, -(phenyl), or —O-(phenyl).

In some embodiments, the compound wherein
Y₁, Y₂, Y₃ and Y₄ are each C; and
R₄, R₅, R₆ and R₇ are each —H.

In some embodiments, the compound wherein
R₄, R₆, and R₇ are each —H; and R₅ is —CH₃, —Cl, —F, —Br, —I, —OCH₃, —OH, —OAc, —SCH₃, —SO₂CH₃, —S(O) CH₃, -(phenyl), or —O-(phenyl).

In some embodiments, the compound wherein
R₄, R₅, and R₇ are each —H; and R₆ is —CH₃, —Cl, —F, —I, —OCH₃, —OH, —OAc, —SCH₃, —SO₂CH₃, —S(O) CH₃, -(phenyl), or —O-(phenyl).

In some embodiments, the compound wherein
Y₁, Y₃ and Y₄ are each C;
Y₂ is N and R₅ is absent;
R₄, R₆ and R₇ are each are each independently —H, —Cl, —Br, —F, —I, —CN, —CF₃, —OCF₃, —OH, —OAc, —(C₁-C₆ alkyl), —O—(C₁-C₆ alkyl), —S—(C₁-C₆ alkyl), —SO₂—(C₁-C₆ alkyl), —S(O)—(C₁-C₆ alkyl), —O-(aryl) or —S-(aryl), or -(aryl).

In some embodiments, the compound wherein
Y₁, Y₃ and Y₄ are each C;
Y₂ is N and R₅ is absent; and
R₄, R₆ and R₇ are each —H.

In some embodiments, the compound wherein
R₂ is —(C₁-C₁₂ alkyl), —(C₁-C₁₂ alkenyl), —(C₁-C₁₂ alkynyl)-(C₁-C₁₂ alkyl)-OH, —(C₁-C₁₂ alkyl)-CO₂H, —(C₁-C₁₂ alkyl)-CO₂—(C₁-C₆ alkyl), —(C₁-C₁₂ alkyl)-C(O)—NH₂, —(C₁-C₁₂ alkyl)-C(O)—NH(C₁-C₆ alkyl), —(C₁-C₁₂ alkyl)-C(O)—NH—(C₁-C₆ hydroxyalkyl), —(C₁-C₁₂ alkyl)-C(O)—N(C₁-C₆ alkyl)₂, —(C₁-C₁₂ alkyl)-C(O)—N(C₁-C₆ hydroxyalkyl)₂, —(C₁-C₁₂ alkyl)-O—(C₁-C₆ alkyl), —(C₁-C₁₂ alkyl)-S—(C₁-C₆ alkyl), —(C₁-C₁₂ alkyl)-CF₃, —(C₁-C₁₂ alkyl)-O—(C₁-C₆ hydroxyalkyl), —(C₁-C₁₂ alkyl)-O—(C₁-C₆ alkyl)-OCH₃, —(C₁-C₁₂ alkyl)-(CH)—(O—(C₁-C₆ alkyl))₂, —(C₁-C₁₂alkyl)-(heterocyclyl), —(C₁-C₁₂ alkyl)-OAc, —(C₁-C₁₂ alkyl)-tetrahydrofuran, —(C₁-C₁₂ alkyl)-pyrrolidine, —(C₁-C₁₂ alkyl)-N-methylpyrrolidine, —(C₁-C₁₂ alkyl)-(1,3-dioxane) or —(C₁-C₁₂ alkyl)-(4,5-dihydrooxazole).

In some embodiments, the compound wherein
R₂ is —(C₁-C₈ alkyl), —(C₁-C₆ alkenyl), —(C₁-C₆ alkynyl)-(C₁-C₆ alkyl)-OH, —(C₁-C₆ alkyl)-CO₂H, —(C₁-C₆ alkyl)-CO₂—(C₁-C₂ alkyl), —(C₁-C₆ alkyl)-C(O)—NH₂, —(C₁-C₆ alkyl)-C(O)—NH(C₁-C₂ alkyl), —(C₁-C₆ alkyl)-C(O)—NH—(C₁-C₂ hydroxyalkyl), —(C₁-C₆ alkyl)-C(O)—N(C₁-C₂ alkyl)₂, —(C₁-C₆ alkyl)-C(O)—N(C₁-C₂ hydroxyalkyl)₂, —(C₁-C₆ alkyl)-O—(C₁-C₂ alkyl), —(C₁-C₆ alkyl)-S—(C₁-C₂ alkyl), —(C₁-C₃ alkyl)-CF₃, —(C₁-C₃ alkyl)-O—(C₁-C₂ hydroxyalkyl), —(C₁-C₃ alkyl)-O—(C₁-C₂ alkyl)-OCH₃, —(C₁-C₆ alkyl)-(CH)—(O—(C₁-C₂ alkyl))₂, —(C₁-C₆ alkyl)-(heterocyclyl), —(C₁-C₆ alkyl)-OAc, —(C₁-C₆ alkyl)-tetrahydrofuran, —(C₁-C₆ alkyl)-pyrrolidine, —(C₁-C₆ alkyl)-N-methylpyrrolidine, —(C₁-C₆ alkyl)-(1,3-dioxane) or —(C₁-C₆ alkyl)-(4,5-dihydrooxazole).

In some embodiments, the compound wherein
R₂ is

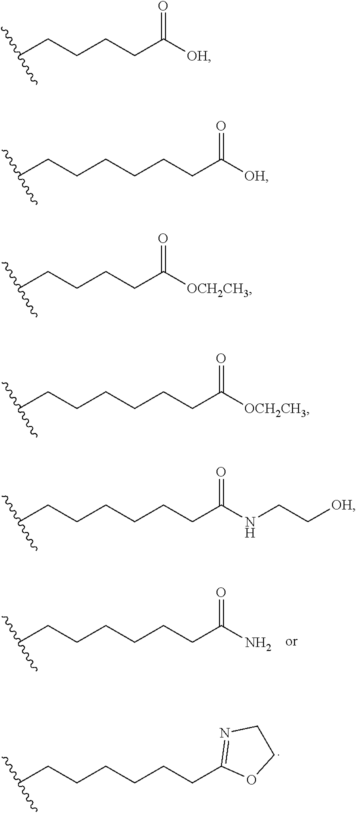

In some embodiments, the compound wherein
R₂ is —CH₃, —CH₂CH₃,

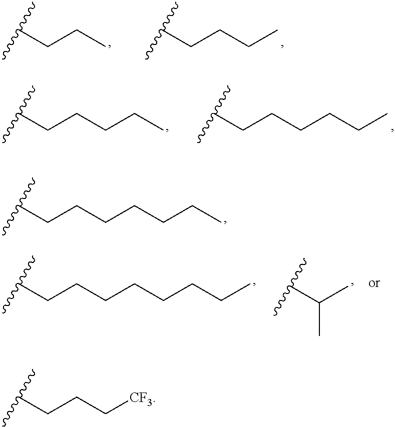

In some embodiments, the compound wherein
R₂ is

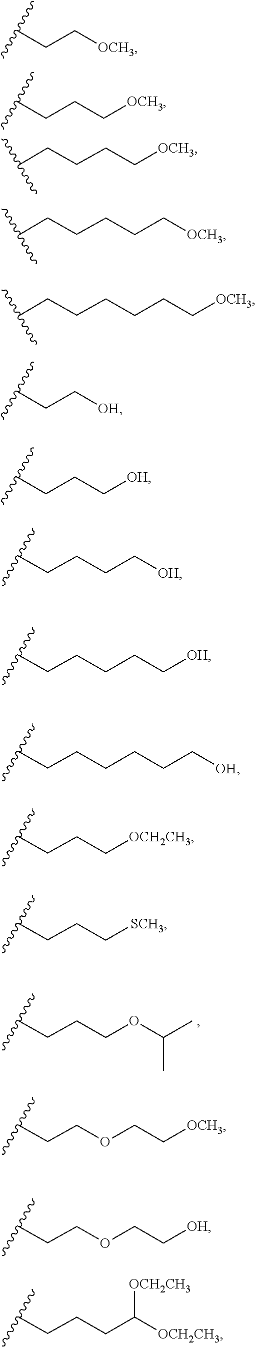

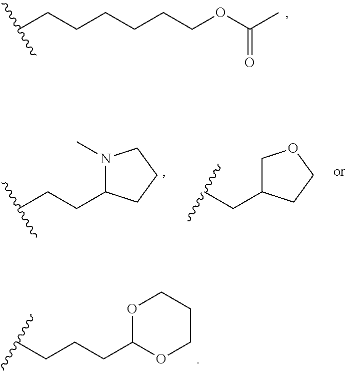

In some embodiments of any of the compounds described herein,
R₂ is

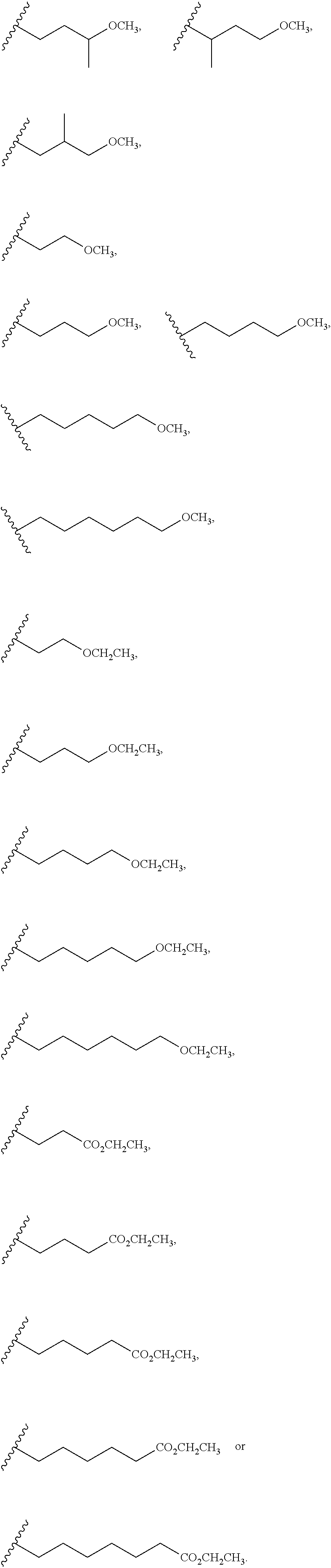

In some embodiments, the compound wherein R₁ is —H or —(C₁-C₆ alkyl).

In some embodiments, the compound wherein R₃ is —H or —(C₁-C₆ alkyl).

In some embodiments, the compound wherein R₁ is —H, —CH₃ or —CH₂CH₃.

In some embodiments, the compound wherein R₃ is —H, —CH₃ or —CH₂CH₃.

In some embodiments, the compound wherein R₁ is —CH₃; and R₃ is —H.

In some embodiments, the compound wherein R₁ is —CH₃; and R₃ is —CH₃.

In some embodiments, the compound wherein R₁ is —CH₂CH₃; and R₃ is H.

In some embodiments, the compound having the structure:

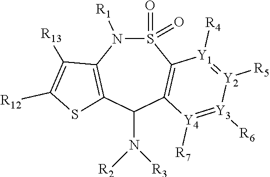

R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-OCH₃, -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl);
R₁₂ and R₁₃ are each independently —H, —Cl, —Br, —F, —I, —CN, —CF₃, —OCF₃, -(alkyl), -(aryl), -(heteroaryl)-(alkenyl), -(alkynyl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present.

In some embodiments, the compound wherein
A is

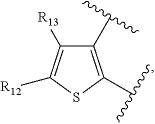

wherein R₁₂ and R₁₃ are each independently —H, —Cl, —Br, —F, —I, —CN, —CF₃, —OCF₃, -(alkyl), -(aryl), -(heteroaryl)-(alkenyl), -(alkynyl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl).

In some embodiments, the compound wherein R₁₂ and R₁₃ are each independently —H, —Cl, —Br, —F, —I, —CN, —CF₃, —OCF₃, —OH, —OAc, —(C₁-C₆ alkyl), —O—(C₁-C₆ alkyl), —S—(C₁-C₆ alkyl), —SO₂—(C₁-C₆ alkyl), —S(O)—(C₁-C₆ alkyl), —O-(aryl) or —S-(aryl), or -(aryl).

In some embodiments, the compound wherein R₁₂ and R₁₃ are each independently —H, —CH₃, —Cl, —Br, —F, —I, —OCH₃, —OH, —OAc, —SCH₃, —SO₂CH₃, —S(O) CH₃, -(phenyl), or —O-(phenyl).

In some embodiments, the compound wherein R₁₂ and R₁₃ are each independently —H, —CH₃, —Cl, —Br, —F, —OCH₃, —SCH₃, or —O-(phenyl).

In some embodiments, the compound wherein R₁₂ and R₁₃ are each —H. In some embodiments, the compound wherein R₁₂ is —H; and R₁₃ is —Br.

In some embodiments, the compound wherein
Y₁, Y₂, Y₃ and Y₄ are each C; and
R₄, R₅, R₆ and R₃ are each are each independently —H, —Cl, —Br, —F, —I, —CN, —CF₃, —OCF₃, —OH, —OAc, —(C₁-C₆ alkyl), —O—(C₁-C₆ alkyl), —S—(C₁-C₆ alkyl), —SO₂—(C₁-C₆ alkyl), —S(O)—(C₁-C₆ alkyl), —O-(aryl) or —S-(aryl), or -(aryl).

In some embodiments, the compound wherein
Y₁, Y₂, Y₃ and Y₄ are each C; and
R₄, R₅, R₆ and R₇ are each independently —H, —CH₃, —Cl, —Br, —F, —I, —OCH₃, —OH, —OAc, —SCH₃, —SO₂CH₃, —S(O) CH₃, -(phenyl), or —O-(phenyl).

In some embodiments, the compound wherein
Y₁, Y₂, Y₃ and Y₄ are each C; and
R₄, R₅, R₆ and R₃ are each —H.

In some embodiments, the compound wherein
Y₁, Y₂, Y₃ and Y₄ are each C; and
R₄, R₆, and R₇ are each —H; and R₅ is —CH₃, —Cl, —Br, —F, —I, —OCH₃, —OH, —OAc, —SCH₃, —SO₂CH₃, —S(O) CH₃, -(phenyl), or —O-(phenyl).

In some embodiments, the compound wherein R₄, R₅, and R₇ are each —H;
and R₆ is —CH₃, —Cl, —Br, —F, —I, —OCH₃, —OH, —OAc, —SCH₃, —SO₂CH₃, —S(O)CH₃, -(phenyl), or —O-(phenyl).

In some embodiments, the compound wherein
Y₁, Y₃ and Y₄ are each C;
Y₂ is N and R₅ is absent; and
R₄, R₆ and R₇ are each are each independently —H, —Cl, —Br, —F, —I, —CN, —CF₃, —OCF₃, —OH, —OAc, —(C₁-C₆ alkyl), —O—(C₁-C₆ alkyl), —S—(C₁-C₆ alkyl), —SO₂—(C₁-C₆ alkyl), —S(O)—(C₁-C₆ alkyl), —O-(aryl) or —S-(aryl), or -(aryl).

In some embodiments, the compound wherein
Y₁, Y₃ and Y₄ are each C;
Y₂ is N and R₅ is absent; and
R₄, R₆ and R₇ are each —H.

In some embodiments, the compound wherein
Y₁, Y₂, Y₃ and Y₄ are each C; and
R₄, R₆, and R₇ are each —H; and R₅ is —Br, —Cl or —O-(phenyl).

In some embodiments, the compound wherein
R₄, R₅, and R₇ are each —H; and R₆ is —CH₃, —Cl, —F, —I, —OCH₃, —OH, —OAc, —SCH₃, —SO₂CH₃, —S(O) CH₃, -(phenyl), or —O-(phenyl).

In some embodiments, the compound wherein
Y₁, Y₂, Y₃ and Y₄ are each C; and
R₄, R₅, and R₇ are each —H; and R₆ is —Br, Cl or —O-(phenyl).

In some embodiments, the compound wherein
wherein R₂ is -(alkyl)-CO₂-(alkyl), -(alkyl)-O-(alkyl) or -(alkyl)-O-(alkyl)-O-(alkyl).

In some embodiments, the compound wherein
R₂ is

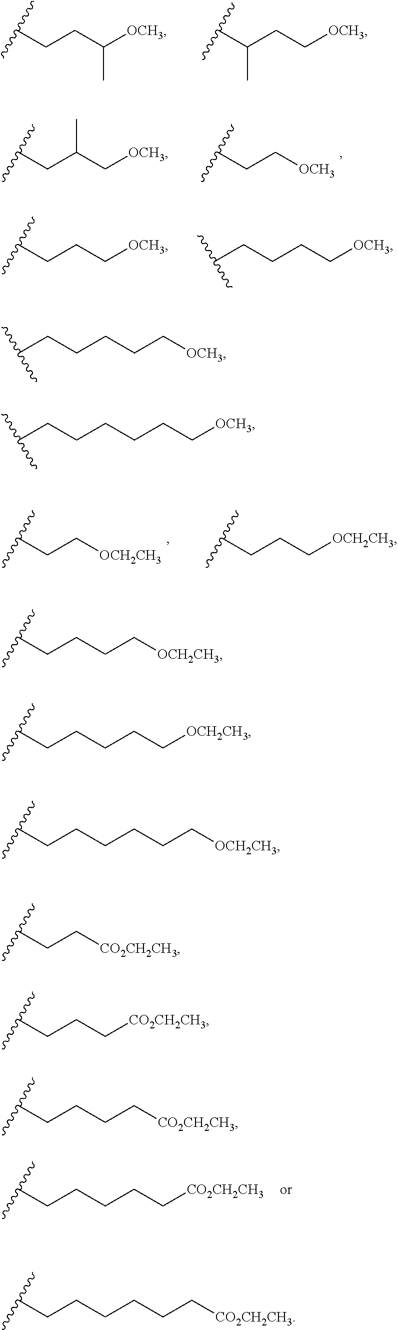

In some embodiments, the compound wherein
R₂ is —(C₁-C₁₂ alkyl), —(C₁-C₁₂ alkenyl), —(C₁-C₁₂ alkynyl)-(C₁-C₁₂ alkyl)-OH, —(C₁-C₁₂ alkyl)-CO₂H, —(C₁-C₁₂ alkyl)-CO₂—(C₁-C₆ alkyl), —(C₁-C₁₂ alkyl)-C(O)—NH₂, —(C₁-C₁₂ alkyl)-C(O)—NH(C₁-C₆ alkyl), —(C₁-C₁₂ alkyl)-C(O)—NH—(C₁-C₆ hydroxyalkyl), —(C₁-C₁₂ alkyl)-C(O)—N(C₁-C₆ alkyl)₂, —(C₁-C₁₂ alkyl)-C(O)—N(C₁-C₆ hydroxyalkyl)₂, —(C₁-C₁₂ alkyl)-O—(C₁-C₆ alkyl), —(C₁-C₁₂ alkyl)-S—(C₁-C₆ alkyl), —(C₁-C₁₂ alkyl)-CF₃, —(C₁-C₁₂ alkyl)-O—(C₁-C₆ hydroxyalkyl), —(C₁-C₁₂ alkyl)-O—(C₁-C₆ alkyl)-OCH₃, —(C₁-C₁₂ alkyl)-(CH)—(O—(C₁-C₆ alkyl))₂, —(C₁-C₁₂alkyl)-(heterocyclyl), —(C₁-C₁₂ alkyl)-OAc, —(C₁-C₁₂ alkyl)-tetrahydrofuran, —(C₁-C₁₂ alkyl)-pyrrolidine, —(C₁-C₁₂ alkyl)-N-methylpyrrolidine, —(C₁-C₁₂ alkyl)-(1,3-dioxane) or —(C₁-C₁₂ alkyl)-(4,5-dihydrooxazole).

In some embodiments, the compound wherein
R₂ is —(C₁-C₈ alkyl), —(C₁-C₆ alkenyl), —(C₁-C₆ alkynyl)-(C₁-C₆ alkyl)-OH, —(C₁-C₆ alkyl)-CO₂H, —(C₁-C₆ alkyl)-CO₂—(C₁-C₂ alkyl),
(C₁-C₆ alkyl)-C(O)—NH₂, —(C₁-C₆ alkyl)-C(O)—NH(C₁-C₂ alkyl), —(C₁-C₆ alkyl)-C(O)—NH—(C₁-C₂ hydroxyalkyl), —(C₁-C₆ alkyl)-C(O)—N(C₁-C₂ alkyl)₂, —(C₁-C₆ alkyl)-C(O)—N(C₁-C₂ hydroxyalkyl)₂, —(C₁-C₆ alkyl)-O—(C₁-C₂ alkyl), —(C₁-C₆ alkyl)-S—(C₁-C₂ alkyl), —(C₁-C₃ alkyl)-CF₃, —(C₁-C₃ alkyl)-O—(C₁-C₂ hydroxyalkyl), —(C₁-C₃ alkyl)-O—(C₁-C₂ alkyl)-OCH₃, —(C₁-C₆ alkyl)-(CH)—(O—(C₁-C₂ alkyl))₂, —(C₁-C₆ alkyl)-(heterocyclyl), —(C₁-C₆ alkyl)-OAc, —(C₁-C₆ alkyl)-tetrahydrofuran, —(C₁-C₆ alkyl)-pyrrolidine, —(C₁-C₆ alkyl)-N-methylpyrrolidine, —(C₁-C₆ alkyl)-(1,3-dioxane) or —(C₁-C₆ alkyl)-(4,5-dihydrooxazole).

In some embodiments, the compound wherein
R₂ is

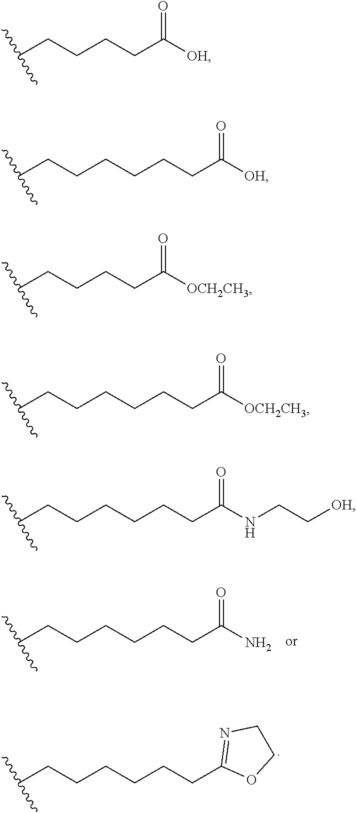

In some embodiments, the compound wherein
R₂ is

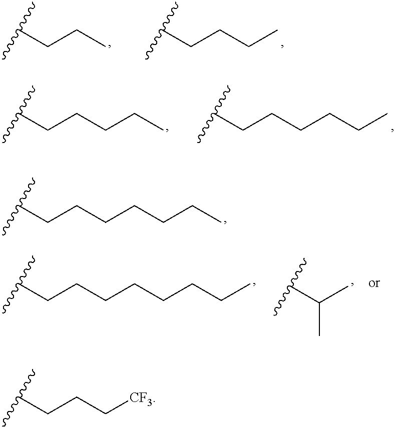

In some embodiments, the compound wherein
R₂ is

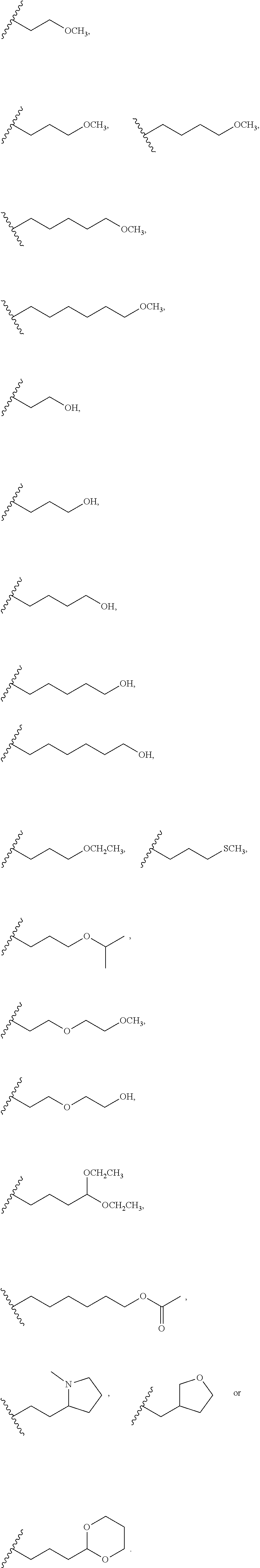

In some embodiments, the compound wherein R₁ is —H or —(C₁-C₆ alkyl).

In some embodiments, the compound wherein R₃ is —H or —(C₁-C₆ alkyl).

In some embodiments, the compound wherein R₁ is —H, —CH₃ or —CH₂CH₃.

In some embodiments, the compound wherein R₃ is —H, —CH₃ or —CH₂CH₃.

In some embodiments, the compound wherein R₁ is —CH₃; and R₃ is —H.

In some embodiments, the compound wherein R₁ is —CH₃; and R₃ is —CH₃.

In some embodiments, the compound wherein R₁ is —CH₂CH₃; and R₃ is H.

The present disclosure further provides the compound having the structure:

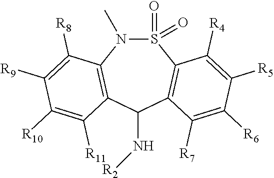

wherein
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₅ is —Cl, —Br, —F, or —I;
R₄, R₆ and R/are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl) or —SO₂-(heteroaryl).

The present disclosure provides a compound having the structure:

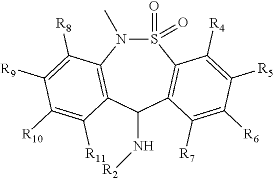

wherein
R₂ is -(alkyl)-O-(alkyl) or -(alkyl)-O-(alkyl)-O-(alkyl);
R₅ is —Cl, —Br, —F, or —I;
R₄, R₆ and R are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl) or —SO₂-(heteroaryl); R₈, R₉, R₁₀ and R₁₁ are each independently —H, —Cl, —Br, —F, —I, —CN, —CF₃, —OCF₃, -(alkyl), -(aryl), -(heteroaryl)-(alkenyl), -(alkynyl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl).

The present disclosure provides a compound having the structure:

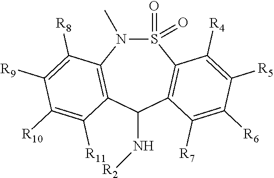

wherein
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrocxazole);
R₅ is —Br, or —I;
R₄, R₆ and R/are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl) or —SO₂-(heteroaryl).

In some embodiments, the compound having the structure:

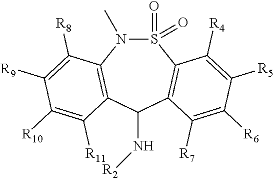

wherein
R₂ is -(alkyl)-CO₂-(alkyl), -(alkyl)-O-(alkyl) or -(alkyl)-O-(alkyl)-O-(alkyl);
R₅ is —Cl, —Br, —F, or —I;
R₄, R₆ and R/are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl) or —SO₂-(heteroaryl);
R₈, R₉, R₁₀ and R₁₁ are each independently —H, —Cl, —Br, —F, —I, —CN, —CF₃, —OCF₃, -(alkyl), -(aryl), -(heteroaryl)-(alkenyl), -(alkynyl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl).

In some embodiments, the compound having the structure:

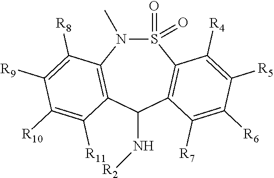

wherein
R₂ is -(alkyl)-CO₂-(alkyl), -(alkyl)-O-(alkyl) or -(alkyl)-O-(alkyl)-O-(alkyl);
R₅ is —Cl, —Br, —F, or —I;
R₄, R₆, R₇, R₈, R₉, R₁₀ and R₁₁ are each —H.

In some embodiments, the compound having the structure:

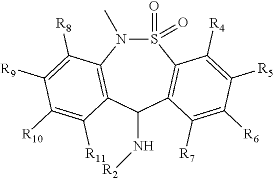

wherein
R₂ is -(alkyl)-CO₂H;
R₅ is —Br, —F, or —I;
R₄, R₆, R₇, R₈, R₉, R₁₀ and R₁₁ are each —H.

In some embodiments, the compound having the structure:

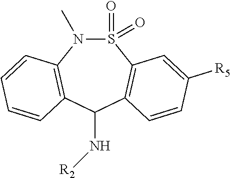

wherein
R₂ is -(alkyl)-CO₂-(alkyl), -(alkyl)-O-(alkyl) or -(alkyl)-O-(alkyl)-O-(alkyl);
R₅ is —F.

In some embodiments, the compound having the structure:

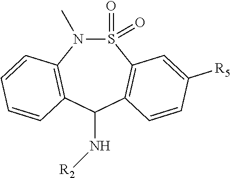

wherein
R₂ is -(alkyl)-CO₂-(alkyl), -(alkyl)-O-(alkyl) or -(alkyl)-O-(alkyl)-O-(alkyl);
R₅ is —Cl.

In some embodiments, the compound having the structure:

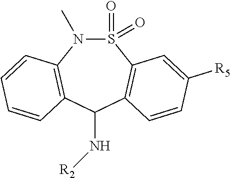

wherein
R₂ is -(alkyl)-CO₂ (alkyl), -(alkyl)-O-(alkyl) or -(alkyl)-O-(alkyl)-O-(alkyl);
R₅ is —Br.

In some embodiments, the compound having the structure:

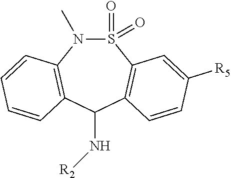

wherein
R₂ is -(alkyl)-CO₂-(alkyl), -(alkyl)-O-(alkyl) or -(alkyl)-O-(alkyl)-O-(alkyl);
R₅ is —I.

In some embodiments, the compound wherein
R₂ is

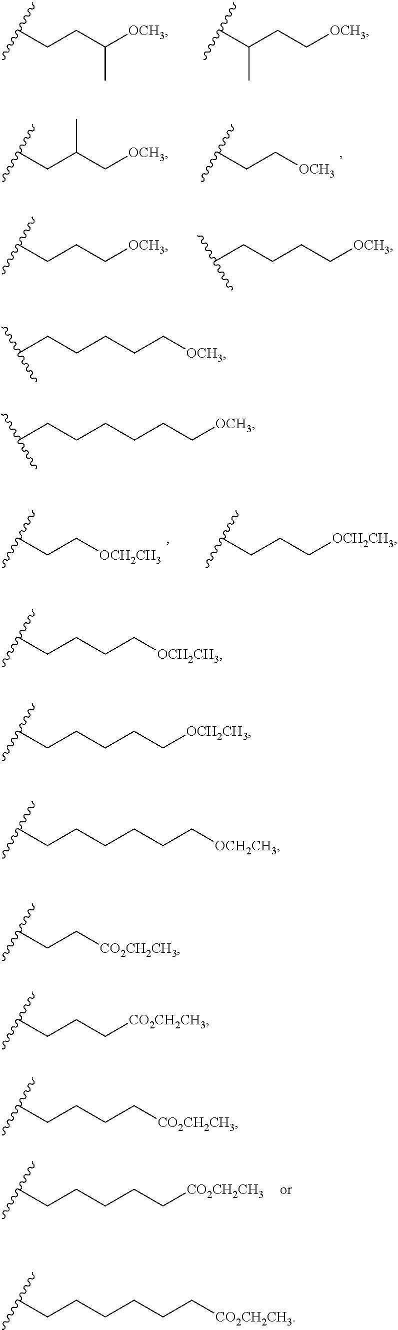

The present disclosure provides a compound having the structure:

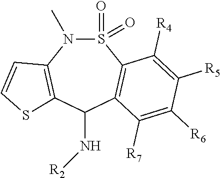

wherein
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₅ is —Cl, —Br, —F, or —I;
R₄, R₆ and R₇ are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl) or —SO₂-(heteroaryl),
   or a pharmaceutically acceptable salt thereof,
   wherein the compound is for treating or preventing a neurological disorder in a subject, wherein the neurological disorder is selected from the group consisting of Huntington's disease, Rett syndrome, and CDKL5 disorder, and the other neurological disorders as described herein.

The present disclosure provides a compound having the structure:

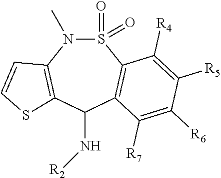

wherein
R₂ is -(alkyl)-CO₂-(alkyl), -(alkyl)-O-(alkyl) or -(alkyl)-O-(alkyl)-O-(alkyl);
R₅ is —Cl, —Br, —F, or —I;
R₄, R₆ and R₇ are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl) or —SO₂-(heteroaryl),
   or a pharmaceutically acceptable salt thereof,
   wherein the compound is for treating or preventing a neurological disorder in a subject, wherein the neurological disorder is selected from the group consisting of Huntington's disease, Rett syndrome, and CDKL5 disorder, and the other neurological disorders as described herein.

The present disclosure provides a compound having the structure:

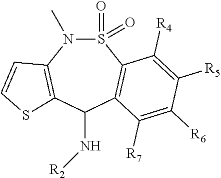

wherein
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrocxazole);
R₅ is —Cl, —Br, —F, or —I;
R₄, R₆ and R/are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl) or —SO₂-(heteroaryl),
   or a pharmaceutically acceptable salt thereof,
   wherein the compound is for treating or preventing a neurological disorder in a subject, wherein the neurological disorder is selected from the group consisting of Huntington's disease, Rett syndrome, and CDKL5 disorder, and the other neurological disorders as described herein.

The present disclosure provides a compound having the structure:

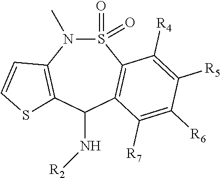

wherein
R₂ is -(alkyl)-CO₂-(alkyl), -(alkyl)-O-(alkyl) or -(alkyl)-O-(alkyl)-O-(alkyl);
R₅ is —Cl, —Br, —F, or —I;
R₄, R₆ and R₇ are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl) or —SO₂-(heteroaryl);
R₈, R₉, R₁₀ and R₁₁ are each independently —H, —Cl, —Br, —F, —I, —CN, —CF₃, —OCF₃, -(alkyl), -(aryl), -(heteroaryl)-(alkenyl), -(alkynyl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl).

In some embodiments, the compound having the structure:

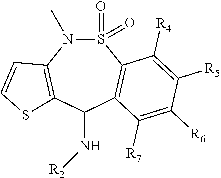

wherein
R₂ is (alkyl)-CO₂H;
R₅ is —Cl, —Br, —F, or —I;
R₄, R₆, and R₇ are each —H.

In some embodiments, the compound having the structure:

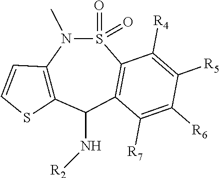

wherein
R₂ is -(alkyl)-CO₂-(alkyl), -(alkyl)-O-(alkyl) or -(alkyl)-O-(alkyl)-O-(alkyl);
R₅ is —Cl, —Br, —F, or —I;
R₄, R₆, and R₇ are each —H.

In some embodiments, the compound having the structure:

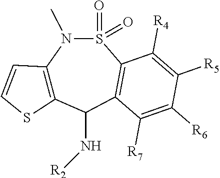

wherein
R₂ is -(alkyl)-CO₂-(alkyl), -(alkyl)-O-(alkyl) or -(alkyl)-O-(alkyl)-O-(alkyl);
R₅ is —F.

In some embodiments, the compound having the structure:

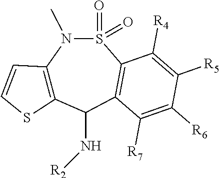

wherein
R₂ is -(alkyl)-CO₂-(alkyl), -(alkyl)-O-(alkyl) or -(alkyl)-O-(alkyl)-O-(alkyl);
R₅ is —Cl.

In some embodiments, the compound having the structure:

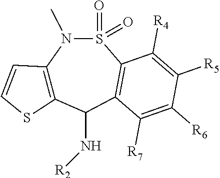

wherein
R₂ is -(alkyl)-CO₂-(alkyl), -(alkyl)-O-(alkyl) or -(alkyl)-O-(alkyl)-O-(alkyl);
R₅ is —Br.

In some embodiments, the compound having the structure:

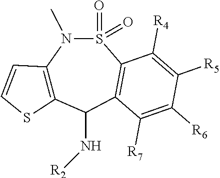

wherein
R₂ is -(alkyl)-CO₂-(alkyl), -(alkyl)-O-(alkyl) or -(alkyl)-O-(alkyl)-O-(alkyl);
R₅ is —I.

In some embodiments, the compound wherein
R₂ is

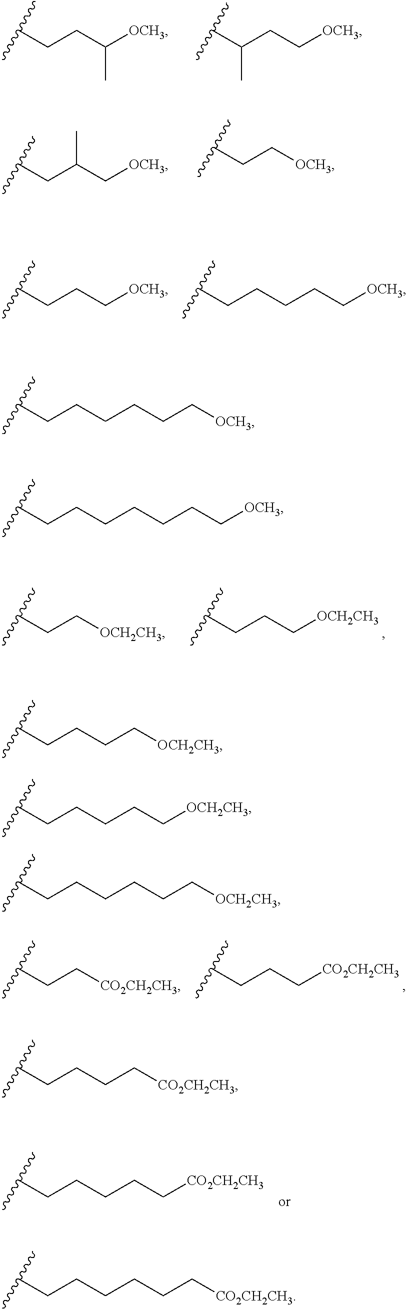

In some embodiments, the compound having the structure:

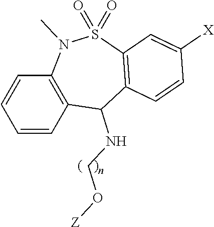

wherein X=F, Cl, Br, I, Me, SMe, OMe or OPh; Z is alkyl; and n=2-10.

In some embodiments, the compound having the structure:

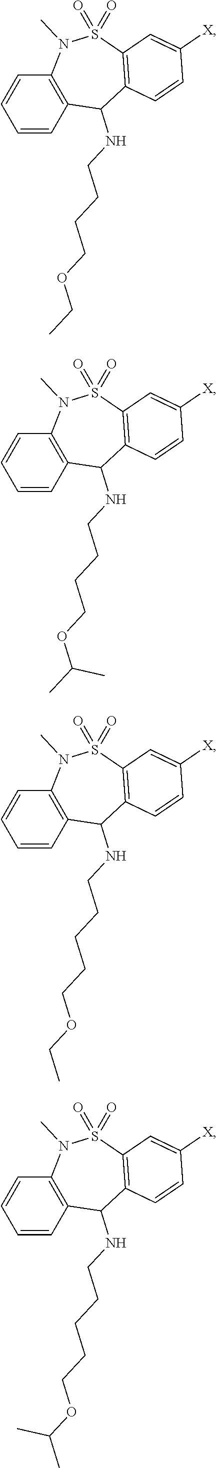

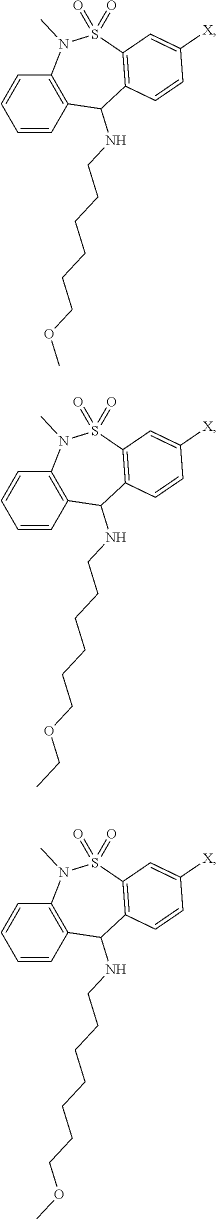

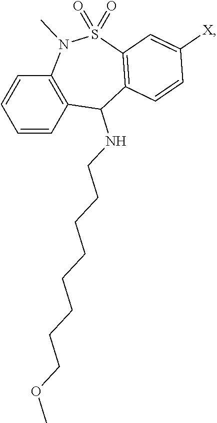

wherein X=X=F, Cl, Br, I, Me, SMe, OMe or OPh.

In some embodiments, the compound is prepared by the following process:

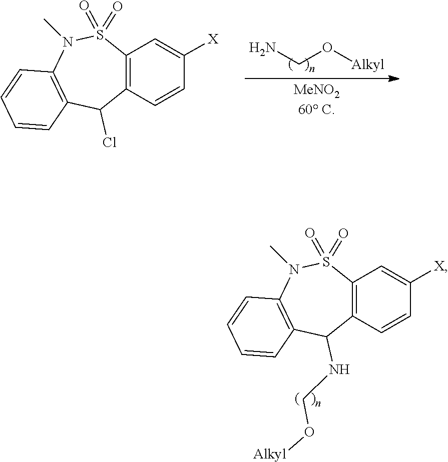

wherein X=F, Cl, Br, I, Me, SMe, OMe or OPh; and n=2-10.

In some embodiments, the compound having the structure:

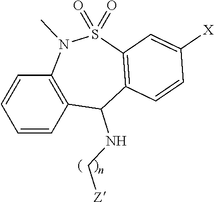

wherein X=F, Cl, Br, I, Me, SMe, OMe or OPh; Z′ is heteroaryl; and 1.5 n=1-10.

In some embodiments, the compound having the structure:

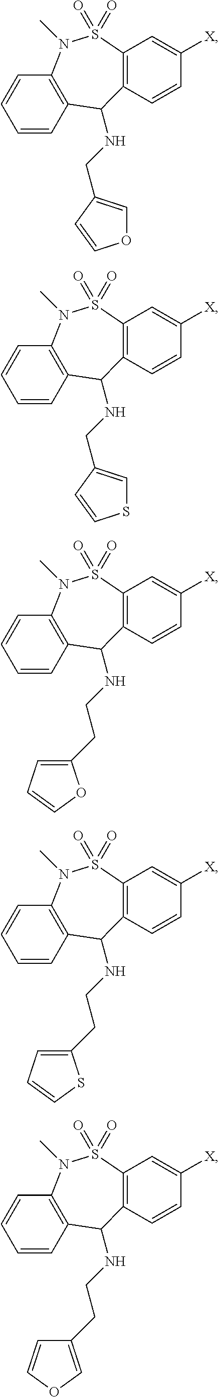

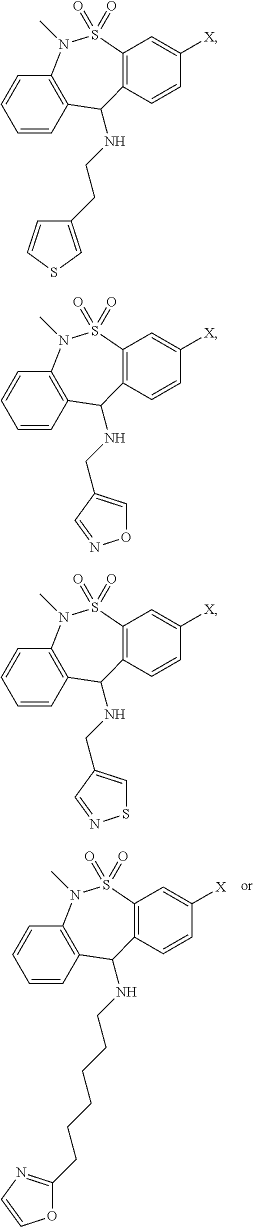

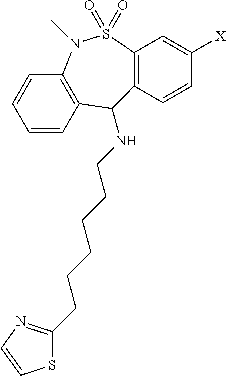

wherein X=F, Cl, Br, I, Me, SMe, OMe or OPh.

In some embodiments, the compound is prepared by the following process:

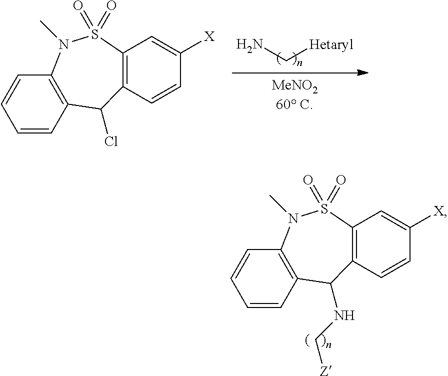

wherein X=F, Cl, Br, I, Me, SMe, OMe or OPh; Z′ is hetero aryl; and n=1-10.

In some embodiments, the heteroaryl is furan, thiophene, imidazole, pyrazole, oxazole, thiazole, isoxazole or isothiazole.

In some embodiments, the compound having the structure:

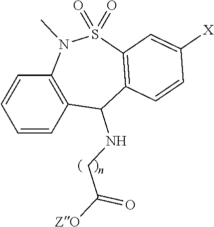

wherein X=F, Cl, Br, I, Me, SMe, OMe or OPh; Z″ is Me, Et, Pr, iPR or Phenyl; and n=2-10.

In some embodiments, the compound having the structure:

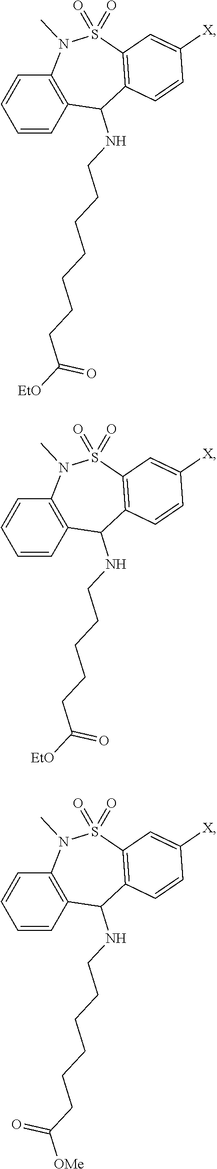

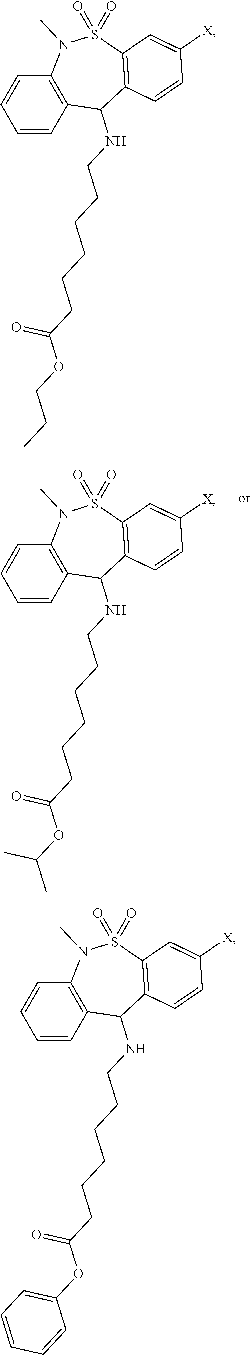

wherein X=F, Cl, Br, I, Me, SMe, OMe or OPh.

In some embodiments, the compound is prepared by the following process:

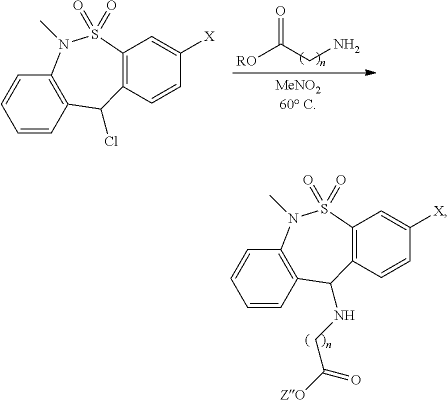

wherein X=F, Cl, Br, I, Me, SMe, OMe or OPh; Z″ is Me, Et, Pr, iPR or Phenyl; and n=2-10.

In some embodiments, the compound having the structure:

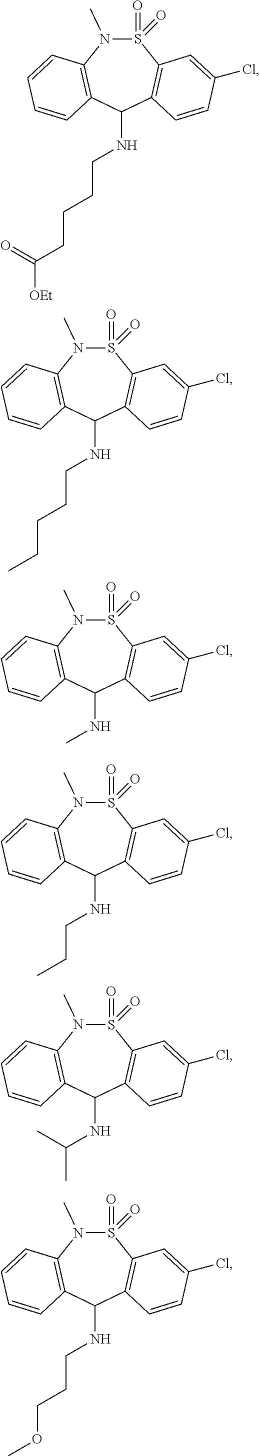

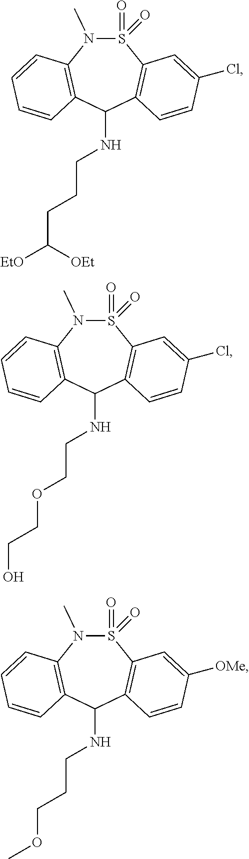

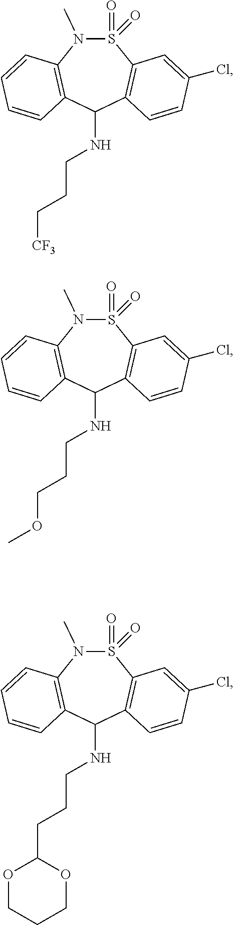 [SMILES: CN1c2ccccc2C(NCCCC(F)(F)F)c2ccc(Cl)cc2S1(=O)=O ; COCCCNC1c2ccccc2N(C)S(=O)(=O)c2cc(Cl)ccc21 ; CN1c2ccccc2C(NCCCC2OCCCO2)c2ccc(Cl)cc2S1(=O)=O]

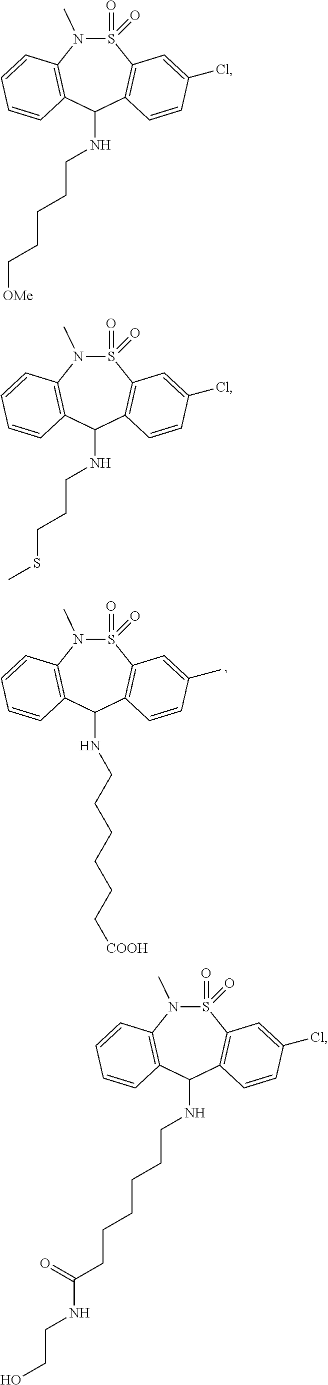 [SMILES: COCCCCCNC1c2ccccc2N(C)S(=O)(=O)c2cc(Cl)ccc21 ; CSCCCNC1c2ccccc2N(C)S(=O)(=O)c2cc(Cl)ccc21 ; Cc1ccc2c(c1)S(=O)(=O)N(C)c1ccccc1C2NCCCCCCC(=O)O ; CN1c2ccccc2C(NCCCCCCC(=O)NCCO)c2ccc(Cl)cc2S1(=O)=O]

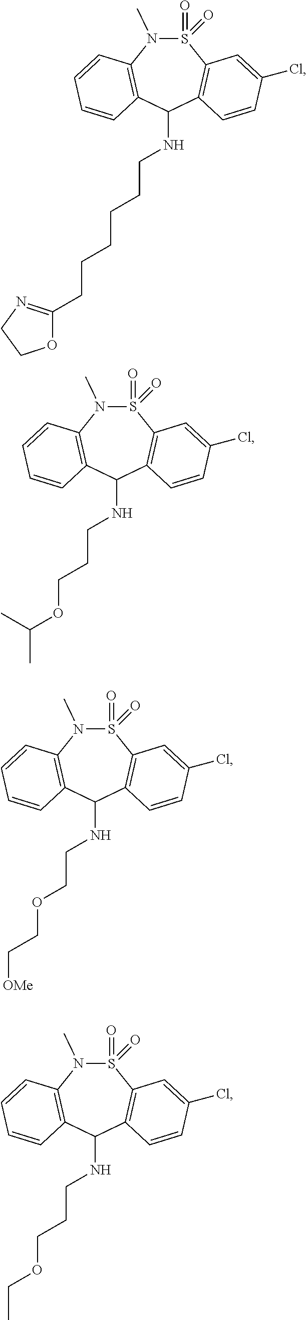

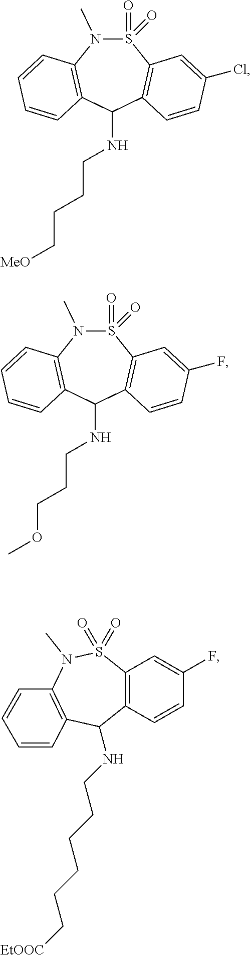 [SMILES: COCCCCNC1c2ccccc2N(C)S(=O)(=O)c2cc(Cl)ccc21 ; COCCCNC1c2ccccc2N(C)S(=O)(=O)c2cc(F)ccc21 ; CCOC(=O)CCCCCCNC1c2ccccc2N(C)S(=O)(=O)c2cc(F)ccc21]

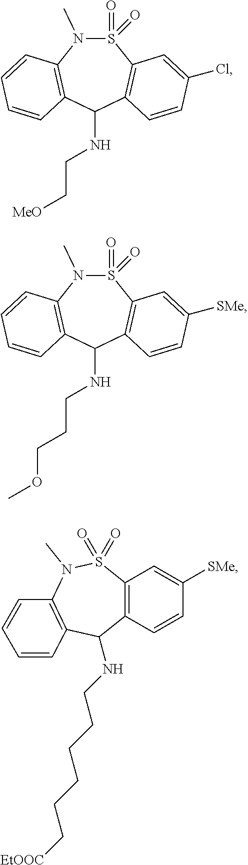

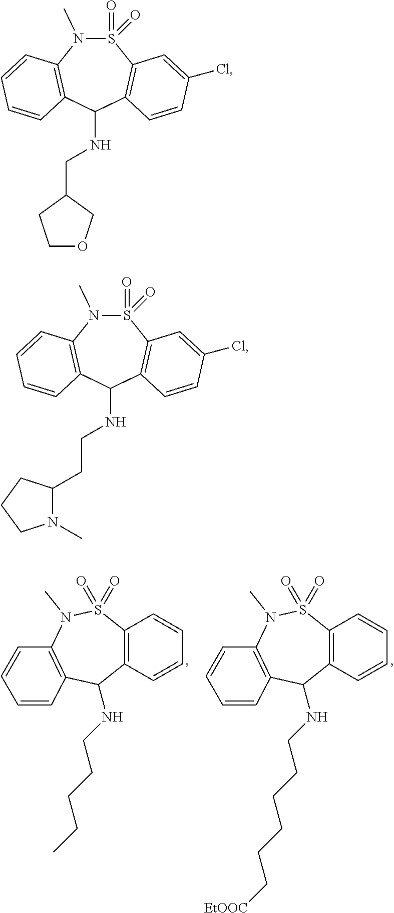

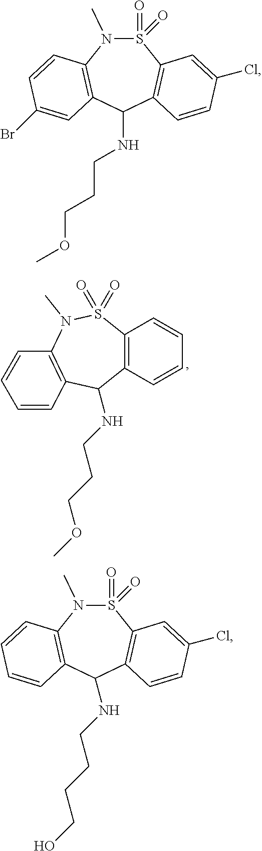

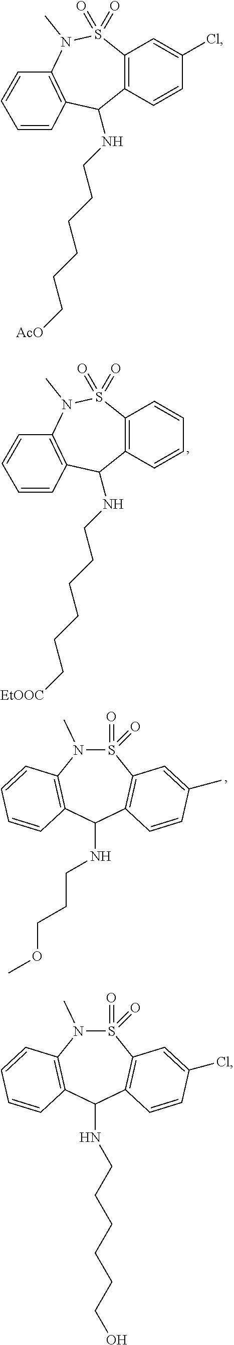

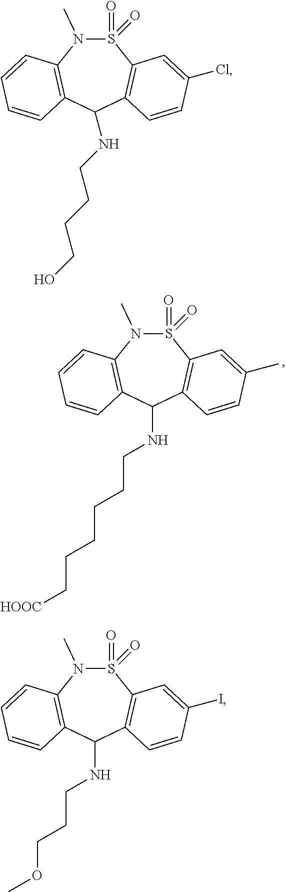

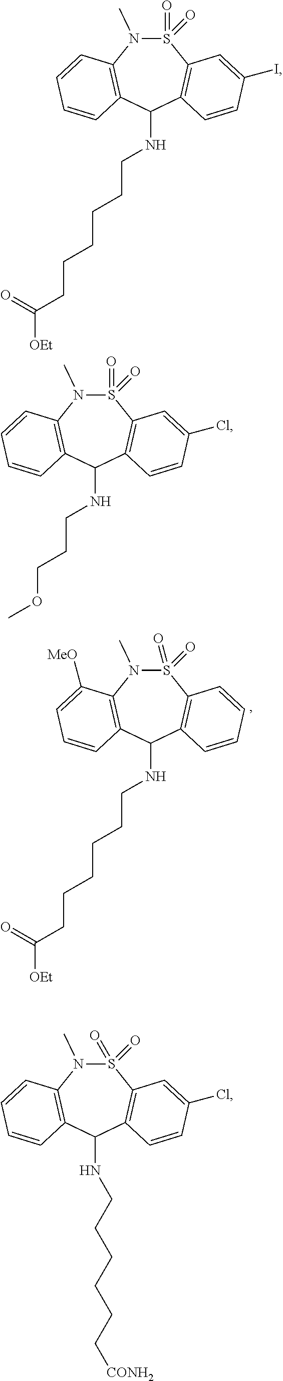

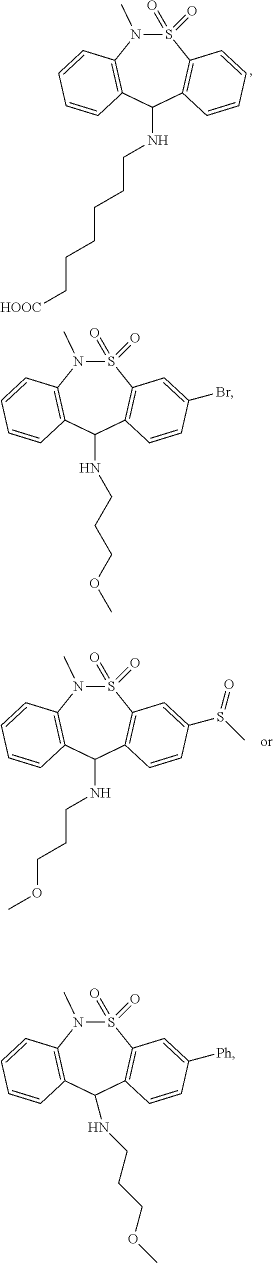

or a pharmaceutically acceptable salt thereof.

In some embodiments, the compound having the structure:

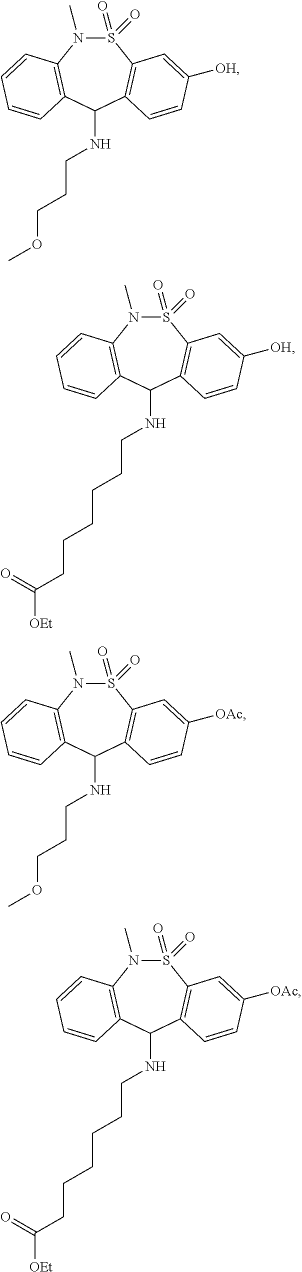

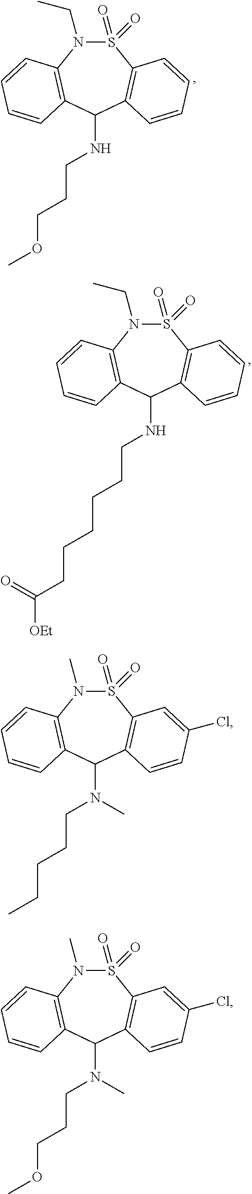

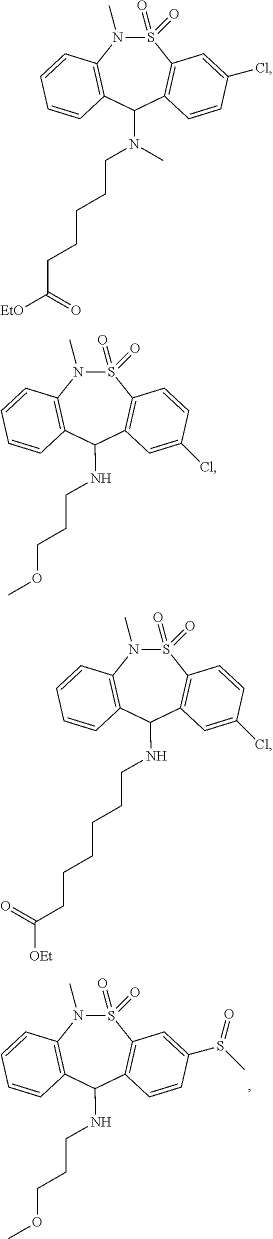

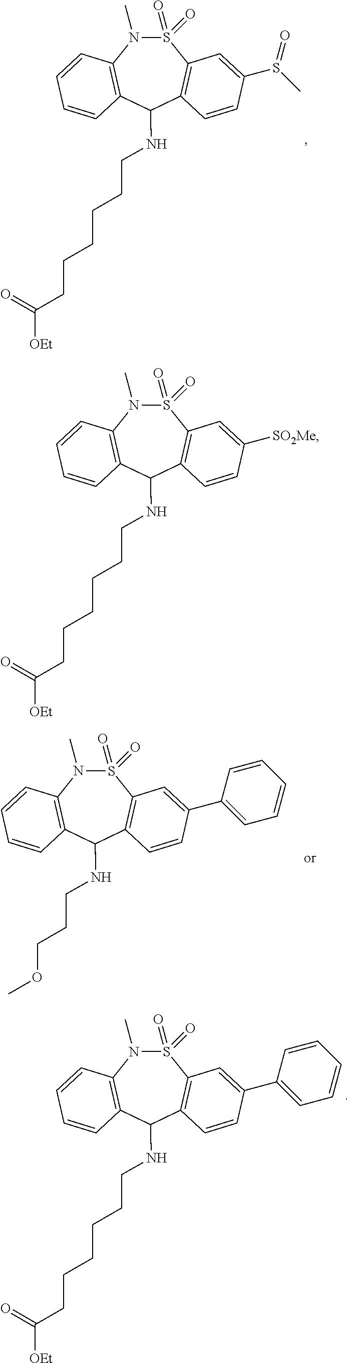

or a pharmaceutically acceptable salt thereof.

In some embodiments, the compound having the structure:

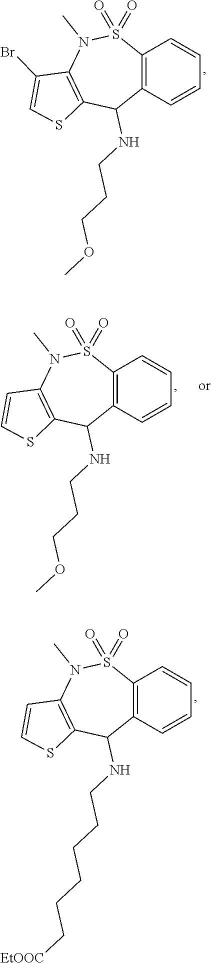 [SMILES: COCCCNC1c2ccccc2S(=O)(=O)N(C)c2c(Br)csc21 ; COCCCNC1c2ccccc2S(=O)(=O)N(C)c2ccsc21 ; CCOC(=O)CCCCCCNC1c2ccccc2S(=O)(=O)N(C)c2ccsc21]

or a pharmaceutically acceptable salt thereof.

In some embodiments, the compound having the structure:

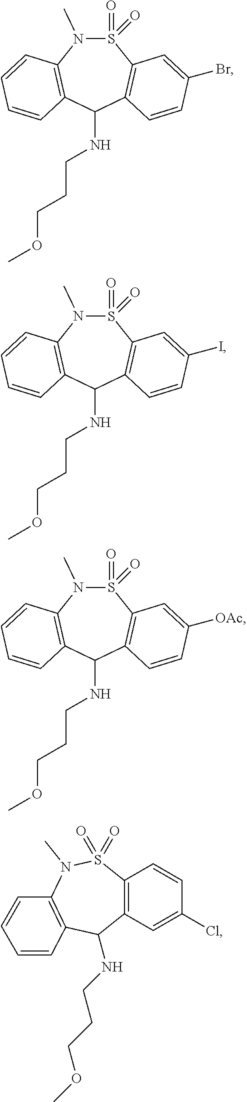

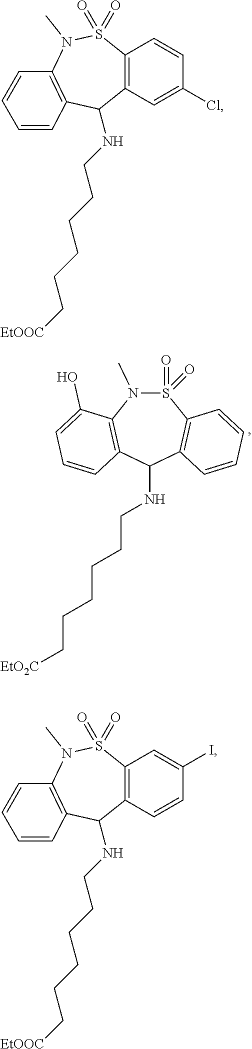 [SMILES: CCOC(=O)CCCCCCNC1c2ccccc2N(C)S(=O)(=O)c2ccc(Cl)cc21 ; CCOC(=O)CCCCCCNC1c2ccccc2S(=O)(=O)N(C)c2c(O)cccc21 ; CCOC(=O)CCCCCCNC1c2ccccc2N(C)S(=O)(=O)c2cc(I)ccc21]

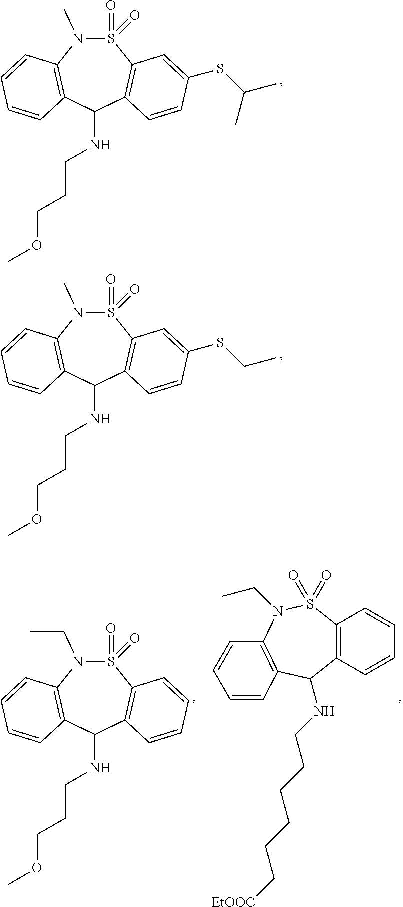

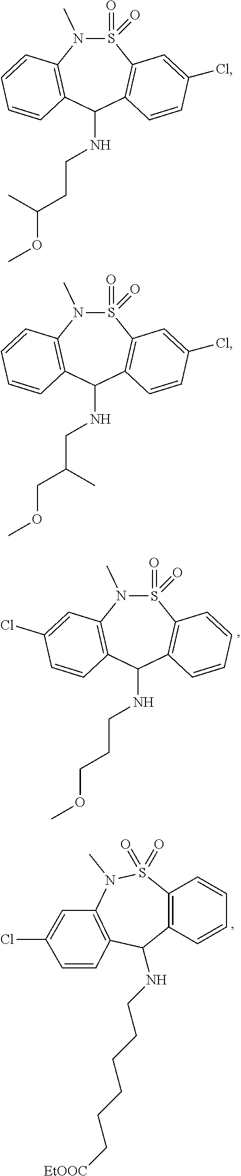

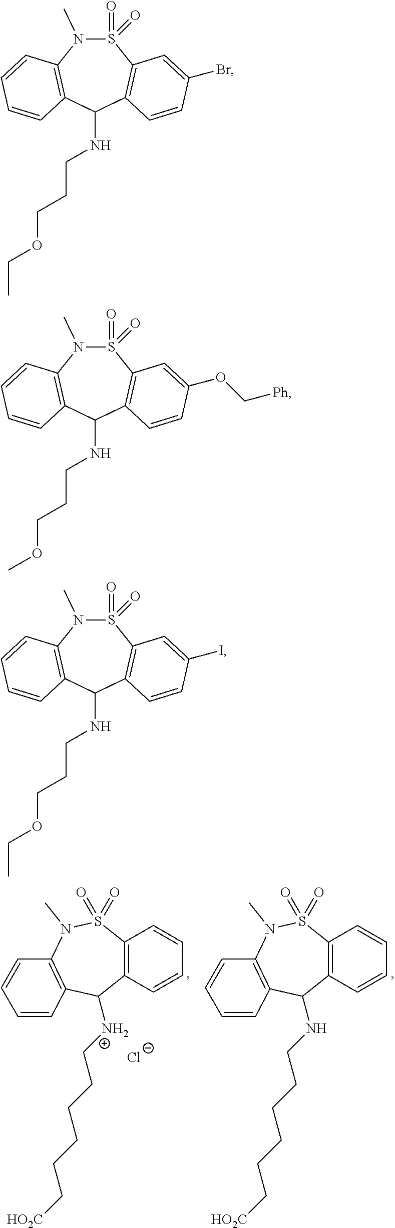

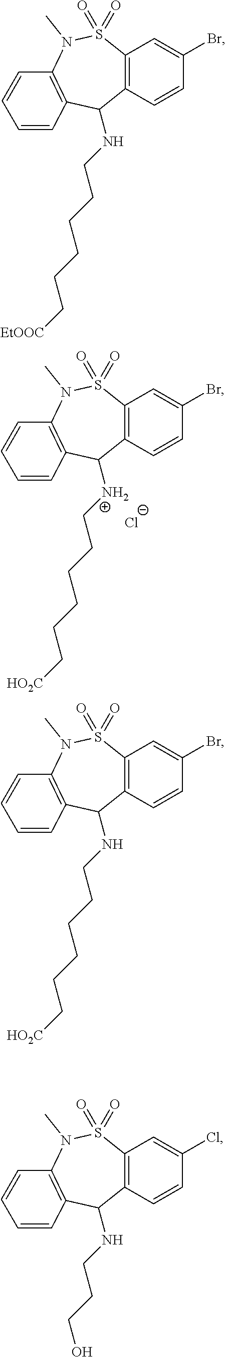 [SMILES: CCOC(=O)CCCCCCNC1c2ccccc2N(C)S(=O)(=O)c2cc(Br)ccc21 ; CN1c2ccccc2C([NH2+]CCCCCCC(=O)O)c2ccc(Br)cc2S1(=O)=O ; [Cl-] ; CN1c2ccccc2C(NCCCCCCC(=O)O)c2ccc(Br)cc2S1(=O)=O ; CN1c2ccccc2C(NCCCO)c2ccc(Cl)cc2S1(=O)=O]

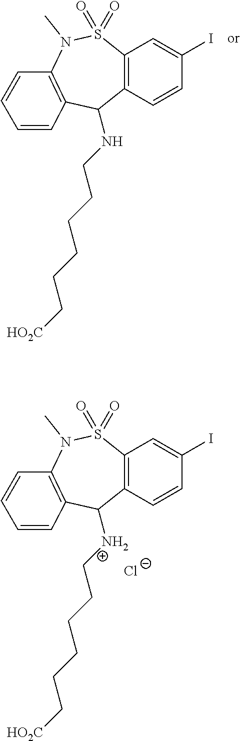 [SMILES: CN1c2ccccc2C(NCCCCCCC(=O)O)c2ccc(I)cc2S1(=O)=O ; CN1c2ccccc2C([NH2+]CCCCCCC(=O)O)c2ccc(I)cc2S1(=O)=O ; [Cl-]]

or a pharmaceutically acceptable salt thereof.

In some embodiments, the compound having the structure:

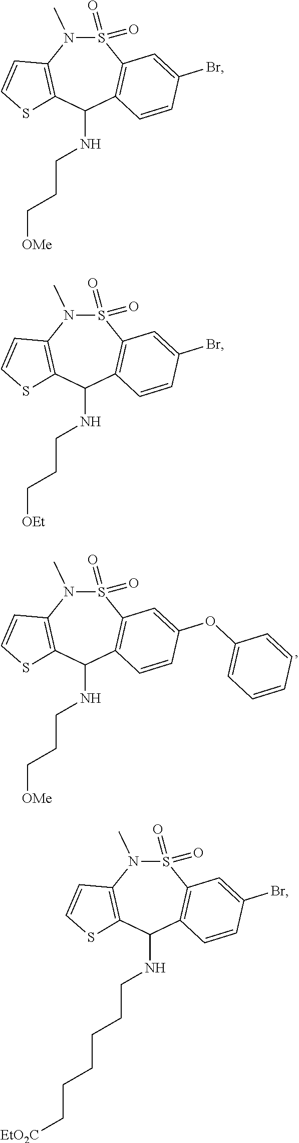 [SMILES: COCCCNC1c2ccc(Br)cc2S(=O)(=O)N(C)c2ccsc21 ; CCOCCCNC1c2ccc(Br)cc2S(=O)(=O)N(C)c2ccsc21 ; COCCCNC1c2ccc(Oc3ccccc3)cc2S(=O)(=O)N(C)c2ccsc21 ; CCOC(=O)CCCCCCNC1c2ccc(Br)cc2S(=O)(=O)N(C)c2ccsc21]

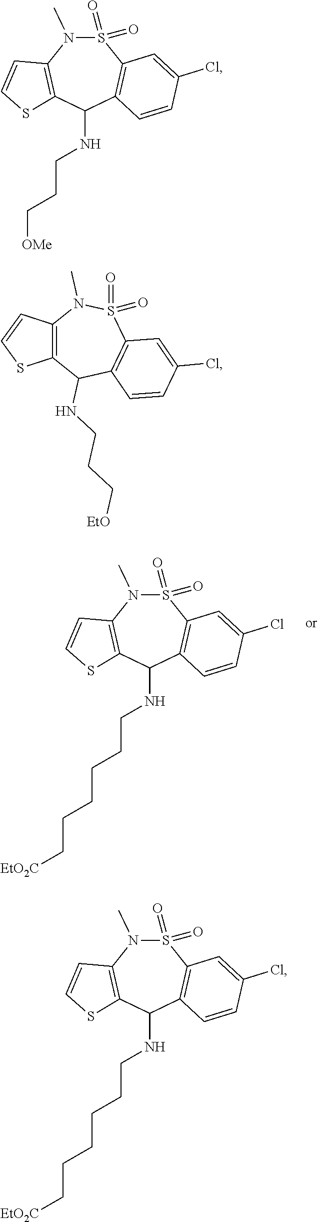

or a pharmaceutically acceptable salt thereof.

In some embodiments, the compound having the structure:

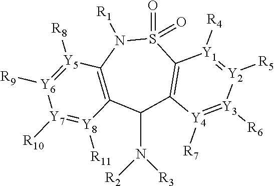

wherein
Y₁, Y₂, Y₃, Y₄, Y₅, Y₆, Y₇, and Y₈ are each C;
R₁ is —CH₃ or —CH₂CH₃;
R₂ is —(C₁-C₈ alkyl), —(C₁-C₆ alkyl)-OH, —(C₁-C₆ alkyl)-CO₂H, —(C₁-C₆ alkyl)-CO₂CH₂CH₃, —(C₁-C₆ alkyl)-OCH₃, —(C₁-C₆ alkyl)-C(O)NH₂,
(C₁-C₆ alkyl)-CF₃, —(C₁-C₆ alkyl)-SCH₃, —(C₁-C₆ alkyl)-OAc, —(C₁-C₆ alkyl)-CH(CH₂CH₃)₂, —(C₁-C₆ alkyl)-O—(C₁-C₆ alkyl), —(C₁-C₆ alkyl)-(1,3-dioxane), —(C₁-C₆ alkyl)-(1,3-dioxane), —(C₁-C₆ alkyl)-(4,5-dihydrooxazole), —(C₁-C₂ alkyl)-O—(C₁-C₂ alkyl)-OCH₃, —(C₁-C₂ alkyl)-O—(C₁-C₂ alkyl)-OH, —(C₁-C₆ alkyl)-C(O)—NH—(C₁-C₂ hydroxyalkyl), —(C₁-C₂ alkyl)-tetrahydrofuran, or —(C₁-C₂ alkyl)-pyrrolidine;
R₃ is —H;
R₈, R₉, R₁₀ and R₁₁ are each independently —H, —OCH₃, or —Br; and
R₄, R₅, R₆, and R₇ are each independently —H, —Cl, —Br, —F, —I, —CH₃, —OCH₃, —OH, —OAc, —SCH₃, —SCH₂CH₃, S-iPr, —SO₂CH₃, —S(O) CH₃, -(phenyl), —O—CH₂(phenyl) or —O-(phenyl),
   or a pharmaceutically acceptable salt thereof.

In some embodiments, the compound having the structure:

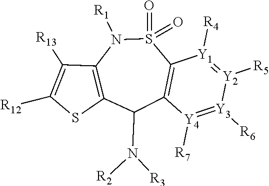

wherein
Y₁, Y₂, Y₃, and Y₄ are each C;
R₁ is —CH₃;
R₂ is —(C₁-C₆ alkyl)-OCH₃, —(C₁-C₆ alkyl)-O—(C₁-C₆ alkyl), —(C₁-C₆ alkyl)-CO₂CH₂CH₃ or —(C₁-C₆ alkyl)-OCH₃;
R₃, R₄, R₅, R₆ and R₁₂ are each —H, —Cl, —Br, —F, —I; and
R₁₃ is —H or —Br,
   or a pharmaceutically acceptable salt thereof.

In some embodiments, the compound having the structure:

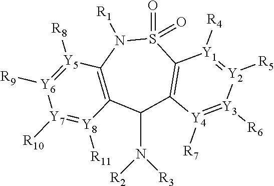

wherein
Y₁, Y₂, Y₃, Y₄, Y₅, Y₆, Y₇, and Y₈ are each C;
R₁ is —CH₃;
R₂ is —(C₁-C₈ alkyl), —(C₁-C₆ alkyl)-OH, —(C₁-C₆ alkyl)-CO₂H, —(C₁-C₆ alkyl)-CO₂CH₂CH₃, —(C₁-C₆ alkyl)-OCH₃, —(C₁-C₆ alkyl)-C(O)NH₂,
(C₁-C₆ alkyl)-CF₃, —(C₁-C₆ alkyl)-SCH₃, —(C₁-C₆ alkyl)-OAc, —(C₁-C₆ alkyl)-CH(CH₂CH₃)₂, —(C₁-C₆ alkyl)-O—(C₁-C₆ alkyl), —(C₁-C₆ alkyl)-(1,3-dioxane), —(C₁-C₆ alkyl)-(1,3-dioxane), —(C₁-C₆ alkyl)-(4,5-dihydrooxazole), —(C₁-C₂ alkyl)-O—(C₁-C₂ alkyl)-OCH₃,
(C₁-C₂ alkyl)-O—(C₁-C₂ alkyl)-OH, —(C₁-C₆ alkyl)-C(O)—NH—(C₁-C₂ hydroxyalkyl), —(C₁-C₂ alkyl)-tetrahydrofuran, or —(C₁-C₂ alkyl)-pyrrolidine;
R₃ is —H;
R₈, R₉, R₁₀ and R₁₁ are each independently —H, —OCH₃, or —Br; and
R₄, R₅, R₆, and R₇ are each independently —H, —Cl, —Br, —F, —I, —CH₃, —OCH₃, —OH, —OAc, —SCH₃, —SO2CH3, —S(O)CH₃, -(phenyl) or —O-(phenyl),
   or a pharmaceutically acceptable salt thereof.

In some embodiments, the compound having the structure:

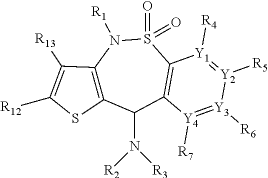

wherein
Y₁, Y₂, Y₃, and Y₄ are each C;
R₁ is —CH₃;
R₂ is —(C₁-C₆ alkyl)-CO₂CH₂CH₃ or —(C₁-C₆ alkyl)-OCH₃;
R₃, R₄, R₅, R₆ and R₁₂ are each —H; and
R₁₃ is —H or Br,
   or a pharmaceutically acceptable salt thereof.

In one embodiment of any of the compounds disclosed herein R₂ is -(alkyl). In one embodiment of any of the compounds disclosed herein R₂ is -(alkenyl). In one embodiment of any of the compounds disclosed herein R₂ is -(alkynyl). In one embodiment of any of the compounds disclosed herein R₂ is -(alkyl)-CO₂H. In one embodiment of any of the compounds disclosed herein R₂ is -(alkyl)-CO₂-(alkyl). In one embodiment of any of the compounds disclosed herein R₂ is -(alkyl)-OH. In one embodiment of any of the compounds disclosed herein R₂ is -(alkyl)-C(O)—NH₂. In one embodiment of any of the compounds disclosed herein R₂ is -(alkyl)-C(O)—NH(alkyl). In one embodiment of any of the compounds disclosed herein R₂ is -(alkyl)-C(O)—NH-(hydroxyalkyl). In one embodiment of any of the compounds disclosed herein R₂ is -(alkyl)-C(O)—N(alkyl)₂. In one embodiment of any of the compounds disclosed herein R₂ is -(alkyl)-C(O)—N(hydroxyalkyl)₂. In one embodiment of any of the compounds disclosed herein R₂ is -(alkyl)-S-(alkyl). In one embodiment of any of the compounds disclosed herein R₂ is -(alkyl)-CF₃. In one embodiment of any of the compounds disclosed herein R₂ is -(alkyl)-O-(hydroxyalkyl), In one embodiment of any of the compounds disclosed herein R₂ is -(alkyl)-O-(alkyl)-O-(alkyl). In one embodiment of any of the compounds disclosed herein R₂ is -(alkyl)-(CH)-(0-(alkyl)). In one embodiment of any of the compounds disclosed herein R₂ is -(alkyl)-(heterocyclyl). In one embodiment of any of the compounds disclosed herein R₂ is -(alkyl)-OAc. In one embodiment of any of the compounds disclosed herein R₂ is -(alkyl)-tetrahydrofuran. In one embodiment of any of the compounds disclosed herein R₂ is -(alkyl)-pyrrolidine. In one embodiment of any of the compounds disclosed herein R₂ is -(alkyl)-N-methylpyrrolidine. In one embodiment of any of the compounds disclosed herein R₂ is -(alkyl)-(1,3-dioxane). In one embodiment of any of the compounds disclosed herein R₂ is -(alkyl)-(4,5-dihydrooxazole).

In one embodiment of any of the compounds disclosed herein R₂ is —(C₁₋₁₂ alkyl)-CO₂H or any combination of any of —(C₁ alkyl)-CO₂H, —(C₂ alkyl)-CO₂H, —(C₃ alkyl)-CO₂H, —(C₄ alkyl)-CO₂H, —(C₄ alkyl)-CO₂H, —(C₅ alkyl)-CO₂H, —(C₆ alkyl)-CO₂H, —(C₇ alkyl)-CO₂H, —(C₈ alkyl)-CO₂H, —(C₅ alkyl)-CO₂H, —(C₁₀ alkyl)-CO₂H, —(C₁₁ alkyl)-CO₂H or —(C₁₂ alkyl)-CO₂H.

In one embodiment of any of the compounds disclosed herein R₂ is —(C₁₋₁₂ alkyl)-CO₂-(alkyl) or any combination of any of —(C₁ alkyl)-CO₂-(alkyl), —(C₂ alkyl)-CO₂-(alkyl), —(C₃ alkyl)-CO₂-(alkyl), —(C₄ alkyl)-CO₂-(alkyl), —(C₄ alkyl)-CO₂-(alkyl), —(C₅ alkyl)-CO₂-(alkyl), —(C₆ alkyl)-CO₂-(alkyl), —(C₇ alkyl)-CO₂-(alkyl), —(C₈ alkyl)-CO₂-(alkyl), —(C₉ alkyl)-CO₂-(alkyl), —(C₁₀ alkyl)-CO₂-(alkyl), —(C₁₁ alkyl)-CO₂-(alkyl) or —(C₁₂ alkyl)-CO₂-(alkyl).

In one embodiment of any of the compounds disclosed herein R₂ is —(C₁₋₁₂ alkyl)-O-(alkyl) or any combination of any of —(C₁ alkyl)-O-(alkyl), —(C₂ alkyl)-O-(alkyl), —(C₃ alkyl)-O-(alkyl), —(C₄ alkyl)-O-(alkyl), —(C₄ alkyl)-O-(alkyl), —(C₅ alkyl)-O-(alkyl), —(C₆ alkyl)-O-(alkyl), —(C₇ alkyl)-O-(alkyl), —(C₆ alkyl)-O-(alkyl), —(C₉ alkyl)-O-(alkyl), —(C₁₀ alkyl)-O-(alkyl), —(C₁₁ alkyl)-O-(alkyl) or —(C₁₂ alkyl)-O-(alkyl).

In one embodiment of any of the compounds disclosed herein R₅ is F. In one embodiment of any of the compounds disclosed herein R₅ is Cl. In one embodiment of any of the compounds disclosed herein R₅ is Br. In one embodiment of any of the compounds disclosed herein R₅ is I.

In one embodiment of any of the compounds disclosed herein R₅ is other than C₁. In one embodiment of any of the compounds disclosed herein R₂ is other than -(alkyl)-CO₂H.

In one embodiment of any of the compounds disclosed, A is phenyl. In one embodiment of any of the compounds disclosed, A is thiophene.

The present disclosure provides a pharmaceutical composition comprising a compound of the present disclosure and a pharmaceutically acceptable carrier.

The present disclosure provides a method of activating mu-opioid receptor comprising contacting the mu-opioid receptor with a compound of the present disclosure.

The present disclosure provides a method of activating delta-opioid receptor comprising contacting the delta-opioid receptor with a compound of the present disclosure.

The present disclosure provides a method of treating or preventing a neurological disorder in a subject, wherein the neurological disorder is selected from the group consisting of Huntington's disease, Rett syndrome, and CDKL5 disorder, and the other neurological disorders as described herein, the method comprising administering an effective amount of a compound of the present disclosure to the subject so as to treat or prevent the neurological disorder.

The present disclosure provides a method of treating a subject afflicted with a neurological disorder such as Huntington's disease comprising administering an effective amount of a compound of the present disclosure to the subject so as to treat the neurological disorder.

The present disclosure provides a method of treating a subject afflicted with a neurological disorder such as Rett syndrome comprising administering an effective amount of a compound of the present disclosure to the subject so as to treat the neurological disorder.

The present disclosure provides a method of treating a subject afflicted with a neurological disorder such as CDKL5 disorder comprising administering an effective amount of a compound of the present disclosure to the subject so as to treat the neurological disorder.

The present disclosure provides a method of treating a subject afflicted with a neurological disorder such as Rett syndrome variant comprising administering an effective amount of a compound of the present disclosure to the subject so as to treat the neurological disorder.

The present disclosure provides a method of activating mu-opioid receptor or delta-opioid receptor comprising contacting the mu-opioid receptor or delta-opioid receptor with a compound having the structure:

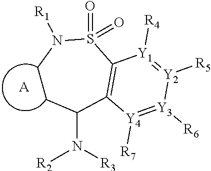

wherein
A is an aryl or heteroaryl, with or without substitution;
R₁ is —H or -(alkyl);
R₂ is -(alkyl), -(alkenyl), -(alkynyl)-(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-OCH₃, -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrocxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —OC(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
      or a pharmaceutically acceptable salt thereof,
      so as to thereby activate the mu-opioid receptor or delta-opioid receptor, and thereby treat or prevent a neurological disorder in a subject, wherein the neurological disorder is selected from the group consisting of Huntington's disease, Rett syndrome, and CDKL5 disorder, and the other neurological disorders as described herein.

In some embodiments, the mu-opioid receptors or delta-opioid receptors are in a human subject.

The present disclosure provides a method of treating a subject afflicted with a neurological disorder, wherein the neurological disorder is selected from the group consisting of Huntington's disease, Rett syndrome, and CDKL5 disorder, and the other neurological disorders as described herein, comprising administering an effective amount of a compound having the structure:

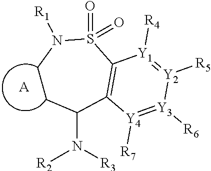

wherein
A is an aryl or heteroaryl, with or without substitution;
R₁ is —H or -(alkyl);
R₂ is -(alkyl), -(alkenyl), -(alkynyl)-(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-OCH₃, -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
      or a pharmaceutically acceptable salt thereof,
      to the subject so as to thereby treat the neurological disorder.

The present disclosure provides a method of treating a subject afflicted with a neurological disorder such as Huntington's disease comprising administering an effective amount of a compound having the structure:

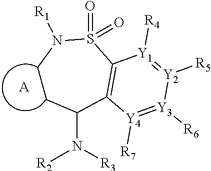

wherein
A is an aryl or heteroaryl, with or without substitution;
R₁ is —H or -(alkyl);
R₂ is -(alkyl), -(alkenyl), -(alkynyl)-(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-OCH₃, -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
      or a pharmaceutically acceptable salt thereof,
      to the subject so as to thereby treat the Huntington's disease.

The present disclosure provides a method of treating a subject afflicted with a neurological disorder such as Rett syndrome comprising administering an effective amount of a compound having the structure:

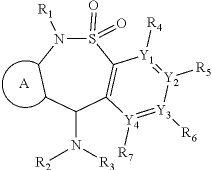

wherein
A is an aryl or heteroaryl, with or without substitution;
R₁ is —H or -(alkyl);
R₂ is -(alkyl), -(alkenyl), -(alkynyl)-(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-OCH₃, -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrocxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
      or a pharmaceutically acceptable salt thereof,
      to the subject so as to thereby treat the neurological disorder.

The present disclosure also provides a method of treating a subject afflicted with a neurological disorder, wherein the neurological disorder is selected from the group consisting of Huntington's disease, Rett syndrome, and CDKL5 disorder, and the other neurological disorders as described herein, comprising administering to the subject an effective amount of an NMDA receptor antagonist, an NMDA receptor partial agonist, a neurokinin 1 receptor antagonist, a neurokinin 2 receptor antagonist or a neurokinin 3 receptor antagonist and an effective amount of a compound having the structure:

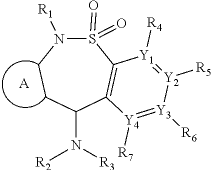

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrocxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkylaryl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
      or a pharmaceutically acceptable salt thereof, so as to thereby treat the neurological disorder.

The present disclosure also provides a method of treating a subject afflicted with a neurological disorder such as Huntington's disease comprising administering to the subject an effective amount of an NMDA receptor antagonist, an NMDA receptor partial agonist, a neurokinin 1 receptor antagonist, a neurokinin 2 receptor antagonist or a neurokinin 3 receptor antagonist and an effective amount of a compound having the structure:

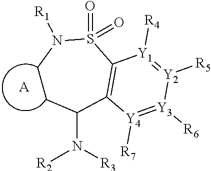

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrocxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkylaryl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
      or a pharmaceutically acceptable salt thereof, so as to thereby treat the neurological disorder.

In some embodiments of the above method, the compound when A is phenyl, R₁ is —CH₃, R₃, R₄, R₆, and R₇ are each —H, and R₅ is Cl, then R₂ is other than —(CH₂)₆CO₂H.

The present disclosure also provides a compound having the structure:

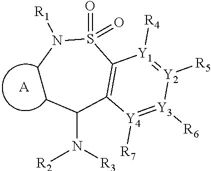

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkylaryl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
or a salt or ester thereof, for use as an add-on therapy or in combination with an NMDA receptor antagonist, an NMDA receptor partial agonist, a neurokinin 1 receptor antagonist, a neurokinin 2 receptor antagonist or a neurokinin 3 receptor antagonist in treating a subject afflicted with a neurological disorder, wherein the neurological disorder is selected from the group consisting of Huntington's disease, Rett syndrome, and CDKL5 disorder, and the other neurological disorders as described herein.

The present disclosure also provides a compound having the structure:

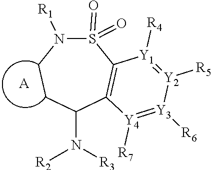

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrocxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkylaryl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
or a salt or ester thereof, for use as an add-on therapy or in combination with an NMDA receptor antagonist, an NMDA receptor partial agonist, a neurokinin 1 receptor antagonist, a neurokinin 2 receptor antagonist or a neurokinin 3 receptor antagonist in treating a subject afflicted with a neurological disorder such as Huntington's disease.

In some embodiments of the above, the compound wherein when A is phenyl, R₁ is —CH₃, R₃, R₄, R₆, and R₇ are each —H, and R₅ is Cl, then R₂ is other than —(CH₂)₆CO₂H.

The present disclosure further provides a pharmaceutical composition comprising an amount of a compound having the structure

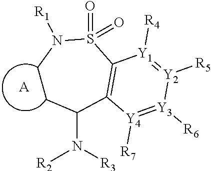

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkylaryl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
or a salt or ester thereof, and an amount of an NMDA receptor antagonist, an NMDA receptor partial agonist, a neurokinin 1 receptor antagonist, a neurokinin 2 receptor antagonist or a neurokinin 3 receptor antagonist for use in treating a subject afflicted with a neurological disorder, wherein the neurological disorder is selected from the group consisting of Huntington's disease, Rett syndrome, and CDKL5 disorder, and the other neurological disorders as described herein.

The present disclosure further provides a pharmaceutical composition comprising an amount of a compound having the structure

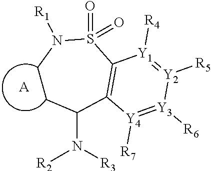

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkylaryl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
or a salt or ester thereof, and an amount of an NMDA receptor antagonist, an NMDA receptor partial agonist, a neurokinin 1 receptor antagonist, a neurokinin 2 receptor antagonist or a neurokinin 3 receptor antagonist for use in treating a subject afflicted with a neurological disorder such as Huntington's disease.

In some embodiments of the above pharmaceutical composition, the compound wherein when A is phenyl, R₁ is —CH₃, R₃, R₄, R₆, and R₇ are each —H, and R₅ is Cl, then R₂ is other than —(CH₂)₆CO₂H.

The present disclosure also provides a method of treating a subject afflicted with a neurological disorder, wherein the neurological disorder is selected from the group consisting of Huntington's disease, Rett syndrome, and CDKL5 disorder, and the other neurological disorders as described herein, comprising administering to the subject an effective amount of an NMDA receptor antagonist, an NMDA receptor partial agonist, a neurokinin 1 receptor antagonist, a neurokinin 2 receptor antagonist or a neurokinin 3 receptor antagonist and an effective amount of a compound having the structure:

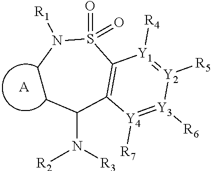

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkylaryl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₃ is present; when Y₃ is N, then R₆ is absent, and when Y₃ is C, then R₆ is present; when Y₄ is N, then R₇ is absent, and when Y₄ is C, then R₇ is present,
      or a pharmaceutically acceptable salt thereof, so as to thereby treat the neurological disorder.

In some embodiments of the above method, the compound when A is phenyl, R₁ is —CH₃, R₃, R₄, R₆, and R₇ are each —H, and R₅ is Cl, then R₂ is other than —(CH₂)₆CO₂H.

The present disclosure also provides a compound having the structure:

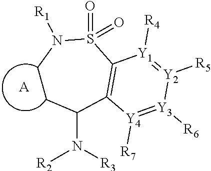

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkylaryl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
or a salt or ester thereof, for use as an add-on therapy or in combination with an NMDA receptor antagonist, an NMDA receptor partial agonist, a neurokinin 1 receptor antagonist, a neurokinin 2 receptor antagonist or a neurokinin 3 receptor antagonist in treating a subject afflicted with a neurological disorder, wherein the neurological disorder is selected from the group consisting of Huntington's disease, Rett syndrome, and CDKL5 disorder, and the other neurological disorders as described herein.

In some embodiments of the above, the compound wherein when A is phenyl, R₁ is —CH₃, R₃, R₄, R₆, and R₇ are each —H, and R₅ is Cl, then R₂ is other than —(CH₂)₆CO₂H.

The present disclosure further provides a pharmaceutical composition comprising an amount of a compound having the structure

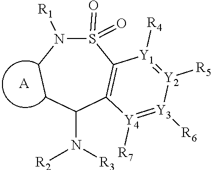

wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃. —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkylaryl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
or a salt or ester thereof, and an amount of an NMDA receptor antagonist, an NMDA receptor partial agonist, a neurokinin 1 receptor antagonist, a neurokinin 2 receptor antagonist or a neurokinin 3 receptor antagonist for use in treating a subject afflicted with a neurological disorder, wherein the neurological disorder is selected from the group consisting of Huntington's disease, Rett syndrome, and CDKL5 disorder, and the other neurological disorders as described herein.

In some embodiments of the above pharmaceutical composition, the compound wherein when A is phenyl, R₁ is —CH₃, R₃, R₄, R₆, and R₇ are each —H, and R₅ is Cl, then R₂ is other than —(CH₂)₆CO₂H.

In some embodiments, a package comprising:
a) a first pharmaceutical composition comprising an amount of an NMDA receptor antagonist, an NMDA receptor partial agonist, a neurokinin 1 receptor antagonist, a neurokinin 2 receptor antagonist or a neurokinin 3 receptor antagonist and a pharmaceutically acceptable carrier;
b) a second pharmaceutical composition comprising an amount of any of the compounds of the present disclosure, or a salt or ester thereof; and
c) instructions for use of the first and second pharmaceutical compositions together to treat a subject afflicted with a neurological disorder, wherein the neurological disorder is selected from the group consisting of Huntington's disease, Rett syndrome, and CDKL5 disorder, and the other neurological disorders as described herein.

In some embodiments, a therapeutic package for dispensing to, or for use in dispensing to, a subject afflicted with a neurological disorder, wherein the neurological disorder is selected from the group consisting of Huntington's disease, Rett syndrome, and CDKL5 disorder, and the other neurological disorders as described herein, which comprises:
a) one or more unit doses, each such unit dose comprising:
   (i) an amount of any of the compounds of the present disclosure, or a salt or ester thereof; and
   (ii) an amount of an NMDA receptor antagonist, an NMDA receptor partial agonist, a neurokinin 1 receptor antagonist, a neurokinin 2 receptor antagonist or a neurokinin 3 receptor antagonist,
   wherein the respective amounts of said compound and said agonist or antagonist in said unit dose are effective, upon concomitant administration to said subject, to treat the subject, and
(b) a finished pharmaceutical container therefor, said container containing said unit dose or unit doses, said container further containing or comprising labeling directing the use of said package in the treatment of said subject.

The therapeutic package of the above embodiment, wherein the respective amounts of said compound and said agonist or antagonist in said unit dose when taken together is more effective to treat the subject than when compared to the administration of said compound in the absence of said agonist or antagonist or the administration of said agonist or antagonist in the absence of said compound.

A pharmaceutical composition in unit dosage form, useful in treating a subject afflicted with a neurological disorder, wherein the neurological disorder is selected from the group consisting of Huntington's disease, Rett syndrome, and CDKL5 disorder, and the other neurological disorders as described herein, which comprises:
(i) an amount of any of the compounds of the present disclosure, or a salt or ester thereof; and
(ii) an amount of an NMDA receptor antagonist, an NMDA receptor partial agonist, a neurokinin 1 receptor antagonist, a neurokinin 2 receptor antagonist or a neurokinin 3 receptor antagonist,
wherein the respective amounts of said compound and said agonist or antagonist in said composition are effective, upon concomitant administration to said subject of one or more of said unit dosage forms of said composition, to treat the disorder.

The pharmaceutical composition of the above embodiment, wherein the respective amounts of said compound and said agonist or antagonist in said unit dose when taken together is more effective to treat the subject than when compared to the administration of said compound in the absence of said agonist or antagonist or the administration of said agonist or antagonist in the absence of said compound.

In some embodiments, a package comprising:
a) a first pharmaceutical composition comprising an amount of an NMDA receptor antagonist, an NMDA receptor partial agonist, a neurokinin 1 receptor antagonist, a neurokinin 2 receptor antagonist or a neurokinin 3 receptor antagonist and a pharmaceutically acceptable carrier;
b) a second pharmaceutical composition comprising an amount of any of the compounds of the present disclosure, or a salt or ester thereof; and
c) instructions for use of the first and second pharmaceutical compositions together to treat a subject afflicted with a neurological disorder such as Huntington's disease.

In some embodiments, a therapeutic package for dispensing to, or for use in dispensing to, a subject afflicted with a neurological disorder such as Huntington's disease, which comprises:
a) one or more unit doses, each such unit dose comprising:
   (i) an amount of any of the compounds of the present disclosure, or a salt or ester thereof; and
   (ii) an amount of an NMDA receptor antagonist, an NMDA receptor partial agonist, a neurokinin 1 receptor antagonist, a neurokinin 2 receptor antagonist or a neurokinin 3 receptor antagonist,
   wherein the respective amounts of said compound and said agonist or antagonist in said unit dose are effective, upon concomitant administration to said subject, to treat the subject, and
(b) a finished pharmaceutical container therefor, said container containing said unit dose or unit doses, said container further containing or comprising labeling directing the use of said package in the treatment of said subject.

The therapeutic package of the above embodiment, wherein the respective amounts of said compound and said agonist or antagonist in said unit dose when taken together is more effective to treat the subject than when compared to the administration of said compound in the absence of said agonist or antagonist or the administration of said agonist or antagonist in the absence of said compound.

A pharmaceutical composition in unit dosage form, useful in treating a subject afflicted with a neurological disorder such as Huntington's disease, which comprises:
(i) an amount of any of the compounds of the present disclosure, or a salt or ester thereof; and
(ii) an amount of an NMDA receptor antagonist, an NMDA receptor partial agonist, a neurokinin 1 receptor antagonist, a neurokinin 2 receptor antagonist or a neurokinin 3 receptor antagonist,
wherein the respective amounts of said compound and said agonist or antagonist in said composition are effective, upon concomitant administration to said subject of one or more of said unit dosage forms of said composition, to treat the subject.

The pharmaceutical composition of the above embodiment, wherein the respective amounts of said compound and said agonist or antagonist in said unit dose when taken together is more effective to treat the subject than when compared to the administration of said compound in the absence of said agonist or antagonist or the administration of said agonist or antagonist in the absence of said compound.

The present disclosure also provides a method of treating a subject afflicted with a neurological disorder, wherein the neurological disorder is selected from the group consisting of Huntington's disease, Rett syndrome, and CDKL5 disorder, and the other neurological disorders as described herein comprising administering to the subject an effective amount of an NMDA receptor antagonist, an NMDA receptor partial agonist, a neurokinin 1 receptor antagonist, a neurokinin 2 receptor antagonist or a neurokinin 3 receptor antagonist and an effective amount of any of the compounds of the present disclosure, or a salt or ester thereof, so as to thereby treat the neurological disorder.

The present disclosure also provides a compound of this disclosure or a salt or ester thereof, for use as an add-on therapy or in combination with an NMDA receptor antagonist, an NMDA receptor partial agonist, a neurokinin 1 receptor antagonist, a neurokinin 2 receptor antagonist or a neurokinin 3 receptor antagonist in treating a subject afflicted with a neurological disorder, wherein the neurological disorder is selected from the group consisting of Huntington's disease, Rett syndrome, and CDKL5 disorder, and the other neurological disorders as described herein.

The present disclosure further provides a pharmaceutical composition comprising an amount of any of the compounds of this disclosure, or a salt or ester thereof, and an amount of an NMDA receptor antagonist, an NMDA receptor partial agonist, a neurokinin 1 receptor antagonist, a neurokinin 2 receptor antagonist or a neurokinin 3 receptor antagonist for use in treating a subject afflicted with a neurological disorder, wherein the neurological disorder is selected from the group consisting of Huntington's disease, Rett syndrome, and CDKL5 disorder, and the other neurological disorders as described herein.

The present disclosure also provides a method of treating a subject afflicted with a neurological disorder, wherein the neurological disorder is selected from the group consisting of Huntington's disease, Rett syndrome, and CDKL5 disorder, and the other neurological disorders as described herein, comprising administering to the subject an effective amount of an NMDA receptor antagonist and an effective amount of any of the compounds of the present disclosure, or a salt or ester thereof, so as to thereby treat the neurological disorder.

The present disclosure also provides a compound of this disclosure or a salt or ester thereof, for use as an add-on therapy or in combination with an NMDA receptor antagonist, in treating a subject afflicted with a neurological disorder, wherein the neurological disorder is selected from the group consisting of Huntington's disease, Rett syndrome, and CDKL5 disorder, and the other neurological disorders as described herein.

The present disclosure further provides a pharmaceutical composition comprising an amount of any of the compounds of this disclosure, or a salt or ester thereof, and an amount of an NMDA receptor antagonist, for use in treating a subject afflicted with a neurological disorder, wherein the neurological disorder is selected from the group consisting of Huntington's disease, Rett syndrome, and CDKL5 disorder, and the other neurological disorders as described herein.

The present disclosure also provides a method of treating a subject afflicted with a neurological disorder, wherein the neurological disorder is selected from the group consisting of Huntington's disease, Rett syndrome, and CDKL5 disorder, and the other neurological disorders as described herein, comprising administering to the subject an effective amount of an NMDA receptor partial agonist, and an effective amount of any of the compounds of the present disclosure, or a salt or ester thereof, so as to thereby treat the neurological disorder.

The present disclosure also provides a compound of this disclosure or a salt or ester thereof, for use as an add-on therapy or in combination with an NMDA receptor partial agonist in treating a subject afflicted with a neurological disorder, wherein the neurological disorder is selected from the group consisting of Huntington's disease, Rett syndrome, and CDKL5 disorder, and the other neurological disorders as described herein.

The present disclosure further provides a pharmaceutical composition comprising an amount of any of the compounds of this disclosure, or a salt or ester thereof, and an amount of an NMDA receptor partial agonist, for use in treating a subject afflicted with a neurological disorder, wherein the neurological disorder is selected from the group consisting of Huntington's disease, Rett syndrome, and CDKL5 disorder, and the other neurological disorders as described herein.

The present disclosure also provides a method of treating a subject afflicted with a neurological disorder, wherein the neurological disorder is selected from the group consisting of Huntington's disease, Rett syndrome, and CDKL5 disorder, and the other neurological disorders as described herein, comprising administering to the subject an effective amount of a neurokinin 1 receptor antagonist and an effective amount of any of the compounds of the present disclosure, or a salt or ester thereof, so as to thereby treat the neurological disorder.

The present disclosure also provides a compound of this disclosure or a salt or ester thereof, for use as an add-on therapy or in combination with a neurokinin 1 receptor antagonist in treating a subject afflicted with a neurological disorder, wherein the neurological disorder is selected from the group consisting of Huntington's disease, Rett syndrome, and CDKL5 disorder, and the other neurological disorders as described herein.

The present disclosure further provides a pharmaceutical composition comprising an amount of any of the compounds of this disclosure, or a salt or ester thereof, and an amount of a neurokinin 1 receptor antagonist for use in treating a subject afflicted with a neurological disorder, wherein the neurological disorder is selected from the group consisting of Huntington's disease, Rett syndrome, and CDKL5 disorder, and the other neurological disorders as described herein.

The present disclosure also provides a method of treating a subject afflicted with a neurological disorder, wherein the neurological disorder is selected from the group consisting of Huntington's disease, Rett syndrome, and CDKL5 disorder, and the other neurological disorders as described herein, comprising administering to the subject an effective amount of a neurokinin 2 receptor antagonist and an effective amount of any of the compounds of the present disclosure, or a salt or ester thereof, so as to thereby treat the neurological disorder.

The present disclosure also provides a compound of this disclosure or a salt or ester thereof, for use as an add-on therapy or in combination with a neurokinin 2 receptor antagonist in treating a subject afflicted with a neurological disorder, wherein the neurological disorder is selected from the group consisting of Huntington's disease, Rett syndrome, and CDKL5 disorder, and the other neurological disorders as described herein.

The present disclosure further provides a pharmaceutical composition comprising an amount of any of the compounds of this disclosure, or a salt or ester thereof, and an amount of a neurokinin 2 receptor antagonist for use in treating a subject afflicted with a neurological disorder, wherein the neurological disorder is selected from the group consisting of Huntington's disease, Rett syndrome, and CDKL5 disorder, and the other neurological disorders as described herein.

The present disclosure also provides a method of treating a subject afflicted with a neurological disorder, wherein the neurological disorder is selected from the group consisting of Huntington's disease, Rett syndrome, and CDKL5 disorder, and the other neurological disorders as described herein, comprising administering to the subject an effective amount of a neurokinin 3 receptor antagonist and an effective amount of any of the compounds of the present disclosure, or a salt or ester thereof, so as to thereby treat the neurological disorder.

The present disclosure also provides a compound of this disclosure or a salt or ester thereof, for use as an add-on therapy or in combination with a neurokinin 3 receptor antagonist in treating a subject afflicted with a neurological disorder, wherein the neurological disorder is selected from the group consisting of Huntington's disease, Rett syndrome, and CDKL5 disorder, and the other neurological disorders as described herein.

The present disclosure further provides a pharmaceutical composition comprising an amount of any of the compounds of this disclosure, or a salt or ester thereof, and an amount of a neurokinin 3 receptor antagonist for use in treating a subject afflicted with a neurological disorder, wherein the neurological disorder is selected from the group consisting of Huntington's disease, Rett syndrome, and CDKL5 disorder, and the other neurological disorders as described herein.

In some embodiments of the present method, compound, package, use or pharmaceutical composition, the compound has the structure:

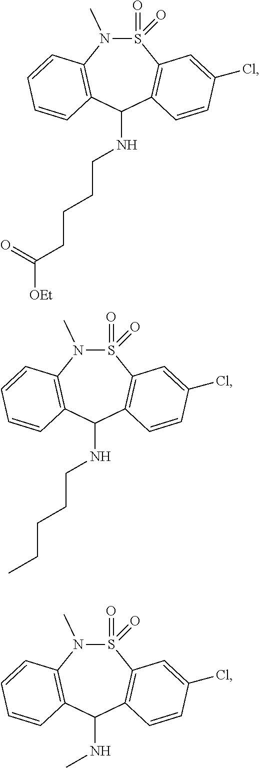

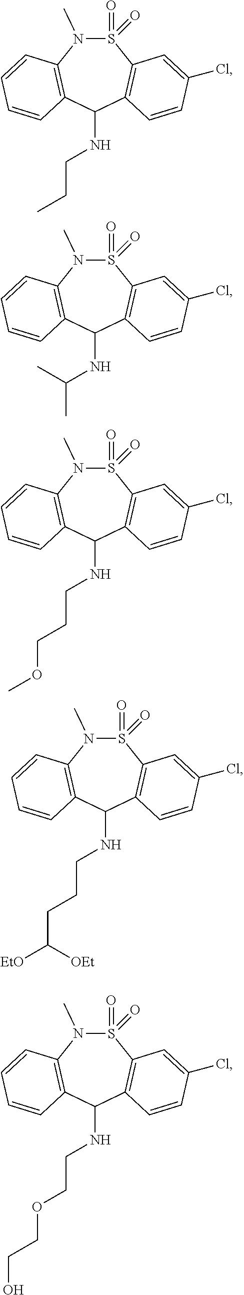

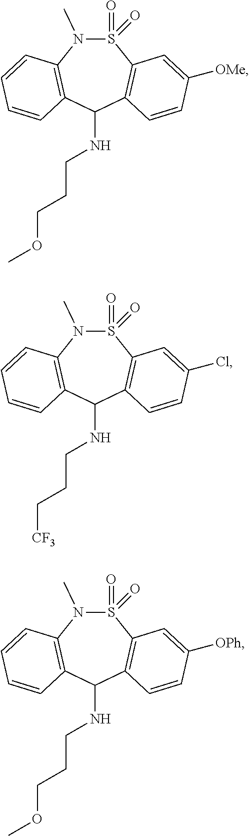 [SMILES: COCCCNC1c2ccccc2N(C)S(=O)(=O)c2cc(OC)ccc21 ; CN1c2ccccc2C(NCCCC(F)(F)F)c2ccc(Cl)cc2S1(=O)=O ; COCCCNC1c2ccccc2N(C)S(=O)(=O)c2cc(Oc3ccccc3)ccc21]

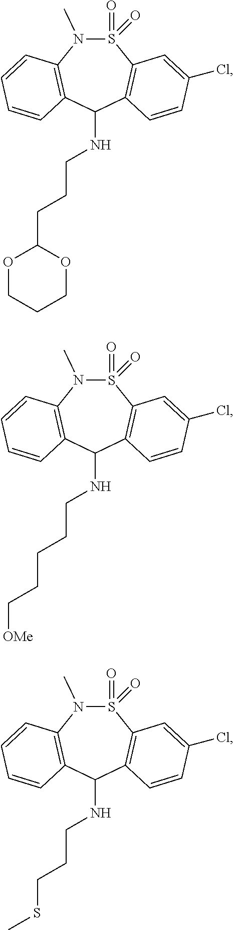

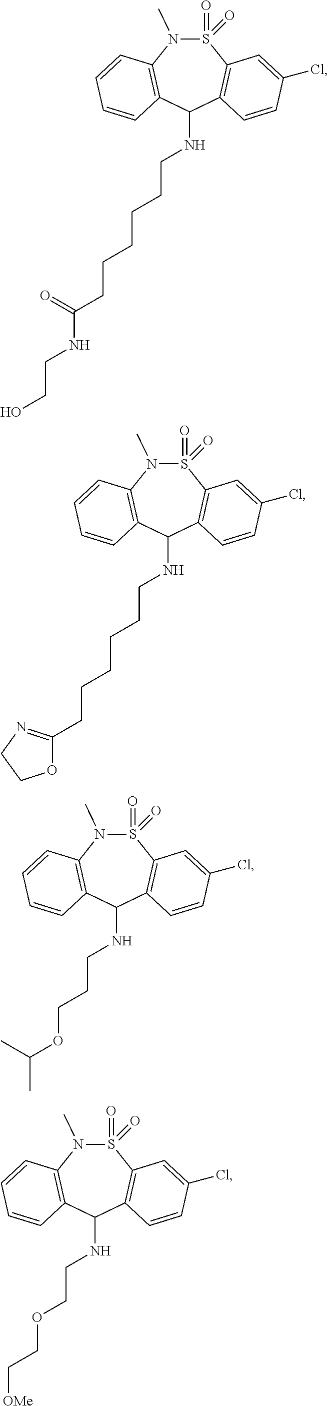

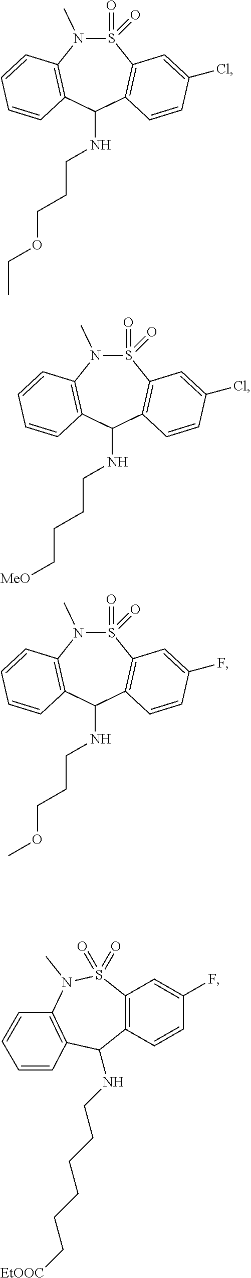

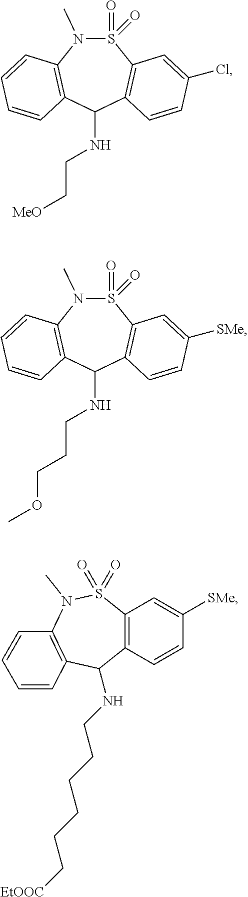 [SMILES: COCCNC1c2ccccc2N(C)S(=O)(=O)c2cc(Cl)ccc21 ; COCCCNC1c2ccccc2N(C)S(=O)(=O)c2cc(SC)ccc21 ; CCOC(=O)CCCCCCNC1c2ccccc2N(C)S(=O)(=O)c2cc(SC)ccc21]

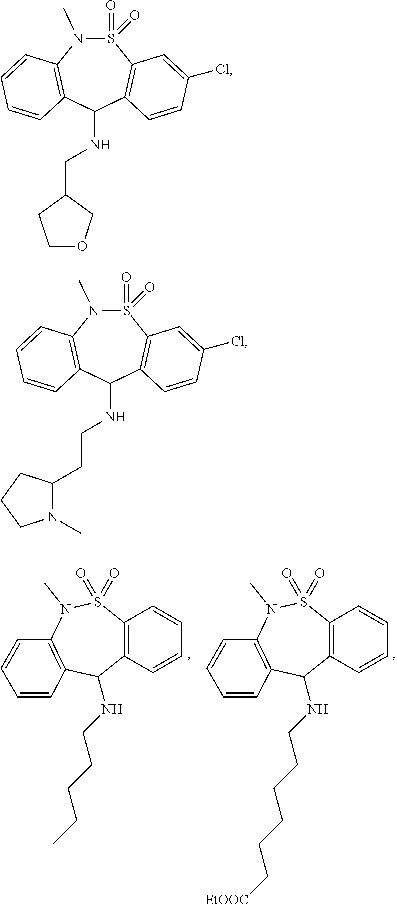

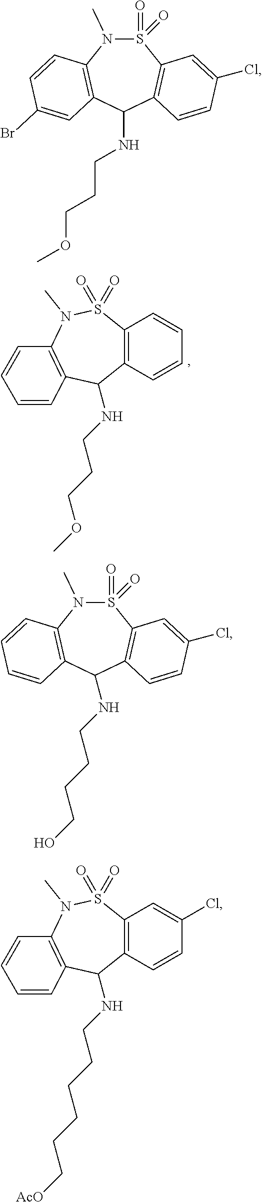

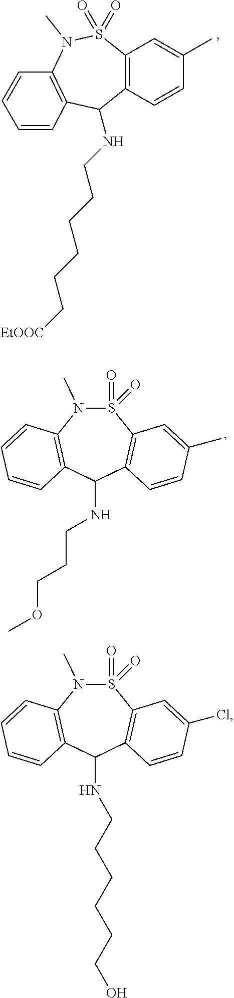 [SMILES: CCOC(=O)CCCCCCNC1c2ccccc2N(C)S(=O)(=O)c2cc(C)ccc21 ; COCCCNC1c2ccccc2N(C)S(=O)(=O)c2cc(C)ccc21 ; CN1c2ccccc2C(NCCCCCCO)c2ccc(Cl)cc2S1(=O)=O]

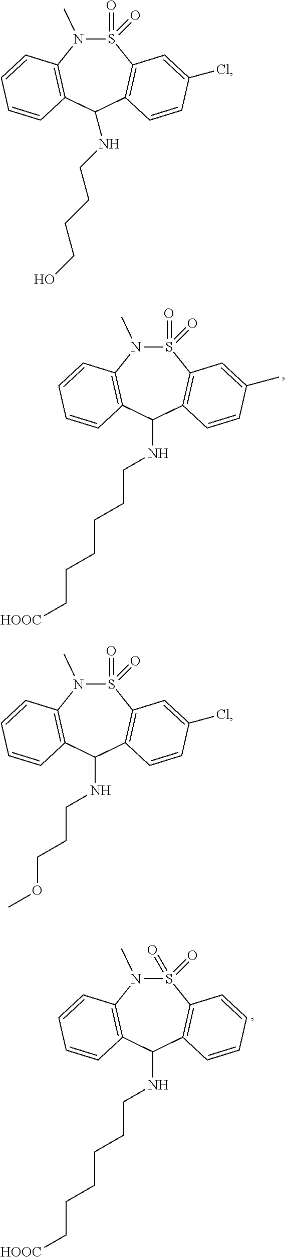

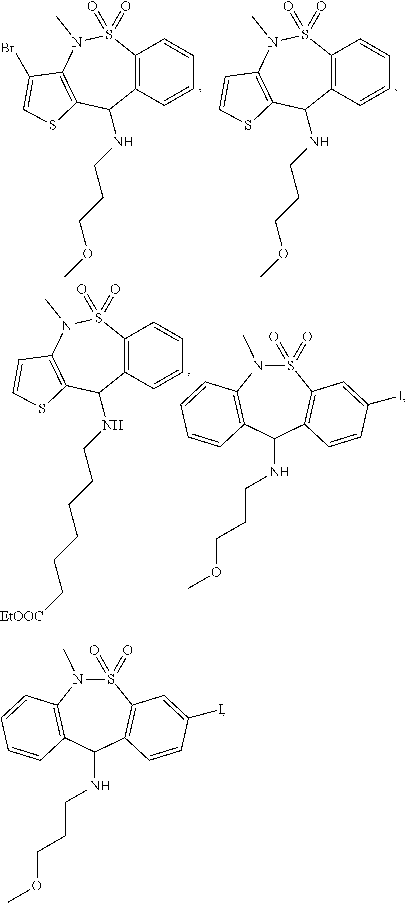

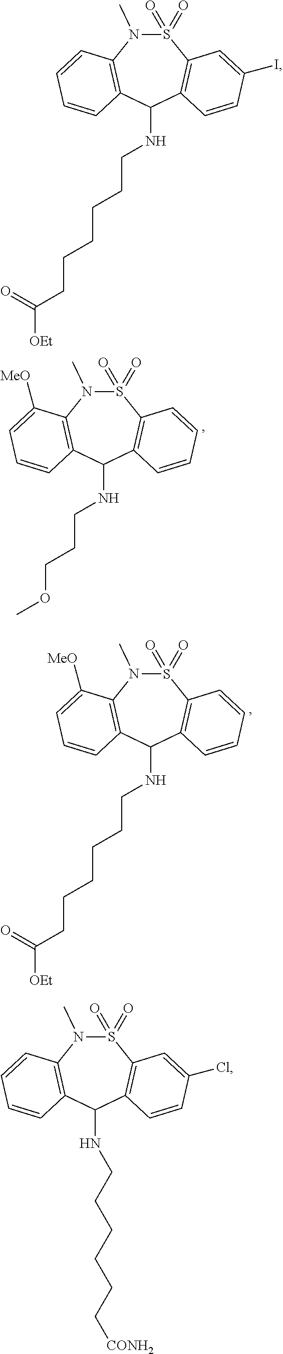

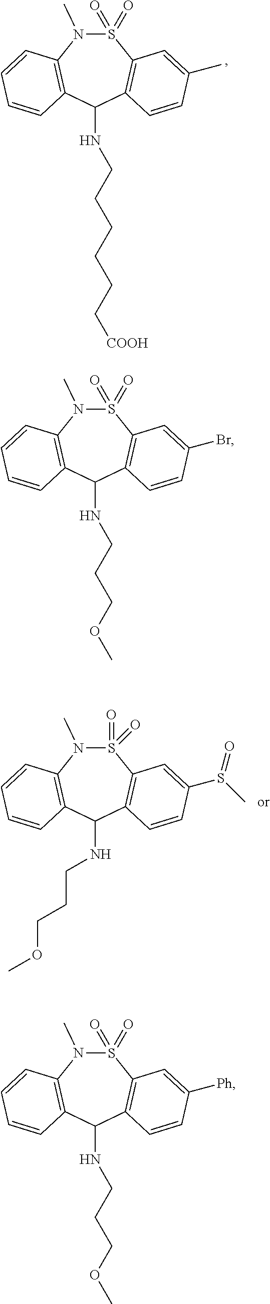

or a pharmaceutically acceptable salt thereof.

In some embodiments of the present method, compound, package, use or pharmaceutical composition, the compound has the structure:

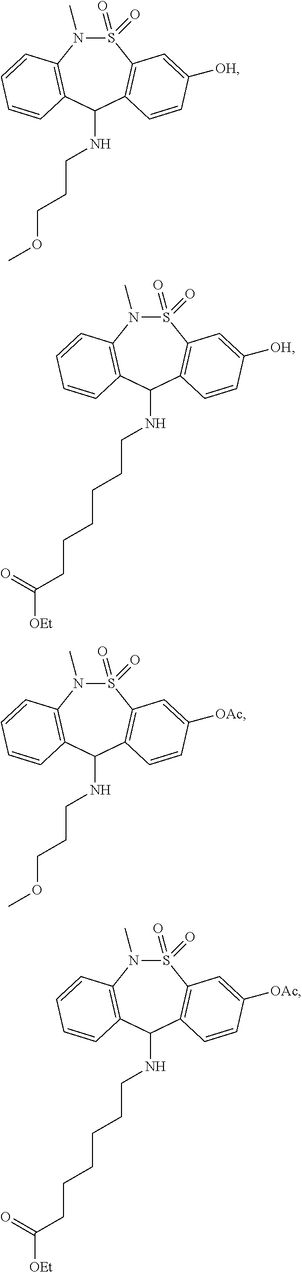

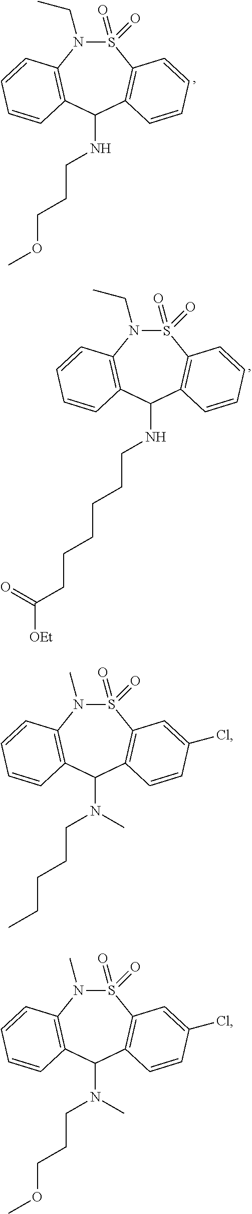 [SMILES: CCN1c2ccccc2C(NCCCOC)c2ccccc2S1(=O)=O ; CCOC(=O)CCCCCCNC1c2ccccc2N(CC)S(=O)(=O)c2ccccc21 ; CCCCCN(C)C1c2ccccc2N(C)S(=O)(=O)c2cc(Cl)ccc21 ; COCCCN(C)C1c2ccccc2N(C)S(=O)(=O)c2cc(Cl)ccc21]

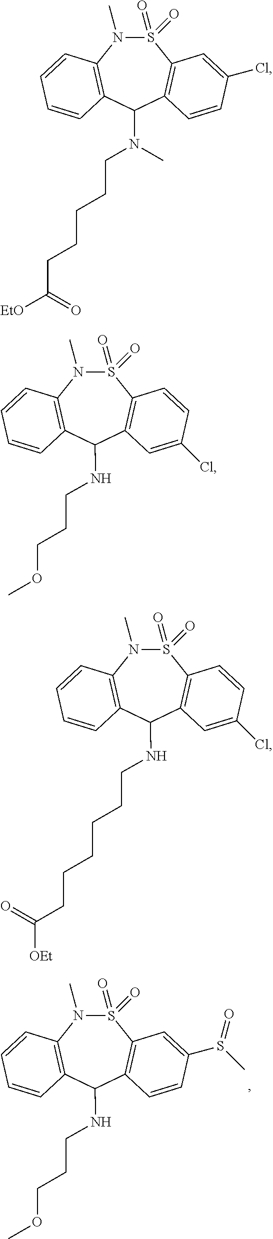

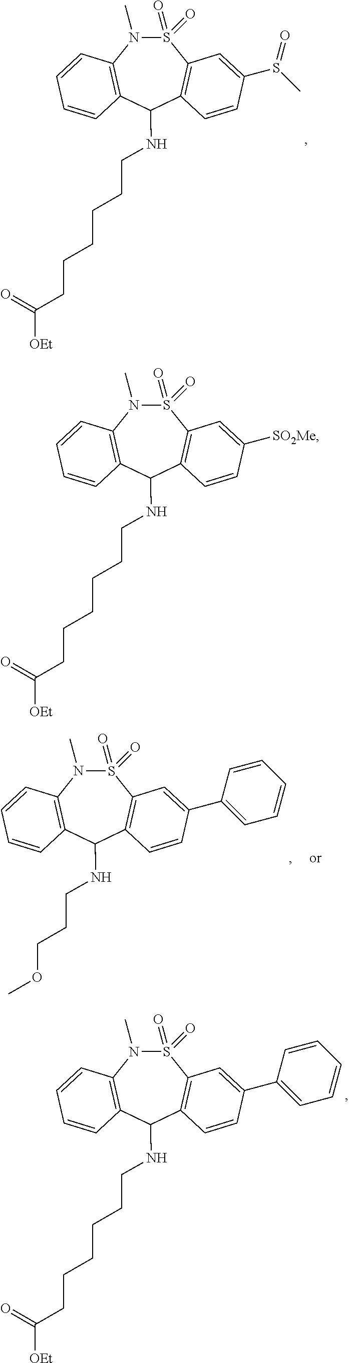

or a pharmaceutically acceptable salt thereof.

In some embodiments of the present method, compound, package, use or pharmaceutical composition, the compound has the structure:

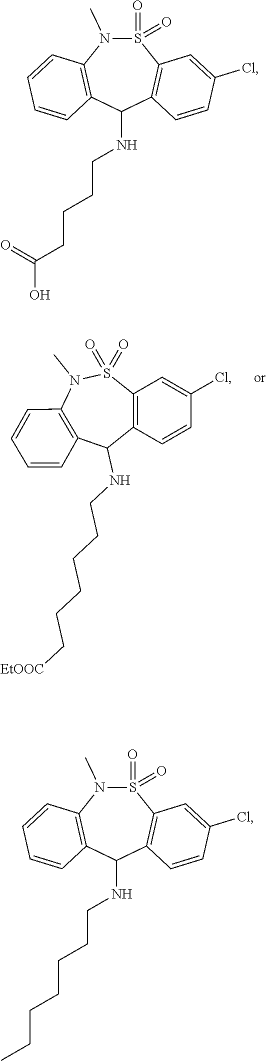 [SMILES: CN1c2ccccc2C(NCCCCC(=O)O)c2ccc(Cl)cc2S1(=O)=O ; CCOC(=O)CCCCCCNC1c2ccccc2N(C)S(=O)(=O)c2cc(Cl)ccc21 ; CCCCCCCNC1c2ccccc2N(C)S(=O)(=O)c2cc(Cl)ccc21]

or a pharmaceutically acceptable salt thereof.

In some embodiments of the present method, compound, package, use or pharmaceutical composition, the compound has the structure:

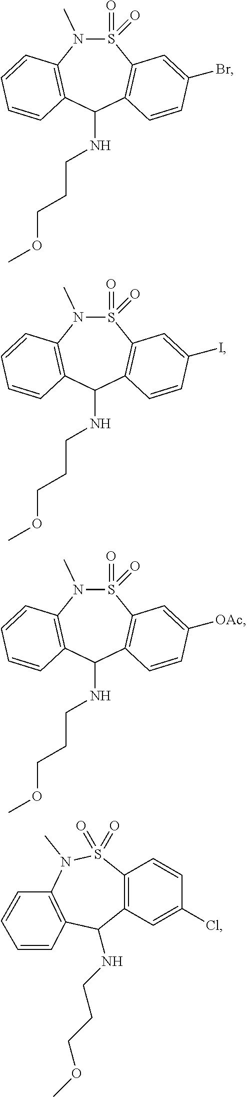

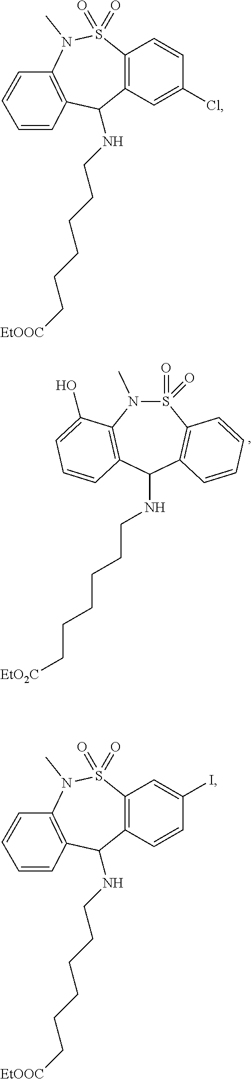 [SMILES: CCOC(=O)CCCCCCNC1c2ccccc2N(C)S(=O)(=O)c2ccc(Cl)cc21 ; CCOC(=O)CCCCCCNC1c2ccccc2S(=O)(=O)N(C)c2c(O)cccc21 ; CCOC(=O)CCCCCCNC1c2ccccc2N(C)S(=O)(=O)c2cc(I)ccc21]

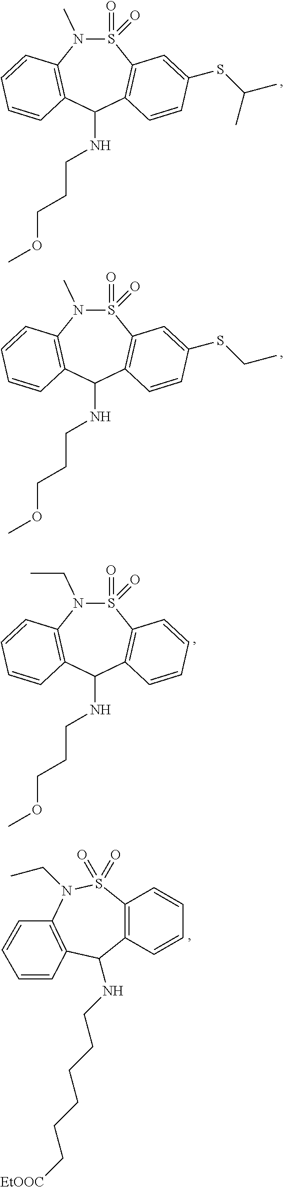

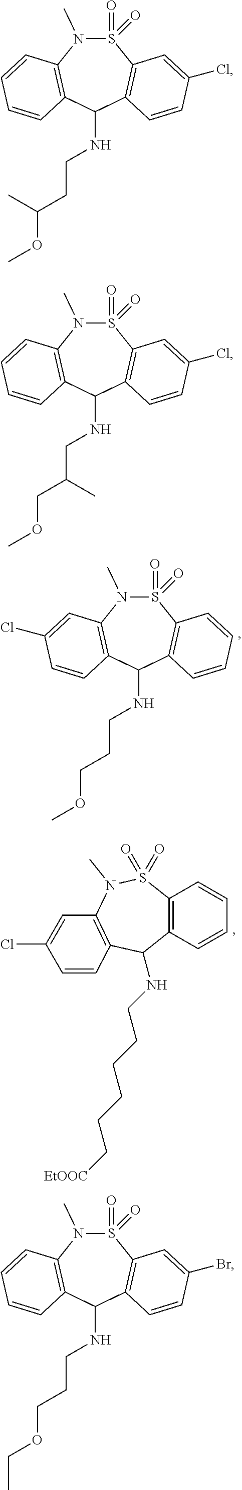

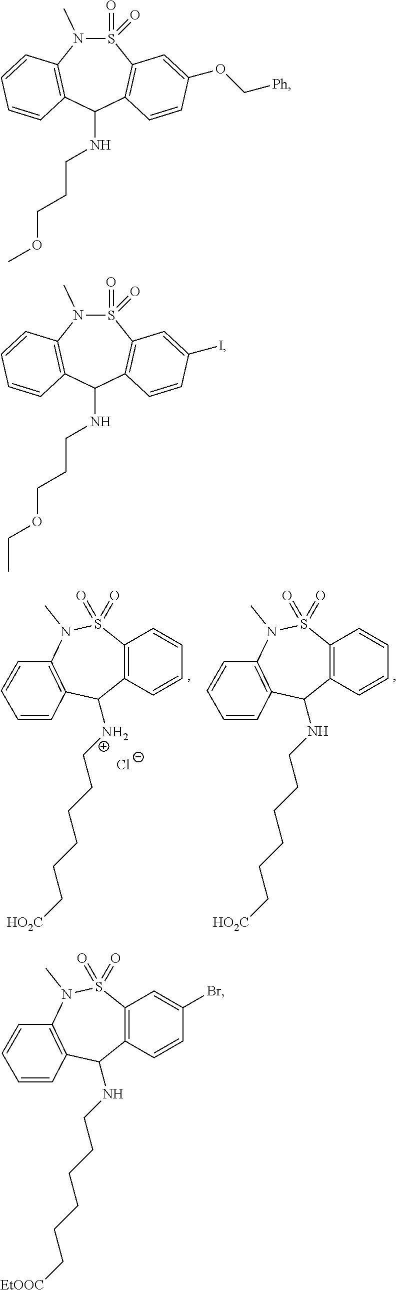 [SMILES: COCCCNC1c2ccccc2N(C)S(=O)(=O)c2cc(OCc3ccccc3)ccc21 ; CCOCCCNC1c2ccccc2N(C)S(=O)(=O)c2cc(I)ccc21 ; CN1c2ccccc2C([NH2+]CCCCCCC(=O)O)c2ccccc2S1(=O)=O ; [Cl-] ; CN1c2ccccc2C(NCCCCCCC(=O)O)c2ccccc2S1(=O)=O ; CCOC(=O)CCCCCCNC1c2ccccc2N(C)S(=O)(=O)c2cc(Br)ccc21]

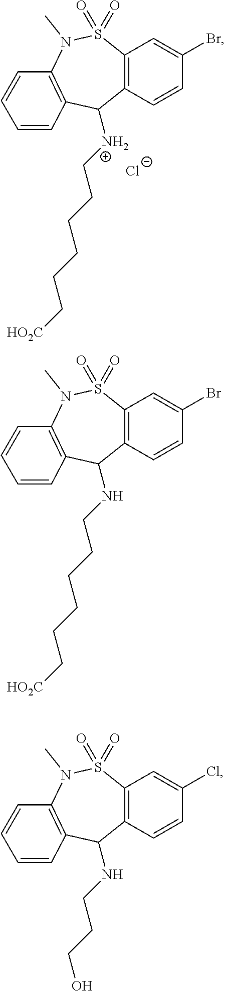 [SMILES: CN1c2ccccc2C([NH2+]CCCCCCC(=O)O)c2ccc(Br)cc2S1(=O)=O ; [Cl-] ; CN1c2ccccc2C(NCCCCCCC(=O)O)c2ccc(Br)cc2S1(=O)=O ; CN1c2ccccc2C(NCCCO)c2ccc(Cl)cc2S1(=O)=O]

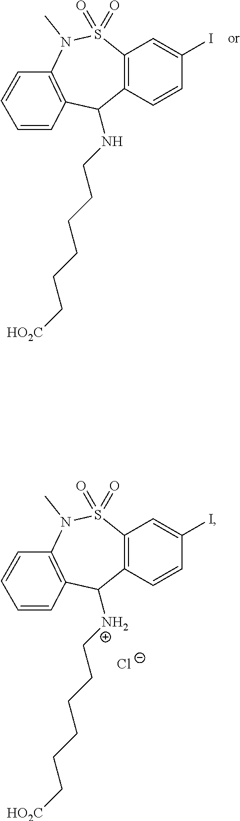 [SMILES: CN1c2ccccc2C(NCCCCCCC(=O)O)c2ccc(I)cc2S1(=O)=O ; CN1c2ccccc2C([NH2+]CCCCCCC(=O)O)c2ccc(I)cc2S1(=O)=O ; [Cl-]]

or a pharmaceutically acceptable salt thereof.

In some embodiments of the present method, compound, package, use or pharmaceutical composition, the compound has the structure:

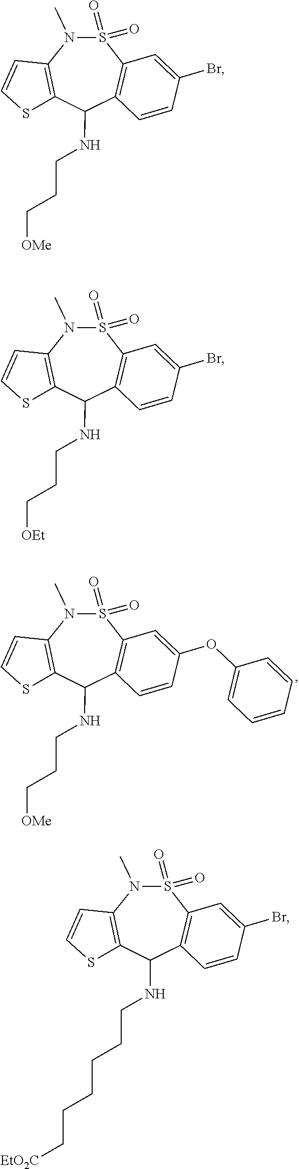 [SMILES: COCCCNC1c2ccc(Br)cc2S(=O)(=O)N(C)c2ccsc21 ; CCOCCCNC1c2ccc(Br)cc2S(=O)(=O)N(C)c2ccsc21 ; COCCCNC1c2ccc(Oc3ccccc3)cc2S(=O)(=O)N(C)c2ccsc21 ; CCOC(=O)CCCCCCNC1c2ccc(Br)cc2S(=O)(=O)N(C)c2ccsc21]

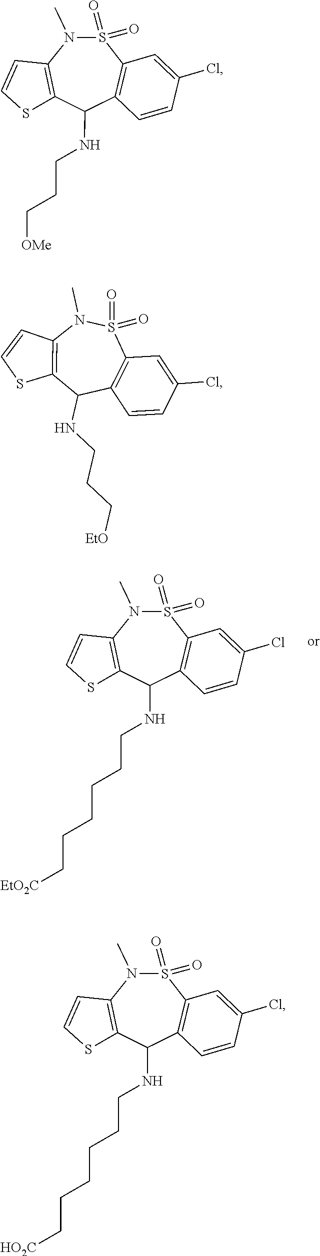

or a pharmaceutically acceptable salt thereof.

The present disclosure provides a compound having the structure:

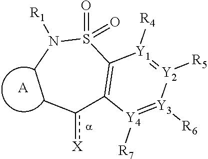

wherein
α is a bond, which may be present or absent;
X is O, OH, OTf, Cl, or Br,
   wherein when a is present, then X is O, and when a is absent, then X is OH, OTf, Cl, or Br; A is an aryl or heteroaryl, with or without substitution;
R₁ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
      or a pharmaceutically acceptable salt thereof.

In some embodiments of the above compound, the compound has the structure:

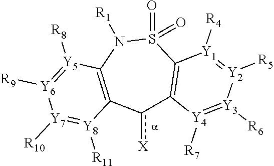

wherein R₈, R₉, R₁₀ and R₁₁ are each absent or present, and when present, are each independently —H, —Cl, —Br, —F, —I, —CN, —CF₃, —OCF₃, -(alkyl), -(aryl), -(heteroaryl)-(alkenyl), -(alkynyl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl);
Y₅, Y₆, Y₇ and Y₈ are each independently N or C,
   wherein when Y₅ is N, then R₈ is absent, and when Y₅ is C, then R₈ is present; when Y₆ is N, then R₉ is absent, and when Y₆ is C, then R₉ is present; when Y₇ is N, then R₁₀ is absent, and when Y₇ is C, then R₁₀ is present; when Y₈ is N, then R₁₁ is absent, and when Y₈ is C, then R₁₁ is present.

In some embodiments of the above compound, the compound has the structure:

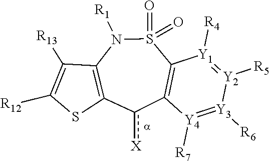

wherein R₁₂ and R₁₃ are each independently —H, —Cl, —Br, —F, —I, —CN, —CF₃, —OCF₃, -(alkyl), -(aryl), -(heteroaryl)-(alkenyl), -(alkynyl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl).

The present disclosure provides a process for producing a compound of this disclosure having the structure:

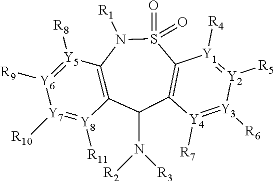

comprising
(a) contacting the compound having the structure:

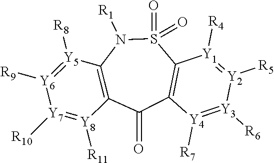

with a reducing agent in a first suitable solvent to produce a compound having the structure:

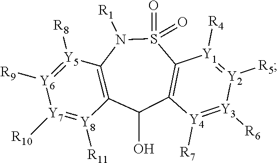

(b) reacting the product of step (a) with a halogenating agent or triflating agent in a second suitable solvent so as to produce a compound having the structure:

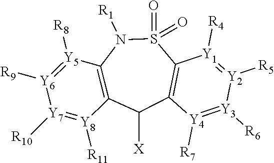

wherein X is OTf, C₁ or Br;
(c) reacting the product of step (b) with an amine in the presence of a base in a third suitable solvent so as to produce the compound having the structure:

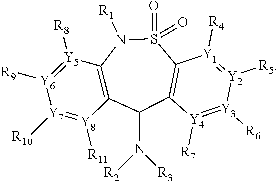

The present disclosure provides a process for producing a compound of this disclosure having the structure:

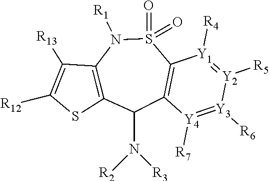

comprising
(a) contacting the compound having the structure:

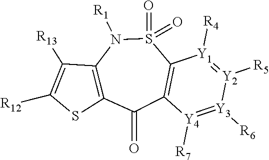

with a reducing agent in a first suitable solvent to produce a compound having the structure:

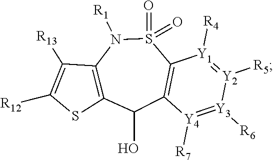

(b) reacting the product of step (a) with a halogenating agent or triflating agent in a second suitable solvent so as to produce a compound having the structure:

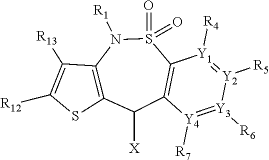

wherein X is OTf, C₁ or Br;
(c) reacting the product of step (b) with an amine in the presence of a base in a third suitable solvent so as to produce the compound having the structure:

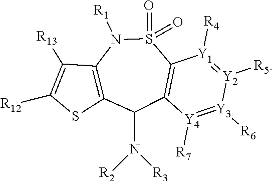

In some embodiments of the process, the reducing agent is sodium borohydride.

In some embodiments of the process, the halogenating agent is sulfonyl chloride or hydrogen chloride.

In some embodiments of the process, the amine is a primary amine or a secondary amine.

In some embodiments of the process, the first suitable solvent is methanol.

In some embodiments of the process, the second suitable solvent is dichloromethane.

In some embodiments of the process, the third suitable solvent is nitromethane.

In some embodiments, the aryl or heteroaryl A is substituted with —H, —Cl, —Br, —F, —I, —CN, —CF₃, —OCF₃, -(alkyl), -(aryl), -(heteroaryl)-(alkenyl), -(alkynyl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl).

In some embodiments, the aryl or heteroaryl A is substituted with Cl, Br, F, I, OH, —OCH₃, —CH₃.

In some embodiments, the aryl or heteroaryl A is substituted with —H, —Cl, —Br, —F, —I, —CN, —CF₃, —OCF₃, —OH, -(alkyl), —O-(alkyl), —S-(alkyl), —O-(aryl) or —S-(aryl).

In some embodiments, a compound having the structure

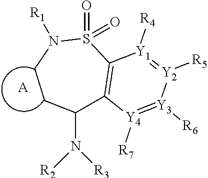

wherein
A is an aryl or heteroaryl, with or without substitution;
R₁ is —H or -(alkyl);
R₂ is —(C₁-C₆-alkyl), —(C₅-C₁₂-alkyl), —(C₁-C₂ alkyl)-CO₂H, —(C₅ alkyl)-CO₂H, —(C₇-C₁₂ alkyl)-CO₂H, —(C₁-C₅ alkyl)-CO₂—(C₁-C₁₂ alkyl), —(C₇-C₁₂ alkyl)-CO₂—(C₁-C₁₂ alkyl), -(alkenyl), -(alkynyl)-(alkyl)-OH, -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-OCH₃, -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O(heteroaryl)-S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
      or a pharmaceutically acceptable salt thereof.

In some embodiments, a compound having the structure

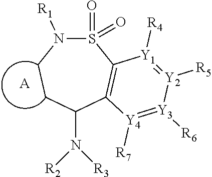

wherein
A is an aryl or heteroaryl, with or without substitution;
R₁ is —H or -(alkyl);
R₂ is —(C₁-C₅-alkyl), —(C₉-C₁₂-alkyl), —(C₁-C₂ alkyl)-CO₂H, —(C₈-C₁₂ alkyl)-CO₂H, —(C₁-C₄ alkyl)-CO₂—(C₁-C₁₂ alkyl), —(C₈-C₁₂ alkyl)-CO₂—(C₁-C₁₂ alkyl), -(alkenyl), -(alkynyl)-(alkyl)-OH, -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-OCH₃, -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl)-S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₃ is present; when Y₃ is N, then R₆ is absent, and when Y₃ is C, then R₆ is present; when Y₄ is N, then R₇ is absent, and when Y₄ is C, then R₇ is present,
      or a pharmaceutically acceptable salt thereof.

In some embodiments, a compound having the structure

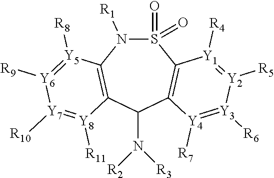

wherein
R₁ is —H or -(alkyl);
R₂ is -(alkyl), -(alkenyl), -(alkynyl)-(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-OCH₃, -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
R₈, R₉, R₁₀ and R₁₁ are each absent or present, and when present, are each independently —H, —Cl, —Br, —F, —I, —CN, —CF₃. —OCF₃, -(alkyl), -(aryl), -(heteroaryl)-(alkenyl), -(alkynyl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), -(0-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); Y₅, Y₆, Y₇ and Y₈ are each independently N or C,
   wherein when Y₅ is N, then R₈ is absent, and when Y₅ is C, then R₈ is present; when Y₆ is N, then R₉ is absent, and when Y₆ is C, then R₉ is present; when Y₇ is N, then R₁₀ is absent, and when Y₇ is C, then R₁₀ is present; when Y₈ is N, then R₁₁ is absent, and when Y₈ is C, then R₁₁ is present.
wherein when R₁ is —CH₃; R₃, R₄, R₆, R₇, R₈, R₉, R₁₀ and R₁₁ are each —H; Y₁, Y₂, Y₃, Y₄, Y₅, Y₆, Y₇ and Y₈ are each C; and R₅ is Cl, then R₂ is other than —(CH₂)₄CO₂H, —(CH₂)₆CO₂H, —(CH₂)₆CO₂CH₂CH₃, or —(CH₂)₆CH₃,
wherein when R₁ is —CH₃; R₃, R₄, R₆, R₇, R₈, R₉, R₁₀ and R₁₁ are each —H; Y₁, Y₂, Y₃, Y₄, Y₅, Y₆, Y₇ and Y₂ are each C; and R₅ is —SO₂CH₃, then R₂ is other than —(CH₂)₃OCH₃,
   or a pharmaceutically acceptable salt thereof.

In some embodiments of the compound, R₂ is —(C₁-C₆-alkyl), -(C₈-C₁₂-alkyl), —(C₁-C₃ alkyl)-CO₂H, —(C₅ alkyl)-CO₂H, —(C₇-C₁₂ alkyl)-CO₂H, —(C₁-C₅ alkyl)-CO₂—(C₁-C₁₂ alkyl), or —(C₇-C₁₂ alkyl)-CO₂—(C₁-C₁₂ alkyl).

In some embodiments of the compound, R₂ is —(C₁-C₅-alkyl), -(C₉-C₁₂-alkyl), —(C₁-C₂ alkyl)-CO₂H, -(C₈-C₁₂ alkyl)-CO₂H, —(C₁-C₄ alkyl)-CO₂—C₁-C₁₂ alkyl), -(C₈-C₁₂ alkyl)-CO₂—(C₁-C₁₂ alkyl).

In some embodiments of the compound, R₅ is —SO₂—(C₂-C₁₂ alkyl).

In some embodiments of the compound, R₅ is —SO₂—(C₃-C₁₂ alkyl)

In some embodiments, a compound having the structure:

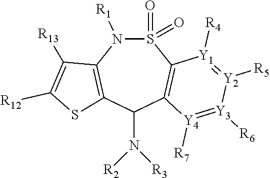

wherein
R₁ is —H or -(alkyl);
R₂ is -(alkyl), -(alkenyl), -(alkynyl)-(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-OCH₃, -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
   wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present; and
R₁₂ and R₁₃ are each independently —H, —Cl, —Br, —F, —I, —CN, —CF₃, —OCF₃, -(alkyl), -(aryl), -(heteroaryl)-(alkenyl), -(alkynyl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl).
   or a pharmaceutically acceptable salt thereof.

In some embodiments, a pharmaceutically acceptable salt of any of the above compounds of the present disclosure.

Various R₁-R₁₃ groups are added to the 6-methyl-6,11-dihydrodibenzo[1,2]thiazepine 5,5-dioxide core of the compounds disclosed herein. Said compounds act as MOR agonists with similar activity to compounds 6-11, 13-29, 31-53 or 55-84.

Various R₁-R₁₃ groups are added to the 4-methyl-4,10-dihydrobenzo[f]thieno[3,2-c][1,2]thiazepine 5,5-dioxide core of the compounds disclosed herein. Said compounds act as MOR agonists with similar activity to compounds 6-11, 13-29, 31-53 or 55-84.

Various R₂ groups replace the R₂ groups found on compounds 6-11, 13-29, 31-37, 29-53 or 55-58. Compounds with such R₂ groups act as MOR agonists with similar activity to compounds 6-11, 13-29, 31-53 or 55-84.

Various R₂ groups with similar chain lengths replace the R₂ groups found on compounds 6-11, 13-29, 31-37, 29-53 or 55-58. Compounds with such R₂ groups act as MOR agonists with similar activity to compounds 6-11, 13-29, 31-53 or 55-84.

Various R₁ groups replace the R₁ groups found on compounds 6-11, 13-29, 31-37, 29-53 or 55-58. Compounds with such R₁ groups act as MOR agonists with similar activity to compounds 6-11, 13-29, 31-53 or 55-84.

Various R₃ groups replace the R₃ groups found on compounds 6-11, 13-29, 31-37, 29-53 or 55-58. Compounds with such R₁ groups act as MOR agonists with similar activity to compounds 6-11, 13-29, 31-53 or 55-84.

Embodiments of the compounds disclosed herein include compounds where R₁ as H, ethyl, propyl, butyl, pentyl or hexyl. Compounds with R₁ as H, ethyl, propyl, butyl, pentyl or hexyl have analogous activity to compounds 6-11, 13-29, 31-53 or 55-84.

Embodiments of the compounds disclosed herein include compounds where R₃ as H, methyl, ethyl, propyl, butyl, pentyl or hexyl. Compounds with R₃ as H, methyl, ethyl, propyl, butyl, pentyl or hexyl have analogous activity to compounds 6-11, 13-29, 31-53 or 55-84.

Embodiments of the compounds disclosed herein include compounds where one or more of Y₁, Y₂, Y₃ or Y₄ is N. Compounds where one or more of Y₁, Y₂, Y₃ or Y₄ is N have analogous activity to compounds 6-11, 13-29, 31-53 or 55-584.

Derivatives of the compounds disclosed herein include compounds where one or more of Y₅, Y₆, Y₇ or Y₈ is N. Compounds where one or more of Y₅, Y₆, Y₇ or Y₈ is N have analogous activity to compounds 6-11, 13-29, 31-53 or 55-58.

In some embodiments, the compound is the structure of any one of compound 59, 60, 61, 62, 63, 64, 65, 66, 67, 68, 69, 70, 71, 72, 73, 74, 75, 76, 77, 78, 79, 80, 81, 82, 83 or 84.

In some embodiments, the compound has the structure of:

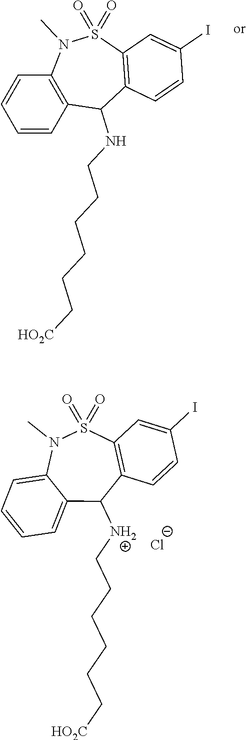 [SMILES: CN1c2ccccc2C(NCCCCCCC(=O)O)c2ccc(I)cc2S1(=O)=O ; "or" ; CN1c2ccccc2C([NH2+]CCCCCCC(=O)O)c2ccc(I)cc2S1(=O)=O ; [Cl-]]

In some embodiments, the compound used in any of the above methods, uses, packages or compositions is any one of compound 59, 60, 61, 62, 63, 64, 65, 66, 67, 68, 69, 70, 71, 72, 73, 74, 75, 76, 77, 78, 79, 80, 81, 82, 83 or 84.

In some embodiments, a salt of a compound of the present disclosure is used in any of the above methods, uses, packages or compositions.

In some embodiments, a pharmaceutically salt of a compound of the present disclosure is used in any of the above methods, uses, packages or compositions.

In some embodiments, an ester of a compound of the present disclosure is used in any of the above methods, uses, packages or compositions.

Any of the above compounds may be used in any of the disclosed methods, uses, packages or pharmaceutical compositions.

Any of the compounds used in the disclosed methods, uses, packages or pharmaceutical compositions may be replaced with any other compound disclosed in the present disclosure.

Any of the above generic compounds may be used in any of the disclosed methods, uses, packages or compositions.

In some embodiments, the methods, uses, packages or pharmaceutical compositions wherein the neurological disorder is a neurodegenerative condition or neurodevelopmental condition, for example, Huntington's disease; Rett syndrome; Rett syndrome variants, such as the Rett syndrome Rolando variant, the Rett syndrome Zappella variant, the Rett syndrome Hanefeld variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; and Lennox-Gastaut syndrome.

In some embodiments, the methods, uses, packages or pharmaceutical compositions wherein the neurological disorder is Huntington's disease; Rett syndrome; or CDKL5 disorder.

In some embodiments, the NMDA receptor antagonist is an aylcyclohexylamine, dextromorphinan or adamantane.

In some embodiments, the NMDA receptor antagonist is dextromethorphan, dextrorphan, dextrallorphan, memantine, amantadine, rimantadine, nitromemantine (YQW-36), ketamine (and its analogs, e.g., tiletamine), phencyclidine (and its analogs, e.g., tenocyclidine, eticyclidine, rolicyclidine), methoxetamine (and its analogs), gacyclidine (GK-11), neramexane, lanicemine (AZD6765), diphenidine, dizocilpine (MK-801), 8a-phenyldecahydroquinoline (8A-PDHQ), remacemide, ifenprodil, traxoprodil (CP-101,606), eliprodil (SL-82.0715), etoxadrol (CL-1848C), dexoxadrol, WMS-2539, NEFA, delucemine (NPS-1506), aptiganel (Cerestat; CNS-1102), midafotel (CPPene; SDZ EAA 494), dexanabinol (HU-211 or ETS2101), selfotel (CGS-19755), 7-chlorokynurenic acid (7-CKA), 5,7-dichlorokynurenic acid (5,7-DCKA), L-683344, L-689560, L-701324, GV150526A, GV196771A, CERC-301 (formerly MK-0657), atomoxetine, LY-235959, CGP 61594, CGP 37849, CGP 40116 (active enantiomer of CG 37849), LY-233536, PEAQX (NVP-AAM077), ibogaine, noribogaine, Ro 25-6981, GW468816, EVT-101, indantadol, perzinfotel (EAA-090), SSR240600, 2-MDP (U-23807A) or AP-7.

In some embodiments, the NMDA receptor partial agonist is a NRX-1074 or rapastinel (GLYX-13).

In some embodiments, the neurokinin 1 receptor antagonist is aprepitant, fosaprepitant, casopitant, maropitant, vestipitant, vofopitant, lanepitant, orvepitant, ezlopitant, netupitant, rolapitant, L-733060, L-703606, L-759274, L-822429, L-760735, L-741671, L-742694, L-732138, CP-122721, RPR-100893, CP-96345, CP-99994, TAK-637, T-2328, CJ-11974, RP 67580, NKP608, VPD-737, GR 205171, LY686017, AV608, SR140333B, SSR240600C, FK 888 or GR 82334.

In some embodiments, the neurokinin 2 receptor antagonist is saredutant, ibodutant, nepadutant, GR-159897 or MEN-10376.

In some embodiments, the neurokinin 3 receptor antagonist is osanetant, talnetant, SB-222200 or SB-218795.

The term “MOR agonist” is intended to mean any compound or substance that activates the mu-opioid receptor (MOR). The agonist may be a partial, full or super agonist.

The term “DOR agonist” is intended to mean any compound or substance that activates the delta-opioid receptor (DOR). The agonist may be a partial, full or super agonist.

Except where otherwise specified, the structure of a compound of this disclosure includes an asymmetric carbon atom, it is understood that the compound occurs as a racemate, racemic mixture, and isolated single enantiomer. All such isomeric forms of these compounds are expressly included in this disclosure. Except where otherwise specified, each stereogenic carbon may be of the R or S configuration. It is to be understood accordingly that the isomers arising from such asymmetry (e.g., all enantiomers and diastereomers) are included within the scope of this disclosure, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, NY, 1981. For example, the resolution may be carried out by preparative chromatography on a chiral column.

The present disclosure is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

In the compounds used in the method of the present disclosure, the substituents may be substituted or unsubstituted, unless specifically defined otherwise.

In the compounds used in the method of the present disclosure, alkyl, heteroalkyl, monocycle, bicycle, aryl, heteroaryl and heterocycle groups can be further substituted by replacing one or more hydrogen atoms with alternative non-hydrogen groups. These include, but are not limited to, halo, hydroxy, mercapto, amino, carboxy, cyano and carbamoyl.

It is understood that substituents and substitution patterns on the compounds used in the method of the present disclosure can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present disclosure, one of ordinary skill in the art will recognize that the various substituents, i.e., R₁, R₂, etc. are to be chosen in conformity with well-known principles of chemical structure connectivity.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2 . . . , n−1 or n carbons in a linear or branched arrangement, and specifically includes methyl, ethyl, propyl, butyl, pentyl, hexyl, heptyl, isopropyl, isobutyl, sec-butyl and so on. An embodiment can be C₁-C₁₂ alkyl, C₂-C₁₂ alkyl, C₃-C₁₂ alkyl, C₄-C₁₂ alkyl and so on. An embodiment can be C₁-C₃ alkyl, C₂-C₈ alkyl, C₃-C₈ alkyl, C₄-C₈ alkyl and so on. “Alkoxy” represents an alkyl group as described above attached through an oxygen bridge.

The term “alkenyl” refers to a non-aromatic hydrocarbon radical, straight or branched, containing at least 1 carbon to carbon double bond, and up to the maximum possible number of non-aromatic carbon-carbon double bonds may be present. Thus, C₂-Cₙ alkenyl is defined to include groups having 1, 2 . . . , n−1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. An embodiment can be C₂-C₁₂ alkenyl or C₂-C₃ alkenyl.

The term “alkynyl” refers to a hydrocarbon radical straight or branched, containing at least 1 carbon to carbon triple bond, and up to the maximum possible number of non-aromatic carbon-carbon triple bonds may be present. Thus, C₂-Cₙ alkynyl is defined to include groups having 1, 2 . . . , n−1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. An embodiment can be a C₂-Cₙ alkynyl. An embodiment can be C₂-C₁₂ alkynyl or C₃-C₅ alkynyl.

As used herein, “hydroxyalkyl” includes alkyl groups as described above wherein one or more bonds to hydrogen contained therein are replaced by a bond to an —OH group. In some embodiments, C₁-C₁₂ hydroxyalkyl or C₁-C₆ hydroxyalkyl. C₁-Cₙ as in “C₁-Cₙ alkyl” is defined to include groups having 1, 2, . . . , n−1 or n carbons in a linear or branched arrangement (e.g., C₁-C₂ hydroxyalkyl, C₁-C₃ hydroxyalkyl, C₁-C₄ hydroxyalkyl, C₁-C₅ hydroxyalkyl, or C₁-C₆ hydroxyalkyl) For example, C₁-C₆, as in “C₁-C₆ hydroxyalkyl” is defined to include groups having 1, 2, 3, 4, 5, or 6 carbons in a linear or branched alkyl arrangement wherein a hydrogen contained therein is replaced by a bond to an —OH group.

As used herein, “heteroalkyl” includes both branched and straight-chain saturated aliphatic hydrocarbon groups having the specified number of carbon atoms and at least 1 heteroatom within the chain or branch.

As used herein, “monocycle” includes any stable polyatomic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polyatomic carbon ring of up to 10 atoms that is fused to a polyatomic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include but are not limited to: phenyl, p-toluenyl (4-methylphenyl), naphthyl, tetrahydro-naphthyl, indanyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridazine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “heterocycle”, “heterocyclyl” or “heterocyclic” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The term “ester” is intended to a mean an organic compound containing the R—O—CO—R′ group.

The term “amide” is intended to a mean an organic compound containing the R—CO—NH—R′ or R—CO—N—R′R″ group.

The term “phenyl” is intended to mean an aromatic six membered ring containing six carbons.

The term “benzyl” is intended to mean a —CH₂R₁ group wherein the R₁ is a phenyl group.

The term “thiophene” is intended to mean a heteroaryl having a five-membered ring containing four carbon atoms and one sulfur atom.

The term “tetrahydrofuran” is intended to mean a heterocyclyl having a five-membered ring containing four carbon atoms and one 0 atom.

The term “pyrrolidine” is intended to mean a heterocyclyl having a five-membered ring containing four carbon atoms and one nitrogen atom.

The term “1,3 dioxane” is intended to mean a heterocyclyl having a six-membered ring containing four carbon atoms and two oxygen atoms.

The term “4,5-dihydrooxazole” is intended to mean a heterocyclyl having a five-membered ring containing 3 carbon atoms, one oxygen atom and one nitrogen atom.

The term “substitution”, “substituted” and “substituent” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropyl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

The compounds used in the method of the present disclosure may be prepared by techniques well known in organic synthesis and familiar to a practitioner ordinarily skilled in the art. However, these may not be the only means by which to synthesize or obtain the desired compounds.

The compounds used in the method of the present disclosure may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

The various R groups attached to the aromatic rings of the compounds disclosed herein may be added to the rings by standard procedures, for example those set forth in Advanced Organic Chemistry: Part B: Reactions and Synthesis, Francis Carey and Richard Sundberg, (Springer) 5th ed. Edition. (2007), the content of which is hereby incorporated by reference.

Another aspect of the disclosure comprises a compound used in the method of the present disclosure as a pharmaceutical composition.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department of Health and Human Services, 30^{th} edition, 2010), which are hereby incorporated by reference. Pharmaceutically active agents which have pendant carboxylic acid groups may be modified in accordance with the present disclosure using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present disclosure may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present disclosure. These salts can be prepared in situ during the final isolation and purification of the compounds of the disclosure, or by separately reacting a purified compound of the disclosure in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptanate, lactobionate, and laurylsulphonate salts and the like. (See, e.g., Berge et al. (1977) “Pharmaceutical Salts”, J. Pharm. Sci. 66:1-19).

Administration of one or more compounds and/or one or more compositions (e.g., pharmaceutical compositions) disclosed herein may be used for preventing, slowing, halting, or reversing the progression of a neurological disorder, as set out herein. Administration may also improve one or more symptoms of the neurological disorder.

The compounds used in the method of the present disclosure may be administered in various forms, including those detailed herein. The treatment with the compounds may be a component of a combination therapy or an adjunct therapy, i.e., the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier.

The dosage of the compounds administered in treatment will vary depending upon factors such as the pharmacodynamic characteristics of a specific therapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present disclosure may comprise a single compound or mixtures thereof with additional antibacterial agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g., by injection, topical application, or other methods, into or onto a site of infection, all using dosage forms well known to those of ordinary skill in the pharmaceutical arts.

The compounds used in the method of the present disclosure can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. Extended release formulations are specifically encompassed. The unit will be in a form suitable for oral, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present disclosure are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present disclosure may also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the method of the present disclosure may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydrcpyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water-soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the method of the present disclosure may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

In specific aspects, a disclosed compound may be administered at a dosage unit of about 0.1 mg to about 1000 mg, or about 1 mg to about 400 mg, or about 5 mg to about 300 mg, about 10 mg to about 200 mg, about 100 mg to about 200 mg, or at least 400 mg, at least 300 mg, at least 200 mg, at least 150 mg, at least 120 mg, at least 100 mg, at least 50 mg, at least 40 mg, at least 30 mg, at least 20 mg, at least 10 mg, at least 9 mg, at least 8.5 mg, at least 8 mg, at least 7.5 mg, at least 7 mg, at least 6.5 mg, at least 6 mg, at least 5.5 mg, at least 5 mg, at least 4.5 mg, at least 4 mg, at least 3.5 mg, at least 3 mg, at least 2.5 mg, at least 2 mg, or at least 1 mg.

In specific exemplifications, for a compound of the structure:

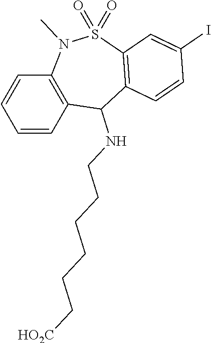 [SMILES: CN1c2ccccc2C(NCCCCCCC(=O)O)c2ccc(I)cc2S1(=O)=O]

or its pharmaceutically acceptable salts or esters thereof,
administration may be carried out at a dosage unit of about 0.5 mg to about 20 mg, or about 1 mg to about 10 mg, or about 2 mg to about 6 mg, or at least 10 mg, at least 9.5 mg, at least 9 mg, at least 8.5 mg, at least 8 mg, at least 7.5 mg, at least 7 mg, at least 6.5 mg, at least 6 mg, at least 5.5 mg, at least 5 mg, at least 4.5 mg, at least 4 mg, at least 3.5 mg, at least 3 mg, at least 2.5 mg, at least 2 mg, or at least 1 mg.

In further exemplifications, for a compound of the structure:

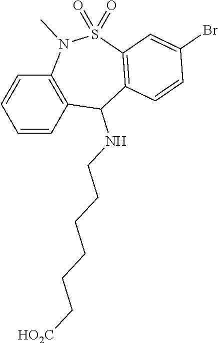 [SMILES: CN1c2ccccc2C(NCCCCCCC(=O)O)c2ccc(Br)cc2S1(=O)=O]

or its pharmaceutically acceptable salts or esters thereof,
administration may be carried out at a dosage unit of about 1 mg to about 60 mg, or about 3 mg to about 40 mg, or about 7.5 mg to about 20 mg, or at least 40 mg, at least 30 mg, at least 20 mg, at least 15 mg, at least 10 mg, at least 9.5 mg, at least 9 mg, at least 8.5 mg, at least 8 mg, at least 7.5 mg, at least 7 mg, at least 6.5 mg, at least 6 mg, at least 5.5 mg, at least 5 mg, at least 4.5 mg, at least 4 mg, at least 3.5 mg, or at least 3 mg.

The dosage units as noted herein may be administered once per day, twice per day, three times per day, four times per day, or more as needed. Administration of a dosage unit twice or three times per day is specifically noted. The dosage and administration regime may be adjusted for pediatric, geriatric, overweight, underweight, or other patients, where required. The dosage and administration regime may also be adapted for extended release formulations. All such modifications can be made in accordance with known methods.

Each embodiment disclosed herein is contemplated as being applicable to each of the other disclosed embodiments. Thus, all combinations of the various elements described herein are within the scope of this disclosure.

This invention will be better understood by reference to the Experimental Details which follow, but those skilled in the art will readily appreciate that the specific experiments detailed are only illustrative and that the invention is described in the claims which follow thereafter.

Experimental Details

Reagents and solvents were obtained from commercial sources and were used without further purification unless otherwise stated. All compounds were prepared in racemic form. All reactions were performed under argon atmosphere unless otherwise stated. All column chromatography was performed on silica gel (40-63 μm). Nuclear magnetic resonance spectra were recorded on Bruker 400 or 500 MHz instruments as indicated. Chemical shifts are reported as 5 values in ppm referenced to CDCl₃ (¹H NMR=7.26 and ¹³C NMR=77.16), Acetone-d₆ (1H NMR=2.05 and ¹³C NMR=29.84), or Methanol-d₄ (¹H NMR=3.31 and ¹³C NMR=49.00). Multiplicity is indicated as follows: s (singlet); d (doublet); t (triplet); q (quartet); p (pentet); h (heptet); dd (doublet of doublets); ddd (doublet of doublet of doublets); dt (doublet of triplets); td (triplet of doublets); m (multiplet); br (broad). For several compounds, spectra are complicated by the presence of conformers, C—F coupling, or the presence of diastereomers. Low-resolution mass spectra were recorded on a JEOL LCmate (ionization mode: APCI+). For compounds 4 and 5 mass spectra are reported for carbocations corresponding to loss of OH or Cl respectively.

Those having ordinary skill in the art of organic synthesis will appreciate that modifications to general procedures and synthetic routes contained in this application can be used to yield additional derivatives and structurally diverse compounds. Suitable organic transformations are described in March's Advanced Organic Chemistry: Reactions, Mechanisms, and Structure (Wiley-Interscience; 6^{th} edition, 2007), the content of which is hereby incorporated by reference.

### Preparation of the Amine Side-Chain (Used in Synthesis of 52)

### 7-Azidoheptanamide

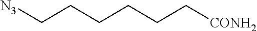 [SMILES: [N-]=[N+]=NCCCCCCC(N)=O]

The azide was prepared according the procedure described by Durand, P. et al. 1998. ¹H NMR (400 MHz, Chloroform-d) δ 5.45 (d, J=28.6 Hz, 2H), 3.26 (t, J=6.8 Hz, 2H), 2.23 (t, J=7.5 Hz, 2H), 1.72-1.54 (m, 4H), 1.47-1.31 (m, 4H).

### 7-Aminoheptanamide

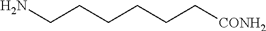

To a solution of the above azide (170 mg, 1.0 mmol) in THF (12 mL) and water (1 mL) was added Ph₃P (262 mg, 1.0 mmol). The resulting mixture was heated to 60° C. under Ar for 18 h. The solvent was removed and the crude product was used in the next step without further purification.

### Preparation of Sulfonyl Chlorides

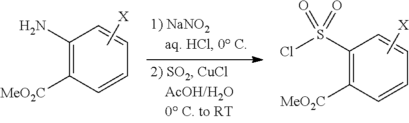

Methyl 4-chloro-2-(chlorosulfonyl)benzoate. A suspension of methyl 2-amino-4-chlorobenzoate (8.35 g, 45.0 mmol) in 20% aqueous HCl (29 mL) was sonicated for several minutes and warmed slightly until all clumps were broken up and the mixture was a uniform suspension of fine particles. This mixture was cooled to 0° C., and a solution of NaNO₂ (3.11 g, 45.0 mmol) in water (7.5 mL) was added dropwise, maintaining the internal temperature below 5° C. The resulting mixture was then stirred for 2 h at 0° C. A solution of SO₂ (23.1 g, 360 mmol) in AcOH (36.0 mL) and water (3.75 mL) was then prepared by bubbling the gas though the mixed solvents at 0° C. until the mass had increased by the required amount. To this SO₂ solution was then added CuCl (1.11 g, 11.25 mmol) followed by the diazonium salt solution portionwise over 30 minutes at 0° C. The resulting mixture was then stirred for 1 h at 0° C. and 1 h at room temperature, poured into ice water (150 mL), and extracted with CH₂Cl₂ (3×50 mL). The combined organics were poured into saturated aqueous NaHCO₃ (75 mL), and solid NaHCO₃ was added carefully until effervescence ceased. The organic phase was then separated, washed with brine (50 mL), dried over Na₂SO₄, and concentrated to provide the crude sulfonyl chloride as a red-brown oil (3.26 g, 81 mass % product by NMR, 22% yield). This material was used in the next step without further purification.

Methyl 4-bromo-2-(chlorosulfonyl)benzoate. Prepared from methyl 2-amino-4-bromobenzoate (10.35 g, 45.0 mmol) according to the procedure described above for methyl 4-chloro-2-(chlorosulfonyl)benzoate. The crude sulfonyl chloride was obtained as a waxy brown solid (5.15 g, 78 mass % product by NMR, 29% yield) and used in the next step without further purification.

Methyl 5-chloro-2-(chlorosulfonyl)benzoate. Prepared from methyl 2-amino-5-chlorobenzoate (5.00 g, 26.9 mmol) according to the procedure described for methyl 4-chloro-2-(chlorosulfonyl)benzoate. The crude sulfonyl chloride was obtained as a yellow oil (3.70 g, 36 mass % product by NMR, 19% yield) and used in the next step without further purification.

### Preparation of Substituted 4-Methylbenzo[f]thieno[3,2-c][1,2]thiazepin-10(4H)-one 5,5-dioxides 3a-d

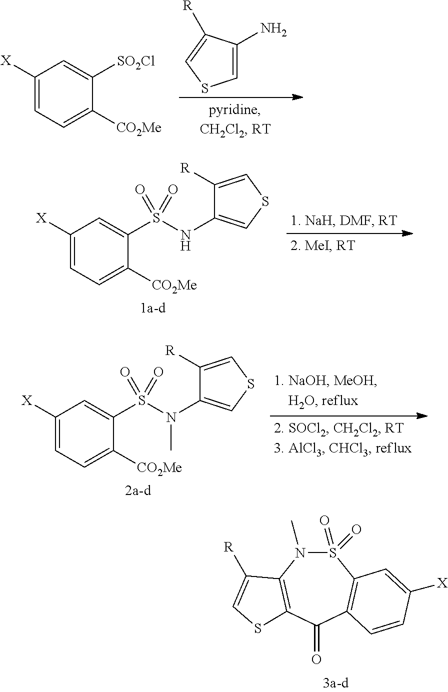

### Methyl 2-(N-(thiophen-3-yl)sulfamoyl)benzoate (1a)

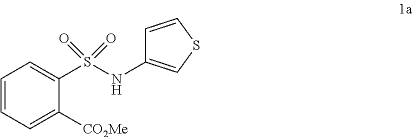 [SMILES: COC(=O)c1ccccc1S(=O)(=O)Nc1ccsc1]

A solution of methyl 2-(chlorosulfonyl)benzoate (1.15 g, 5.00 mmol) in anhydrous CH₂Cl₂ (10 mL) was added dropwise to a solution of thiophen-3-amine oxalate (1.04 g, 5.50 mmol) and pyridine (0.55 mL, 6.5 mmol) in dry CH₂Cl₂ (15 mL). The resulting mixture was stirred overnight at room temperature. The reaction mixture was then poured on ice and extracted with CH₂Cl₂. The combined organic layers were washed with HCl (1.0 M) followed by sat. NaHCO₃ and dried over Na₂SO₄. The crude product was purified by column chromatography (CH₂Cl₂+2% acetone). The product 1a was obtained as a yellowish oil (1.22 g, 82%). ¹H NMR (400 MHz, Acetone-d₆) δ ¹H NMR (400 MHz, Acetone-d₆) δ 7.92-7.84 (m, 1H), 7.81 (dd, J=7.6, 1.4 Hz, 1H), 7.73 (td, J=7.5, 1.3 Hz, 1H), 7.65 (td, J=7.7, 1.4 Hz, 1H), 7.35 (dd, J=5.2, 3.2 Hz, 1H), 7.04 (dd, J=3.2, 1.4 Hz, 1H), 6.96 (dd, J=5.2, 1.4 Hz, 1H), 3.98 (s, 3H); ¹³C NMR (101 MHz, Acetone-d₆) δ 168.8, 138.4, 136.0, 133.8, 132.7, 132.0, 131.0, 130.6, 126.4, 124.0, 113.6, 53.7; LR-MS calcd. for C₁₂H₁₂NO₄S₂ [M+H]⁺ 298.02, found 298.55.

### Methyl 2-(N-(4-bromothiophen-3-yl)sulfamoyl)benzoate (1b)

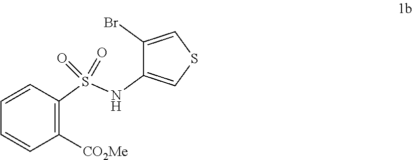 [SMILES: COC(=O)c1ccccc1S(=O)(=O)Nc1cscc1Br]

To a solution of 3-amino-4-bromothiophene (prepared according to the procedure described in Uy, R. et al. 2011) (2.01 g, 11.29 mmol) in anhydrous pyridine (8.0 mL) was carefully added methyl 2-(chlorosulfonyl)benzoate (2.52 g, 10.75 mmol) and the resulting solution was left to stir at room temperature for 15 min. The reaction was then diluted with CH₂Cl₂ (75 mL) and washed with 7% aq. HCl (2×75 mL), brine (50 mL), saturated aq. NaHCO₃ (75 mL), and brine again (50 mL). After drying over Na₂SO₄ and concentration the crude product was obtained as a black solid. This material was purified by column chromatography (CH₂Cl₂:hexanes—8:2) to yield sulfonamide 1b as a tan, crystalline solid (2.37 g, 59%). ¹H NMR (400 MHz, CDCl₃) δ 8.34 (s, 1H), 7.95 (dd, J=7.7, 1.3 Hz, 1H), 7.87 (dd, J=7.5, 1.4 Hz, 1H), 7.63 (td, J=7.6, 1.4 Hz, 1H), 7.57 (td, J=7.6, 1.5 Hz, 1H), 7.29 (d, J=3.6 Hz, 1H), 7.13 (d, J=3.6 Hz, 1H), 4.04 (s, 3H); ¹³C NMR (101 MHz, CDCl₃) δ 167.6, 138.7, 133.00, 132.97, 131.8, 131.2, 130.7, 130.0, 122.5, 113.4, 106.0, 53.6; LR-MS calcd. for C₁₂H₁₁BrNO₄S₂ [M+H]⁺ 377.93, found 378.38.

### Methyl 4-chloro-2-(N-(thiophen-3-yl)sulfamoyl)benzoate (1c)

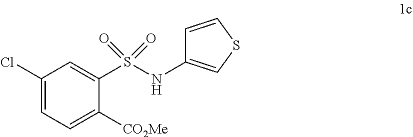 [SMILES: COC(=O)c1ccc(Cl)cc1S(=O)(=O)Nc1ccsc1]

To a suspension of thiophen-3-amine oxalate (2.01 g, 10.63 mmol) in anhydrous CH₂Cl₂ (10 mL) at 0° C. under argon was added anhydrous pyridine (7.0 mL) followed by a solution of crude methyl 4-chloro-2-(chlorosulfonyl)benzoate (3.22 g, 81% pure, 9.66 mmol) in anhydrous CH₂Cl₂ (10 mL) over ˜2 minutes. The resulting dark red-brown solution was then allowed to warm to room temperature and stirred for 1 h. The reaction mixture was then diluted with CH₂Cl₂ (100 mL) and washed with 3% aqueous HCl (2×50 mL), brine (50 mL), saturated aqueous NaHCO₃ (50 mL), and brine again (50 mL), dried over Na₂SO₄, and concentrated to give a dark-red oil (3.33 g). This material was purified by column chromatography (hexanes:EtOAc—8:2) to provide white crystals contaminated with oily brown impurities (2.33 g). These solids were washed 3× with small portions of 8:2 hexanes:EtOAc (removing the supernatant each time by pipette, impurities dissolve) to provide pure sulfonamide 1c as off-white crystals (2.13 g, 66%). ¹H NMR (500 MHz, CDCl₃) δ 8.21 (s, 1H), 7.89 (d, J=2.1 Hz, 1H), 7.80 (d, J=8.3 Hz, 1H), 7.55 (dd, J=8.3, 2.1 Hz, 1H), 7.18 (dd, J=5.1, 3.2 Hz, 1H), 6.94 (dd, J=3.2, 1.3 Hz, 1H), 6.89 (dd, J=5.2, 1.4 Hz, 1H), 4.03 (s, 3H); ¹³C NMR (126 MHz, CDCl₃) δ 167.5, 140.3, 138.5, 134.2, 132.7, 132.3, 130.8, 128.6, 125.9, 123.6, 114.7, 53.8; LR-MS calcd. for C₁₂H₁₁ClNO₄S₂ [M+H]⁺ 331.99, found 332.49.

### Methyl 4-bromo-2-(N-(thiophen-3-yl)sulfamoyl)benzoate (1d)

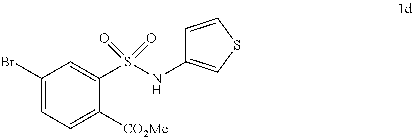 [SMILES: COC(=O)c1ccc(Br)cc1S(=O)(=O)Nc1ccsc1]

To a solution of crude methyl 4-bromo-2-(chlorosulfonyl)benzoate (5.10 g, 78% pure, 12.72 mmol) in anhydrous pyridine (9.6 mL) was added thiophen-3-amine oxalate (2.65 g, 13.99 mmol) at room temperature, and the resulting dark-red solution was stirred for 1 h. The reaction mixture was then diluted with CH₂Cl₂ (100 mL) and washed with 7% aqueous HCl (2×50 mL), brine (50 mL), saturated aqueous NaHCO₃ (50 mL), and brine again (50 mL), dried over Na₂SO₄, and concentrated to give a dark-red oil (2.41 g). This material was purified by column chromatography (hexanes:EtOAc—9:1, 2 column volumes→8:2, 2 column volumes→7:3, 2 column volumes) to provide off-white crystals contaminated with oily brown impurities (1.34 g). These solids were washed 2× with small portions of 7:3 hexanes:EtOAc (removing the supernatant each time by pipette, impurities dissolve) to provide pure sulfonamide 1d as tan crystals (1.16 g, 24%). ¹H NMR (500 MHz, CDCl₃) δ 8.19 (s, 1H), 8.04 (s, 1H), 7.75-7.69 (m, 2H), 7.18 (dd, J=5.1, 3.2 Hz, 1H), 6.94 (dd, J=3.2, 1.3 Hz, 1H), 6.89 (dd, J=5.1, 1.3 Hz, 1H), 4.02 (s, 3H); ¹³C NMR (126 MHz, CDCl₃) δ 167.6, 140.2, 135.8, 134.2, 133.5, 132.3, 129.1, 126.6, 125.9, 123.6, 114.7, 53.9; LR-MS calcd. for C₁₂H₁₁BrNO₄S₂[M+H]⁺ 375.93, found 376.29.

### Methyl 2-(N-methyl-N-(thiophen-3-yl) sulfamoyl)benzoate (2a)

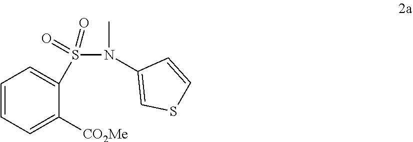 [SMILES: COC(=O)c1ccccc1S(=O)(=O)N(C)c1ccsc1]

A solution of sulfonamide 1a (1.2 g, 4.04 mmol) in anhydrous DMF (5 mL) was added dropwise to an ice-cold suspension of sodium hydride (60% dispersion in mineral oil, 350 mg, 8.1 mmol) in dry DMF (3 mL). After stirring for 1 h at room temperature, methyl iodide (0.5 mL, 8.1 mmol) was added dropwise, and the mixture was stirred overnight. The reaction mixture was poured on ice and the solid was filtered, washed with water, and dried. The crude product 2a was obtained as a white solid (in sufficient purity) and was used in the next step without further purification.

### Methyl 2-(N-(4-bromothiophen-3-yl)-N-methylsulfamoyl)benzoate (2b)

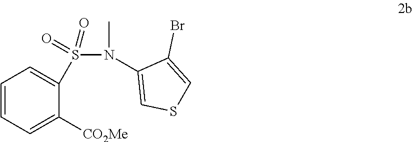 [SMILES: COC(=O)c1ccccc1S(=O)(=O)N(C)c1cscc1Br]

To a suspension of sodium hydride (60% dispersion in mineral oil, 498 mg, 12.44 mmol) in anhydrous DMF (9.0 mL) was added a solution of sulfonamide 1b (2.34 g, 6.22 mmol) in anhydrous DMF (9.0 mL) dropwise over 5 minutes and the resulting mixture was left to stir at room temperature for 1.75 h. Methyl iodide (1.77 g, 0.776 mL, 12.44 mmol) was then added and the mixture stirred for 2 h and then quenched with ice water (125 mL) and extracted with CH₂Cl₂ (2×50 mL, 25 mL). The combined organics were washed with water (2×50 mL), dried over Na₂SO₄, and concentrated to yield a dark-brown oil still containing residual DMF. This material was re-dissolved in Et₂O (50 mL), washed with water (4×50 mL), dried over Na₂SO₄, and concentrated to provide a biphasic oil. This crude material was purified by column chromatography (CH₂Cl₂) to yield sulfonamide 2b as an orange oil, which slowly crystallized into a waxy, orange solid (1.38 g, 57%). ¹H NMR (400 MHz, CDCl₃) δ 7.70 (dd, J=8.3, 0.9 Hz, 1H), 7.60 (td, J=7.6, 1.2 Hz, 1H), 7.53-7.47 (m, 2H), 7.28 (d, J=3.6 Hz, 1H), 7.22 (d, J=3.6 Hz, 1H), 3.86 (s, 3H), 3.33 (s, 3H); ¹³C NMR (101 MHz, CDCl₃) δ 168.3, 137.5, 136.9, 133.5, 132.7, 130.2, 129.7, 128.5, 125.4, 123.2, 111.8, 53.2, 39.0; LR-MS calcd. for C₁₃H₁₃BrNO₄S₂[M+H]⁺ 391.94, found 392.42.

### Methyl 4-chloro-2-(N-methyl-N-(thiophen-3-yl)sulfamoyl)benzoate (2c)

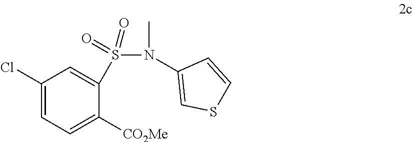 [SMILES: COC(=O)c1ccc(Cl)cc1S(=O)(=O)N(C)c1ccsc1]

To a suspension of sodium hydride (60% dispersion in mineral oil, 506 mg, 12.66 mmol) in anhydrous DMF (9 mL) was added a solution of sulfonamide 1c (2.1 g, 6.32 mmol) in anhydrous DMF (9 mL) dropwise over 5 minutes, and the resulting mixture was left to stir at room temperature for 1.5 h. Methyl iodide (1.79 g, 0.788 mL, 12.66 mmol) was then added, and the resulting mixture was stirred for 2.5 h and quenched with ice water (125 mL). This aqueous layer was extracted with CH₂Cl₂ (2×50 mL, 25 mL). The combined organics were washed with water (2×50 mL), dried over MgSO₄, and concentrated to yield a dark brown oil. This oil was washed with 3 portions of boiling hexanes (5 mL), cooling and carefully removing the supernatant by pipette each time, and dried in vacuo to yield sulfonamide 2c (1.83 g, 84%). ¹H NMR (300 MHz, CDCl₃) δ 7.53 (dd, J=8.2, 2.0 Hz, 1H), 7.44 (s, 1H), 7.41-7.39 (m, 1H), 7.29-7.25 (m, 1H), 7.05-7.01 (m, 2H), 3.88 (s, 3H), 3.27 (s, 3H); ¹³C NMR (75 MHz, CDCl₃) δ 167.9, 139.6, 137.1, 136.7, 133.1, 132.3, 130.14, 130.08, 125.9, 125.5, 119.1, 53.8, 39.3.

LR-MS cald. for C₁₃H₁₃ClNO₄S₂[M+H]⁺ 346.00, found 346.89.

### Methyl 4-bromo-2-(N-methyl-N-(thiophen-3-yl) sulfamoyl)benzoate (2d)

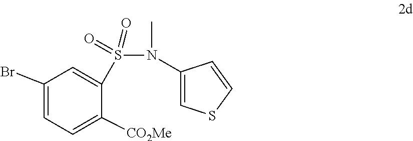 [SMILES: COC(=O)c1ccc(Br)cc1S(=O)(=O)N(C)c1ccsc1]

To a suspension of sodium hydride (60% dispersion in mineral oil, 244 mg, 6.12 mmol) in anhydrous DMF (4.5 mL) at 0° C. was added a solution of sulfonamide 1d (1.15 g, 3.06 mmol) in anhydrous DMF (4.5 mL) dropwise over 5 minutes, and the resulting mixture was allowed to warm to room temperature and stirred for 1.5 h. Methyl iodide (869 mg, 381 μL, 6.12 mmol) was then added dropwise over 2 minutes and the mixture was stirred for 2 h before quenching with ice water (50 mL) and extracting with Et₂O (3×25 mL). The combined organics were washed with water (2×25 mL) and brine (25 mL), dried over Na₂SO₄, and concentrated to provide a biphasic, pale-brown oil (1.26 g). The material was washed 3× with small portions of boiling hexanes, cooling and carefully removing the supernatant by pipette each time. The residue was then dried in vacuo to provide pure sulfonamide 2d as an orange-brown oil (1095 mg, 92%). ¹H NMR (500 MHz, CDCl₃) δ 7.69 (dd, J=8.2, 1.7 Hz, 1H), 7.52 (d, J=1.6 Hz, 1H), 7.35 (d, J=8.2 Hz, 1H), 7.30-7.27 (m, 1H), 7.04-7.01 (m, 2H), 3.88 (s, 3H), 3.26 (s, 3H); ¹³C NMR (126 MHz, CDCl₃) δ 167.6, 139.2, 136.4, 135.6, 132.5, 132.3, 129.7, 125.5, 125.1, 124.0, 118.7, 53.5, 38.8; LR-MS calcd. for C₁₃H₁₃BrNO₄S₂[M+H]⁺ 389.95, found 390.25.

### 4-Methylbenzo[f]thieno[3,2-c][1,2]thiazepin-10(4H)-one 5,5-dioxide (3a)

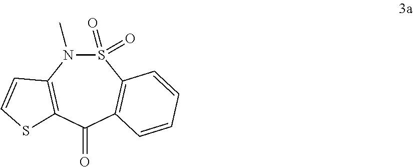 [SMILES: CN1c2ccsc2C(=O)c2ccccc2S1(=O)=O]

Sodium hydroxide (0.64 g) was added to a solution of crude sulfonamide 2a (1.2 g, 3.85 mmol) in a MeOH/water (2:1) solution (30 mL). After stirring for 2 h at reflux, the reaction mixture was cooled to 0° C. and acidified (pH 1-2) with 10% HCl. The solution was extracted with CH₂Cl₂ and the combined organic layers were dried over Na₂SO₄, and concentrated. The carboxylic acid was dissolved in anhydrous CH₂Cl₂ (20 mL) and thionyl chloride (1.16 mL, 16.0 mmol) was added. After stirring for 16 h at room temperature, the reaction mixture was concentrated and the residue was taken up in CHCl₃ (25 mL). Aluminium chloride (1.6 g, 12.0 mmol) was added and the mixture was refluxed for 1 h. The solvent was evaporated and water was added to the ice-cooled residue followed by extraction with CH₂Cl₂. The combined organic layers were dried over Na₂SO₄, filtered, and concentrated. The crude product was purified by column chromatography (CH₂Cl₂:hexanes—2:1). The product 3a was obtained as a yellowish solid (640 mg, 58% over 3 steps). ¹H NMR (400 MHz, Acetone-d₆) δ 8.15-8.03 (m, 3H), 8.00-7.91 (m, 2H), 7.34 (d, J=5.4 Hz, 1H), 3.48 (s, 3H); ¹³C NMR (101 MHz, Acetone-d₆) δ 183.6, 143.0, 137.5, 136.1, 135.0, 134.8, 134.2, 132.1, 126.5, 124.6, 38.2; LR-MS calcd. for C₁₂H₁₀NO₃S₂[M+H]⁺ 280.01, found 280.60.

### 3-Bromo-4-methylbenzo[f]thieno[3,2-c][1,2]thiazepin-10(4H)-one 5,5-dioxide (3b)

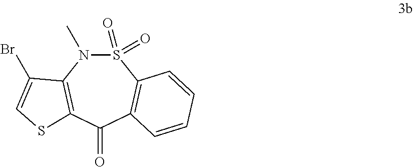 [SMILES: CN1c2c(Br)csc2C(=O)c2ccccc2S1(=O)=O]

To a solution of sulfonamide 2b (1.22 g, 3.13 mmol) in MeOH (12.5 mL) was added water (6.25 mL) and NaOH (376 mg, 9.39 mmol), and the mixture was refluxed for 1 h. At this time, 10% aq. HCl (5.0 mL) was added and the dense white cake of precipitate which had formed was broken up and washed from the reaction vessel with water. After stirring to break up clumps, the fine, white crystals were collected by vacuum filtration and dried to yield the carboxylic acid (1076 mg), which was used in the next step without further purification. The carboxylic acid (1072 mg, 2.85 mmol) was dissolved in thionyl chloride (12 mL), and the resulting solution left to stir for 13 h at room temperature. The volatiles were then removed to yield the crude acyl chloride as a light-brown solid which was used in the next step without further purification. The acyl chloride was re-dissolved in CHCl₃ (13 mL), aluminum chloride (1.22 g, 9.12 mmol) was added, and the resulting mixture was refluxed for 1 h. The reaction was then quenched with ice water (100 mL), stirred until all the brown sludge had broken up into a white suspension, and then extracted with CH₂Cl₂ (3×50 mL). The combined organics were washed with water (50 mL), dried over Na₂SO₄, and concentrated to yield a dark-brown solid. This crude material was purified by column chromatography (CH₂Cl₂:hexanes—6:4) to yield the pure ketone 3b as a tan, crystalline solid (653 mg, 58% over three steps). ¹H NMR (400 MHz, CDCl₃) δ 8.24-8.18 (m, 1H), 8.16-8.09 (m, 1H), 7.86-7.78 (m, 2H), 7.73 (s, 1H), 3.20 (s, 3H); ¹³C NMR (126 MHz, CDCl₃) δ 181.2, 140.7, 136.2, 136.0, 134.0, 133.6, 133.2, 132.7, 128.3, 110.2, 38.3; LR-MS calcd. for C₁₂H₉BrNO₃S₂[M+H]⁺ 359.92, found 360.08.

### 7-Chloro-4-methylbenzo[f]thieno[3,2-c][1,2]thiazepin-10(4H)-one 5,5-dioxide (3c)

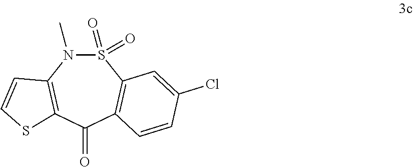 [SMILES: CN1c2ccsc2C(=O)c2ccc(Cl)cc2S1(=O)=O]

To a solution of sulfonamide 2c (1.83 g, 5.29 mmol) in MeOH (13 mL) and water (6.5 mL) was added sodium hydroxide (396 mg, 15.9 mmol). The resulting mixture was refluxed for 1.5 h after which time the reaction was cooled to room temperature and 10% HCl (10 mL) was added. The reaction was concentrated to remove most of the MeOH, and to this aqueous residue was added water (10 mL), and the mixture was then extracted with CH₂Cl₂ (30 mL, 2×20 mL). The combined organics were dried over Mg₂SO₄ and concentrated to yield the intermediate carboxylic acid as a tan crystalline solid (1.47 g, 84%) that was used without further purification. The carboxylic acid (1.47 g, 4.44 mmol) was dissolved in SOCl₂ (13.8 g, 8.47 mL, 116 mmol) and allowed to stir overnight under argon. After 14 h, the reaction mixture was concentrated to obtain the intermediate acyl chloride, and the resulting dark brown residue was dissolved in CHCl₃ (19.4 mL). Aluminum chloride (1.89 g, 14.21 mmol) was added, and the reaction mixture was refluxed for 1 hour after which time the solution was cooled to room temperature and quenched with ice water (100 mL). The resulting aqueous layer was extracted with CH₂Cl₂ (3×50 mL), and the combined organics were washed with water (50 mL), dried over MgSO₄, and concentrated. The crude product was purified by column chromatography (2:1 CH₂Cl₂:hexane→CH₂Cl₂) to give a yellow solid (686 mg, 49%). ¹H NMR (400 MHz, CDCl₃) δ 8.12 (d, J=8.4 Hz, 1H), 8.04 (d, J=2.1 Hz, 1H), 7.77-7.71 (m, 2H), 7.04 (d, J=5.4 Hz, 1H), 3.45 (s, 3H); ¹³C NMR (101 MHz, CDCl₃) δ 181.4, 144.2, 141.3, 139.7, 135.5, 133.7, 133.5, 132.2, 125.9, 122.8, 38.1; LR-MS cald. for C₁₂H₉ClNO₃S₂[M+H]⁺ 313.97, found 314.81.

### 7-Bromo-4-methylbenzo[f]thieno[3,2-c][1,2]thiazepin-10(4H)-one 5,5-dioxide (3d)

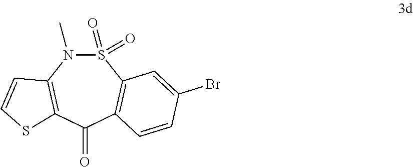 [SMILES: CN1c2ccsc2C(=O)c2ccc(Br)cc2S1(=O)=O]

To a solution of sulfonamide 2d (1085 mg, 2.78 mmol) in MeOH (7.0 mL) was added water (3.5 mL) and NaOH (334 mg, 8.34 mmol) and the mixture was refluxed for 1 h. The reaction was then cooled to room temperature, acidified with 10% aqueous HCl (5 mL), and most of the MeOH was removed in vacuo. The remaining aqueous residue was diluted with water (10 mL) and extracted with CH₂Cl₂ (20 mL, 2×10 mL). The combined organics were dried over Na₂SO₄ and concentrated to provide the carboxylic acid as a tan, crystalline solid (1.00 g), which was used in the next step without further purification. The carboxylic acid (990 mg, 2.63 mmol) was dissolved in thionyl chloride (5.0 mL) and the solution was stirred for 16 h at room temperature. The volatiles were then removed to provide the crude acyl chloride as a brown oil. This material was dissolved in CHCl₃ (11.5 mL), aluminum chloride (1.12 g, 8.42 mmol) was added, and the mixture was refluxed for 1 h. The reaction was then cooled to room temperature, quenched with ice water (50 mL), stirred until all the brown sludge had broken up, and then extracted with CH₂Cl₂ (3×25 mL). The combined organics were washed with water (25 mL), dried over Na₂SO₄, and concentrated to give a brown solid. This material was purified by column chromatography (CH₂Cl₂:hexanes—2:1, 5 column volumes→CH₂Cl₂, 3 column volumes) to provide pure ketone 3d as a pale-yellow, crystalline solid (579 mg, 59% over three steps). ¹H NMR (500 MHz, CDCl₃) δ 8.20 (d, J=1.9 Hz, 1H), 8.03 (d, J=8.3 Hz, 1H), 7.90 (dd, J=8.3, 2.0 Hz, 1H), 7.75 (d, J=5.4 Hz, 1H), 7.04 (d, J=5.4 Hz, 1H), 3.46 (s, 3H); ¹³C NMR (126 MHz, CDCl₃) δ 181.6, 141.3, 138.2, 136.8, 135.5, 133.4, 132.6, 131.6, 128.7, 127.8, 122.8, 38.1; LR-MS calcd. for C₁₂H₉BrNO₃S₂[M+H]⁺ 357.92, found 358.49.

### Preparation of Substituted 6-alkyldibenzo[c,f][1,2]thiazepin-11(6H)-one 5,5-dioxides 3f-3ac

Ketones 3f-3ac were prepared according to the procedures described below.

### 3-Chloro-6-methyldibenzo[c,f][1,2]thiazepin-11(6H)-one 5,5-dioxide (3f)

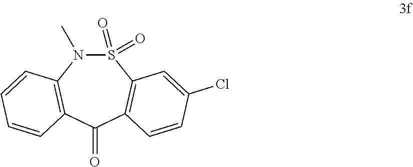 [SMILES: CN1c2ccccc2C(=O)c2ccc(Cl)cc2S1(=O)=O]

Ketone 3f was purchased from Ark Pharm, Inc. (Libertyville, IL) and used without further purification.

### 3-Fluoro-6-methyldibenzo[c,f][1,2]thiazepin-11(6H)-one 5,5-dioxide (3g)

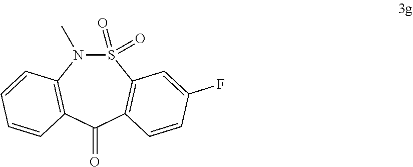 [SMILES: CN1c2ccccc2C(=O)c2ccc(F)cc2S1(=O)=O]

Ketone 3f (462 mg, 1.50 mmol) and cesium fluoride (684 mg, 4.50 mmol) were combined, anhydrous DMSO (3.0 mL) was added, and the mixture was heated to 180° C. for 20 min. After cooling to room temperature, the reaction was diluted with water (60 mL) and extracted with CH₂Cl₂ (20 mL, 2×15 mL). The combined organics were washed with water (50 mL), dried over Na₂SO₄, and concentrated to give a yellow glass. This was purified by column chromatography (CH₂Cl₂:Hexanes—8:2) to yield ketone 3g as a white solid (215 mg, 49%). ¹H NMR (400 MHz, CDCl₃) δ 8.32 (dd, J=8.1, 1.6 Hz, 1H), 8.03 (dd, J=8.6, 5.1 Hz, 1H), 7.71-7.62 (m, 2H), 7.43-7.34 (m, 3H), 3.36 (s, 3H); ¹³C NMR (126 MHz, CDCl₃) (additional peaks due to C—F coupling) δ 189.4, 165.2, 163.2, 141.3, 139.5, 139.4, 135.1, 134.8, 134.8, 132.5, 132.3, 131.2, 126.4, 124.9, 120.5, 120.3, 113.3, 113.1, 39.2; LR-MS calcd. for C₁₄H₁₁FNO₃S [M+H]⁺ 292.04, found 292.12.

### 3-Methoxy-6-methyldibenzo[c,f][1,2]thiazepin-11(6H)-one 5,5-dioxide (3h)

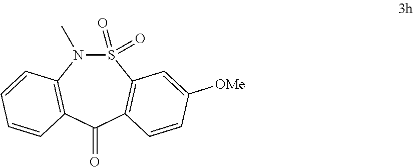 [SMILES: COc1ccc2c(c1)S(=O)(=O)N(C)c1ccccc1C2=O]

To a solution of sodium metal (115 mg, 5.00 mmol) in anhydrous MeOH (5.0 mL) was added ketone 3f (308 mg, 1.00 mmol) and the mixture was heated to 100° C. for 2 h in a sealed pressure vial. The reaction was then cooled to room temperature, diluted with water (10 mL), and extracted with CH₂Cl₂ (3×10 mL). The combined organics were washed with water (2×10 mL), dried over Na₂SO₄, and concentrated to yield a yellow crystalline solid. This material was recrystallized from MeOH to yield ketone 3h as white prisms (158 mg, 52%). ¹H NMR (500 MHz, CDCl₃) δ 8.33 (dd, J=8.1, 1.6 Hz, 1H), 8.04 (d, J=8.7 Hz, 1H), 7.63 (ddd, J=8.0, 7.3, 1.7 Hz, 1H), 7.46 (d, J=2.6 Hz, 1H), 7.42-7.34 (m, 2H), 7.18 (dd, J=8.7, 2.6 Hz, 1H), 3.96 (s, 3H), 3.33 (s, 3H); ¹³C NMR (126 MHz, CDCl₃) δ 189.0, 162.8, 141.2, 139.1, 134.6, 134.5, 132.4, 128.3, 126.6, 125.5, 118.9, 110.8, 56.3, 39.4; LR-MS calcd. for C₁₅H₁₄NO₄S [M+H]⁺ 304.06, found 303.91.

### 6-Methyl-3-phenoxydibenzo[c,f][1,2]thiazepin-11(6H)-one 5,5-dioxide (3i)

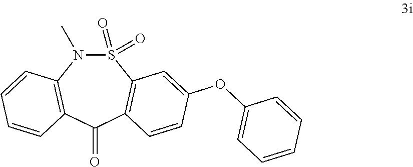 [SMILES: CN1c2ccccc2C(=O)c2ccc(Oc3ccccc3)cc2S1(=O)=O]

Ketone 3f (308 mg, 1.00 mmol), phenol (2.82 g, 30.0 mmol), and K₂CO₃ (691 mg, 5.00 mmol) were combined and heated to 150° C. for 2 h and then to 170° C. for 3.25 h. The hot reaction mixture was then carefully diluted with 10% aq. NaOH and extracted with Et₂O (3×30 mL). The combined organics were washed with water (30 mL) and brine (30 mL), dried over Na₂SO₄, and concentrated to yield a yellow foam. This material was purified by column chromatography (CH₂Cl₂:Hexanes—1:1, 3 column volumes→6:4, 3 column volumes) to give a pale-yellow foam still contaminated with impurities. This material was re-dissolved in CH₂Cl₂ and concentrated again to yield a yellow foam. A small quantity of Et₂O was then added to this material causing complete dissolution followed immediately by crystallization of the product as a cake of fine white crystals. After cooling on ice, the supernatant was removed by pipette and the mass of crystals was washed with small portions of ice-cold Et₂O and hexanes. After drying, the pure ketone 3i was obtained as short pale-yellow needles (288 mg, 79%). ¹H NMR (400 MHz, CDCl₃) δ 8.32 (dd, J=8.1, 1.6 Hz, 1H), 8.00 (d, J=8.6 Hz, 1H), 7.63 (ddd, J=8.0, 7.4, 1.7 Hz, 1H), 7.53 (d, J=2.5 Hz, 1H), 7.47-7.41 (m, 2H), 7.40-7.33 (m, 2H), 7.29-7.24 (m, 1H), 7.22 (dd, J=8.6, 2.5 Hz, 1H), 7.14-7.09 (m, 2H), 3.33 (s, 3H); ¹³C NMR (101 MHz, CDCl₃) δ 189.3, 161.3, 154.8, 141.2, 139.1, 134.7, 134.4, 132.2, 131.8, 130.5, 129.9, 126.4, 125.6, 125.1, 121.3, 120.4, 114.2, 39.2; LR-MS calcd. for C₂₀H₁₆NO₄S [M+H]⁺ 366.08, found 365.84.

### 3,6-Dimethyldibenzo[c,f][1,2]thiazepin-11(6H)-one 5,5-dioxide (3j)

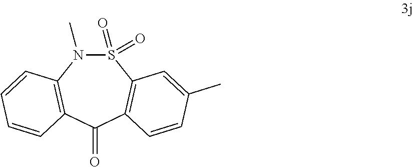 [SMILES: Cc1ccc2c(c1)S(=O)(=O)N(C)c1ccccc1C2=O]

The coupling was conducted according to the procedure of Dreher, S. D. et al. 2009 To a mixture of ketone 3f (308 mg, 1.0 mmol), potassium methyltrifluoroborate (135 mg, 1.1 mmol), K₂CO₃ (420 mg, 3.0 mmol), Pd(OAc)₂ (5.2 mg, 0.02 mmol), and RuPhos (19.2 mg, 0.04 mmol) was added toluene (4.5 mL) and H₂O (0.5 mL) (both solvents were de-oxygenated prior to use via standard “freeze-pump-thaw” cycle). The sealed reaction vial was heated at 85° C. under Ar for 36 h, and then cooled to room temperature. A saturated aqueous solution of NH₄Cl (10 mL) was added, and the resulting mixture was extracted with CH₂Cl₂. The combined organic layers were dried over Na₂SO₄ and concentrated. The crude product was purified by column chromatography (hexane:CH₂Cl₂—2:3->1:2). The product 3j was obtained as a white solid (280 mg, 97%). ¹H NMR (400 MHz, Acetone-d₆) δ 8.24 (dd, J=8.1, 1.6 Hz, 1H), 7.84 (d, J=7.9 Hz, 1H), 7.78 (s, 1H), 7.74 (ddd, J=8.2, 7.3, 1.7 Hz, 1H), 7.70-7.64 (m, 1H), 7.55 (dd, J=8.1, 0.9 Hz, 1H), 7.43 (td, J=7.7, 7.3, 1.1 Hz, 1H), 3.35 (s, 3H), 2.56 (s, 3H); ¹³C NMR (101 MHz, Acetone-d₆) δ 191.5, 144.5, 143.0, 137.4, 135.6, 135.0, 134.9, 132.2, 132.0, 131.8, 126.6, 126.3, 125.9, 39.4, 21.4; LR-MS calcd. for C₁₅H₁₄NO₃S [M+H]⁺ 288.07, found 288.59.

### 9-Bromo-3-chloro-6-methyldibenzo[c,f][1,2]thiazepin-11(6H)-one 5,5-dioxide (3k)

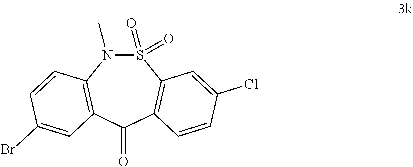 [SMILES: CN1c2ccc(Br)cc2C(=O)c2ccc(Cl)cc2S1(=O)=O]

N-bromosuccinimide (187 mg, 1.05 mmol) was added portionwise to a solution of ketone 3f (308 mg, 1.0 mmol) and FeCl₃ (324 mg, 2.0 mmol) in CH₂Cl₂ (10 mL) and CH₃CN (5 mL), and the reaction mixture was stirred at room temperature for 3 h. Additional N-bromosuccinimide (187 mg, 1.05 mmol) was then added and the reaction mixture was stirred at room temperature for a further 14 h. The reaction mixture was washed with water and brine and dried over NaSO₄. The crude product was purified by column chromatography (CH₂Cl₂:hexane—2:1) followed by crystallization from MeOH. The product 3k was obtained as a white solid (290 mg, 75%). ¹H NMR (400 MHz, Acetone-d₆) δ 8.31 (d, J=2.5 Hz, 1H), 8.00-7.96 (m, 1H), 7.95-7.89 (m, 3H), 7.58 (d, J=8.7 Hz, 1H), 3.44 (s, 3H); ¹³C NMR (101 MHz, Acetone-d₆) δ 189.6, 142.0, 139.1, 139.0, 138.4, 135.3, 134.7, 134.3, 132.8, 127.9, 125.8, 119.4, 39.3; LR-MS calcd. for C₁₄H₁₀BrClNO₃S [M+H]⁺ 387.92, found 388.63.

### 6-Methyl-3-(methylthio)dibenzo[c,f][1,2]thiazepin-11(6H)-one 5,5-dioxide (3l)

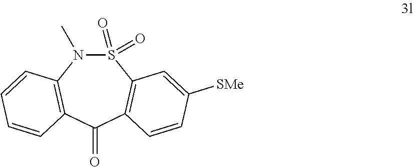 [SMILES: CSc1ccc2c(c1)S(=O)(=O)N(C)c1ccccc1C2=O]

To a solution of sodium thiomethoxide (116 mg, 1.65 mmol) in anhydrous DMF (2.0 mL) was added ketone 3f (462 mg, 1.50 mmol) and the resulting yellow suspension was stirred at room temperature for 1 h. Additional sodium thiomethoxide (26.3 mg, 0.375 mmol) was then added and stirring continued for a further 15 min. The reaction was then quenched with water (10 mL) and extracted with CH₂Cl₂ (10 mL, 2×5 mL). The combined organics were washed with water (20 mL), dried over Na₂SO₄, and concentrated to yield a yellow oil containing residual DMF. This crude was diluted with Et₂O and chilled on ice causing the product to crystallize as pale-yellow needles. These crystals were washed with several small portions of ice-cold Et₂O and dried to give the pure ketone 3l (331 mg, 69%). ¹H NMR (500 MHz, CDCl₃) δ 8.31 (dd, J=8.1, 1.6 Hz, 1H), 7.92 (d, J=8.3 Hz, 1H), 7.73 (d, J=2.0 Hz, 1H), 7.62 (ddd, J=8.0, 7.4, 1.7 Hz, 1H), 7.48 (dd, J=8.3, 2.0 Hz, 1H), 7.40-7.33 (m, 2H), 3.33 (s, 3H), 2.59 (s, 3H); ¹³C NMR (126 MHz, CDCl₃) δ 189.5, 146.5, 141.4, 137.7, 134.7, 132.4, 132.2, 131.8, 131.7, 129.2, 126.4, 125.2, 121.4, 39.3, 15.0. LR-MS calcd. for C₁₅H₁₄NO₃S₂[M+H]⁺ 320.04, found 320.75.

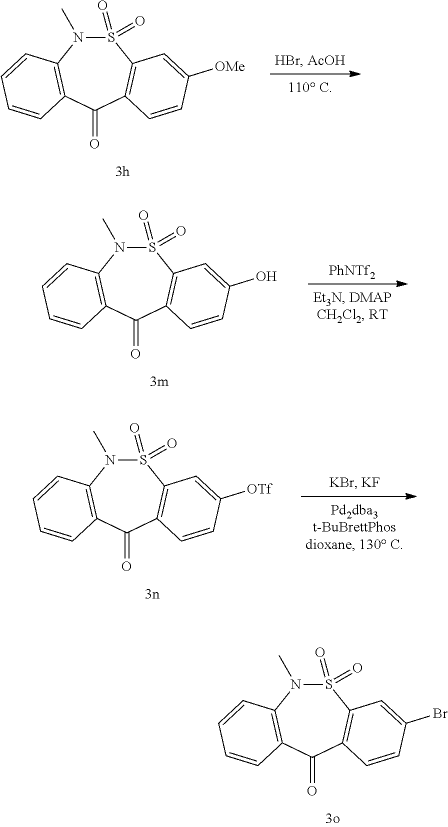

### 3-Hydroxy-6-methyldibenzo[c,f][1,2]thiazepin-11(6H)-one 5,5-dioxide (3m)

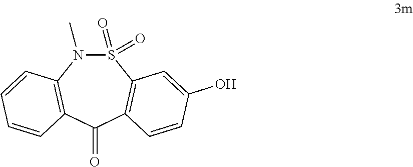 [SMILES: CN1c2ccccc2C(=O)c2ccc(O)cc2S1(=O)=O]

A solution of ketone 3f (330 mg, 1.09 mmol) in acetic acid (3 mL) and concentrated aqueous HBr (3 mL) was heated to 115° C. for 40 h. The reaction mixture was then poured into an ice-water mixture, and the precipitate was filtered and dissolved in CH₂Cl₂. The organic layer was dried over Na₂SO₄ and concentrated. The crude product was purified by column chromatography (CH₂Cl₂+3% acetone). The ketone 3m was obtained as a colorless oil (240 mg, 76%). ¹H NMR (400 MHz, Acetone-d₆) δ 9.82 (s, 1H), 8.28 (dd, J=8.1, 1.6 Hz, 1H), 7.91 (d, J=8.5 Hz, 1H), 7.72 (ddd, J=8.2, 7.2, 1.7 Hz, 1H), 7.55 (dd, J=8.1, 1.0 Hz, 1H), 7.43 (ddd, J=8.2, 7.2, 1.2 Hz, 1H), 7.40 (d, J=2.5 Hz, 1H), 7.25 (dd, J=8.5, 2.5 Hz, 1H), 3.36 (s, 3H); ¹³C NMR (101 MHz, Acetone-d₆) δ 189.9, 161.8, 142.6, 139.6, 135.4, 135.2, 132.5, 132.2, 128.5, 126.8, 126.4, 120.8, 112.8, 39.5. LR-MS calcd. for C₁₄H₁₂NO₄S [M+H]⁺ 290.05, found 289.99.

### 6-Methyl-5,5-dioxido-11-oxo-6,11-dihydrodibenzo[c,f][1,2]thiazepin-3-yl trifluoromethanesulfonate (3n)

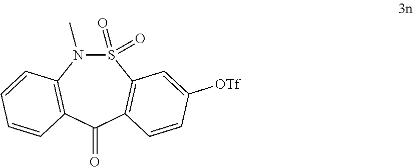 [SMILES: CN1c2ccccc2C(=O)c2ccc(OS(=O)(=O)C(F)(F)F)cc2S1(=O)=O]

To an ice-cold solution of ketone 3m (110 mg, 0.38 mmol), Et₃N (0.11 mL), and 4-dimethyl-aminopyridine (10 mg) in dry CH₂Cl₂ (10 mL) was added solid N-phenyl-bis(trifluoromethanesulfonimide) (170 mg, 0.49 mmol). The resulting reaction mixture was stirred at room temperature for 2.5 h. The mixture was concentrated and the crude product was purified by column chromatography (hexanes:EtOAc—3:1). The ketone 3n was obtained as a white solid (130 mg, 81%). ¹H NMR (400 MHz, Acetone-d₆) δ 8.24 (dd, J=8.1, 1.6 Hz, 1H), 8.16 (d, J=8.5 Hz, 1H), 8.03 (d, J=2.4 Hz, 1H), 8.00 (dd, J=8.5, 2.5 Hz, 1H), 7.84-7.76 (m, 1H), 7.62 (d, J=8.0 Hz, 1H), 7.53-7.45 (m, 1H), 3.43 (s, 3H); ¹³C NMR (101 MHz, Acetone-d₆) δ 190.7, 151.80, 142.7, 139.6, 137.6, 136.2, 135.3, 132.2, 131.2, 127.6, 127.0, 125.9, 119.4, 39.4; LR-MS calcd. for C₁₅H₁₁F₃NO₆S₂[M+H]⁺ 422.00, found 421.93.

### 3-Bromo-6-methyldibenzo[c,f][1,2]thiazepin-11(6H)-one 5,5-dioxide (3o)

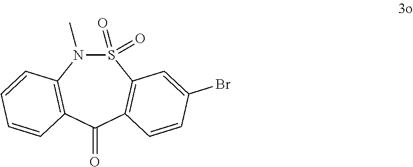 [SMILES: CN1c2ccccc2C(=O)c2ccc(Br)cc2S1(=O)=O]

The halide exchange was conducted using the procedure of Pan, J. et al. 2011. In a glove-box, triflate 3n (230 mg, 0.55 mmol), KBr (130 mg, 1.1 mmol), and KF (16 mg, 0.27 mmol) were added to a reaction vial and sealed with a screw-cap equipped with a teflon septum. To another reaction vial were added Pd₂(dba)₃ (10.2 mg, 2.0 mol %) and t-BuBrettPhos (15.9 mg, 6.0 mol %). The vial was sealed with a screw-cap equipped with a teflon septum. 1,4-Dioxane (0.7 mL) (de-oxygenated before use by standard “freeze-pump-thaw” technique) was added via syringe to the vial with the Pd-catalyst and phosphine, and the mixture was stirred at 120° C. in a preheated oil bath for 5 min. The catalyst solution was allowed to cool to room temperature and added to the reaction vial containing triflate 3n, KBr, and KF via syringe, followed by addition of dioxane (2 mL, de-oxygenated). The resulting mixture was stirred at 130° C. in a preheated oil bath for 8 h. The reaction mixture was poured on ice and extracted with CH₂Cl₂. The combined organic layers were washed with brine, dried over Na₂SO₄ and concentrated. The crude product was purified by column chromatography (CH₂Cl₂:hexane—2:1). The ketone 3o was obtained as a white solid (70 mg, 36%). ¹H NMR (400 MHz, Acetone-d₆) δ 8.23 (ddd, J=8.1, 1.7, 0.3 Hz, 1H), 8.10-8.04 (m, 2H), 7.88 (dt, J=8.4, 0.8 Hz, 1H), 7.77 (ddd, J=8.2, 7.2, 1.7 Hz, 1H), 7.59 (dd, J=8.2, 1.0 Hz, 1H), 7.46 (ddd, J=8.3, 7.2, 1.2 Hz, 1H), 3.42 (s, 3H); ¹³C NMR (101 MHz, Acetone) δ 190.1, 141.9, 138.1, 136.6, 135.5, 135.0, 133.2, 131.2, 130.5, 127.6, 125.9, 125.0, 38.5. LR-MS calcd. for C₁₄H₁₁BrNO₃S [M+H]⁺ 351.96, found 351.83.

### 6-Methyl-3-(trimethylsilyl)dibenzo[c,f][1,2]thiazepin-11(6H)-one 5,5-dioxide (3p)

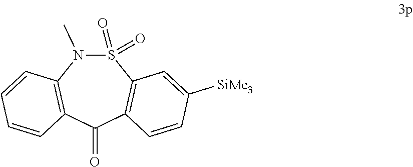 [SMILES: CN1c2ccccc2C(=O)c2ccc([Si](C)(C)C)cc2S1(=O)=O]

Ketone 3p was prepared from the aryl chloride utilizing the trimethylsilylation procedure of McNiell, E. et al. 2007. Ketone 3f (462 mg, 1.50 mmol), Pd₂dba₃ (20.6 mg, 0.0225 mmol), t-BuDavePhos (2′-(Di-tert-butylphosphino)-N,N-dimethylbiphenyl-2-amine, 46.1 mg, 0.135 mmol), and LiOAc (495 mg, 7.50 mmol) were combined under argon. Anhydrous DMF (4.5 mL), water (54 μL, 3.00 mmol), and hexamethyldisilane (369 μL, 1.80 mmol) were then added, and the resulting orange-brown mixture was heated to 100° C. for 33 h. After cooling to room temperature, the reaction mixture was diluted with water (20 mL) and extracted with Et₂O (3×10 mL). The combined organics were washed with water (10 mL), dried over Na₂SO₄, and concentrated to yield a yellow crystalline solid. This crude was recrystallized from MeOH to obtain pure ketone 3p as fine yellow needles (301 mg, 58%). ¹H NMR (500 MHz, CDCl₃) δ 8.30 (dd, J=8.1, 1.6 Hz, 1H), 8.04 (d, J=0.8 Hz, 1H), 7.91 (d, J=7.5 Hz, 1H), 7.85 (dd, J=7.6, 1.1 Hz, 1H), 7.63 (ddd, J=8.1, 7.3, 1.7 Hz, 1H), 7.41-7.29 (m, 2H), 3.35 (s, 3H), 0.36 (s, 9H); ¹³C NMR (126 MHz, CDCl₃) δ 191.2, 147.4, 141.9, 138.3, 136.6, 136.0, 134.8, 132.1, 131.2, 130.5, 129.7, 126.0, 124.6, 39.1, -1.2; LR-MS calcd. for C₁₇H₂₀NO₃SSi [M+H]⁺ 346.09, found 345.86.

### 3-Iodo-6-methyldibenzo[c,f][1,2]thiazepin-11(6H)-one 5,5-dioxide (3q)

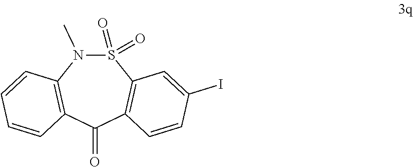 [SMILES: CN1c2ccccc2C(=O)c2ccc(I)cc2S1(=O)=O]

To a solution of ketone 3p (108 mg, 0.313 mmol) in anhydrous CH₂Cl₂ (0.94 mL) at 0° C. was added a solution of iodine monochloride (173 mg, 1.06 mmol) in anhydrous CH₂Cl₂ (0.63 mL) dropwise over 3 min. The resulting dark-brown solution was allowed to warm to room temperature, stirred for 35 min (extended reaction times produce polyiodinated byproducts), and quenched with saturated aqueous Na₂S₂O₃ (3 mL). The resulting mixture was diluted with water (15 mL) and extracted with CH₂Cl₂ (2×15 mL). The combined organics were washed with water (15 mL), dried over Na₂SO₄, and concentrated to yield a yellow solid. This material was purified by column chromatography (CH₂Cl₂:Hexanes—1:1) to yield impure product. This crude product was recrystallized from MeOH and the resulting fine-white needles were dissolved in CH₂Cl₂ and concentrated, causing a second crystallization to occur once most of the solvent had been removed. The powdery white crystals thus obtained were washed with ice-cold MeOH and dried to yield the pure ketone 3q (68.4 mg, 55%). ¹H NMR (400 MHz, CDCl₃) δ 8.32-8.27 (m, 2H), 8.06 (dd, J=8.1, 1.7 Hz, 1H), 7.69-7.62 (m, 2H), 7.38 (ddd, J=8.2, 7.3, 1.1 Hz, 1H), 7.34 (dd, J=8.1, 0.9 Hz, 1H), 3.35 (s, 3H); ¹³C NMR (101 MHz, CDCl₃) δ 190.1, 142.4, 141.5, 138.1, 135.6, 135.1, 133.9, 133.1, 132.2, 131.0, 126.3, 124.7, 98.7, 39.2; LR-MS calcd. for C₁₄H₁₁INO₃S [M+H]⁺ 399.95, found 399.78.

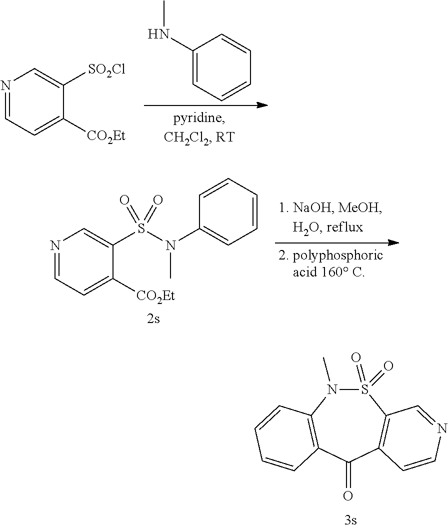

### Ethyl 3-(N-methyl-N-phenylsulfamoyl) isonicotinate (2s)

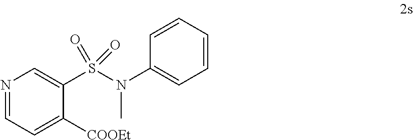 [SMILES: CCOC(=O)c1ccncc1S(=O)(=O)N(C)c1ccccc1]

A solution of ethyl 3-(chlorosulfonyl)isonicotinate (300 mg, 1.27 mmol) in anhydrous CH₂Cl₂ (8 mL) was added dropwise to a solution of N-methylaniline (0.17 mL, 1.52 mmol) and pyridine (0.15 mL, 1.65 mmol) in dry CH₂Cl₂ (10 mL). The resulting mixture was stirred overnight at room temperature. The reaction mixture was then poured on ice and extracted with CH₂Cl₂. The combined organic layers were dried over Na₂SO₄. The crude product was purified by column chromatography (CH₂Cl₂->CH₂Cl₂+2% acetone). The product 2s was obtained as a colorless oil (330 mg, 81%). ¹H NMR (400 MHz, Acetone-d₆) δ 8.87 (d, J=5.0 Hz, 1H), 8.47 (s, 1H), 7.61 (d, J=5.0 Hz, 1H), 7.44-7.32 (m, 3H), 7.31-7.24 (m, 2H), 4.36 (q, J=7.1 Hz, 2H), 3.34 (s, 3H), 1.33 (t, J=7.1 Hz, 3H); ¹³C NMR (101 MHz, Acetone-d₆) δ 166.4, 154.6, 150.7, 142.0, 141.8, 131.2, 130.1, 128.7, 128.0, 122.8, 63.3, 39.1, 14.2; LR-MS calcd. for C₁₅H₁₇N₂O₄S [M+H]⁺ 321.09, found 321.15.

### 11-Methylbenzo[f]pyrido[3,4-c][1,2]thiazepin-5(11H)-one 10,10-dioxide (3s)

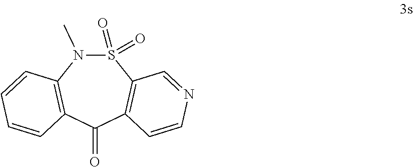 [SMILES: CN1c2ccccc2C(=O)c2ccncc2S1(=O)=O]

Sodium hydroxide (0.43 g) was added to a solution of sulfonamide 2s (0.35 g, 1.09 mmol) in a MeOH/water (1:1) solution (10 mL). After stirring for 1.5 h at reflux, the reaction mixture was cooled to 0° C. and acidified (pH 1-2) with 10% HCl. The white solid was filtered and dried. Polyphosphoric acid (11 g) was added to a high-pressure reaction flask containing the crude product and sealed with a teflon cap. The reaction mixture was heated to 160° C. for 5 h and while still hot poured on ice, basified by NaOH (15%) to pH ˜12, and extracted with ethyl acetate. The combined organic layers were washed with brine and dried over Na₂SO₄. The crude product was purified by column chromatography (ethyl acetate). The product 3s was obtained as a white solid (100 mg, 33%). ¹H NMR (400 MHz, Acetone-d₆) δ 9.04 (s, 1H), 8.99 (d, J=4.9 Hz, 1H), 8.03 (dd, J=8.1, 1.2 Hz, 1H), 7.86-7.76 (m, 3H), 7.55 (ddd, J=8.4, 6.9, 1.6 Hz, 1H), 3.68 (s, 3H); ¹³C NMR (101 MHz, Acetone-d₆) δ 165.3, 156.5, 144.9, 141.3, 139.7, 138.0, 137.5, 136.2, 127.9, 127.9, 127.4, 126.0, 39.4; LR-MS calcd. for C₁₃H₁₁N₂O₃S [M+H]⁺ 275.30, found 274.90.

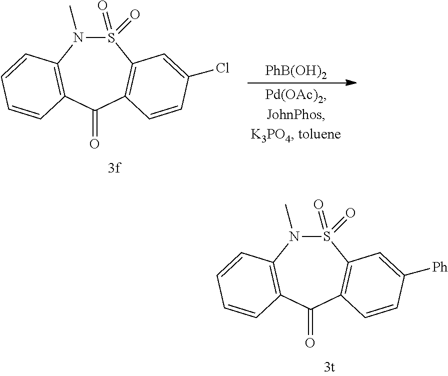 [SMILES: "PhB(OH)2" ; CN1c2ccccc2C(=O)c2ccc(Cl)cc2S1(=O)=O ; CN1c2ccccc2C(=O)c2ccc(-c3ccccc3)cc2S1(=O)=O]

### 6-Methyl-3-phenyldibenzo[c,f][1,2]thiazepin-11(6H)-one 5,5-dioxide (3t)

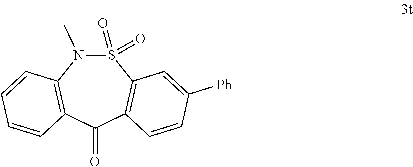 [SMILES: CN1c2ccccc2C(=O)c2ccc(-c3ccccc3)cc2S1(=O)=O]

The coupling was conducted according to the procedure of Buchwald et al. 2004. Ketone 3f (308 mg, 1.0 mmol), phenylboronic acid (228 mg, 1.5 mmol), K₃PO₄ (425 mg, 2.0 mmol), Pd(OAc)₂ (2.2 mg, 1 mcl %) and 2-(di-tert-butylphosphino)biphenyl (JohnPhos) (6.0 mg, 2 mcl %) were added to a reaction vial and sealed with a screw-cap equipped with a teflon septum. The vial was evacuated and backfilled with argon (3×), and toluene (3 mL, de-oxygenated before use by standard “freeze-pump-thaw” technique) was added via syringe, and the reaction mixture was stirred at 100° C. for 18 h. The reaction mixture was diluted with CH₂Cl₂, washed with water, dried over Na₂SO₄ and concentrated. The crude product was crystallized from MeOH/ethyl acetate (˜5:1). The ketone 3t was obtained as orange crystals (315 mg, 90%). ¹H NMR (400 MHz, Acetone-d₆) δ 8.27 (dd, J=8.1, 1.7 Hz, 1H), 8.18 (dd, J=1.9, 0.3 Hz, 1H), 8.15 (dd, J=8.0, 1.9 Hz, 1H)), 8.04 (dd, J=8.0, 0.4 Hz, 1H), 7.89-7.83 (m, 2H)), 7.76 (ddd, J=8.2, 7.2, 1.7 Hz, 1H), 7.61-7.54 (m, 3H), 7.51 (dd, J=7.3, 1.3 Hz, 1H), 7.45 (ddd, J=8.2, 7.2, 1.2 Hz, 1H), 3.42 (s, 3H)); ¹³C NMR (101 MHz, Acetone-d₆) δ 191.5, 145.8, 143.1, 139.0, 138.2, 136.0, 135.7, 133.0, 132.4, 132.1, 131.7, 130.2, 129.9, 128.2, 126.6, 125.9, 124.0, 39.4; LR-MS calcd. for C₂₀H₁₆NO₃S [M+H]⁺ 350.08, found 350.14.

### 6-Methyl-3-(methylsulfonyl)dibenzo[c,f][1,2]thiazepin-11(6H)-one 5,5-dioxide (3u)

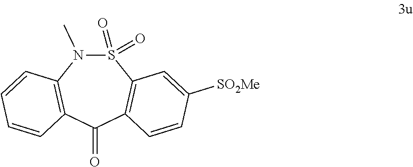 [SMILES: CN1c2ccccc2C(=O)c2ccc(S(C)(=O)=O)cc2S1(=O)=O]

To a suspension of ketone 3l (222 mg, 0.695 mmol) in MeOH (3.0 mL) at 0° C. was added a solution of Oxone (855 mg, 1.39 mmol) in water (3.0 mL), and the resulting white suspension was allowed to warm to room temperature and stirred for 18.5 h. The reaction mixture was then diluted with water (10 mL) and extracted with CH₂Cl₂ (3×10 mL). The combined organics were washed with water (10 mL), dried over Na₂SO₄, and concentrated to yield a white solid. This crude material was purified by column chromatography (CH₂Cl₂:Et₂O—20:1) to provide the pure ketone 3u as a white crystalline solid (220 mg, 90%). ¹H NMR (400 MHz, CDCl₃) δ 8.49 (d, J=1.7 Hz, 1H), 8.31-8.24 (m, 2H), 8.11 (d, J=8.0 Hz, 1H), 7.69 (ddd, J=8.1, 7.3, 1.7 Hz, 1H), 7.44-7.38 (m, 1H), 7.36 (dd, J=8.2, 0.8 Hz, 1H), 3.40 (s, 3H), 3.16 (s, 3H); ¹³C NMR (101 MHz, CDCl₃) δ 190.1, 144.0, 141.6, 140.9, 138.6, 135.5, 133.0, 132.2, 132.0, 130.1, 126.3, 124.4, 124.1, 44.5, 39.0; LR-MS calcd. for C₁₅H₁₄NO₅S₂[M+H]⁺ 352.03, found 352.31.

### 6-Methyl-3-(methylsulfinyl)dibenzo[c,f][1,2]thiazepin-11(6H)-one 5,5-dioxide (3v)

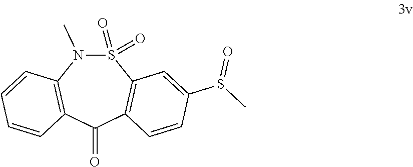 [SMILES: CN1c2ccccc2C(=O)c2ccc(S(C)=O)cc2S1(=O)=O]

To a suspension of ketone 3l (216 mg, 0.676 mmol) in MeOH (3.0 mL) at 0° C. was added a solution of Oxone (229 mg, 0.372 mmol) in water (3.0 mL), and the resulting pale-yellow suspension was allowed to warm to room temperature and stirred for 2.5 h. The reaction mixture was then diluted with water (10 mL) and extracted with CH₂Cl₂ (3×10 mL). The combined organics were washed with water (10 mL), dried over Na₂SO₄, and concentrated to yield an off-white foam. This crude material was purified by column chromatography (CH₂Cl₂:Et₂O—8:2) to provide the pure ketone 3v as an off-white crystalline solid (157 mg, 69%). ¹H NMR (400 MHz, Acetone-d₆) δ 8.26 (d, J=1.5 Hz, 1H), 8.23 (dd, J=8.1, 1.6 Hz, 1H), 8.14 (dd, J=8.0, 1.6 Hz, 1H), 8.09 (d, J=8.0 Hz, 1H), 7.78 (ddd, J=8.8, 7.3, 1.7 Hz, 1H), 7.59 (dd, J=8.2, 0.8 Hz, 1H), 7.49-7.43 (m, 1H), 3.42 (s, 3H), 2.90 (s, 3H); ¹³C NMR (101 MHz, Acetone-d₆) δ 191.6, 153.4, 143.0, 139.2, 138.4, 136.0, 133.0, 132.1, 131.2, 129.5, 126.7, 125.7, 121.1, 44.1, 39.4; LR-MS calcd. for C₁₅H₁₄NO₄S₂[M+H]⁺ 336.04, found 336.23.

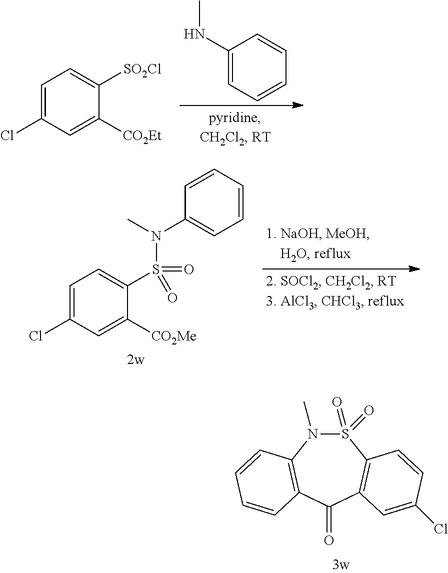

### 2-Chloro-6-methyldibenzo[c,f][1,2]thiazepin-11(6H)-one 5,5-dioxide (3w)

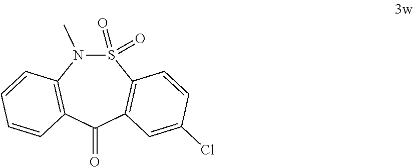 [SMILES: CN1c2ccccc2C(=O)c2cc(Cl)ccc2S1(=O)=O]

To a solution of N-methylaniline (0.65 mL, 6.0 mmol) and anhydrous pyridine (0.55 mL, 6.5 mmol) in anhydrous CH₂Cl₂ (20 mL) was added a solution of methyl 5-chloro-2-(chlorosulfonyl)benzoate (3.70 g of crude material, 36 mass % pure=1.35 g, 5.0 mmol) in anhydrous CH₂Cl₂ (10 mL), and the mixture was allowed to stir at room temperature overnight. The reaction was then diluted with water and extracted with CH₂Cl₂ (3×30 mL). The combined organics were washed with 1M aqueous HCl (30 mL) and saturated aqueous NaHCO₃ (30 mL), dried over Na₂SO₄, and concentrated to provide the crude product. This material was purified by column chromatography (hexanes:EtOAc—3:1) to provide sulfonamide 2w as an orange oil still containing slight impurities (1.30 g), which was used in the next step without further purification. To a solution of sulfonamide 2w (1.30 g) in MeOH (20 mL) was added water (10 mL) and NaOH (0.66 g, 13.5 mmol), and the resulting mixture was refluxed for 1.5 h. After cooling to room temperature, the mixture was made strongly acidic with 10% aqueous HCl and extracted with CH₂Cl₂. The combined organics were dried over Na₂SO₄ and concentrated. The crude carboxylic acid thus obtained was dissolved in anhydrous CH₂Cl₂ (20 mL), thionyl chloride (2.0 mL, 27.4 mmol) was added, and the solution was stirred for 6 h at room temperature before concentrating in vacuo. The crude acyl chloride thus obtained was dissolved in CHCl₃ (20 mL), aluminum chloride (1.50 g, 11.2 mmol) was added, and the resulting mixture was refluxed for 1.5 h. The volatiles were then removed in vacuo and the residue was quenched with cold water and extracted with CH₂Cl₂. The combined organics were dried over Na₂SO₄ and concentrated to provide the crude product. This material was purified by column chromatography (1:1 CH₂Cl₂:hexanes—1:1) followed by recrystallization from methanol to provide the pure ketone 3w as a pinkish solid (450 mg, 29% from sulfonyl chloride). ¹H NMR (400 MHz, Acetone) δ 8.21 (dd, J=8.1, 1.6 Hz, 1H), 8.00-7.95 (m, 1H), 7.93-7.88 (m, 2H), 7.77 (ddd, J=8.3, 7.3, 1.7 Hz, 1H), 7.58 (dd, J=8.2, 0.9 Hz, 1H), 7.48-7.42 (m, 1H), 3.40 (s, 3H); ¹³C NMR (101 MHz, Acetone) δ 190.8, 143.0, 140.0, 139.2, 136.2, 136.0, 133.0, 132.2, 131.8, 131.2, 128.0, 126.7, 125.8, 39.4; LR-MS calcd. for C₁₄H₁₁ClNO₃S [M+H]⁺ 308.01, found 308.37.

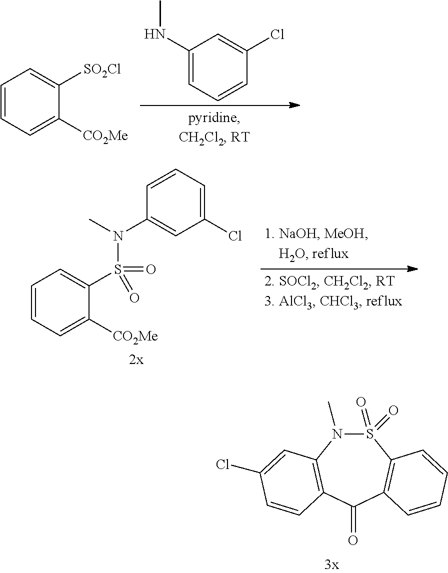

### 8-Chloro-6-methyldibenzo[c,f][1,2]thiazepin-11(6H)-one 5,5-dioxide (3x)

To a solution of N-methyl-3-chloroaniline (643 μL, 5.25 mmol) in anhydrous pyridine (3.8 mL) was added methyl 2-(chlorosulfonyl)benzoate (1.17 g, 5.00 mmol), and the resulting mixture was stirred for 3 h at room temperature. The reaction mixture was then diluted with CH₂Cl₂ (35 mL), washed with 7% aqueous HCl (2×35 mL), brine (35 mL), saturated aqueous NaHCO₃ (35 mL), and brine again (35 mL), dried over Na₂SO₄, and concentrated to provide pure sulfonamide 2× as a viscous yellow oil (1.14 g, 67%). To a solution of sulfonamide 2× (1.09 g, 3.21 mmol) in MeOH (8.2 mL) was added water (4.1 mL) and NaOH (385 mg, 3.21 mmol), and the resulting mixture was refluxed for 45 minutes. The solution was diluted with water (50 mL), washed with Et₂O (30 mL), made strongly acidic with 10% aqueous HCl, and extracted with CH₂Cl₂ (30 mL, 2×20 mL). The combined organics were washed with water (20 mL), dried over Na₂SO₄, and concentrated to provide the pure carboxylic acid intermediate as a yellow solid (1.04 g, 99%). The carboxylic acid (1.00 g, 3.07 mmol) was dissolved in thionyl chloride (6.6 mL), and the resulting solution was stirred for 2 h and then concentrated in vacuo to provide the intermediate acyl chloride as a tan solid. This material was dissolved in CHCl₃ (13 mL), aluminum chloride (1.31 g, 9.82 mmol) was added, and the resulting mixture was refluxed for 1 h. The reaction was then quenched with ice water (100 mL) and extracted with CH₂Cl₂ (50 mL, 2×25 mL). The combined organics were washed with water (50 mL), dried over Na₂SO₄, and concentrated to give the crude product as a viscous brown oil. When a small quantity of MeOH was added to this oil, tan crystals formed. These were crushed up and the supernatant was removed by pipet. The crystals were washed with an additional small portion of ice-cold MeOH and then recrystallized from MeOH to provide pure ketone 3× as glistening tan needles (400 mg, 42%). ¹H NMR (500 MHz, CDCl₃) δ 8.27 (d, J=8.6 Hz, 1H), 7.97-7.91 (m, 2H), 7.76-7.70 (m, 2H), 7.35-7.30 (m, 2H), 3.35 (s, 3H); ¹³C NMR (126 MHz, CDCl₃) δ 189.9, 142.8, 140.8, 136.8, 136.3, 133.6, 133.5, 132.3, 131.7, 129.2, 126.2, 125.3, 124.1, 38.8; LR-MS calcd. for C₁₄H₁₁ClNO₃S [M+H]⁺ 308.01, found 308.47.

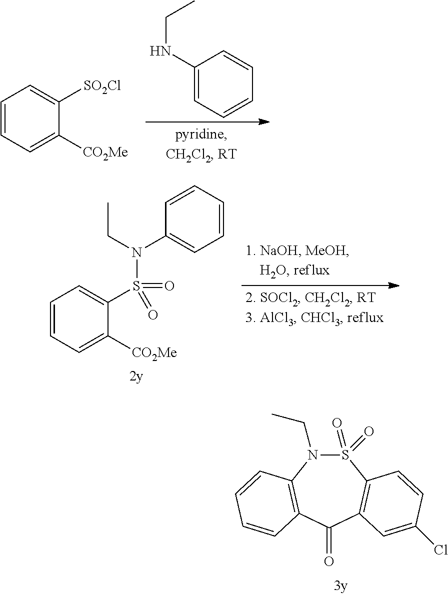

### 6-Ethyldibenzo[c,f][1,2]thiazepin-11(6H)-one 5,5-dioxide (3y)

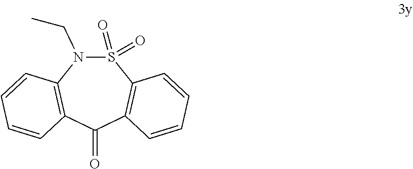 [SMILES: CCN1c2ccccc2C(=O)c2ccccc2S1(=O)=O]

Sulfonamide 2y was synthesized from 2-(chlorosulfonyl)benzoate (1.17 g, 5.00 mmol) and N-ethylaniline (663 μL, 5.25 mmol) according to the procedure described for 2× and obtained as a viscous, yellow-orange oil (1.20 g, 75%). Sulfonamide 2y (1.14 g, 3.58 mmol) was converted to the corresponding carboxylic acid according to the procedure described under the synthesis of ketone 3x, and obtained as a viscous brown oil (1.09 g, 99%). This carboxylic acid (1.06 g, 3.47 mmol) was dissolved in thionyl chloride (7.5 mL), stirred for 2 h at room temperature, and then concentrated in vacuo to provide the acyl chloride intermediate as a viscous yellow oil. This material was dissolved in CHCl₃ (15 mL), aluminum chloride (1.48 g, 11.1 mmol) was added, and the resulting mixture was refluxed for 1.5 h. The reaction was then quenched with ice water (100 mL) and extracted with CH₂Cl₂ (50 mL, 2×25 mL). The combined organics were washed with water (50 mL), dried over Na₂SO₄, and concentrated to give the crude product as a dark-green glass. This material was purified by column chromatography (CH₂Cl₂:hexanes—8:2) to provide a tan solid (297 mg) still containing some impurities. This was then recrystallized from MeOH to provide pure ketone 3y as off-white crystals (231 mg, 23%) ¹H NMR (400 MHz, CDCl₃) δ 8.31-8.27 (m, 1H), 8.02-7.96 (m, 2H), 7.74-7.68 (m, 2H), 7.66-7.60 (m, 1H), 7.42-7.36 (m, 2H), 3.84 (q, J=7.2 Hz, 2H), 0.98 (t, J=7.2 Hz, 3H); ¹³C NMR (101 MHz, CDCl₃) δ 191.0, 140.1, 139.1, 136.1, 134.7, 133.2, 132.9, 132.4, 132.2, 131.8, 126.5, 125.5, 125.4, 47.5, 14.1; LR-MS calcd. for C₁₅H₁₄NO₃S [M+H]⁺ 288.07, found 288.08.

### 3-(Ethylthio)-6-methyldibenzo[c,f][1,2]thiazepin-11(6H)-one 5,5-dioxide (3z)

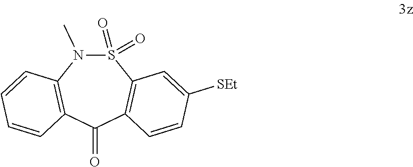

To a suspension of NaH (60% dispersion in mineral oil, 28.0 mg, 0.700 mmol) in anhydrous DMF (0.67 mL) was added ethanethiol (50 μL, 0.675 mmol), and the resulting mixture was stirred at room temperature for 15 minutes. Ketone 3f (154 mg, 0.500 mmol) was then added, and the mixture was stirred for 1.75 h at room temperature. The reaction was quenched with water (10 mL) and extracted with CH₂Cl₂ (10 mL, 2×5 mL). The combined organics were washed with water (20 mL), dried over Na₂SO₄ and concentrated to give a yellow oil still containing residual DMF (mostly removed by heating under high vacuum). When a small quantity of Et₂O (˜3 mL) was added to this oil, fine white needles crystallized from the mixture. After cooling on ice, the supernatant was removed by pipette and the crystals were washed with several small portions of ice-cold Et₂O and then dried in vacuo to provide pure ketone 3z as fine white needles (108 mg, 65%). ¹H NMR (400 MHz, CDCl₃) δ 8.30 (dd, J=8.0, 1.1 Hz, 1H), 7.89 (d, J=8.2 Hz, 1H), 7.75 (d, J=1.6 Hz, 1H), 7.64-7.58 (m, 1H), 7.49 (dd, J=8.2, 1.7 Hz, 1H), 7.40-7.31 (m, 2H), 3.32 (s, 3H), 3.09 (q, J=7.4 Hz, 2H), 1.40 (t, J=7.4 Hz, 3H); ¹³C NMR (101 MHz, CDCl₃) δ 189.6, 145.3, 141.4, 137.5, 134.7, 132.3, 132.2, 132.0, 131.63, 130.3, 126.3, 125.1, 122.5, 39.2, 26.3, 13.8; LR-MS calcd. for C₁₆H₁₆NO₃S₂[M+H]⁺ 334.06, found 334.24.

### 3-(Isopropylthio)-6-methyldibenzo[c,f][1,2]thiazepin-11(6H)-one 5,5-dioxide (3aa)

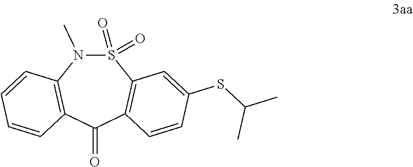 [SMILES: CC(C)Sc1ccc2c(c1)S(=O)(=O)N(C)c1ccccc1C2=O]

Ketone 3aa was prepared from ketone 3f (308 mg, 1.00 mmol) and 2-propanethiol according to the procedure described for ketone 3z and obtained as powdery yellow crystals (264 mg, 76%). ¹H NMR (400 MHz, CDCl₃) δ 8.30 (dd, J=8.1, 1.6 Hz, 1H), 7.89 (d, J=8.2 Hz, 1H), 7.80 (d, J=1.9 Hz, 1H), 7.64-7.59 (m, 1H), 7.54 (dd, J=8.2, 1.9 Hz, 1H), 7.39-7.31 (m, 2H), 3.65 (hept, J=6.7 Hz, 1H), 3.32 (s, 3H), 1.40 (d, J=6.7 Hz, 6H); ¹³C NMR (101 MHz, CDCl₃) δ 189.7, 144.5, 141.4, 137.4, 134.8, 132.5, 132.3, 132.2, 132.1, 131.5, 126.3, 125.0, 124.3, 39.2, 37.0, 22.9; LR-MS calcd. for C₁₇H₁NO₃S₂[M+H]⁺ 348.07, found 348.08.

### 3-(Benzyloxy)-6-methyldibenzo[c,f][1,2]thiazepin-11(6H)-one 5,5-dioxide (3ab)

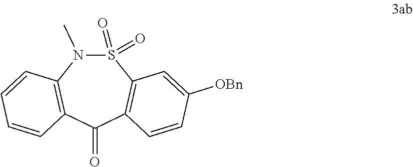 [SMILES: CN1c2ccccc2C(=O)c2ccc(OCc3ccccc3)cc2S1(=O)=O]

To a mixture of ketone 3m (145 mg, 0.500 mmol) and K₂CO₃ (138 mg, 1.00 mmol) in anhydrous acetone (1.0 mL) was added benzyl bromide (89.2 μL, 0.750 mmol), and the mixture was refluxed for 3 h. The mixture was then cooled to room temperature, diluted with acetone (10 mL), and filtered, washing the filter cake with additional acetone. The combined filtrates were concentrated to provide a yellow-orange oil. This material was recrystallized from MeOH to provide pure ketone 3ab as off-white crystals (125 mg, 66%). ¹H NMR (500 MHz, CDCl₃) δ 8.33 (dd, J=8.1, 1.4 Hz, 1H), 8.02 (d, J=8.7 Hz, 1H), 7.63 (td, J=8.0, 1.6 Hz, 1H), 7.55 (d, J=2.6 Hz, 1H), 7.47-7.34 (m, 7H), 7.24 (dd, J=8.7, 2.6 Hz, 1H), 5.22 (s, 2H), 3.31 (s, 3H); ¹³C NMR (126 MHz, CDCl₃) δ 189.1, 161.8, 141.2, 139.0, 135.5, 134.7, 134.5, 132.4, 132.3, 129.0, 128.7, 128.5, 127.8, 126.6, 125.5, 119.6, 111.7, 71.0, 39.3; LR-MS calcd. for C₂₁H₁₈NO₄S [M+H]⁺ 380.10, found 380.39.

### 6-Methyl-5,5-dioxido-11-oxo-6,11-dihydrodibenzo[c,f][1,2]thiazepin-3-yl acetate (3ac)

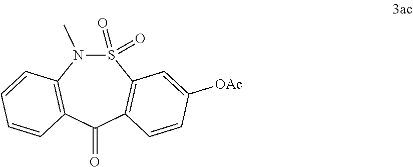 [SMILES: CC(=O)Oc1ccc2c(c1)S(=O)(=O)N(C)c1ccccc1C2=O]

To a solution of ketone 3m (230 mg, 0.795 mmol) and pyridine (0.30 mL) in anhydrous CH₂Cl₂ (10 mL) was added acetyl chloride (0.50 mL) at 0° C. The mixture was then allowed to warm to room temperature and stirred for 1 h. The reaction was then quenched with ice water and extracted with CH₂Cl₂. The combined organics were dried over Na₂SO₄ and concentrated to provide the crude product. This material was purified by cold crystallization from MeOH to give the pure ketone 3ac as a white solid (210 mg, 80%). ¹H NMR (400 MHz, Acetone-d₆) δ 8.25 (dd, J=8.1, 1.6 Hz, 1H), 8.01 (d, J=8.4 Hz, 1H), 7.79-7.73 (m, 2H), 7.63 (dd, J=8.4, 2.3 Hz, 1H), 7.58 (dd, J=8.2, 1.0 Hz, 1H), 7.45 (ddd, J=8.2, 7.2, 1.2 Hz, 1H), 3.39 (s, 3H), 2.37 (s, 3H); ¹³C NMR (101 MHz, Acetone-d₆) δ 190.92, 169.23, 154.35, 142.82, 138.73, 135.81, 134.59, 134.05, 132.16, 131.62, 127.81, 126.78, 125.97, 119.67, 39.44, 21.01; LR-MS calcd. for C₁₆H₁₄NO₅S [M+H]⁺ 332.06, found 331.72.

### Preparation of N-Substituted 11-amino-6-alkyl-6,11-dihydrodiaryl [c,f][1,2]thiazepine 5,5-dioxides

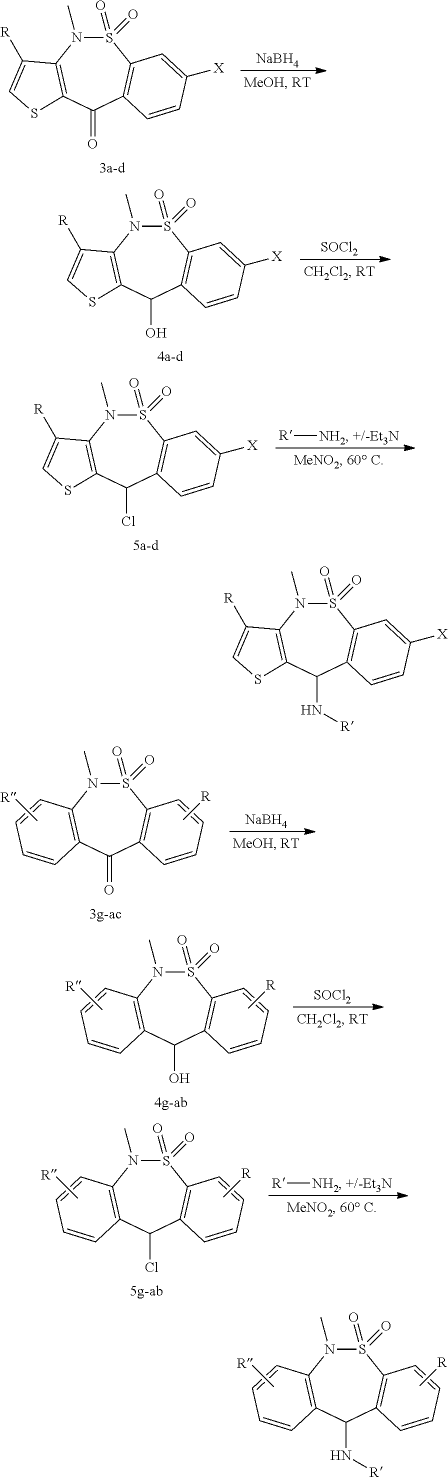

General Procedure for Preparation of Alcohols 4a-4ab. Sodium borohydride (2.0 mmol) was added to an ice-cooled solution (or suspension) of ketone 3 (1.0 mmol) in MeOH (7 mL). After stirring for 2-3 h at room temperature, the reaction was quenched by adding saturated aqueous ammonium chloride (5 mL) and saturated aqueous NaHCO₃ (5 mL). MeOH was evaporated and the precipitate was filtered, washed with water, and dried (alternatively, the residue was extracted with EtOAc and the combined organic layers were washed with water, dried over Na₂SO₄, filtered, and concentrated). The crude product was crystallized from MeOH/water or used in the next step without further purification.

### 10-Hydroxy-4-methyl-4,10-dihydrobenzo[f]thieno[3,2-c][1,2]thiazepine 5,5-dioxide (4a)

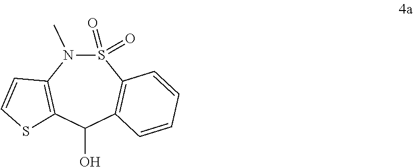 [SMILES: CN1c2ccsc2C(O)c2ccccc2S1(=O)=O]

The product 4a was obtained as a white solid (270 mg, 89%). ¹H NMR (400 MHz, Acetone-d₆) δ 8.03 (d, J=7.8 Hz, 1H), 7.87 (d, J=7.7 Hz, 1H), 7.85-7.77 (m, 1H), 7.59 (t, J=7.6 Hz, 1H), 7.37 (d, J=5.5 Hz, 1H), 7.00 (d, J=5.5 Hz, 1H), 6.65 (d, J=6.8 Hz, 1H), 6.15 (d, J=7.1 Hz, 1H), 3.02 (s, 3H); ¹³C NMR (101 MHz, Acetone-d₆) δ 139.2, 135.8, 135.0, 134.0, 131.0, 128.7, 128.0, 126.9, 125.6, 124.1, 69.2, 39.8; LR-MS calcd. for C₁₂H₁₀NO₂S₂[M−OH]⁺264.01, found 264.68.

### 3-Bromo-10-hydroxy-4-methyl-4,10-dihydrobenzo[f]thieno[3,2-c][1,2]thiazepine 5,5-dioxide (4b)

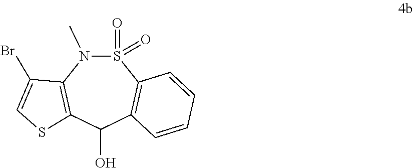 [SMILES: CN1c2c(Br)csc2C(O)c2ccccc2S1(=O)=O]

The product 4b was obtained as a pale-yellow glass (176 mg, 98%). ¹H NMR (500 MHz, CDCl₃) δ 7.97 (d, J=7.7 Hz, 1H), 7.75-7.66 (m, 2H), 7.55 (t, J=7.5 Hz, 1H), 7.30 (s, 1H), 6.05 (d, J=10.1 Hz, 1H), 4.67 (dd, J=9.8, 5.4 Hz, 1H), 2.86 (s, 3H); ¹³C NMR (126 MHz, CDCl₃) δ 136.0, 134.9, 134.6, 133.9, 132.6, 129.7, 129.4, 128.9, 123.2, 108.9, 71.5, 39.2; LR-MS calcd. for C₁₂H₉BrNO₂S₂[M−OH]⁺343.92, found 344.69.

### 7-Chloro-10-hydroxy-4-methyl-4,10-dihydrobenzo[f]thieno[3,2-c][1,2]thiazepine 5,5-dioxide (4c)

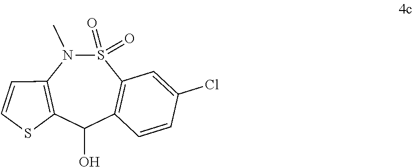 [SMILES: CN1c2ccsc2C(O)c2ccc(Cl)cc2S1(=O)=O]

The product 4c was obtained as a tan solid (335.7 mg, 98%). ¹H NMR (400 MHz, CDCl₃) δ 7.94 (d, J=2.0 Hz, 1H), 7.68-7.60 (m, 2H), 7.29 (d, J=5.4 Hz, 1H), 6.84 (d, J=5.5 Hz, 1H), 6.10 (d, J=10.0 Hz, 1H), 4.61 (d, J=10.1 Hz, 1H), 3.10 (s, 3H); ¹³C NMR (101 MHz, CDCl₃) δ 136.1, 135.8, 135.0, 135.0, 134.2, 131.1, 127.9, 127.8, 125.2, 124.1, 71.0, 39.3; LR-MS cald. for C₁₂H₉ClNO₂S₂[M−OH]⁺297.98, found 298.00.

### 7-Bromo-10-hydroxy-4-methyl-4,10-dihydrobenzo[f]thieno[3,2-c][1,2]thiazepine 5,5-dioxide (4d)

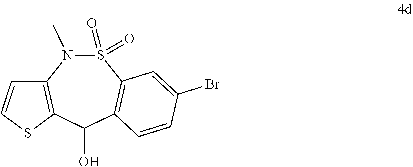 [SMILES: CN1c2ccsc2C(O)c2ccc(Br)cc2S1(=O)=O]

The product 4d was obtained as a yellowish-tan solid (579 mg, 100%) ¹H NMR (500 MHz, CDCl₃) δ 8.10 (d, J=2.0 Hz, 1H), 7.80 (dd, J=8.1, 2.0 Hz, 1H), 7.57 (d, J=8.2 Hz, 1H), 7.31 (d, J=5.4 Hz, 1H), 6.85 (d, J=5.5 Hz, 1H), 6.05 (d, J=10.6 Hz, 1H), 4.49 (d, J=10.6 Hz, 1H), 3.11 (s, 3H); ¹³C NMR (126 MHz, CDCl₃) δ 137.2, 136.4, 136.0, 135.3, 131.5, 130.6, 127.5, 125.4, 124.1, 122.8, 71.3, 39.3; LR-MS cald. for C₁₂H₉BrNO₂S₂[M−OH]⁺341.93, found 342.47.

### 3-Fluoro-11-hydroxy-6-methyl-6,11-dihydrodibenzo[c,f][1,2]thiazepine 5,5-dioxide (4g)

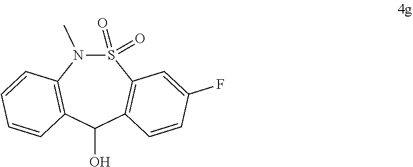 [SMILES: CN1c2ccccc2C(O)c2ccc(F)cc2S1(=O)=O]

The product 4g was obtained as a white crystalline solid (215 mg, 100%). ¹H NMR (400 MHz, CDCl₃) δ 7.69 (dt, J=8.7, 4.3 Hz, 2H), 7.61 (dd, J=7.6, 1.6 Hz, 1H), 7.41 (td, J=7.6, 1.7 Hz, 1H), 7.37-7.26 (m, 3H), 5.93 (d, J=9.7 Hz, 1H), 4.25 (d, J=9.8 Hz, 1H), 3.19 (s, 3H); ¹³C NMR (101 MHz, CDCl₃) (additional peaks due to C—F coupling) δ 163.3, 160.8, 138.9, 135.3, 134.0, 133.9, 132.6, 132.6, 131.8, 130.1, 127.9, 127.1, 120.3, 120.1, 115.9, 115.7, 39.5; LR-MS calcd. for C₁₄H₁₁FNO₂S [M−OH]⁺276.05, found 276.12.

### 11-Hydroxy-3-methoxy-6-methyl-6,11-dihydrodibenzo[c,f][1,2]thiazepine 5,5-dioxide (4h)

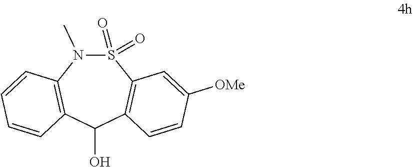 [SMILES: COc1ccc2c(c1)S(=O)(=O)N(C)c1ccccc1C2O]

The product 4h was obtained as a white crystalline solid (153 mg, 100%). ¹H NMR (500 MHz, CDCl₃) δ 7.60 (dd, J=7.7, 1.5 Hz, 1H), 7.56 (d, J=8.5 Hz, 1H), 7.49 (d, J=2.7 Hz, 1H), 7.38 (td, J=7.6, 1.6 Hz, 1H), 7.32 (td, J=7.5, 1.4 Hz, 1H), 7.28 (dd, J=7.9, 1.3 Hz, 1H), 7.09 (dd, J=8.4, 2.7 Hz, 1H), 5.81 (d, J=10.3 Hz, 1H), 4.43 (d, J=10.3 Hz, 1H), 3.88 (s, 3H), 3.14 (s, 3H); ¹³C NMR (126 MHz, CDCl₃) δ 159.7, 139.4, 138.0, 135.1, 132.6, 132.5, 129.9, 129.6, 127.6, 127.1, 119.0, 113.5, 56.0, 39.8; LR-MS calcd. for C₁₅H₁₄NO₃S [M−OH]⁺ 288.07, found 287.93.

### 11-Hydroxy-6-methyl-3-phenoxy-6,11-dihydrodibenzo[c,f][1,2]thiazepine 5,5-dioxide (4i)

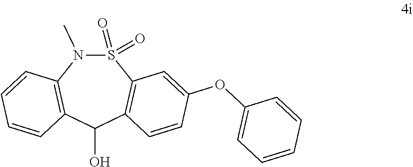 [SMILES: CN1c2ccccc2C(O)c2ccc(Oc3ccccc3)cc2S1(=O)=O]

The product 4i was obtained as a pale-yellow glass (291 mg, 100%). ¹H NMR (400 MHz, CDCl₃) δ 7.63-7.58 (m, 3H), 7.42-7.36 (m, 3H), 7.32 (ddd, J=15.8, 7.8, 1.5 Hz, 2H), 7.19 (ddd, J=11.0, 5.2, 1.8 Hz, 2H), 7.08-7.03 (m, 2H), 5.89 (d, J=9.5 Hz, 1H), 4.41 (d, J=9.8 Hz, 1H), 3.17 (s, 3H); ¹³C NMR (101 MHz, CDCl₃) δ 157.9, 155.8, 139.1, 138.4, 135.4, 132.4, 132.0, 131.9, 130.3, 129.9, 127.7, 127.1, 124.8, 122.3, 119.8, 117.8, 76.7, 39.5; LR-MS calcd. for C₂₀H₁₆NO₃S [M−OH]⁺ 350.09, found 350.01.

### 11-Hydroxy-3,6-dimethyl-6,11-dihydrodibenzo[c,f][1,2]thiazepine 5,5-dioxide (4j)

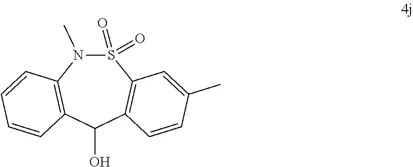 [SMILES: Cc1ccc2c(c1)S(=O)(=O)N(C)c1ccccc1C2O]

The product 4j was obtained as a white solid (220 mg, 91%). ¹H NMR (400 MHz, Acetone-d₆) δ 7.82 (d, J=8.0 Hz, 1H), 7.68 (dd, J=7.4, 1.6 Hz, 1H), 7.64 (s, 1H), 7.50-7.41 (m, 2H), 7.41-7.30 (m, 2H), 6.40 (d, J=5.6 Hz, 1H), 5.41 (d, J=5.7 Hz, 1H), 3.36 (s, 3H), 2.39 (s, 3H); ¹³C NMR (101 MHz, Acetone-d₆) δ 143.1, 139.0, 139.0, 138.6, 138.2, 133.8, 129.5, 128.6, 128.5, 128.3, 127.4, 127.3, 70.8, 37.8, 20.7; LR-MS calcd. for C₁₅H₁₄NO₂S [M−OH]⁺272.07, found 272.75.

### 9-Bromo-3-chloro-11-hydroxy-6-methyl-6,11-dihydrodibenzo[c,f][1,2]thiazepine 5,5-dioxide (4k)

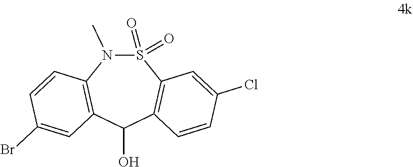 [SMILES: CN1c2ccc(Br)cc2C(O)c2ccc(Cl)cc2S1(=O)=O]

The crude product was crystallized from MeOH/H₂O. The product 4k was obtained as a white solid (280 mg, 96%). ¹H NMR (400 MHz, Acetone-d₆) δ 8.01 (dd, J=8.5, 0.7 Hz, 1H), 7.89 (dd, J=2.4, 0.6 Hz, 1H), 7.79 (d, J=2.2 Hz, 1H), 7.70 (dd, J=8.5, 2.2 Hz, 1H), 7.59 (ddd, J=8.5, 2.4, 0.4 Hz, 1H), 7.46 (d, J=8.5 Hz, 1H), 6.50 (d, J=5.4 Hz, 1H), 5.83 (d, J=5.6 Hz, 1H), 3.39 (s, 3H); ¹³C NMR (101 MHz, Acetone-d₆) δ 144.4, 140.6, 139.1, 137.7, 134.4, 133.3, 132.8, 130.6, 130.1, 129.2, 127.7, 122.2, 69.2, 38.2; LR-MS calcd. for C₁₄H₁₀BrClNO₂S [M−OH]⁺371.93, found 372.86.

### 11-Hydroxy-6-methyl-3-(methylthio)-6,11-dihydrodibenzo[c,f][1,2]thiazepine 5,5-dioxide (4l)

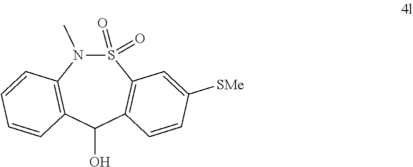 [SMILES: CSc1ccc2c(c1)S(=O)(=O)N(C)c1ccccc1C2O]

The product 4l was obtained as a pale-yellow crystalline solid (323 mg, 100%). ¹H NMR (500 MHz, CDCl₃) δ 7.75 (d, J=2.0 Hz, 1H), 7.57 (dd, J=7.6, 1.3 Hz, 1H), 7.54 (d, J=8.2 Hz, 1H), 7.37 (ddd, J=9.2, 7.9, 1.9 Hz, 2H), 7.32-7.27 (m, 2H), 5.89 (d, J=9.4 Hz, 1H), 4.44 (d, J=9.4 Hz, 1H), 3.16 (s, 3H), 2.51 (s, 3H); ¹³C NMR (126 MHz, CDCl₃) δ 140.8, 138.8, 137.4, 136.0, 134.0, 131.2, 130.4, 130.3, 129.7, 127.7, 127.0, 124.8, 75.9, 39.2, 15.4; LR-MS calcd. for C₁₅H₁₄NO₂S₂[M−OH]⁺304.05, found 304.71.

### 3,11-Dihydroxy-6-methyl-6,11-dihydrodibenzo[c,f][1,2]thiazepine 5,5-dioxide (4m)

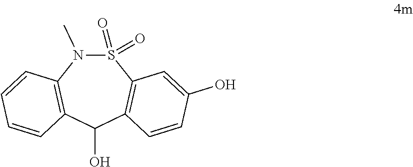 [SMILES: CN1c2ccccc2C(O)c2ccc(O)cc2S1(=O)=O]

To an ice-cold suspension of ketone 3ac (95 mg, 0.287 mmol) in MeOH (5 mL) was added NaBH₄ (210 mg), and the effervescent mixture was allowed to immediately warm to room temperature and stirred for 1.5 h. The reaction was then cooled back to 0° C., additional NaBH₄ (100 mg) was added, and the mixture was again allowed to warm to room temperature and stirred for a further 1.5 h. The reaction mixture was then poured into saturated aqueous NH₄Cl (20 mL), and the pH was adjusted to ˜3 with 1M aqueous HCl. The mixture was then extracted with CH₂Cl₂ and the combined organics were dried over Na₂SO₄ and concentrated to give the crude product. This material was purified by column chromatography (CH₂Cl₂+2% MeOH) to provide pure alcohol 4m as an off-white solid (72 mg, 86%). ¹H NMR (400 MHz, Acetone-d₆) δ 8.91 (s, 1H), 7.75 (d, J=8.6 Hz, 1H), 7.67 (dd, J=7.5, 1.7 Hz, 1H), 7.45 (dd, J=7.7, 1.5 Hz, 1H), 7.41-7.30 (m, 3H), 7.07 (dd, J=8.6, 2.6 Hz, 1H), 6.26 (d, J=5.7 Hz, 1H), 5.29 (d, J=6.0 Hz, 1H), 3.34 (s, 3H); ¹³C NMR (101 MHz, Acetone-d₆) (spectrum complicated by conformers) δ 157.8, 142.6, 140.2, 138.9, 131.6, 129.8, 129.6, 128.63, 128.57, 128.1, 120.0, 119.9, 114.73, 114.65, 71.8, 71.6, 38.4; LR-MS calcd. for C₁₄H₁₂NO₃S [M−OH]⁺274.05, found 274.45.

### 3-Bromo-11-hydroxy-6-methyl-6,11-dihydrodibenzo[c,f][1,2]thiazepine 5,5-dioxide (4o)

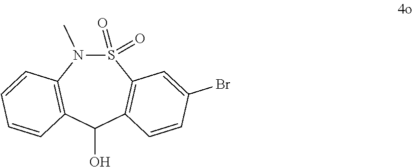 [SMILES: CN1c2ccccc2C(O)c2ccc(Br)cc2S1(=O)=O]

The product 4o was obtained as a white solid (70 mg, 88%). ¹H NMR (400 MHz, Acetone-d₆) δ 7.95-7.90 (m, 2H), 7.81 (dd, J=8.5, 2.1 Hz, 1H), 7.73-7.69 (m, 1H), 7.50 (dd, J=7.6, 1.6 Hz, 1H), 7.41 (dd, J=7.2, 5.2 Hz, 1H), 7.38 (dd, J=7.1, 1.7 Hz, 1H), 6.44 (d, J=5.4 Hz, 1H), 5.62 (d, J=5.4 Hz, 1H), 3.41 (s, 3H); ¹³C NMR (101 MHz, Acetone-d₆) δ 142.8, 141.4, 140.4, 138.1, 136.0, 130.6, 129.9, 129.5, 129.1, 128.7, 127.2, 121.8, 70.1, 38.1; LR-MS calcd. for C₁₄H₁₁BrNO₂S [M−OH]⁺335.97, found 335.93.

### 11-Hydroxy-3-iodo-6-methyl-6,11-dihydrodibenzo[c,f][1,2]thiazepine 5,5-dioxide (4q)

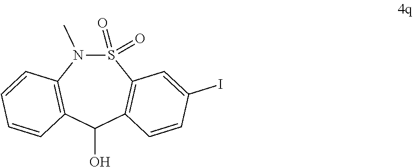 [SMILES: CN1c2ccccc2C(O)c2ccc(I)cc2S1(=O)=O]

The product 4q was obtained as a white solid (74.8 mg, 95%). ¹H NMR (500 MHz, CDCl₃) (observed as a -4:1 ratio of 2 conformers resulting in partial integrals) δ 8.27 (d, J=1.8 Hz, 1H), 7.99 (dd, J=7.7, 1.1 Hz, 0.2H), 7.93 (dd, J=8.1, 1.8 Hz, 0.8H), 7.68 (d, J=7.2 Hz, 0.2H), 7.65-7.58 (m, 1H), 7.53 (td, J=7.6, 1.3 Hz, 0.2H), 7.44-7.37 (m, 1.8H), 7.37-7.29 (m, 1.8H), 5.92 (s, 1H), 4.40 (d, J=9.6 Hz, 0.2H), 4.15 (d, J=7.5 Hz, 0.8H), 3.20 (s, 2.4H), 3.14 (s, 0.6H); ¹³C NMR (126 MHz, CDCl₃) (additional peaks due to conformers) δ 142.4, 138.8, 138.7, 137.5, 136.5, 135.4, 133.6, 132.1, 131.7, 131.4, 130.6, 130.1, 130.0, 128.9, 128.4, 127.9, 127.6, 127.0, 126.9, 93.6, 76.2, 39.3; LR-MS calcd. for C₁₄H₁₁INO₂S [M−OH]⁺383.96, found 383.71.

### 11-Hydroxy-7-methoxy-6-methyl-6,11-dihydrodibenzo[c,f][1,2]thiazepine 5,5-dioxide (4r)

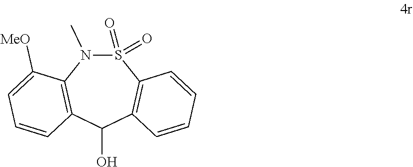 [SMILES: COc1cccc2c1N(C)S(=O)(=O)c1ccccc1C2O]

Alcohol 4r was synthesized according to the following scheme via the procedures described below.

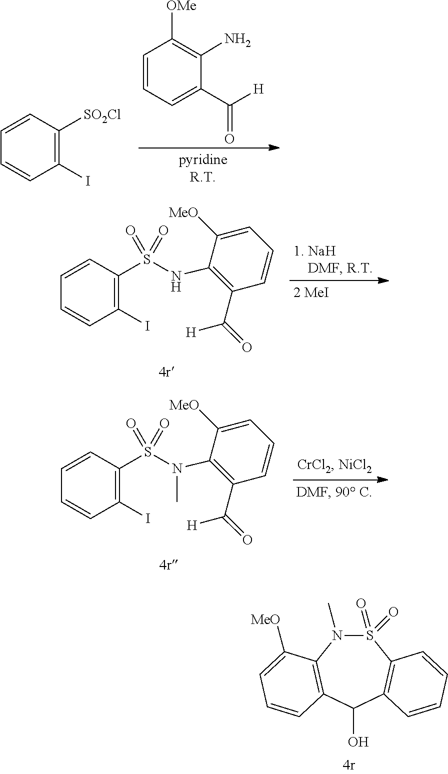 [SMILES: O=S(=O)(Cl)c1ccccc1I ; COc1cccc(C=O)c1N ; COc1cccc(C=O)c1NS(=O)(=O)c1ccccc1I ; COc1cccc(C=O)c1N(C)S(=O)(=O)c1ccccc1I ; COc1cccc2c1N(C)S(=O)(=O)c1ccccc1C2O]

To a solution of 2-amino-3-methoxybenzaldehyde (800 mg, 5.29 mmol) in anhydrous pyridine (3.75 mL) was added 2-iodobenzenesulfonyl chloride (1.52 g, 5.04 mmol), and the orange-brown solution was stirred at room temperature for 1 h. The reaction mixture was then diluted with CH₂Cl₂ (50 mL), washed with 7% aqueous HCl (2×50 mL), brine (50 mL), saturated aqueous NaHCO₃ (50 mL), and brine again (50 mL), dried over Na₂SO₄, and concentrated to yield an orange-brown oil. This crude material was purified by repeated column chromatography (column 1: CH₂Cl₂, 4 column volumes→CH₂Cl₂:Et₂O—1:1, 2 column volumes/column 2:hexanes:EtOAc—7:3, 2 column volumes→1:1, until finished) to obtain pure sulfonamide 4r′ as a tan crystalline solid (668 mg, 32%). ¹H NMR (400 MHz, CDCl₃) δ 10.32 (s, 1H), 8.19 (s, 1H), 8.10 (dd, J=7.8, 0.9 Hz, 1H), 7.76 (dd, J=7.9, 1.5 Hz, 1H), 7.48 (d, J=7.8 Hz, 1H), 7.31 (dd, J=11.1, 4.2 Hz, 1H), 7.27 (t, J=8.0 Hz, 1H), 7.17 (dd, J=7.7, 1.6 Hz, 1H), 6.91 (dd, J=8.2, 1.1 Hz, 1H), 3.42 (s, 3H); ¹³C NMR (101 MHz, CDCl₃) δ 191.1, 153.1, 142.5, 142.2, 133.5, 132.7, 131.0, 127.9, 127.7, 127.0, 121.8, 116.1, 93.1, 55.3; LR-MS calcd. for C₁₄H₁₃INO₄S [M+H]⁺ 417.96, found 418.52.

To a suspension of sodium hydride (60% dispersion in mineral oil, 120 mg, 3.00 mmol) in anhydrous DMF (2.2 mL) was added a solution of sulfonamide 4r′ (626 mg, 1.50 mmol) in anhydrous DMF (2.2 mL) dropwise over 4 minutes, and the resulting lemon-yellow solution was stirred for 30 minutes at room temperature. Methyl iodide (187 μL, 3.00 mmol) was then added, and the mixture was stirred for 1 h and then quenched with ice water (30 mL). The resulting white precipitate was collected, washed with hexanes and Et₂O, and dried to yield the first crop of pure sulfonamide 4r′ as an off-white crystalline solid (231 mg). The filtrate was then washed with hexanes and extracted with 4:1 Et₂O:CH₂Cl₂ (2×25 mL), adding the CH₂Cl₂ first to ensure dissolution and then diluting with Et₂O. The combined organics were washed with water (2×50 mL), dried over Na₂SO₄, and concentrated to yield a pale-yellow oily solid. Upon addition of a small quantity of Et₂O to this material abundant crystals formed. After removal of the supernatant these crystals were washed with three small portions of ice-cold Et₂O and dried to obtain a second crop of pure sulfonamide 4r″ (281 mg). Total yield of 4r″ was 512 mg (79%). ¹H NMR (400 MHz, CDCl₃) δ 10.36 (d, J=0.8 Hz, 1H), 8.11 (dd, J=7.8, 1.2 Hz, 1H), 7.79 (dd, J=8.0, 1.6 Hz, 1H), 7.56 (dd, J=7.8, 1.4 Hz, 1H), 7.43-7.38 (m, 1H), 7.38-7.34 (m, 1H), 7.14 (td, J=7.7, 1.7 Hz, 1H), 6.99 (dd, J=8.2, 1.3 Hz, 1H), 3.53 (s, 3H), 3.39 (s, 3H); ¹³C NMR (101 MHz, CDCl₃) δ 190.3, 157.3, 143.1, 143.0, 136.8, 133.1, 131.9, 130.9, 130.1, 128.0, 119.9, 116.7, 91.9, 55.4, 40.0; LR-MS calcd. for C₁₅H₁₅INO₄S [M+H]⁺ 431.98, found 432.66.

To a dark-green suspension of CrCl₂ (474 mg, 3.86 mmol) and NiCl₂ (50.0 mg, 0.386 mmol) in anhydrous DMF (4.8 mL) was added a solution of sulfonamide 4r″ (416 mg, 0.965 mmol) in anhydrous DMF (4.8 mL) dropwise over 4 minutes. The resulting nearly black mixture was heated to 90° C. for 1.5 h then quenched with water (50 mL) and extracted with Et₂O (50 mL, 2×25 mL). The combined organics were washed with brine (50 mL), dried over Na₂SO₄, and concentrated to yield a yellow oil. This crude was purified by column chromatography (CH₂Cl₂, 2 column volumes→CH₂Cl₂:Et₂O—80:1, 2 column volumes→40:1, 2 column volumes→20:1, 2 column volumes) to obtain impure product as a white foam. On addition of a small quantity of Et₂O, crystallization occurred. After washing the crystals, this crystallization procedure (concentration from CH₂Cl₂ solution followed by addition of Et₂O) was repeated to yield the pure alcohol 4r as fine white crystals (132 mg, 45%). ¹H NMR (500 MHz, CDCl₃) (significant broadening of some peaks was observed due to conformers) δ 7.99 (d, J=7.8 Hz, 1H), 7.70 (br s, 1H), 7.58 (t, J=6.9 Hz, 1H), 7.49 (t, J=7.7 Hz, 1H), 7.32 (t, J=8.0 Hz, 1H), 7.21 (br s, 1H), 6.95 (d, J=7.7 Hz, 1H), 6.01 (br s, 1H), 4.10 (br s, 1H), 3.93 (s, 3H), 3.05 (br s, 3H); ¹³C NMR (126 MHz, CDCl₃) δ 156.0, 139.5, 138.4, 137.5, 133.1, 129.1, 128.8, 128.6, 126.7, 121.5, 112.1, 74.9, 56.3, 37.1; LR-MS calcd. for C₁₅H₁₄NO₃S [M−OH]⁺288.07, found 288.72.

### 11-Hydroxy-6-methyl-3-phenyl-6,11-dihydrodibenzo[c,f][1,2]thiazepine 5,5-dioxide (4t)

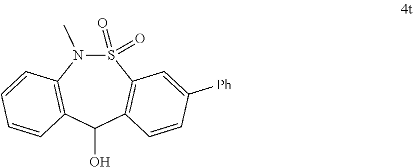 [SMILES: CN1c2ccccc2C(O)c2ccc(-c3ccccc3)cc2S1(=O)=O]

The product 4t was obtained as a white solid (235 mg, 94%). ¹H NMR (400 MHz, Acetone-d₆) δ 8.08-8.03 (m, 2H), 7.91 (dd, J=8.2, 1.9 Hz, 1H), 7.74 (dd, J=7.3, 1.7 Hz, 1H), 7.72-7.66 (m, 2H), 7.54-7.46 (m, 3H), 7.45-7.34 (m, 3H), 6.51 (d, J=5.5 Hz, 1H), 5.55 (d, J=5.7 Hz, 1H), 3.41 (s, 3H); ¹³C NMR (101 MHz, Acetone-d₆) δ 142.8, 141.8, 140.0, 139.9, 139.7, 138.6, 131.4, 130.0, 129.7, 129.0, 128.8, 128.4, 128.0, 127.8, 127.4, 126.4, 70.6, 38.0; LR-MS calcd. for C₂₀H₁₆NO₂S [M−OH]⁺334.09, found 334.15.

### 11-Hydroxy-6-methyl-3-(methylsulfonyl)-6,11-dihydrodibenzo[c,f][1,2]thiazepine 5,5-dioxide (4u)

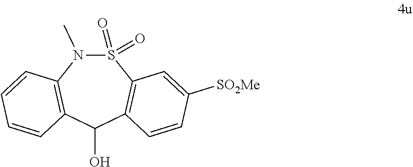 [SMILES: CN1c2ccccc2C(O)c2ccc(S(C)(=O)=O)cc2S1(=O)=O]

The product 4u was obtained as a white solid (217 mg, 99%). ¹H NMR (400 MHz, Acetone-d₆) δ 8.32 (d, J=1.8 Hz, 1H), 8.28 (d, J=8.3 Hz, 1H), 8.18 (dd, J=8.3, 1.8 Hz, 1H), 7.75 (dd, J=7.2, 1.8 Hz, 1H), 7.56-7.52 (m, 1H), 7.41 (pd, J=7.3, 1.6 Hz, 2H), 6.62 (d, J=5.3 Hz, 1H), 5.81 (d, J=5.3 Hz, 1H), 3.45 (s, 3H), 3.22 (s, 3H); ¹³C NMR (101 MHz, Acetone-d₆) δ 146.5, 142.6, 142.2, 140.6, 137.7, 131.6, 130.0, 129.3, 128.6, 128.2, 127.2, 126.9, 69.7, 44.1, 37.9; LR-MS calcd. for C₁₅H₁₄NO₄S₂[M−OH]⁺336.04, found 336.19.

### 11-Hydroxy-6-methyl-3-(methylsulfinyl)-6,11-dihydrodibenzo[c,f][1,2]thiazepine 5,5-dioxide (4v)

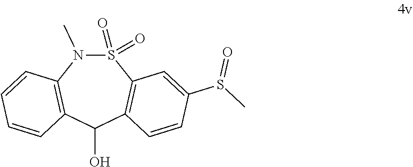 [SMILES: CN1c2ccccc2C(O)c2ccc(S(C)=O)cc2S1(=O)=O]

The product 4v was obtained as a white foam (152 mg, 98%, 1:1 mixture of diastereomers). ¹H NMR (400 MHz, CDCl₃) (partial integrals due to mixture of diastereomers) δ 8.15 (d, J=1.5 Hz, 0.5H), 8.03 (d, J=1.5 Hz, 0.5H), 7.98-7.85 (m, 1.5H), 7.76 (dd, J=8.1, 1.6 Hz, 0.5H), 7.63 (d, J=7.4 Hz, 1H), 7.41-7.29 (m, 3H), 6.21 (s, 1H), 4.58 (br s, 1H), 3.29 (s, 1.5H), 3.29 (s, 1.5H), 2.73 (s, 1.5H), 2.73 (s, 1.5H); ¹³C NMR (101 MHz, CDCl₃) (additional peaks due to mixture of diastereomers) δ 146.4, 142.2, 142.1, 139.2, 138.8, 138.3, 138.1, 137.5, 129.8, 129.7, 129.3, 129.1, 128.8, 128.3, 127.9, 127.6, 127.2, 123.5, 123.3, 73.0, 72.8, 43.8, 38.5, 38.4; LR-MS calcd. for C₁₅H₁₄NO₃S₂ [M−OH]⁺320.04, found 320.20.

### 2-Chloro-11-hydroxy-6-methyl-6,11-dihydrodibenzo[c,f][1,2]thiazepine 5,5-dioxide (4w)

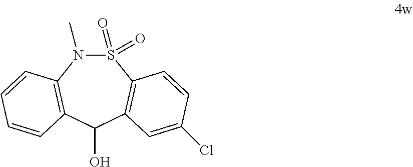 [SMILES: CN1c2ccccc2C(O)c2cc(Cl)ccc2S1(=O)=O]

The product 4w was obtained as a white solid (195 mg, 95%). ¹H NMR (400 MHz, Acetone-d₆) δ 8.00 (d, J=1.5 Hz, 1H), 7.86 (d, J=8.5 Hz, 1H), 7.74 (dd, J=7.1, 1.8 Hz, 1H), 7.58-7.49 (m, 2H), 7.46-7.36 (m, 2H), 6.53 (d, J=5.3 Hz, 1H), 5.74 (d, J=5.4 Hz, 1H), 3.42 (s, 3H); ¹³C NMR (101 MHz, Acetone-d₆) δ 143.6, 142.8, 138.7, 138.2, 138.1, 130.3, 129.8, 129.0, 128.9, 128.4, 126.8, 126.6, 69.4, 37.7; LR-MS calcd. for C₁₄H₁₁ClNO₂S [M−OH]⁺292.02, found 292.38.

### 8-Chloro-11-hydroxy-6-methyl-6,11-dihydrodibenzo[c,f][1,2]thiazepine 5,5-dioxide (4x)

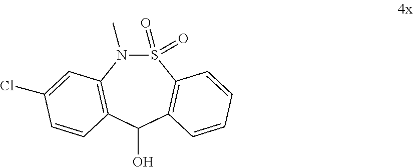 [SMILES: CN1c2cc(Cl)ccc2C(O)c2ccccc2S1(=O)=O]

The product 4× was obtained as an off-white foam (384 mg, 100%). ¹H NMR (500 MHz, CDCl₃) δ 7.96 (dd, J=7.8, 1.1 Hz, 1H), 7.68 (d, J=7.5 Hz, 1H), 7.62 (td, J=7.5, 1.3 Hz, 1H), 7.58-7.55 (m, 1H), 7.52 (td, J=7.6, 1.2 Hz, 1H), 7.30-7.26 (m, 2H), 5.94 (d, J=9.0 Hz, 1H), 4.50 (d, J=9.5 Hz, 1H), 3.14 (s, 3H); ¹³C NMR (126 MHz, CDCl₃) δ 140.2, 137.6, 136.6, 135.0, 134.0, 133.8, 132.8, 130.1, 128.9, 128.1, 127.7, 126.6, 75.9, 39.1; LR-MS calcd. for C₁₄H₁₁ClNO₂S [M−OH]⁺ 292.02, found 292.51.

### 6-Ethyl-11-hydroxy-6,11-dihydrodibenzo[c,f][1,2]thiazepine 5,5-dioxide (4y)

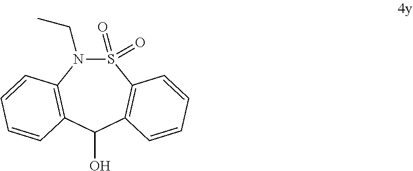 [SMILES: CCN1c2ccccc2C(O)c2ccccc2S1(=O)=O]

The product 4y was obtained as a pale-yellow glass (226 mg, 100%). ¹H NMR (500 MHz, CDCl₃) δ 7.98 (dd, J=7.8, 1.1 Hz, 1H), 7.68 (d, J=7.5 Hz, 1H), 7.65-7.57 (m, 2H), 7.50 (td, J=7.6, 1.2 Hz, 1H), 7.41-7.32 (m, 3H), 5.99 (s, 1H), 4.22 (d, J=6.6 Hz, 1H), 3.77-3.68 (m, 2H), 0.90 (t, J=7.2 Hz, 3H); ¹³C NMR (126 MHz, CDCl₃) δ 139.0, 138.1, 137.2, 137.0, 133.3, 131.9, 129.9, 129.8, 128.9, 127.9, 127.8, 127.4, 76.6, 47.0, 13.9; LR-MS calcd. for C₁₅H₁₄NO₂S [M−OH]⁺272.07, found 271.97.

### 3-(Ethylthio)-11-hydroxy-6-methyl-6,11-dihydrodibenzo[c,f][1,2]thiazepine 5,5-dioxide (4z)

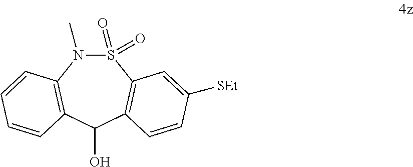

The product 4z was obtained as a yellow glass (106 mg, 98%). ¹H NMR (400 MHz, CDCl₃) δ 7.84 (s, 1H), 7.63-7.53 (m, 2H), 7.46 (d, J=7.6 Hz, 1H), 7.42-7.26 (m, 3H), 5.92 (d, J=9.0 Hz, 1H), 4.47 (d, J=9.2 Hz, 1H), 3.18 (s, 3H), 3.02 (dd, J=14.2, 7.0 Hz, 2H), 1.36 (t, J=7.2 Hz, 3H); ¹³C NMR (101 MHz, CDCl₃) δ 139.3, 138.8, 137.4, 135.9, 134.6, 132.1, 131.2, 130.4, 129.8, 127.7, 127.0, 126.6, 75.8, 39.2, 27.0, 14.0; LR-MS calcd. for C₁₆H₁₆NO₂S₂[M−OH]⁺318.06, found 318.29.

### 11-Hydroxy-3-(isopropylthio)-6-methyl-6,11-dihydrodibenzo[c,f][1,2]thiazepine 5,5-dioxide (4aa)

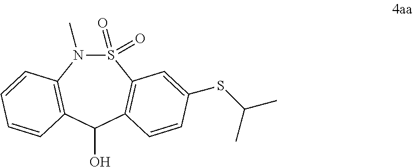 [SMILES: CC(C)Sc1ccc2c(c1)S(=O)(=O)N(C)c1ccccc1C2O]

The product 4aa was obtained as a yellow glass (264 mg, 100%). ¹H NMR (500 MHz, CDCl₃) δ 7.88 (s, 1H), 7.60-7.54 (m, 2H), 7.49 (dd, J=8.0, 0.8 Hz, 1H), 7.36-7.25 (m, 3H), 5.94 (d, J=8.5 Hz, 1H), 4.47 (d, J=8.8 Hz, 1H), 3.52-3.40 (m, 1H), 3.16 (s, 3H), 1.31 (dd, J=6.7, 3.0 Hz, 6H); ¹³C NMR (126 MHz, CDCl₃) δ 138.5, 138.0, 137.4, 136.5, 135.6, 134.6, 130.6, 129.9, 129.6, 129.0, 127.7, 127.0, 75.0, 39.0, 37.8, 22.9; LR-MS calcd. for C₁₇H₁NO₂S₂[M−OH]⁺332.08, found 332.32.

### 3-(Benzyloxy)-11-hydroxy-6-methyl-6,11-dihydrodibenzo[c,f][1,2]thiazepine 5,5-dioxide (4ab)

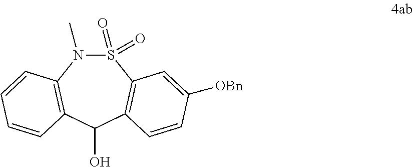 [SMILES: CN1c2ccccc2C(O)c2ccc(OCc3ccccc3)cc2S1(=O)=O]

The product 4ab was obtained as a powdery white solid (115 mg, 97%). ¹H NMR (500 MHz, CDCl₃) δ 7.62-7.58 (m, 2H), 7.56 (d, J=8.4 Hz, 1H), 7.46-7.30 (m, 7H), 7.28 (dd, J=7.9, 1.1 Hz, 1H), 7.17 (dd, J=8.4, 2.7 Hz, 1H), 5.80 (d, J=10.6 Hz, 1H), 5.15 (s, 2H), 4.41 (d, J=10.6 Hz, 1H), 3.12 (s, 3H); ¹³C NMR (126 MHz, CDCl₃) δ 158.8, 139.6, 138.0, 136.0, 134.8, 132.9, 132.8, 129.9, 129.8, 128.9, 128.5, 127.8, 127.6, 127.1, 119.8, 114.5, 70.8, 39.9; LR-MS calcd. for C₂₁H₁₈NO₃S [M−OH]⁺364.10, found 364.41.

General Procedure for Preparation of Chlorides 5a-5ab

Thionyl chloride (4.0-6.0 mmol) was added dropwise to a solution of alcohol 4 (1.0 mmol) in anhydrous CH₂Cl₂ (7 mL) (in some cases neat thionyl chloride was used). The reaction mixture was stirred for 2-18 h at room temperature and then concentrated to give the chloride 5 as a white or off-white solid, which was used directly in the following reaction without further purification.

### 10-Chloro-4-methyl-4,10-dihydrobenzo[f]thieno[3,2-c][1,2]thiazepine 5,5-dioxide (5a)

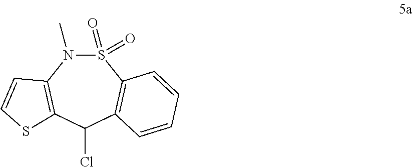 [SMILES: CN1c2ccsc2C(Cl)c2ccccc2S1(=O)=O]

Full conversion was not achieved despite use of an excess of thionyl chloride and a prolonged reaction time. The crude product was obtained as a brown solid (170 mg, mixture of 4a and 5a—1:2 determined by ¹H NMR) and was used in the next step without further purification. ¹H NMR (400 MHz Acetone-d₆) δ 8.03 (d, J=7.8 Hz, 1H), 7.93 (d, J=7.6 Hz, 1H), 7.82 (dd, J=7.5, 1.4 Hz, 1H), 7.74 (td, J=7.6, 1.3 Hz, 1H), 7.64 (d, J=5.5 Hz, 1H), 7.13 (s, 1H), 7.03 (d, J=5.5 Hz, 1H), 3.08 (s, 3H).

### 3-Bromo-10-chloro-4-methyl-4,10-dihydrobenzo[f]thieno[3,2-c][1,2]thiazepine 5,5-dioxide (5b)

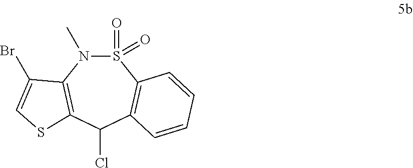 [SMILES: CN1c2c(Br)csc2C(Cl)c2ccccc2S1(=O)=O]

The product 5b was obtained as a pale-tan solid (168 mg, 94%). ¹H NMR (400 MHz, CDCl₃) δ 8.08 (dd, J=7.7, 1.2 Hz, 1H), 7.78 (d, J=7.6 Hz, 1H), 7.70 (td, J=7.6, 1.3 Hz, 1H), 7.61 (td, J=7.6, 1.2 Hz, 1H), 7.36 (s, 1H), 6.74 (s, 1H), 2.97 (s, 3H); ¹³C NMR (101 MHz, CDCl₃) δ 136.6, 136.0, 134.2, 132.6, 131.5, 131.0, 129.9, 129.8, 123.8, 110.0, 58.1, 39.2; LR-MS calcd. for C₁₂H₉BrNO₂S₂[M−Cl]⁺ 343.92, found 344.71.

### 7,10-Dichloro-4-methyl-4,10-dihydrobenzo[f]thieno[3,2-c][1,2]thiazepine 5,5-dioxide (5c)

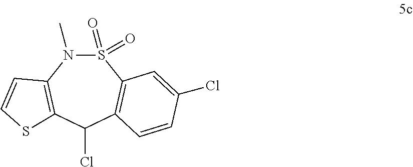 [SMILES: CN1c2ccsc2C(Cl)c2ccc(Cl)cc2S1(=O)=O]

The product 5c was obtained as a dark brown foam in quantitative yield. ¹H NMR (400 MHz, CDCl₃) δ 7.99 (d, J=2.1 Hz, 1H), 7.74 (d, J=8.3 Hz, 1H), 7.64 (dd, J=8.2, 2.2 Hz, 1H), 7.32 (d, J=5.3 Hz, 1H), 6.85 (d, J=4.7 Hz, 2H), 3.13 (s, 3H); ¹³C NMR (101 MHz, CDCl₃) δ 137.28, 137.05, 135.80, 133.93, 131.73, 131.36, 128.10, 126.22, 125.84, 124.62, 57.62, 39.39. LR-MS cald. for C₁₂H₉ClNO₂S₂[M−Cl]⁺ 297.98, found 298.00.

### 7-Bromo-10-chloro-4-methyl-4,10-dihydrobenzo[f]thieno[3,2-c][1,2]thiazepine 5,5-dioxide (5d)

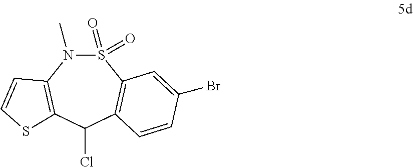 [SMILES: CN1c2ccsc2C(Cl)c2ccc(Br)cc2S1(=O)=O]

The product 5d was obtained as a brown solid containing 6% starting material by NMR (577 mg, 92% corrected for starting material impurity). ¹H NMR (500 MHz, CDCl₃) δ 8.14 (d, J=2.0 Hz, 1H), 7.80 (dd, J=8.3, 2.0 Hz, 1H), 7.67 (d, J=8.3 Hz, 1H), 7.32 (d, J=5.5 Hz, 1H), 6.85 (d, J=6.8 Hz, 1H), 6.84 (s, 1H), 3.13 (s, 3H); ¹³C NMR (126 MHz, CDCl₃) δ 137.3, 137.04, 137.00, 131.9, 131.8, 130.9, 126.1, 125.8, 124.6, 123.6, 57.7, 39.4; LR-MS cald. for C₁₂H₉BrNO₂S₂[M−Cl]⁺ 341.93, found 342.47.

### 11-Chloro-6-methyl-6,11-dihydrodibenzo[c,f][1,2]thiazepine 5,5-dioxide (5e)

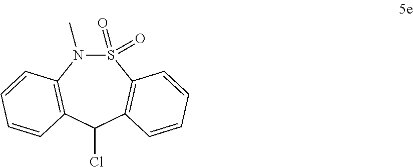 [SMILES: CN1c2ccccc2C(Cl)c2ccccc2S1(=O)=O]

The chloride 5e was prepared according the procedure described in Gilleron et al. 2007. ¹H NMR (500 MHz, Acetone-d₆) δ 7.97 (dd, J=7.3, 1.8 Hz, 1H), 7.83-7.78 (m, 1H), 7.74-7.63 (m, 4H), 7.59 (t, J=7.6 Hz, 1H), 7.43 (t, J=7.5 Hz, 1H), 6.65 (s, 1H), 3.56 (s, 3H).

### 3,11-Dichloro-6-methyl-6,11-dihydrodibenzo[c,f][1,2]thiazepine 5,5-dioxide (5f)

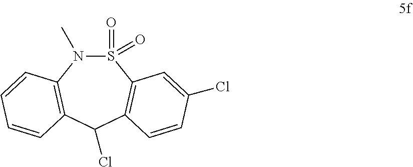 [SMILES: CN1c2ccccc2C(Cl)c2ccc(Cl)cc2S1(=O)=O]

Chloride 5f was purchased from Ark Pharm, Inc. (Libertyville, IL) and used without further purification.

### 11-Chloro-3-fluoro-6-methyl-6,11-dihydrodibenzo[c,f][1,2]thiazepine 5,5-dioxide (5g)

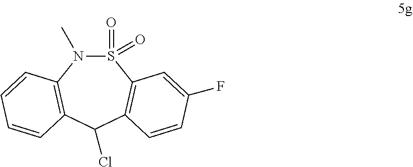 [SMILES: CN1c2ccccc2C(Cl)c2ccc(F)cc2S1(=O)=O]

The product 5g was obtained as a white solid (219 mg, 99%). ¹H NMR (500 MHz, CDCl₃) δ 7.71 (dd, J=8.1, 2.7 Hz, 1H), 7.58-7.49 (m, 3H), 7.43 (d, J=7.5 Hz, 1H), 7.39-7.34 (m, 1H), 7.26-7.21 (m, 1H), 6.14 (s, 1H), 3.59 (s, 3H); ¹³C NMR (126 MHz, CDCl₃) (additional peaks due to C—F coupling) δ 163.9, 161.8, 142.5 139.3 137.8 133.8, 133.8, 131.7, 131.3, 130.1, 129.5, 129.0, 119.8, 119.7, 115.5, 115.3, 63.7, 39.3; LR-MS calcd. for C₁₄H₁₁FNO₂S [M−Cl]⁺ 276.05, found 275.78.

### 11-Chloro-3-methoxy-6-methyl-6,11-dihydrodibenzo[c,f][1,2]thiazepine 5,5-dioxide (5h)

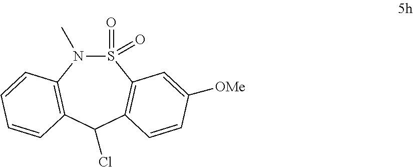 [SMILES: COc1ccc2c(c1)S(=O)(=O)N(C)c1ccccc1C2Cl]

The product 5h was obtained as an off-white solid (158 mg, 99%). ¹H NMR (500 MHz, CDCl₃) δ 7.57-7.39 (m, 5H), 7.34 (t, J=7.4 Hz, 1H), 7.05 (dd, J=8.6, 2.6 Hz, 1H), 6.14 (s, 1H), 3.88 (s, 3H), 3.58 (s, 3H); ¹³C NMR (126 MHz, CDCl₃) δ 160.7, 141.6, 139.5, 138.3, 133.4, 131.4, 130.0, 129.5, 128.8, 127.1, 119.3, 112.0, 64.7, 56.0, 39.3; LR-MS calcd. for C₁₅H₁₄NO₃S [M−Cl]⁺ 288.07, found 287.94.

### 11-Chloro-6-methyl-3-phenoxy-6,11-dihydrodibenzo[c,f][1,2]thiazepine 5,5-dioxide (5i)

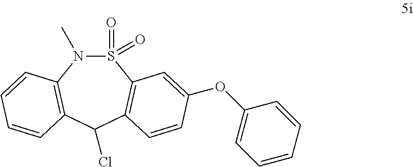 [SMILES: CN1c2ccccc2C(Cl)c2ccc(Oc3ccccc3)cc2S1(=O)=O]

The product 5i was obtained as an off-white solid (283 mg, 97%). ¹H NMR (500 MHz, CDCl₃) δ 7.57 (d, J=2.6 Hz, 1H), 7.55-7.47 (m, 3H), 7.45-7.33 (m, 4H), 7.21 (t, J=7.4 Hz, 1H), 7.11 (dd, J=8.6, 2.6 Hz, 1H), 7.09-7.03 (m, 2H), 6.14 (s, 1H), 3.58 (s, 3H); ¹³C NMR (126 MHz, CDCl₃) δ 159.3, 155.3, 142.0, 139.4, 138.1, 133.5, 131.5, 130.4, 130.0, 129.5, 129.0, 128.9, 125.1, 121.5, 120.2, 116.7, 64.4, 39.3; LR-MS calcd. for C₂₀H₁₆NO₃S [M−Cl]⁺ 350.09, found 349.75.

### 11-Chloro-3,6-dimethyl-6,11-dihydrodibenzo[c,f][1,2]thiazepine 5,5-dioxide (5j)

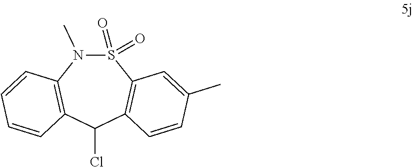 [SMILES: Cc1ccc2c(c1)S(=O)(=O)N(C)c1ccccc1C2Cl]

The product 5j was obtained as an off-white solid (220 mg, 94%). ¹H NMR (500 MHz, CDCl₃) δ 7.83 (s, 1H), 7.56-7.47 (m, 2H), 7.46-7.40 (m, 2H), 7.37-7.31 (m, 2H), 6.13 (s, 1H), 3.58 (s, 3H), 2.42 (s, 3H); ¹³C NMR (126 MHz, CDCl₃) δ 141.0, 140.2, 139.5, 138.2, 133.3, 132.3, 131.5, 131.4, 130.0, 129.4, 128.8, 128.4, 64.4, 39.1, 21.2; LR-MS calcd. for C₁₅H₁₄NO₂S [M−Cl]⁺ 272.07, found 272.75.

### 9-Bromo-3,11-dichloro-6-methyl-6,11-dihydrodibenzo[c,f][1,2]thiazepine 5,5-dioxide (5k)

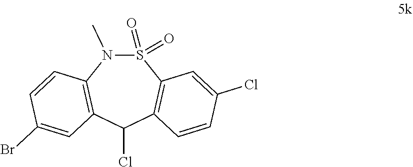 [SMILES: CN1c2ccc(Br)cc2C(Cl)c2ccc(Cl)cc2S1(=O)=O]

The product 5k was obtained as an off-white solid (270 mg, 99%). ¹H NMR (400 MHz, Acetone-d₆) δ 7.91 (s, 1H), 7.90 (s, 1H), 7.82 (d, J=8.4 Hz, 1H), 7.79-7.73 (m, 2H), 7.64 (d, J=8.5 Hz, 1H), 6.67 (s, 1H), 3.54 (s, 3H); ¹³C NMR (101 MHz, Acetone-d₆) δ 143.0, 140.4, 139.9, 136.6, 135.2, 135.0, 134.4, 134.0, 133.6, 132.1, 127.9, 122.1, 62.6, 39.4; LR-MS calcd. for C₁₄H₁₀BrClNO₂S [M−Cl]⁺ 371.93, found 372.84.

### 11-Chloro-6-methyl-3-(methylthio)-6,11-dihydrodibenzo[c,f][1,2]thiazepine 5,5-dioxide (5l)

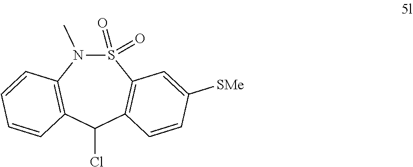 [SMILES: CSc1ccc2c(c1)S(=O)(=O)N(C)c1ccccc1C2Cl]

The product 5l was obtained as an off-white solid (329 mg, 98%)¹H NMR (400 MHz, CDCl₃) δ 7.78 (s, 1H), 7.56-7.47 (m, 2H), 7.45-7.40 (m, 2H), 7.38-7.32 (m, 2H), 6.12 (s, 1H), 3.58 (s, 3H), 2.53 (s, 3H). ¹³C NMR (101 MHz, CDCl₃) δ 142.9, 140.9, 139.4, 137.9, 131.9, 131.5, 130.9, 130.1, 129.6, 129.5, 128.9, 124.1, 64.5, 39.3, 15.3; LR-MS calcd. for C₁₅H₁₄NO₂S₂ [M−Cl]⁺ 304.05, found 304.74.

### 3-Bromo-11-chloro-6-methyl-6,11-dihydrodibenzo[c,f][1,2]thiazepine 5,5-dioxide (5o)

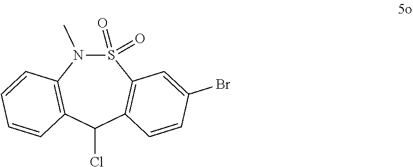 [SMILES: CN1c2ccccc2C(Cl)c2ccc(Br)cc2S1(=O)=O]

The product 5o was obtained as a white solid (60 mg, 95%). ¹H NMR (400 MHz, CDCl₃) δ 8.14 (d, J=2.0 Hz, 1H), 7.66 (dd, J=8.3, 2.1 Hz, 1H), 7.54-7.50 (m, 2H), 7.45-7.40 (m, 2H), 7.38-7.33 (m, 1H), 6.10 (s, 1H), 3.58 (s, 3H); ¹³C NMR (101 MHz, CDCl₃) δ 142.0, 139.2, 137.5, 135.6, 134.1, 133.0, 131.7, 131.0, 130.2, 129.5, 129.0, 124.3, 63.7, 39.3; LR-MS calcd. for C₁₄H₁₁BrNO₂S [M−Cl]⁺ 335.97, found 336.01.

### 11-Chloro-3-iodo-6-methyl-6,11-dihydrodibenzo[c,f][1,2]thiazepine 5,5-dioxide (5q)

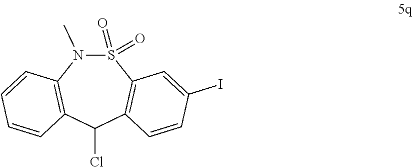 [SMILES: CN1c2ccccc2C(Cl)c2ccc(I)cc2S1(=O)=O]

The product 5q was obtained as a gray solid (73.4 mg, 96%). ¹H NMR (400 MHz, CDCl₃) δ 8.32 (d, J=1.8 Hz, 1H), 7.87 (dd, J=8.2, 1.8 Hz, 1H), 7.57-7.48 (m, 2H), 7.43 (d, J=7.1 Hz, 1H), 7.39-7.33 (m, 1H), 7.27 (d, J=7.1 Hz, 1H), 6.08 (s, 1H), 3.57 (s, 3H); ¹³C NMR (101 MHz, CDCl₃) δ 141.8, 141.5, 139.3, 137.6, 136.7, 134.7, 132.9, 131.7, 130.1, 129.5, 129.0, 95.4, 63.8, 39.3; LR-MS calcd. for C₁₄H₁₁INO₂S [M−Cl]⁺ 383.96, found 383.70.

### 11-Chloro-7-methoxy-6-methyl-6,11-dihydrodibenzo[c,f][1,2]thiazepine 5,5-dioxide (5r)

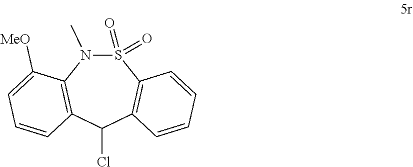 [SMILES: COc1cccc2c1N(C)S(=O)(=O)c1ccccc1C2Cl]

The product 5r was obtained as a white solid (70.4 mg, quantitative). ¹H NMR (400 MHz, CDCl₃) δ 8.04-7.97 (m, 1H), 7.56-7.47 (m, 3H), 7.31 (t, J=8.0 Hz, 1H), 7.05 (d, J=8.4 Hz, 1H), 6.99 (d, J=7.7 Hz, 1H), 6.03 (s, 1H), 3.97 (s, 3H), 3.54 (s, 3H); ¹³C NMR (101 MHz, CDCl₃) δ 158.4, 141.2, 140.1, 135.1, 132.2, 131.7, 130.4, 130.0, 128.1, 127.4, 121.3, 114.1, 64.7, 56.4, 38.1; LR-MS calcd. for C₁₅H₁₄NO₃S [M−Cl]⁺ 288.07, found 288.07.

### 11-Chloro-6-methyl-3-phenyl-6,11-dihydrodibenzo[c,f][1,2]thiazepine 5,5-dioxide (5t)

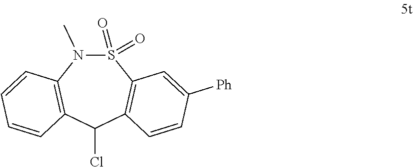 [SMILES: CN1c2ccccc2C(Cl)c2ccc(-c3ccccc3)cc2S1(=O)=O]

The product 5t was obtained as a white solid (235 mg, 99%). ¹H NMR (400 MHz, CDCl₃) δ 8.23 (d, J=1.9 Hz, 1H), 7.76 (dd, J=8.1, 2.0 Hz, 1H), 7.65-7.59 (m, 3H), 7.56 (dd, J=7.9, 1.4 Hz, 1H), 7.52 (td, J=7.9, 7.5, 1.5 Hz, 1H), 7.49-7.44 (m, 3H), 7.44-7.38 (m, 1H), 7.36 (td, J=7.6, 1.5 Hz, 1H), 6.21 (s, 1H), 3.61 (s, 3H); ¹³C NMR (101 MHz, CDCl₃) δ 143.5, 140.9, 139.5, 138.6, 138.0, 133.7, 132.1, 131.5, 130.9, 130.1, 129.5, 129.2, 128.8, 128.7, 127.3, 126.6, 64.3, 39.2; LR-MS calcd. for C₂₀H₁₆NO₂S [M−Cl]⁺ 334.09, found 334.15.

### 11-Chloro-6-methyl-3-(methylsulfonyl)-6,11-dihydrodibenzo[c,f][1,2]thiazepine 5,5-dioxide (5u)

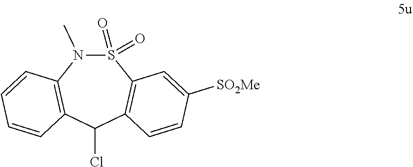 [SMILES: CN1c2ccccc2C(Cl)c2ccc(S(C)(=O)=O)cc2S1(=O)=O]

Neat thionyl chloride was used. The product 5u was obtained as an off-white solid (210 mg, 94%). ¹H NMR (400 MHz, CDCl₃) δ 8.57 (d, J=1.8 Hz, 1H), 8.10 (dd, J=8.1, 1.9 Hz, 1H), 7.79 (d, J=8.2 Hz, 1H), 7.58-7.51 (m, 2H), 7.47 (d, J=7.5 Hz, 1H), 7.43-7.35 (m, 1H), 6.21 (s, 1H), 3.60 (s, 3H), 3.10 (s, 3H); ¹³C NMR (101 MHz, CDCl₃) δ 142.5, 142.0, 140.4, 139.0, 136.9, 132.6, 132.1, 130.9, 130.3, 129.5, 129.2, 127.7, 63.0, 44.5, 39.2; LR-MS calcd. for C₁₅H₁₄NO₄S₂[M−Cl]⁺ 336.04, found 336.18.

### 11-Chloro-6-methyl-3-(methylsulfinyl)-6,11-dihydrodibenzo[c,f][1,2]thiazepine 5,5-dioxide (5v)

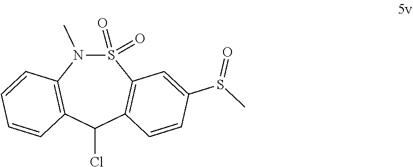 [SMILES: CN1c2ccccc2C(Cl)c2ccc(S(C)=O)cc2S1(=O)=O]

Chloride 5v was prepared by an alternative procedure described as follows. To a solution of alcohol 4v (74.9 mg, 0.222 mmol) in anhydrous CH₂Cl₂ (3.4 mL) was added a 2.0 M solution of HCl in anhydrous Et₂O (0.67 mL, 1.33 mmol) and the resulting solution was stirred at room temperature for 1.25 h. It was then concentrated directly in vacuo to yield the pure chloride 5v as an off-white solid (76.0 mg, 96%, 1:1 mixture of diastereomers). ¹H NMR (400 MHz, CDCl₃) (partial integrals due to mixture of diastereomers) δ 8.13 (s, 1H), 7.95 (dt, J=8.1, 2.1 Hz, 1H), 7.77 (d, J=7.7 Hz, 1H), 7.58-7.50 (m, 2H), 7.47 (d, J=7.1 Hz, 1H), 7.41-7.35 (m, 1H), 6.20 (s, 0.5H), 6.19 (s, 0.5H), 3.61 (s, 1.5H), 3.60 (s, 1.5H), 2.80 (s, 1.5H), 2.77 (s, 1.5H); ¹³C NMR (101 MHz, CDCl₃) (additional peaks due to mixture of diastereomers) 148.8, 141.6, 139.1, 137.7, 137.4, 132.8, 132.7, 131.9, 130.3, 130.2, 129.5, 129.5, 129.1, 127.3, 123.7, 123.5, 63.5, 63.5, 44.0, 39.2; LR-MS calcd. for C₁₅H₁₄NO₃S₂[M−Cl]⁺ 320.04, found 320.20.

### 2,11-Dichloro-6-methyl-6,11-dihydrodibenzo[c,f][1,2]thiazepine 5,5-dioxide (5w)

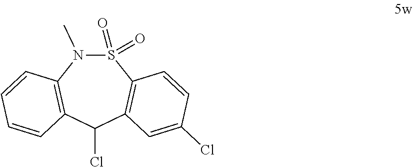 [SMILES: CN1c2ccccc2C(Cl)c2cc(Cl)ccc2S1(=O)=O]

The product 5w was obtained as a white solid (195 mg, 99%). ¹H NMR (400 MHz, CDCl₃) δ 7.97 (d, J=8.5 Hz, 1H), 7.60-7.50 (m, 4H), 7.46 (d, J=7.4 Hz, 1H), 7.41-7.35 (m, 1H), 6.11 (s, 1H), 3.60 (s, 3H); ¹³C NMR (101. MHz, CDCl₃) δ 139.1, 138.7, 138.3, 137.3, 136.9, 131.6, 130.9, 130.1, 129.9, 129.6, 129.2, 128.8, 63.1, 38.9; LR-MS calcd. for C₁₄H₁₁ClNO₂S [M−Cl]⁺ 292.02, found 292.38.

### 8,11-Dichloro-6-methyl-6,11-dihydrodibenzo[c,f][1,2]thiazepine 5,5-dioxide (5x)

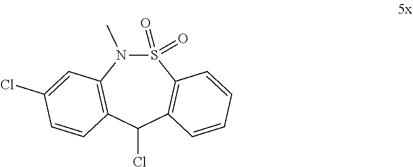 [SMILES: CN1c2cc(Cl)ccc2C(Cl)c2ccccc2S1(=O)=O]

The product 5× was obtained as an off-white solid (390 mg, 100%). ¹H NMR (500 MHz, CDCl₃) δ 8.04-7.99 (m, 1H), 7.59-7.51 (m, 4H), 7.39 (d, J=8.3 Hz, 1H), 7.32 (dd, J=8.3, 2.0 Hz, 1H), 6.14 (s, 1H), 3.55 (s, 3H); ¹³C NMR (126 MHz, CDCl₃) δ 140.7, 140.2, 136.9, 136.4, 134.8, 132.8, 131.5, 131.1, 130.4, 129.5, 129.0, 128.1, 63.6, 39.0; LR-MS calcd. for C₁₄H₁₁ClNO₂S [M−Cl]⁺ 292.02, found 292.56.

### 11-Chloro-6-ethyl-6,11-dihydrodibenzo[c,f][1,2]thiazepine 5,5-dioxide (5y)

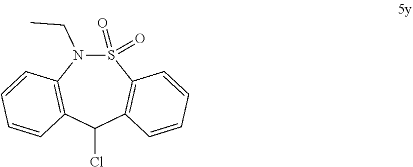 [SMILES: CCN1c2ccccc2C(Cl)c2ccccc2S1(=O)=O]

The product 5y was obtained as a white solid (230 mg, 98%). ¹H NMR (500 MHz, CDCl₃) δ 8.04-8.00 (m, 1H), 7.59-7.45 (m, 6H), 7.37 (td, J=7.6, 1.3 Hz, 1H), 6.22 (s, 1H), 4.12 (dq, J=14.4, 7.2 Hz, 1H), 3.83 (dq, J=14.2, 7.1 Hz, 1H), 1.45 (t, J=7.2 Hz, 3H); ¹³C NMR (126 MHz, CDCl₃) δ 140.7, 138.4, 138.1, 135.1, 132.5, 131.4, 131.2, 130.8, 130.4, 130.2, 129.0, 128.3, 64.1, 48.0, 16.3; LR-MS calcd. for C₁₅H₁₄NO₂S [M−Cl]⁺ 272.07, found 271.97.

### 11-Chloro-3-(ethylthio)-6-methyl-6,11-dihydrodibenzo[c,f][1,2]thiazepine 5,5-dioxide (5z)

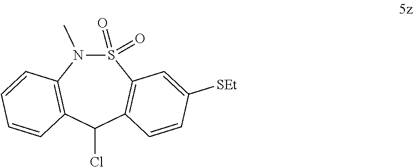

The product 5z was obtained as a pale-pinkish solid (109 mg, 100%). ¹H NMR (500 MHz, CDCl₃) δ 7.83 (d, J=1.5 Hz, 1H), 7.55-7.47 (m, 2H), 7.44-7.37 (m, 3H), 7.37-7.32 (m, 1H), 6.11 (s, 1H), 3.58 (s, 3H), 3.03 (q, J=7.4 Hz, 2H), 1.36 (t, J=7.4 Hz, 3H); ¹³C NMR (126 MHz, CDCl₃) δ 141.6, 140.9, 139.4, 137.9, 131.9, 131.5, 131.4, 131.0, 130.1, 129.5, 128.9, 125.7, 64.4, 39.3, 26.8, 14.0; LR-MS calcd. for C₁H₁₆NO₂S₂[M−Cl]⁺ 318.06, found 318.11.

### 11-Chloro-3-(isopropylthio)-6-methyl-6,11-dihydrodibenzo[c,f][1,2]thiazepine 5,5-dioxide (5aa)

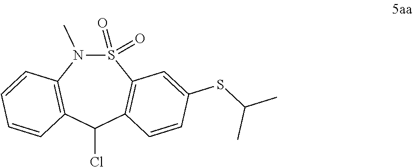 [SMILES: CC(C)Sc1ccc2c(c1)S(=O)(=O)N(C)c1ccccc1C2Cl]

The product 5aa was obtained as a pale-orange solid (265 mg, 98%). ¹H NMR (500 MHz, CDCl₃) δ 7.91 (s, 1H), 7.56-7.47 (m, 2H), 7.47-7.41 (m, 3H), 7.35 (td, J=7.6, 1.5 Hz, 1H), 6.12 (s, 1H), 3.58 (s, 3H), 3.57-3.49 (m, 1H), 1.35 (t, J=6.2 Hz, 6H); ¹³C NMR (126 MHz, CDCl₃) δ 140.8, 140.7, 139.4, 137.9, 133.2, 132.1, 131.9, 131.5, 130.1, 129.5, 128.9, 128.1, 64.3, 39.2, 37.5, 23.1; LR-MS calcd. for C₁₇H₁₈NO₂S₂ [M−Cl]⁺ 332.08, found 332.30.

### 3-(Benzyloxy)-11-chloro-6-methyl-6,11-dihydrodibenzo[c,f][1,2]thiazepine 5,5-dioxide (5ab)

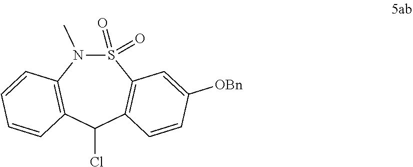 [SMILES: CN1c2ccccc2C(Cl)c2ccc(OCc3ccccc3)cc2S1(=O)=O]

The product 5ab was obtained as an off-white solid (113 mg, 99%). ¹H NMR (500 MHz, CDCl₃) δ 7.60 (d, J=2.7 Hz, 1H), 7.53 (dd, J=7.8, 1.3 Hz, 1H), 7.49 (td, J=7.5, 1.4 Hz, 1H), 7.46 (d, J=8.7 Hz, 1H), 7.44-7.38 (m, 5H), 7.37-7.32 (m, 2H), 7.11 (dd, J=8.6, 2.7 Hz, 1H), 6.14 (s, 1H), 5.12 (s, 2H), 3.58 (s, 3H); ¹³C NMR (126 MHz, CDCl₃) δ 159.86, 141.66, 139.48, 138.21, 135.86, 133.39, 131.41, 130.03, 129.50, 128.88, 128.81, 128.54, 127.78, 127.30, 119.70, 113.13, 70.75, 64.67, 39.30; LR-MS calcd. for C₂₁H₁NO₃S [M−Cl]⁺ 364.10, found 364.44.

### General Procedures for Preparation of N-Substituted 11-amino-6-alkyl-6,11-dihydrodiaryl[c,f][1,2]thiazepine 5,5-dioxides

N-substituted 11-amino-6-alkyl-6,11-dihydrodiaryl[c,f][1,2]thiazepine 5,5-dioxides were prepared via amination of the corresponding chlorides 5 with amines according to general methods A, B, or C.

Method A:

An amine (0.26 mmol) and Et₃N (0.40 mmol) were added to a solution of the chloride 5 (0.2 mmol) in nitromethane (1 mL). The reaction vial was sealed under Ar and heated to 60° C. for 20 min. The reaction mixture was concentrated, and the crude product was purified by column chromatography.

Method B:

To a suspension of chloride 5 (0.500 mmol) in nitromethane (1.0 mL) was added an amine (1.00 mmol), and the mixture was warmed to 60° C. and left to stir until TLC indicated that the reaction was complete (typically <30 min.). The reaction mixture was concentrated, and the residue partitioned between Et₂O (10 mL) and water (10 mL). The ethereal layer was separated and the aqueous extracted again with Et₂O (10 mL). The combined organics were washed with H₂O (10 mL) and 10% NH₄OH (10 mL), dried over Na₂SO₄, and concentrated to yield the product. If necessary, the product was further purified by column chromatography. Alternatively, in some cases no extraction was performed and the concentrated reaction was purified directly by column chromatography.

Method C:

To a suspension of chloride 5 (0.500 mmol) in nitromethane (1.0 mL) was added an amine hydrochloride (0.600 mmol) and Et₃N (1.20 mmol) and the mixture was warmed to 60° C. and left to stir until TLC indicated that the reaction was complete (typically <30 min.). The reaction mixture was concentrated and the residue partitioned between Et₂O (10 mL) and water (10 mL). The ethereal layer was separated and the aqueous extracted again with Et₂O (10 mL). The combined organics were washed with H₂O (10 mL) and 10% NH₄OH (10 mL), dried over Na₂SO₄, and concentrated to yield the product. If necessary, the product was further purified by column chromatography. Alternatively, in some cases no extraction was performed and the concentrated reaction was purified directly by column chromatography.

### Ethyl 7-((4-methyl-5,5-dioxido-4,10-dihydrobenzo[f]thieno[3,2-c][1,2]thiazepin-10-yl)amino)heptanoate (6)

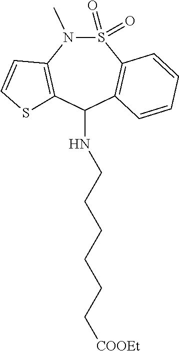 [SMILES: CCOC(=O)CCCCCCNC1c2ccccc2S(=O)(=O)N(C)c2ccsc21]

Method A. The crude product was purified by column chromatography (hexanes:ethyl acetate—2:1). The product 6 was obtained as a viscous yellow oil (96 mg, 56% after two steps). A second round of column chromatography was performed (hexanes:ethyl acetate—1:1) to remove the intense yellow color (54 mg of a yellowish solid was obtained after the second purification). ¹H NMR (400 MHz, Acetone-d₆) δ 7.98-7.92 (m, 1H), 7.80 (td, J=7.6, 1.2 Hz, 1H), 7.74 (d, J=7.2 Hz, 1H), 7.61 (td, J=7.6, 1.2 Hz, 1H), 7.33 (d, J=5.5 Hz, 1H), 6.96 (d, J=5.5 Hz, 1H), 5.55 (d, J=9.4 Hz, 1H), 4.07 (q, J=7.1 Hz, 2H), 2.96 (s, 3H), 2.78 (t, J=8.2 Hz, 2H, partially overlaps with the residual acetone peak), 2.69-2.58 (m, 1H) 2.27 (t, J=7.4 Hz, 2H), 1.67-1.51 (m, 4H), 1.48-1.31 (m, 4H), 1.20 (t, J=7.1 Hz, 3H); ¹³C NMR (101 MHz, Acetone-d₆) δ 173.6, 137.2, 136.2, 136.2, 134.8, 132.0, 129.2, 129.0, 128.7, 125.7, 124.6, 60.9, 60.4, 48.3, 39.8, 34.6, 30.7, 27.6, 25.7; LR-MS calcd. for C₂₁H₂₉N₂O₄S₂ [M+H]⁺ 437.16, found 438.13.

### 10-((3-Methoxypropyl)amino)-4-methyl-4,10-dihydrobenzo[f]thieno[3,2-c][1,2]thiazepine 5,5-dioxide (7)

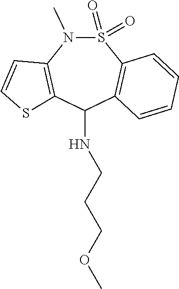 [SMILES: COCCCNC1c2ccccc2S(=O)(=O)N(C)c2ccsc21]

Method A. The crude product was purified by column chromatography (hexanes:ethyl acetate—1:2). The product 7 was obtained as a viscous blue oil (43 mg, 54% after two steps). A second round of column chromatography was performed (hexane:ethyl acetate—1:1) to remove the intense blue color (23 mg of a yellowish solid was obtained after the second purification). ¹H NMR (400 MHz, Acetone-d₆) δ 7.95 (dd, J=7.7, 1.0 Hz, 1H), 7.80 (td, J=7.6, 1.2 Hz, 1H), 7.74 (d, J=7.3 Hz, 1H), 7.61 (td, J=7.6, 1.2 Hz, 1H), 7.33 (d, J=5.5 Hz, 1H), 6.97 (d, J=5.5 Hz, 1H), 5.55 (br d, 1H), 3.50 (t, J=6.2 Hz, 2H), 3.28 (s, 3H), 3.19-3.06 (m, J=1H), 2.96 (s, 3H), 2.91-2.68 (m, 2H, partially overlaps with the residual acetone peak), 1.78 (p, J=6.4 Hz, 2H); ¹³C NMR (101 MHz, Acetone-d₆) δ 137.2, 136.2, 136.1, 134.8, 131.9, 129.0, 128.9, 128.7, 125.8, 124.5, 71.4, 60.9, 58.6, 45.7, 39.8, 31.0; LR-MS calcd. for C₁H₂₁N₂O₃S₂[M+H]⁺ 353.10, found 353.86.

### 6-Methyl-11-(pentylamino)-6,11-dihydrodibenzo[c,f][1,2]thiazepine 5,5-dioxide (8)

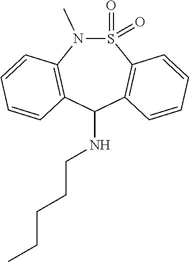 [SMILES: CCCCCNC1c2ccccc2N(C)S(=O)(=O)c2ccccc21]

Method A. The crude product was purified by column chromatography (hexanes:ethyl acetate—2:1). The product 8 was obtained as a viscous colorless oil (65 mg, 94%). ¹H NMR (400 MHz, Methanol-d₄) δ 7.93 (d, J=7.7 Hz, 1H), 7.63-7.59 (m, 2H), 7.56-7.46 (m, 2H), 7.45-7.37 (m, 2H), 7.32 (td, J=7.3, 1.8 Hz, 1H), 5.08 (s, 1H), 3.27 (s, 3H), 2.53-2.36 (m, 2H), 1.57-1.40 (m, 2H), 1.31-1.16 (m, 4H), 0.86 (t, J=6.9 Hz, 3H); ¹³C NMR (101 MHz, Methanol-d₄) δ 140.6, 139.9, 139.3, 139.2, 133.8, 132.0, 132.0, 130.4, 129.6, 129.4, 128.9, 128.7, 67.9, 38.9, 30.5, 30.2, 23.5, 14.3; LR-MS calcd. for C₁₉H₂₅N₂O₂S [M+H]⁺ 345.16, found 345.91.

### Ethyl 7-((6-methyl-5,5-dioxido-6,11-dihydrodibenzo[c,f][1,2]thiazepin-11-yl)amino)heptanoate (9)

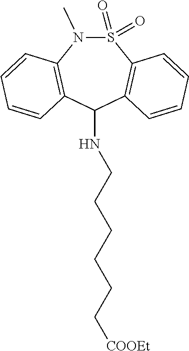 [SMILES: CCOC(=O)CCCCCCNC1c2ccccc2N(C)S(=O)(=O)c2ccccc21]

Method A. The crude product was purified by column chromatography (hexanes:ethyl acetate—2:1). The product 9 was obtained as a viscous colorless oil (68 mg, 79%). ¹H NMR (400 MHz, Methanol-d₄) δ 7.93 (d, J=7.7 Hz, 1H), 7.65-7.58 (m, 2H), 7.56-7.46 (m, 2H), 7.46-7.36 (m, 2H), 7.33 (dd, J=7.4, 1.7 Hz, 1H), 5.09 (s, 1H), 4.09 (q, J=7.1 Hz, 2H), 3.28 (s, 3H), 2.54-2.36 (m, 2H), 2.26 (t, J=7.4 Hz, 2H), 1.61-1.42 (m, 4H), 1.31-1.18 (m, 7H); ¹³C NMR (101 MHz, Methanol-d₄) δ 175.5, 140.6, 139.9, 139.4, 139.2, 133.8, 132.0, 132.0, 130.4, 129.6, 129.4, 128.9, 128.8, 67.9, 61.4, 48.5, 38.9, 35.0, 30.3, 29.8, 27.9, 25.9, 14.5; LR-MS calcd. for C₂₃H₃₁N₂O₄S [M+H]⁺ 431.20, found 432.32.

### 11-((3-Methoxypropyl)amino)-6-methyl-6,11-dihydrodibenzo[c,f][1,2]thiazepine 5,5-dioxide (10)

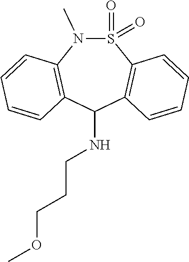 [SMILES: COCCCNC1c2ccccc2N(C)S(=O)(=O)c2ccccc21]

Method A. The crude product was purified by column chromatography (hexanes:ethyl acetate—1:2). The product 10 was obtained as a viscous colorless oil (59 mg, 85%). ¹H NMR (400 MHz, Acetone-d₆) δ 7.89 (dd, J=7.8, 1.2 Hz, 1H), 7.68 (d, J=7.7 Hz, 1H), 7.60 (td, J=7.5, 1.3 Hz, 1H), 7.52 (dt, J=7.6, 2.0 Hz, 2H), 7.46 (dd, J=7.8, 1.2 Hz, 1H), 7.38 (td, J=7.6, 1.6 Hz, 1H), 7.32 (td, J=7.5, 1.4 Hz, 1H), 5.20 (s, 1H), 3.44-3.35 (m, 5H), 3.22 (s, 3H), 2.58 (t, J=6.7 Hz, 2H), 1.73 (p, J=6.6 Hz, 2H); ¹³C NMR (101 MHz, Acetone-d₆) δ 141.0, 140.1, 140.1, 139.9, 133.0, 130.7, 130.6, 129.7, 128.9, 128.8, 128.5, 71.6, 66.7, 58.5, 46.1, 38.6, 30.8; LR-MS calcd. for C₁₈H₂₃N₂O₃S [M+H]⁺ 347.14, found 347.90.

### 9-Bromo-3-chloro-11-((3-methoxypropyl)amino)-6-methyl-6,11-dihydrodibenzo[c,f][1,2]thiazepine 5,5-dioxide (11)

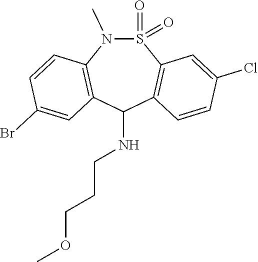 [SMILES: COCCCNC1c2cc(Br)ccc2N(C)S(=O)(=O)c2cc(Cl)ccc21]

Method A. The crude product was purified by column chromatography (hexanes:ethyl acetate—2:1). The product 11 was obtained as a white solid (75 mg, 82%). ¹H NMR (400 MHz, Acetone-d₆) δ 7.83 (d, J=2.2 Hz, 1H), 7.74-7.70 (m, 2H), 7.66 (dd, J=8.3, 2.2 Hz, 1H), 7.58 (dd, J=8.5, 2.4 Hz, 1H), 7.44 (d, J=8.5 Hz, 1H), 5.23 (s, 1H), 3.42-3.38 (m, 5H), 3.23 (s, 3H), 2.80 (br s, 1H), 2.60 (td, J=6.8, 2.1 Hz, 2H), 1.79-1.69 (m, 2H); ¹³C NMR (101 MHz, Methanol-d₄) δ 141.8, 141.7, 139.6, 137.5, 135.4, 134.6, 133.6, 133.5, 130.9, 128.9, 122.4, 101.4, 72.2, 66.5, 58.9, 46.3, 39.0, 30.5; LR-MS calcd. for C₁₈H₂₁BrClN₂O₃S [M+H]⁺ 461.01, found 462.07.

### 3-Chloro-11-((6-hydroxyhexyl)amino)-6-methyl-6,11-dihydrodibenzo[c,f][1,2]thiazepine 5,5-dioxide (12)

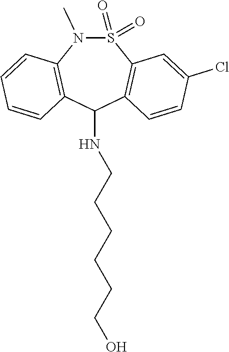 [SMILES: CN1c2ccccc2C(NCCCCCCO)c2ccc(Cl)cc2S1(=O)=O]

Method B. The product 12 was obtained as a viscous colorless oil (230 mg, 92%). ¹H NMR (400 MHz, Methanol-d₄) δ 7.88 (t, J=1.2 Hz, 1H), 7.62 (s, 1H), 7.62 (s, 1H), 7.47 (td, J=7.2, 1.5 Hz, 1H), 7.44-7.38 (m, 2H), 7.37-7.31 (m, 1H), 5.08 (s, 1H), 3.50 (t, J=6.6 Hz, 2H), 3.33 (s, 3H), 2.53-2.38 (m, 2H), 1.57-1.43 (m, 4H), 1.35-1.23 (m, 4H); ¹³C NMR (101 MHz, Acetone-d₆) δ 142.4, 140.6, 139.8, 138.6, 134.2, 133.0, 132.8, 131.0, 130.0, 129.4, 128.8, 128.2, 66.5, 62.4, 48.7, 39.2, 33.7, 30.8, 27.9, 26.6; LR-MS calcd. for C₂₀H₂₆ClN₂O₃S [M+H]⁺ 409.13, found 410.05.

### 6-((3-Chloro-6-methyl-5,5-dioxido-6,11-dihydrodibenzo[c,f][1,2]thiazepin-11-yl)amino)hexyl acetate (13)

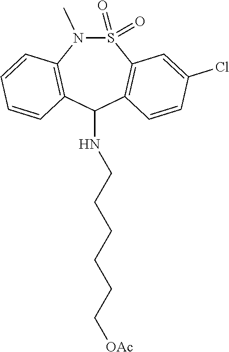 [SMILES: CC(=O)OCCCCCCNC1c2ccccc2N(C)S(=O)(=O)c2cc(Cl)ccc21]

Ester 13 was prepared by O-acetylation of alcohol 12 according to the following procedure. Compound 12 (200 mg, 0.49 mmol) was dissolved in Et₂O (3 mL) and a solution of HCl in Et₂O (1 mL, 2.0 M) was added (to form an HCl salt of 12). Et₂O and the excess of HCl were evaporated. The residue was dissolved in AcOH (3 mL), and acetyl chloride (0.5 mL) was added. The reaction mixture was stirred at room temperature for 14 h, concentrated, diluted with ice-cold water, and basified with NaOH (1.0 M) to pH ˜10. The mixture was then extracted with CH₂Cl₂ and the combined organic layers were dried over Na₂SO₄. After removing the solvent, the product 13 was obtained as a colorless viscous oil (190 mg, 86%). ¹H NMR (400 MHz, Methanol-d₄) δ 7.88 (t, J=0.9 Hz, 1H), 7.62 (s, 1H), 7.61 (s, 1H), 7.50-7.37 (m, 3H), 7.37-7.30 (m, 1H), 5.08 (s, 1H), 4.01 (t, J=6.6 Hz, 2H), 3.32 (s, 3H), 2.54-2.37 (m, 2H), 2.00 (s, 3H), 1.63-1.53 (m, 2H), 1.53-1.43 (m, 2H), 1.34-1.21 (m, 4H); ¹³C NMR (101 MHz, Methanol-d₄) δ 153.8, 122.7, 121.1, 119.8, 118.7, 116.1, 114.6, 114.3, 112.9, 111.4, 110.0, 109.7, 48.1, 46.4, 29.4, 20.1, 11.2, 10.4, 8.7, 7.6, 1.7; LR-MS calcd. for C₂₂H₂₈ClN₂O₄S [M+H]⁺ 451.15, found 452.16.

### Ethyl 7-((3,6-dimethyl-5,5-dioxido-6,11-dihydrodibenzo[c,f][1,2]thiazepin-11-yl)amino)heptanoate (14)

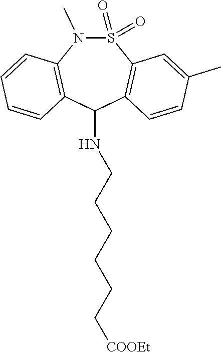 [SMILES: CCOC(=O)CCCCCCNC1c2ccccc2N(C)S(=O)(=O)c2cc(C)ccc21]

Method A. The crude product was purified by column chromatography (hexanes:ethyl acetate—2:1). The product 14 was obtained as a viscous colorless oil (59 mg, 66%). ¹H NMR (400 MHz, Acetone-d₆) δ 7.69 (s, 1H), 7.54 (d, J=7.9 Hz, 1H), 7.50 (dd, J=7.6, 1.6 Hz, 1H), 7.45 (dd, J=7.8, 1.3 Hz, 1H), 7.42-7.33 (m, 2H), 7.30 (td, J=7.4, 1.4 Hz, 1H), 5.13 (s, 1H), 4.06 (q, J=7.1 Hz, 2H), 3.35 (s, 3H), 2.49 (t, J=7.0 Hz, 2H), 2.40 (s, 3H), 2.37 (br s, 1H), 2.24 (t, J=7.4 Hz, 2H), 1.63-1.43 (m, 4H), 1.38-1.23 (m, 4H), 1.19 (t, J=7.1 Hz, 3H); ¹³C NMR (101 MHz, Acetone-d₆) δ 173.6, 141.0, 140.2, 140.1, 139.1, 136.9, 133.6, 131.0, 131.1, 129.6, 129.0, 129.0, 128.4, 66.8, 60.4, 48.6, 38.8, 34.6, 30.6, 27.7, 25.6, 20.8, 14.6; LR-MS calcd. for C₂₄H₃₃N₂O₄S [M+H]⁺ 445.22, found 446.22.

### 11-((3-Methoxypropyl)amino)-3,6-dimethyl-6,11-dihydrodibenzo[c,f][1,2]thiazepine 5,5-dioxide (15)

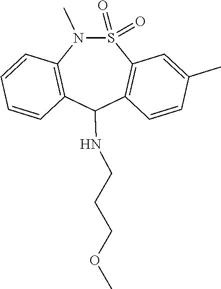 [SMILES: COCCCNC1c2ccccc2N(C)S(=O)(=O)c2cc(C)ccc21]

Method A. The crude product was purified by column chromatography (hexanes:ethyl acetate—2:1->1:1). The product 15 was obtained as a viscous colorless oil (47 mg, 65%). ¹H NMR (400 MHz, Acetone-d₆) δ 7.68 (s, 1H), 7.53 (d, J=7.9 Hz, 1H), 7.49 (dd, J=7.6, 1.6 Hz, 1H), 7.45 (dd, J=7.8, 1.3 Hz, 1H), 7.42-7.33 (m, 2H), 7.30 (td, J=7.4, 1.4 Hz, 1H), 5.13 (s, 1H), 3.39 (t, J=6.2 Hz, 2H), 3.36 (s, 3H), 3.22 (s, 3H), 2.57 (t, J=6.8 Hz, 2H), 2.46 (br s, 1H), 2.39 (s, 3H), 1.72 (p, J=6.6 Hz, 2H); ¹³C NMR (101 MHz, Acetone-d₆) δ 141.1, 140.2, 139.1, 136.9, 133.6, 130.8, 129.6, 129.0, 128.4, 71.6, 66.7, 58.5, 46.1, 38.7, 30.9, 20.8; LR-MS calcd. for C₁₉H₂₅N₂O₃S [M+H]⁺ 361.16, found 361.96.

### 7-((3,6-Dimethyl-5,5-dioxido-6,11-dihydrodibenzo[c,f][1,2]thiazepin-11-yl)amino)heptanoic acid hydrochloride salt (16a)

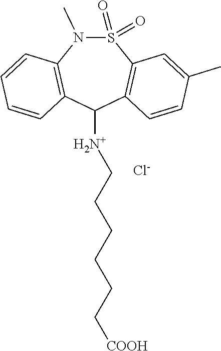 [SMILES: Cc1ccc2c(c1)S(=O)(=O)N(C)c1ccccc1C2[NH2+]CCCCCCC(=O)O ; [Cl-]]

Compound 14 (25 mg, 0.056 mmol) was heated in HCl (0.5 M, 2 mL) to 70° C. for 2 h. The reaction mixture was then concentrated and dried. The product 16a was obtained as a viscous yellowish oil (25 mg, quant. yield). ¹H NMR (400 MHz, Methanol-d₄) δ 7.93 (s, 1H), 7.80 (d, J=7.8 Hz, 1H), 7.76 (d, J=7.7 Hz, 1H), 7.67 (d, J=7.7 Hz, 1H), 7.63 (t, J=7.6 Hz, 1H), 7.55 (d, J=7.9 Hz, 1H), 7.47 (t, J=7.5 Hz, 1H), 5.95 (s, 1H), 3.18 (s, 3H), 3.02-2.67 (m, 2H), 2.53 (s, 3H), 2.26 (t, J=7.3 Hz, 2H), 1.76-1.49 (m, 4H), 1.37-1.25 (m, 4H); ¹³C NMR (101 MHz, Methanol-d₄) δ 177.4, 144.1, 142.9, 139.7, 135.8, 135.5, 134.8, 133.3, 130.1, 128.9, 128.6, 126.1, 68.0, 47.9, 39.8, 34.6, 29.4, 27.1, 26.7, 25.6, 21.2. LR-MS calcd. for C₂₂H₂₉N₂O₄S [M]⁺417.18, found 418.20.

### 7-((3,6-Dimethyl-5,5-dioxido-6,11-dihydrodibenzo[c,f][1,2]thiazepin-11-yl)amino)heptanoic acid (16b)

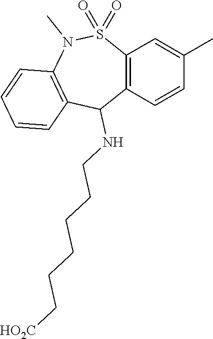 [SMILES: Cc1ccc2c(c1)S(=O)(=O)N(C)c1ccccc1C2NCCCCCCC(=O)O]

The free base compound 16b is synthesized according to the procedure used to prepare compound 18.

### Ethyl 5-((3-chloro-6-methyl-5,5-dioxido-6,11-dihydrodibenzo[c,f][1,2]thiazepin-11-yl)amino)pentanoate (17)

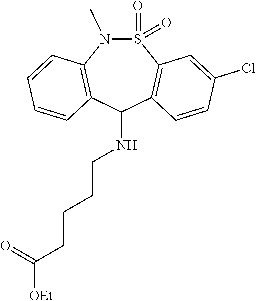 [SMILES: CCOC(=O)CCCCNC1c2ccccc2N(C)S(=O)(=O)c2cc(Cl)ccc21]

Method C. The product 17 was purified by column chromatography (CH₂Cl₂:Et₂O—40:1) and obtained as a viscous pale-yellow oil (130 mg, 60%). ¹H NMR (500 MHz, CDCl₃) δ 7.96 (d, J=2.1 Hz, 1H), 7.50-7.34 (m, 5H), 7.30 (dd, J=7.2, 1.8 Hz, 1H), 5.00 (s, 1H), 4.11 (q, J=7.1 Hz, 2H), 3.36 (s, 3H), 2.48 (t, J=7.0 Hz, 2H), 2.27 (t, J=7.4 Hz, 2H), 1.70-1.58 (m, 2H), 1.52 (ddd, J=11.9, 7.0, 3.7 Hz, 2H), 1.24 (t, J=7.1 Hz, 3H); ¹³C NMR (126 MHz, CDCl₃) δ 173.6, 140.4, 138.7, 136.8, 134.5, 132.4, 131.4, 130.3, 129.5, 128.6, 128.2, 128.1, 66.3, 60.4, 47.8, 38.9, 34.2, 29.6, 22.8, 14.4; LR-MS calcd. for C₂₁H₂₆ClN₂O₄S [M+H]⁺ 437.13, found 437.51.

### 5-((3-Chloro-6-methyl-5,5-dioxido-6,11-dihydrodibenzo[c,f][1,2]thiazepin-11-yl)amino)pentanoic acid (18)

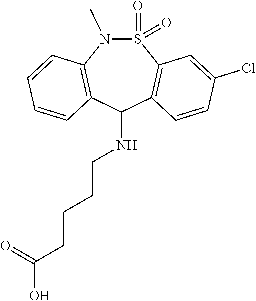 [SMILES: CN1c2ccccc2C(NCCCCC(=O)O)c2ccc(Cl)cc2S1(=O)=O]

Acid 18 was prepared by hydrolysis of ester 17 according to the following procedure. To a solution of 17 (120 mg, 0.275 mmol) in ethanol (0.75 mL) and H₂O (0.25 mL) was added NaOH (11.8 mg, 0.296 mmol), and the resulting solution was refluxed for 1 h. Most of the ethanol was removed in vacuo and the mixture was diluted with water (5 mL) and washed with Et₂O (2×5 mL). It was then carefully acidified to pH 4-5 with 10% aq. HCl, saturated with (NH₄)₂SO₄, and extracted with CHCl₃ (2×5 mL). The combined organics were washed with H₂O (3 mL), dried over Na₂SO₄, and concentrated to yield the product 18 as a foamy off-white solid (105 mg, 94%). ¹H NMR (500 MHz, CDCl₃) δ 7.97 (d, J=1.9 Hz, 1H), 7.51 (ddd, J=8.4, 7.4, 5.7 Hz, 3H), 7.44-7.38 (m, 1H), 7.36 (dd, J=8.0, 1.3 Hz, 1H), 7.30 (td, J=7.6, 1.4 Hz, 1H), 5.27 (s, 1H), 4.42 (br s, 2H), 3.27 (s, 3H), 2.55 (dd, J=11.0, 8.2 Hz, 1H), 2.42 (dd, J=9.9, 6.9 Hz, 1H), 2.25 (dd, J=10.9, 5.0 Hz, 2H), 1.71-1.45 (m, 4H); ¹³C NMR (126 MHz, CDCl₃) δ 177.3, 140.2, 139.5, 135.3, 134.0, 133.2, 132.9, 132.0, 130.3, 128.6, 128.1, 127.6, 66.3, 46.9, 39.2, 34.2, 28.4, 22.6; LR-MS calcd. for C₁₉H₂₂ClN₂O₄S [M+H]⁺ 409.10, found 408.69.

### 3-Chloro-6-methyl-11-(pentylamino)-6,11-dihydrodibenzo [c,f][1,2]thiazepine 5,5-dioxide (19)

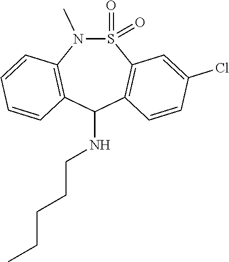 [SMILES: CCCCCNC1c2ccccc2N(C)S(=O)(=O)c2cc(Cl)ccc21]

Method B. The product 19 was purified by column chromatography (CH₂Cl₂:hexanes—8:2, 4 column volumes→CH₂Cl₂, 3 column volumes) and obtained as a viscous colorless oil (52.9 mg, 28%). ¹H NMR (400 MHz, CDCl₃) δ 7.96 (d, J=1.7 Hz, 1H), 7.48-7.42 (m, 2H), 7.41-7.32 (m, 3H), 7.32-7.27 (m, 1H), 5.01 (s, 1H), 3.38 (s, 3H), 2.46 (t, J=7.2 Hz, 2H), 2.01 (br s, 1H), 1.57-1.37 (m, 2H), 1.35-1.20 (m, 4H), 0.86 (t, J=7.0 Hz, 3H); ¹³C NMR (101 MHz, CDCl₃) δ 140.5, 139.0, 138.6, 137.1, 134.3, 132.3, 131.2, 130.1, 129.4, 128.5, 128.3, 128.1, 66.2, 48.3, 38.8, 29.9, 29.6, 22.7, 14.1; LR-MS calcd. for C₁₉H₂₄ClN₂O₂S [M+H]⁺ 379.12, found 378.75.

### 3-Chloro-6-methyl-11-(methylamino)-6,11-dihydrodibenzo [c,f][1,2]thiazepine 5,5-dioxide (20)

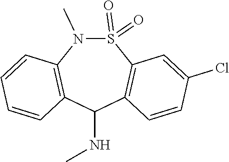 [SMILES: CNC1c2ccccc2N(C)S(=O)(=O)c2cc(Cl)ccc21]

Compound 20 was prepared according to a modified Method C using 10 equivalents of methylamine HCl and 20 equivalents of Et₂N. The product 20 was purified by column chromatography (EtOAc:Hexanes—6:4, 5 column volumes→EtOAc, 2 column volumes) and obtained as a pale-yellow glass (134 mg, 83%). ¹H NMR (400 MHz, CDCl₃) δ 7.97 (d, J=2.1 Hz, 1H), 7.50-7.27 (m, 6H), 4.88 (s, 1H), 3.34 (s, 3H), 2.34 (s, 3H), 2.10 (br s, 1H); ¹³C NMR (101 MHz, CDCl₃) δ 140.4, 138.8, 138.1, 136.5, 134.5, 132.3, 131.6, 130.6, 129.5, 128.6, 128.2, 128.1, 68.4, 38.9, 35.0; LR-MS calcd. for C₁₅H₁₆ClN₂O₂S [M+H]⁺ 323.06, found 323.53.

### 3-Chloro-6-methyl-11-(propylamino)-6,11-dihydrodibenzo [c,f][1,2]thiazepine 5,5-dioxide (21)

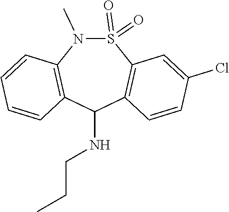 [SMILES: CCCNC1c2ccccc2N(C)S(=O)(=O)c2cc(Cl)ccc21]

Compound 21 was prepared according to a modified Method B using 10 equivalents of n-propylamine. The product 21 was obtained as a viscous pale-yellow oil (169 mg, 96%). ¹H NMR (400 MHz, CDCl₃) δ 7.98-7.93 (m, 1H), 7.48-7.26 (m, 6H), 5.01 (s, 1H), 3.38 (s, 3H), 2.44 (t, J=7.1 Hz, 2H), 2.02 (br s, 1H), 1.57-1.44 (m, 2H), 0.89 (t, J=7.4 Hz, 3H); ¹³C NMR (101 MHz, CDCl₃) δ 140.6, 139.1, 138.6, 137.1, 134.3, 132.2, 131.3, 130.1, 129.4, 128.5, 128.3, 128.2, 66.2, 50.1, 38.8, 23.3, 11.9; LR-MS calcd. for C₁₇H₂₀ClN₂O₂S [M+H]⁺ 351.09, found 351.51.

### 3-Chloro-11-(isopropylamino)-6-methyl-6,11-dihydrodibenzo [c,f][1,2]thiazepine 5,5-dioxide (22)

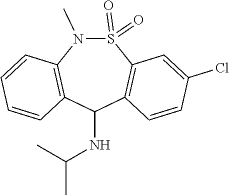 [SMILES: CC(C)NC1c2ccccc2N(C)S(=O)(=O)c2cc(Cl)ccc21]

Compound 22 was prepared according to a modified Method B using 10 equivalents of isopropylamine. The product 22 was obtained as a yellow glass (173 mg, 99%). ¹H NMR (500 MHz, CDCl₃) δ 7.95 (d, J=2.1 Hz, 1H), 7.47-7.40 (m, 2H), 7.39-7.32 (m, 3H), 7.28 (td, J=7.3, 1.6 Hz, 1H), 5.09 (s, 1H), 3.37 (s, 3H), 2.57 (hept, J=6.2 Hz, 1H), 2.15 (br s, 1H), 1.08 (d, J=6.2 Hz, 3H), 1.04 (d, J=6.2 Hz, 3H); ¹³C NMR (126 MHz, CDCl₃) δ 140.5, 138.8, 138.6, 137.2, 134.2, 132.2, 131.6, 130.4, 129.3, 128.4, 128.1, 128.0, 63.2, 45.4, 38.7, 22.92, 22.86; LR-MS calcd. for C₁₇H₂₃ClN₂O₂S [M+H]⁺ 351.09, found 351.81.

### 3-Chloro-11-((3-methoxypropyl)amino)-6-methyl-6,11-dihydrodibenzo [c,f][1,2]thiazepine 5,5-dioxide (23)

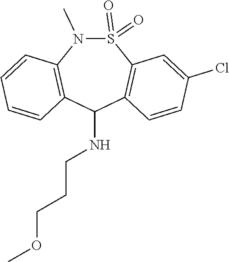 [SMILES: COCCCNC1c2ccccc2N(C)S(=O)(=O)c2cc(Cl)ccc21]

Method B. The product 23 was obtained as a viscous pale-yellow oil (188 mg, 99%). ¹H NMR (500 MHz, CDCl₃) δ 7.94 (t, J=1.2 Hz, 1H), 7.44 (d, J=1.3 Hz, 2H), 7.41-7.37 (m, 2H), 7.37-7.33 (m, 1H), 7.28 (td, J=7.3, 1.6 Hz, 1H), 5.02 (s, 1H), 3.45-3.39 (m, 2H), 3.37 (s, 3H), 3.29 (s, 3H), 2.64-2.52 (m, 2H), 2.18 (br s, 1H), 1.82-1.68 (m, 2H); ¹³C NMR (126 MHz, CDCl₃) δ 140.5, 139.0, 138.6, 137.1, 134.3, 132.3, 131.1, 130.0, 129.4, 128.5, 128.3, 128.1, 71.3, 66.0, 58.8, 45.8, 38.6, 30.0; LR-MS calcd. for C₁₈H₂₂ClN₂O₃S [M+H]⁺ 381.10, found 380.79.

### 3-Chloro-11-((4, 4-diethoxybutyl)amino)-6-methyl-6,11-dihydrodibenzo[c,f][1,2]thiazepine 5,5-dioxide (24)

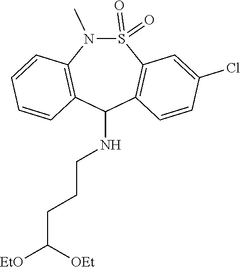 [SMILES: CCOC(CCCNC1c2ccccc2N(C)S(=O)(=O)c2cc(Cl)ccc21)OCC]

Method B. The product 24 was purified by column chromatography (CH₂Cl₂:Et₂O—20:1, 4 column volumes to 8:2, 2 column volumes) and obtained as a viscous pale-yellow oil (204 mg, 90%). ¹H NMR (500 MHz, CDCl₃) δ 7.94 (d, J=1.5 Hz, 1H), 7.46-7.41 (m, 2H), 7.35 (tdd, J=15.0, 7.1, 2.3 Hz, 3H), 7.27 (td, J=7.3, 1.8 Hz, 1H), 5.00 (s, 1H), 4.43 (t, J=5.5 Hz, 1H), 3.64-3.56 (m, 2H), 3.49-3.40 (m, 2H), 3.36 (s, 3H), 2.49 (t, J=6.9 Hz, 2H), 2.04 (br s, 1H), 1.65-1.58 (m, 2H), 1.58-1.50 (m, 2H), 1.16 (td, J=7.0, 2.7 Hz, 6H); ¹³C NMR (126 MHz, CDCl₃) δ 140.5, 138.9, 138.6, 137.0, 134.3, 132.2, 131.2, 130.1, 129.4, 128.4, 128.2, 128.1, 102.8, 66.0, 65.9, 61.3, 61.2, 47.9, 38.7, 31.5, 25.3, 15.4; LR-MS calcd. for C₂₂H₃₃ClN₂O₄S [M+H]⁺ 453.16, found 452.98.

### 3-Chloro-11-((2-(2-hydroxyethoxy)ethyl)amino)-6-methyl-6,11-dihydrodibenzo[c,f][1,2]thiazepine 5,5-dioxide (25)

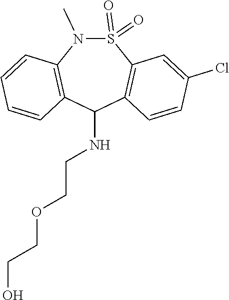 [SMILES: CN1c2ccccc2C(NCCOCCO)c2ccc(Cl)cc2S1(=O)=O]

Method B. In this case, CH₂Cl₂ was used as the extraction solvent. The product 25 was purified by column chromatography (CH₂Cl₂:Et₂O—1:1 for 4 column volumes, then 2% Et₃N added to eluent) and obtained as a viscous colorless oil (158 mg, 80%). ¹H NMR (500 MHz, CDCl₃) δ 7.95 (t, J=1.0 Hz, 1H), 7.45 (d, J=1.2 Hz, 2H), 7.42-7.33 (m, 3H), 7.31-7.26 (m, 1H), 5.03 (s, 1H), 3.67-3.63 (m, 2H), 3.61-3.53 (m, 2H), 3.50 (dd, J=5.8, 3.4 Hz, 2H), 3.36 (s, 3H), 2.68-2.63 (m, 2H), 2.60 (br s, 2H); ¹³C NMR (126 MHz, CDCl₃) δ 140.5, 138.6, 138.4, 136.7, 134.5, 132.4, 131.5, 130.3, 129.6, 128.5, 128.3, 128.0, 72.3, 70.4, 66.2, 61.8, 47.5, 38.7; LR-MS calcd. for C₁₈H₂₂ClN₂O₄S [M+H]⁺ 397.10, found 396.93.

### 3-Chloro-11-(heptylamino)-6-methyl-6,11-dihydrodibenzo [c,f][1,2]thiazepine 5,5-dioxide (26)

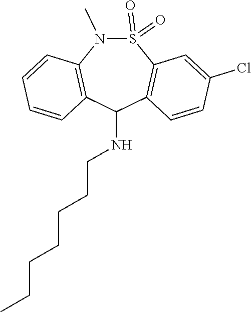 [SMILES: CCCCCCCNC1c2ccccc2N(C)S(=O)(=O)c2cc(Cl)ccc21]

Method B. The product 26 was purified by column chromatography (hexanes:EtOAc—9:1+2% Et₃N) and obtained as viscous colorless oil (161 mg, 79%). ¹H NMR (500 MHz, CDCl₃) δ 7.96 (d, J=1.9 Hz, 1H), 7.47-7.42 (m, 2H), 7.41-7.33 (m, 3H), 7.29 (td, J=7.3, 1.5 Hz, 1H), 5.01 (s, 1H), 3.38 (s, 3H), 2.46 (t, J=7.1 Hz, 2H), 1.99 (s, 1H), 1.54-1.41 (m, 2H), 1.32-1.17 (m, 8H), 0.86 (t, J=7.0 Hz, 3H); ¹³C NMR (126 MHz, CDCl₃) δ 140.6, 139.1, 138.6, 137.2, 134.3, 132.3, 131.2, 130.1, 129.4, 128.5, 128.3, 128.1, 66.2, 48.3, 38.7, 31.9, 30.2, 29.3, 27.4, 22.7, 14.2; LR-MS calcd. for C₂₁H₂₈ClN₂O₂S [M+H]⁺ 407.16, found 406.94.

### 3-Methoxy-11-((3-methoxypropyl)amino)-6-methyl-6,11-dihydrodibenzo [c,f][1,2]thiazepine 5,5-dioxide (27)

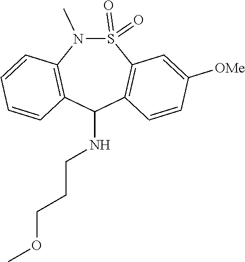 [SMILES: COCCCNC1c2ccccc2N(C)S(=O)(=O)c2cc(OC)ccc21]

Method B. The product 27 was purified by column chromatography (CH₂Cl₂:Et₂O—8:2, 2 column volumes→1:1, 3 column volumes) and obtained as a viscous colorless oil. (32.1 mg, 85%). ¹H NMR (500 MHz, CDCl₃) δ 7.47 (d, J=2.7 Hz, 1H), 7.42-7.36 (m, 3H), 7.34 (td, J=7.5, 1.6 Hz, 1H), 7.27 (dt, J=7.5, 1.6 Hz, 1H), 7.01 (dd, J=8.5, 2.7 Hz, 1H), 4.92 (s, 1H), 3.84 (s, 3H), 3.40 (pd, J=9.4, 6.3 Hz, 2H), 3.35 (s, 3H), 3.29 (s, 3H), 2.55 (t, J=6.8 Hz, 2H), 2.17 (br s, 1H), 1.80-1.67 (m, 2H); ¹³C NMR (126 MHz, CDCl₃) δ 159.3, 139.9, 139.2, 139.1, 131.8, 130.7, 130.4, 129.2, 128.1, 128.0, 118.7, 112.9, 71.3, 67.0, 58.8, 55.9, 45.6, 38.9, 30.1; LR-MS calcd. for C₁₉H₂₅N₂O₄S [M+H]⁺ 377.15, found 376.95.

### 3-Chloro-6-methyl-1-((4,4,4-trifluorobutyl)amino)-6,11-dihydrodibenzo[c,f][1,2]thiazepine 5,5-dioxide (28)

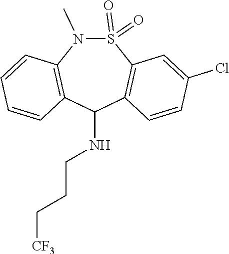 [SMILES: CN1c2ccccc2C(NCCCC(F)(F)F)c2ccc(Cl)cc2S1(=O)=O]

Method B. The product 28 was purified by column chromatography (hexanes:EtOAc—8:2+2% Et₃N) and obtained as a viscous pale-yellow oil (168 mg, 80%). ¹H NMR (500 MHz, CDCl₃) δ 7.97 (d, J=2.2 Hz, 1H), 7.48 (dd, J=8.2, 2.2 Hz, 1H), 7.42-7.34 (m, 4H), 7.33-7.28 (m, 1H), 4.97 (s, 1H), 3.30 (s, 3H), 2.58-2.46 (m, 2H), 2.26 (br s, 1H), 2.21-2.08 (m, 2H), 1.71 (dq, J=13.9, 6.9 Hz, 2H); ¹³C NMR (126 MHz, CDCl₃) (additional peaks due to C—F coupling) δ 140.1, 139.0, 137.4, 136.4, 134.5, 132.4, 131.8, 131.0, 129.6, 128.7, 128.4, 128.1, 127.9, 126.2, 66.7, 46.6, 39.0, 31.6 (q, 1C), 22.6; LR-MS calcd. for C₁₈H₁₉C₁F₃N₂O₂S [M+H]⁺ 419.08, found 418.88.

### 11-((3-Methoxypropyl)amino)-6-methyl-3-phenoxy-6,11-dihydrodibenzo [c,f][1,2]thiazepine 5,5-dioxide (29)

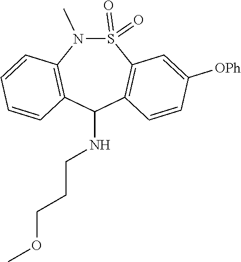 [SMILES: COCCCNC1c2ccccc2N(C)S(=O)(=O)c2cc(Oc3ccccc3)ccc21]

Method B. The product 29 was purified by column chromatography (CH₂Cl₂:Et₂O—9:1, 2 column volumes to 8:2, 2 column volumes) and obtained as a viscous pale-yellow oil (37.4 mg, 85%). ¹H NMR (500 MHz, CDCl₃) δ 7.59 (d, J=2.2 Hz, 1H), 7.47-7.33 (m, 6H), 7.29 (t, J=7.3 Hz, 1H), 7.16 (t, J=7.1 Hz, 1H), 7.09 (dd, J=8.4, 2.3 Hz, 1H), 7.02 (d, J=8.3 Hz, 2H), 4.98 (s, 1H), 3.47-3.40 (m, 2H), 3.38 (s, 3H), 3.30 (s, 3H), 2.59 (t, J=6.7 Hz, 2H), 2.07 (br s, 1H), 1.84-1.71 (m, 2H); ¹³C NMR (126 MHz, CDCl₃) δ 157.5, 156.0, 140.5, 139.3, 138.9, 132.7, 131.7, 130.3, 130.2, 129.3, 128.1, 124.5, 121.7, 119.7, 117.9, 71.3, 66.6, 58.8, 45.8, 38.7, 30.1; LR-MS calcd. for C₂₄H₂₇N₂O₄S [M+H]⁺ 439.17, found 438.88.

### 3-Chloro-11-((5-hydroxypentyl)amino)-6-methyl-6,11-dihydrodibenzo [c,f][1,2]thiazepine 5,5-dioxide (30)

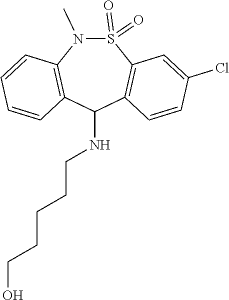 [SMILES: CN1c2ccccc2C(NCCCCCO)c2ccc(Cl)cc2S1(=O)=O]

Method B. The product 30 was purified by column chromatography (CH₂Cl₂:Et₂O—6:4) and obtained as a viscous pale-yellow oil (317 mg, 80%). ¹H NMR (500 MHz, CDCl₃) δ 7.96 (s, 1H), 7.51-7.26 (m, 6H), 5.00 (s, 1H), 3.63-3.54 (m, 2H), 3.36 (d, J=2.1 Hz, 3H), 2.55-2.43 (m, 2H), 1.97 (br s, 2H), 1.58-1.46 (m, 4H), 1.43-1.31 (m, 2H); ¹³C NMR (126 MHz, CDCl₃) δ 140.4, 138.7, 138.5, 136.9, 134.3, 132.3, 131.5, 130.3, 129.5, 128.5, 128.2, 128.0, 66.4, 62.6, 48.0, 38.8, 32.5, 29.8, 23.5; LR-MS calcd. for C₁₉H₂₄ClN₂O₃S [M+H]⁺ 395.12, found 394.85.

### 11-((3-(1, 3-Dioxan-2-yl) propyl)amino)-3-chloro-6-methyl-6,11-dihydrodibenzo[c,f][1,2]thiazepine 5,5-dioxide (31)

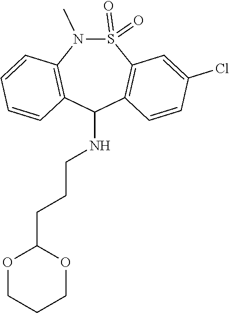 [SMILES: CN1c2ccccc2C(NCCCC2OCCCO2)c2ccc(Cl)cc2S1(=O)=O]

Acetal 31 was prepared by transacetalization of compound 24 according to the following procedure. Diethyl acetal 24 (22.6 mg, 0.0500 mmol) was dissolved in 1,3-propanediol (0.50 mL), p-toluenesulfonic acid monohydrate (14.3 mg, 0.0750 mmol) was added, and the solution was stirred for 3 days at room temperature. The mixture was then diluted with water (5 mL), basified with saturated NH₄OH, and extracted with Et₂O (3×5 mL). The combined organics were washed with water (5 mL), dried over Na₂SO₄, and concentrated to yield a pale-yellow glass. This was purified by repeated (2×) column chromatography (hexanes:EtOAc -1:1, 5 column volumes→EtOAc, 2 column volumes) to yield the pure product 31 as a viscous pale-yellow oil (8.6 mg, 39%). ¹H NMR (500 MHz, CDCl₃) δ 7.95 (t, J=1.1 Hz, 1H), 7.49-7.42 (m, 2H), 7.42-7.33 (m, 3H), 7.29 (td, J=7.4, 1.5 Hz, 1H), 5.04 (s, 1H), 4.49 (t, J=4.5 Hz, 1H), 4.07 (ddd, J=8.5, 4.3, 1.2 Hz, 2H), 3.76-3.67 (m, 2H), 3.39 (s, 3H), 2.54-2.45 (m, 2H), 2.10-1.95 (m, 1H), 1.73 (br s, 1H), 1.66-1.55 (m, 4H), 1.35-1.29 (m, 1H); ¹³C NMR (126 MHz, CDCl₃) δ 140.6, 138.6, 137.2, 134.6, 134.4, 132.3, 131.1, 129.9, 129.5, 128.5, 128.3, 128.1, 102.1, 67.0, 65.6, 47.9, 38.7, 33.0, 25.9, 24.4; LR-MS calcd. for C₂₁H₂₆ClN₂O₄S [M+H]⁺ 437.13, found 436.99.

### 3-Chloro-11-((5-methoxypentyl)amino)-6-methyl-6,11-dihydrodibenzo [c,f][1,2]thiazepine 5,5-dioxide (32)

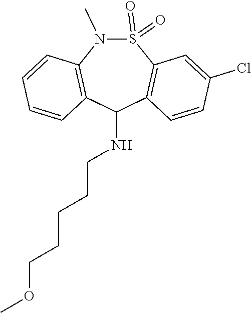 [SMILES: COCCCCCNC1c2ccccc2N(C)S(=O)(=O)c2cc(Cl)ccc21]

Compound 32 was prepared by O-methylation of alcohol 30 according to the following procedure. To a mixture of 30 (118 mg, 0.300 mmol) and NaH (12.0 mg of 60% dispersion in mineral oil, 0.300 mmol) was added anhydrous THF (0.38 mL, freshly distilled from Na/benzophenone), and the yellow-orange mixture was stirred for 1 h at room temperature. Dimethyl sulfate (28.6 μL, 0.300 mmol) was then added to the red-orange mixture and the reaction was stirred for 1 h at room temperature. The reaction was then quenched with water (10 mL) and extracted with Et₂O (3×5 mL). The combined organics were washed with 10% NH₄OH (5 mL), dried over Na₂SO₄, and concentrated to yield a thick yellow oil. This crude was purified by column chromatography (hexanes:EtOAc—6:4) to yield the pure product 32 as a viscous pale-yellow oil (29.4 mg, 24%). ¹H NMR (500 MHz, CDCl₃) δ 7.95 (d, J=2.0 Hz, 1H), 7.48-7.33 (m, 5H), 7.31-7.27 (m, 1H), 5.00 (s, 1H), 3.37 (s, 3H), 3.33 (t, J=6.5 Hz, 2H), 3.30 (s, 3H), 2.47 (t, J=7.1 Hz, 2H), 1.95 (br s, 1H), 1.58-1.46 (m, 4H), 1.39-1.30 (m, 2H); ¹³C NMR (126 MHz, CDCl₃) δ 140.5, 138.8, 138.7, 137.0, 134.4, 132.3, 131.3, 130.2, 129.5, 128.6, 128.3, 128.1, 72.8, 66.3, 58.7, 48.2, 38.8, 30.0, 29.6, 24.0; LR-MS calcd. for C₂₀H₂₆ClN₂O₃S [M+H]⁺ 409.14, found 409.03.

### 3-Chloro-6-methyl-11-((3-(methylthio) propyl)amino)-6,11-dihydrodibenzo[c,f][1,2]thiazepine 5,5-dioxide (33)

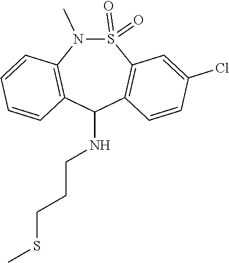 [SMILES: CSCCCNC1c2ccccc2N(C)S(=O)(=O)c2cc(Cl)ccc21]

Method B. The product 33 was obtained as a viscous pale-yellow oil (194 mg, 98%). ¹H NMR (400 MHz, CDCl₃) δ 7.96 (d, J=2.1 Hz, 1H), 7.50-7.27 (m, 6H), 5.00 (s, 1H), 3.34 (s, 3H), 2.65-2.46 (m, 4H), 2.07 (br s, 1H) 2.06 (s, 3H), 1.77 (p, J=7.0 Hz, 2H); ¹³C NMR (101 MHz, CDCl₃) δ 140.3, 138.8, 138.3, 136.8, 134.4, 132.4, 131.5, 130.5, 129.5, 128.6, 128.2, 128.0, 66.4, 46.9, 38.9, 32.1, 29.4, 15.7; LR-MS calcd. for C₁₈H₂₂ClN₂O₂S₂ [M+H]⁺ 397.08, found 396.84.

### Ethyl 7-((3-chloro-6-methyl-5, 5-dioxido-6,11-dihydrodibenzo[c,f][1,2]thiazepin-11-yl)amino)heptanoate (34)

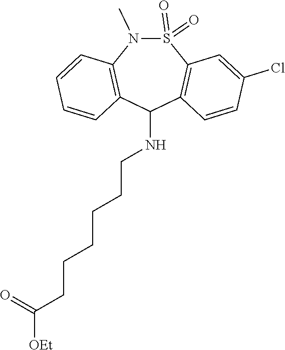 [SMILES: CCOC(=O)CCCCCCNC1c2ccccc2N(C)S(=O)(=O)c2cc(Cl)ccc21]

Method C. The product 34 was purified by column chromatography (CH₂C₁₋₂:Et₂O, 40:1, 6 column volumes→1:1, 2 column volumes) and obtained as a viscous pale-yellow oil (91 mg, 78%). ¹H NMR (400 MHz, CDCl₃) δ 7.95 (d, J=2.0 Hz, 1H), 7.48-7.26 (m, 6H), 5.00 (s, 1H), 4.10 (q, J=7.1 Hz, 2H), 3.36 (s, 3H), 2.45 (t, J=7.1 Hz, 2H), 2.26 (t, J=7.5 Hz, 2H), 2.03 (br s, 1H), 1.65-1.53 (m, 2H), 1.53-1.41 (m, 2H), 1.35-1.26 (m, 4H), 1.23 (t, J=7.1 Hz, 3H); ¹³C NMR (101 MHz, CDCl₃) δ 173.8, 140.5, 138.9, 138.6, 137.0, 134.3, 132.3, 131.3, 130.1, 129.4, 128.5, 128.2, 128.1, 66.2, 60.3, 48.2, 38.8, 34.3, 30.0, 29.1, 27.0, 24.9, 14.4; LR-MS calcd. for C₂₃H₃₀ClN₂O₄S [M+H]⁺ 465.16, found 465.13.

### 3-Chloro-11-((3-isopropoxypropyl)amino)-6-methyl-6,11-dihydrodibenzo [c,f][1,2]thiazepine 5,5-dioxide (35)

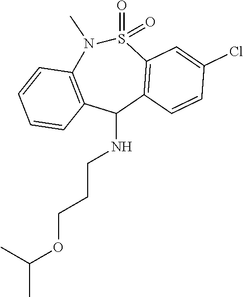 [SMILES: CC(C)OCCCNC1c2ccccc2N(C)S(=O)(=O)c2cc(Cl)ccc21]

Method B. The product 35 was obtained as a viscous pale-yellow oil (197 mg, 96%). ¹H NMR (500 MHz, CDCl₃) δ 7.94 (d, J=1.9 Hz, 1H), 7.51-7.26 (m, 6H), 5.06 (s, 1H), 3.57-3.43 (m, 3H), 3.39 (s, 3H), 2.59 (t, J=6.5 Hz, 2H), 2.26 (s, 1H), 1.83-1.68 (m, 2H), 1.10 (t, J=6.5 Hz, 6H); ¹³C NMR (126 MHz, CDCl₃) δ 140.5, 139.4, 138.5, 137.3, 134.2, 132.2, 130.8, 129.6, 129.3, 128.5, 128.7, 128.1, 71.6, 66.6, 65.5, 46.0, 38.6, 30.4, 22.2; LR-MS calcd. for C₂₀H₂₆ClN₂O₃S [M+H]⁺ 409.14, found 409.24.

### 3-Chloro-11-((2-(2-methoxyethoxy)ethyl)amino)-6-methyl-6,11-dihydrodibenzo[c,f][1,2]thiazepine 5,5-dioxide (36)

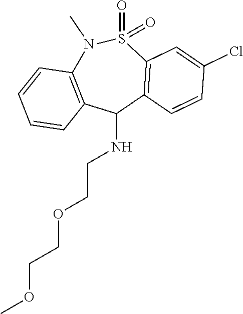 [SMILES: COCCOCCNC1c2ccccc2N(C)S(=O)(=O)c2cc(Cl)ccc21]

Compound 36 was prepared by O-methylation of alcohol 25 according to the procedure described for compound 32. The product 36 was purified by column chromatography (EtOAc) and obtained as a viscous pale-yellow oil (21.6 mg, 26%). ¹H NMR (400 MHz, CDCl₃) δ 7.94 (d, J=2.1 Hz, 1H), 7.52-7.26 (m, 6H), 5.08 (s, 1H), 3.66-3.48 (m, 6H), 3.44 (s, 3H), 3.34 (s, 3H), 2.75-2.62 (m, 2H), 2.34 (br s, 1H); ¹³C NMR (101 MHz, CDCl₃) δ 141.0, 139.6, 138.5, 137.1, 134.5, 132.3, 131.1, 129.7, 129.6, 128.4, 128.3, 72.0, 70.7, 70.5, 65.6, 59.1, 47.6, 38.5; LR-MS calcd. for C₁₉H₂₄ClN₂O₄S [M+H]⁺ 411.11, found 411.35.

### 3-Chloro-11-((3-ethoxypropyl)amino)-6-methyl-6,11-dihydrodibenzo [c,f][1,2]thiazepine 5,5-dioxide (37)

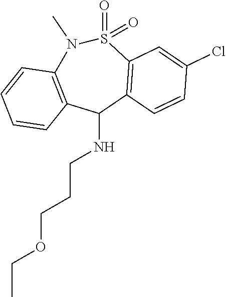 [SMILES: CCOCCCNC1c2ccccc2N(C)S(=O)(=O)c2cc(Cl)ccc21]

Method B. The product 37 was purified by column chromatography (hexanes:EtOAc—1:1, 4 column volumes→EtOAc, 2 column volumes) and obtained as a viscous pale-yellow oil (176 mg, 89%). ¹H NMR (500 MHz, CDCl₃) δ 7.93 (s, 1H), 7.51-7.23 (m, 6H), 5.05 (s, 1H), 3.53-3.40 (m, 4H), 3.37 (s, 3H), 2.59 (t, J=6.4 Hz, 2H), 2.35 (br s, 1H), 1.83-1.70 (m, 2H), 1.14 (t, J=6.9 Hz, 3H); ¹³C NMR (126 MHz, CDCl₃) δ 140.5, 139.1, 138.5, 137.1, 134.2, 132.2, 131.0, 129.8, 129.3, 128.4, 128.2, 128.1, 69.1, 66.3, 65.6, 45.9, 38.6, 30.0, 15.2; LR-MS calcd. for C₁₉H₂₄ClN₂C₃S [M+H]⁺ 395.12, found 394.49.

### 3-Chloro-11-((4-hydroxybutyl)amino)-6-methyl-6,11-dihydrodibenzo[c,f][1,2]thiazepine 5,5-dioxide (38)

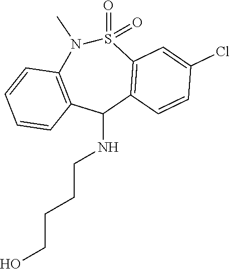 [SMILES: CN1c2ccccc2C(NCCCCO)c2ccc(Cl)cc2S1(=O)=O]

Method B. The product 38 was purified by column chromatography (CH₂Cl₂:Et₂O—6:4, 4 column volumes→CH₂Cl₂:Et₂O:MeOH—12:8:1, 2 column volumes) and obtained as a viscous colorless oil (148 mg, 78%). ¹H NMR (400 MHz, CDCl₃) δ 7.96 (d, J=2.2 Hz, 1H), 7.49 (dd, J=8.2, 2.2 Hz, 1H), 7.44-7.39 (m, 2H), 7.39-7.27 (m, 3H), 4.97 (s, 1H), 3.61-3.47 (m, 2H), 3.28 (br s, 2H), 3.24 (s, 3H), 2.44 (t, J=5.7 Hz, 2H), 1.70-1.53 (m, 3H), 1.53-1.42 (m, 1H); ¹³C NMR (101 MHz, CDCl₃) δ 139.9, 139.2, 135.7, 135.5, 134.6, 132.6, 131.6, 129.7, 128.6, 128.0, 127.7, 67.3, 62.6, 47.9, 39.3, 31.7, 27.8; LR-MS calcd. for C₁₈H₂₂ClN₂O₃S [M+H]⁺381.10, found 380.97.

### 3-Chloro-11-((4-methoxybutyl)amino)-6-methyl-6,11-dihydrodibenzo [c,f][1,2]thiazepine 5,5-dioxide (39)

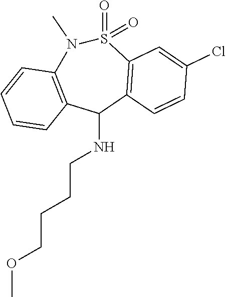 [SMILES: COCCCCNC1c2ccccc2N(C)S(=O)(=O)c2cc(Cl)ccc21]

Compound 39 was prepared by O-methylation of alcohol 38 according to the procedure described for compound 32. The product was purified by repeated column chromatography (column 1:hexanes:EtOAc—1:1/column 2:hexanes:EtOAc—1:1+2% Et₃N, 6 column volumes→EtOAc+2% Et₃N, 2 column volumes) and obtained as a viscous pale-yellow oil (3.4 mg, 4.3%). The N-methylation byproduct was also obtained (4.6 mg, 5.8%). ¹H NMR (400 MHz, CDCl₃) δ 7.96 (d, J=1.9 Hz, 1H), 7.50-7.27 (m, 6H), 5.02 (s, 1H), 3.38 (s, 3H), 3.34 (t, J=6.0 Hz, 2H), 3.30 (s, 3H), 2.50 (t, J=6.7 Hz, 2H), 2.07 (br s, 1H), 1.67-1.46 (m, 4H); LR-MS calcd. for C₁₉H₂₄ClN₂O₃S [M+H]⁺ 395.12, found 394.97.

### 3-Fluoro-11-((3-methoxypropyl)amino)-6-methyl-6,11-dihydrodibenzo[c,f][1,2]thiazepine 5,5-dioxide (40)

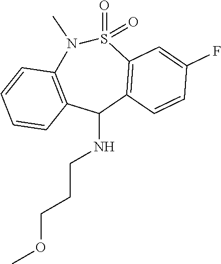 [SMILES: COCCCNC1c2ccccc2N(C)S(=O)(=O)c2cc(F)ccc21]

Method B. The product 40 was obtained as a viscous pale-yellow oil (33.8 mg, 93%). ¹H NMR (400 MHz, CDCl₃) δ 7.68 (dd, J=8.2, 2.7 Hz, 1H), 7.48 (dd, J=8.6, 5.2 Hz, 1H), 7.41-7.33 (m, 3H), 7.32-7.27 (m, 1H), 7.23-7.14 (m, 1H), 5.01 (s, 1H), 3.42 (td, J=6.2, 2.5 Hz, 2H), 3.38 (s, 3H), 3.29 (s, 3H), 2.63-2.49 (m, 2H), 2.25 (br s, 1H), 1.83-1.66 (m, 2H); ¹³C NMR (101 MHz, CDCl₃) (additional peaks due to C—F coupling) δ 163.0, 160.5, 140.9, 140.8, 139.1, 138.7, 134.6, 131.9, 130.2, 129.4, 128.3, 128.2, 119.3, 119.1, 116.0, 115.7, 71.3, 66.3, 58.8, 45.8, 38.8, 30.1; LR-MS calcd. for C₁₈H₂₂FN₂O₃S [M+H]⁺ 365.13, found 365.52.

### Ethyl 7-((3-fluoro-6-methyl-5, 5-dioxido-6,11-dihydrodibenzo[c,f][1,2]thiazepin-11-yl)amino)heptanoate (41)

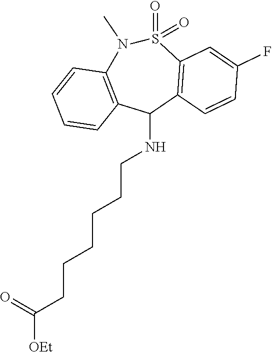 [SMILES: CCOC(=O)CCCCCCNC1c2ccccc2N(C)S(=O)(=O)c2cc(F)ccc21]

Method C. The product 41 was obtained as a viscous pale-yellow oil (39.0 mg, 87%). ¹H NMR (400 MHz, CDCl₃) δ 7.68 (dd, J=8.2, 2.7 Hz, 1H), 7.47 (dd, J=8.6, 5.2 Hz, 1H), 7.36 (qd, J=7.7, 4.0 Hz, 3H), 7.32-7.26 (m, 1H), 7.19 (ddd, J=8.5, 7.7, 2.7 Hz, 1H), 4.99 (s, 1H), 4.11 (q, J=7.1 Hz, 2H), 3.37 (s, 3H), 2.45 (t, J=7.1 Hz, 2H), 2.26 (t, J=7.5 Hz, 2H), 2.05 (br s, 1H), 1.64-1.53 (m, 2H), 1.52-1.42 (m, 2H), 1.35-1.26 (m, 4H), 1.24 (t, J=7.1 Hz, 3H); ¹³C NMR (101 MHz, CDCl₃) (additional peaks due to C—F coupling) δ 173.8, 163.0, 160.5, 140.9, 140.8, 139.0, 138.7, 134.5, 132.1, 132.0, 130.3, 129.5, 128.2, 128.2, 119.3, 119.1, 116.0, 115.7, 66.5, 60.3, 48.2, 38.9, 34.4, 30.0, 29.1, 27.1, 25.0, 14.4; LR-MS calcd. for C₂₃H₃₀FN₂O₄S [M+H]⁺ 449.19, found 449.81.

### 3-Chloro-11-((2-methoxyethyl)amino)-6-methyl-6,11-dihydrodibenzo [c,f][1,2]thiazepine 5,5-dioxide (42)

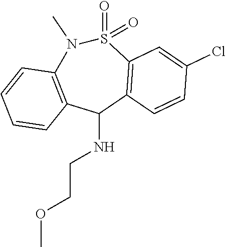 [SMILES: COCCNC1c2ccccc2N(C)S(=O)(=O)c2cc(Cl)ccc21]

Method B. The product 42 was obtained as a viscous pale-yellow oil (184 mg, 100%). ¹H NMR (400 MHz, CDCl₃) δ 7.94 (d, J=2.1 Hz, 1H), 7.49-7.33 (m, 5H), 7.29 (td, J=7.4, 1.5 Hz, 1H), 5.07 (s, 1H), 3.53-3.44 (m, 2H), 3.43 (s, 3H), 3.32 (s, 3H), 2.73-2.59 (m, 2H), 2.43 (br s, 1H); ¹³C NMR (101 MHz, CDCl₃) δ 141.0, 139.5, 138.4, 137.1, 134.4, 132.2, 131.0, 129.6, 129.5, 128.4, 128.4, 128.3, 72.1, 65.6, 58.9, 47.6, 38.4; LR-MS calcd. for C₁₇H₂₀ClN₂O₃S [M+H]⁺ 367.09, found 367.50.

### 3-Bromo-10-((3-methoxypropyl)amino)-4-methyl-4, 10-dihydrobenzo[f]thieno[3,2-c][1,2]thiazepine 5,5-dioxide (43)

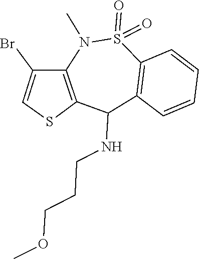 [SMILES: COCCCNC1c2ccccc2S(=O)(=O)N(C)c2c(Br)csc21]

Method B. The product 43 was purified by repeated column chromatography (column 1:CH₂Cl₂:Et₂O—20:1, 3 column volumes→15:1, 2 column volumes/column 2:Hexanes:EtOAc, 1:1) and obtained as a viscous yellow oil (74.9 mg, 58%). ¹H NMR (500 MHz, CDCl₃) δ 8.04 (dd, J=7.7, 1.2 Hz, 1H), 7.69 (td, J=7.6, 1.3 Hz, 1H), 7.53 (td, J=7.6, 0.8 Hz, 1H), 7.48 (d, J=7.6 Hz, 1H), 7.23 (s, 1H), 5.35 (s, 1H), 3.54-3.45 (m, 2H), 3.34 (s, 3H), 2.83 (s, 3H), 2.81-2.74 (m, 1H), 2.74-2.67 (m, 1H), 1.88-1.71 (m, 2H); ¹³C NMR (126 MHz, CDCl₃) δ 135.5, 135.2, 135.1, 134.3, 134.0, 130.1, 128.9, 128.1, 122.5, 109.0, 71.2, 61.4, 58.8, 45.2, 39.5, 30.0; LR-MS calcd. for C₁₆H₂₀BrN₂O₃S₂ [M+H]⁺ 433.01, found 434.02.

### 11-((3-Methoxypropyl)amino)-6-methyl-3-(methylthio)-6,11-dihydrodibenzo[c,f][1,2]thiazepine 5,5-dioxide (44)

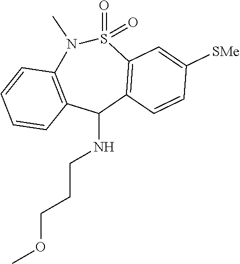 [SMILES: COCCCNC1c2ccccc2N(C)S(=O)(=O)c2cc(SC)ccc21]

Method B. The product 44 was purified by column chromatography (hexanes:EtOAc—1:1, 2 column volumes→EtOAc, 3 column volumes) and obtained as a viscous pale-yellow oil (31.1 mg, 79%). ¹H NMR (400 MHz, CDCl₃) δ 7.78 (d, J=1.7 Hz, 1H), 7.43-7.31 (m, 5H), 7.31-7.25 (m, 1H), 4.97 (s, 1H), 3.45-3.38 (m, 2H), 3.35 (s, 3H), 3.29 (s, 3H), 2.57 (t, J=6.7 Hz, 2H), 2.51 (s, 3H), 2.29 (br s, 1H), 1.82-1.68 (m, 2H). ¹³C NMR (101 MHz, CDCl₃) (unusual broadening of some carbon peaks) δ 140.0, 139.5, 139.1, 138.9, 134.8, 130.4, 130.4, 129.9, 129.3, 128.1, 128.0, 125.3, 71.3, 66.6, 58.8, 45.7, 38.8, 30.1, 15.6; LR-MS calcd. for C₁₉H₂₅N₂O₃S₂[M+H]⁺ 393.13, found 392.74.

### Ethyl 7-((6-methyl-3-(methylthio)-5, 5-dioxido-6,11-dihydrodibenzo[c,f][1,2]thiazepin-11-yl)amino)heptanoate (45)

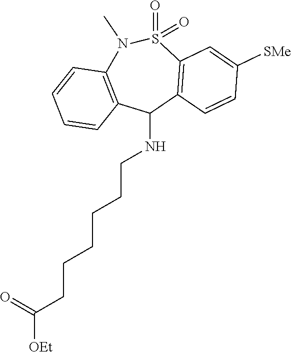 [SMILES: CCOC(=O)CCCCCCNC1c2ccccc2N(C)S(=O)(=O)c2cc(SC)ccc21]

Method C. The product 45 was obtained as a viscous pale-yellow oil (44.0 mg, 92%). ¹H NMR (400 MHz, CDCl₃) δ 7.78 (d, J=1.3 Hz, 1H), 7.42-7.31 (m, 5H), 7.31-7.24 (m, 1H), 4.95 (s, 1H), 4.10 (q, J=7.1 Hz, 2H), 3.34 (s, 3H), 2.50 (s, 3H), 2.45 (t, J=7.1 Hz, 2H), 2.25 (t, J=7.5 Hz, 2H), 2.13 (br s, 1H), 1.64-1.53 (m, 2H), 1.52-1.40 (m, 2H), 1.33-1.24 (m, 4H), 1.23 (t, J=7.1 Hz, 3H); ¹³C NMR (101 MHz, CDCl₃) δ 173.8, 140.0, 139.5, 139.0, 138.9, 134.7, 130.5, 130.5, 129.8, 129.3, 128.0, 125.3, 66.8, 60.3, 48.1, 38.9, 34.4, 30.0, 29.1, 27.1, 25.0, 15.6, 14.4; LR-MS calcd. for C₂₄H₃₃N₂O₄S₂[M+H]⁺ 477.19, found 476.70.

### 3-chloro-6-methyl-11-(((tetrahydrofuran-3-yl)methyl)amino)-6,11-dihydrodibenzo[c,f][1,2]thiazepine 5,5-dioxide (46)

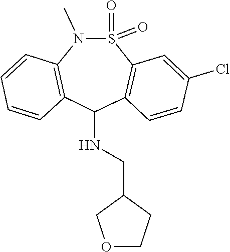 [SMILES: CN1c2ccccc2C(NCC2CCOC2)c2ccc(Cl)cc2S1(=O)=O]

Method B. The product 46 was obtained as a colorless glass (92.8 mg, 95%). ¹H NMR (400 MHz, CDCl₃) (additional splitting due to conformers) δ 7.97 (dd, J=4.3, 2.2 Hz, 1H), 7.51-7.27 (m, 6H), 4.98 (d, J=11.9 Hz, 1H), 3.86-3.76 (m, 2H), 3.74-3.65 (m, 1H), 3.54-3.42 (m, 1H), 3.31 (d, J=14.5 Hz, 3H), 2.55-2.27 (m, 3H), 2.30 (br s, 1H), 2.07-1.94 (m, 1H), 1.62-1.44 (m, 1H); ¹³C NMR (101 MHz, CDCl₃) (all peaks split due to conformers) δ 140.2, 140.0, 139.1, 138.9, 134.5, 132.5, 131.9, 131.6, 131.3, 130.6, 129.6, 128.8, 128.7, 128.12, 128.06, 128.0, 127.9, 72.1, 72.0, 67.92, 67.89, 67.4, 66.8, 51.72, 51.65, 39.8, 39.2, 38.9, 30.7, 30.6; LR-MS calcd. for C₁₉H₂₂ClN₂O₃S [M+H]⁺ 393.10, found 392.69.

### 3-chloro-6-methyl-11-((2-(1-methylpyrrolidin-2-yl)ethyl)amino)-6,11-dihydrodibenzo[c,f][1,2]thiazepine 5,5-dioxide (47)

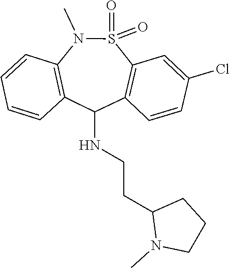 [SMILES: CN1CCCC1CCNC1c2ccccc2N(C)S(=O)(=O)c2cc(Cl)ccc21]

Method B. The product 47 was obtained as a viscous yellow oil (206 mg, 98%). ¹H NMR (400 MHz, CDCl₃) (additional splitting due to conformers) δ 7.95 (s, 1H), 7.48-7.26 (m, 6H), 5.02 (d, J=4.7 Hz, 1H), 3.38 (d, J=9.9 Hz, 3H), 3.07-2.99 (m, 1H), 2.63-2.43 (m, 2H), 2.28 (d, J=8.8 Hz, 3H), 2.28 (br s, 1H), 2.16-2.02 (m, 2H), 1.89-1.76 (m, 2H), 1.76-1.59 (m, 2H), 1.56-1.33 (m, 2H); ¹³C NMR (101 MHz, CDCl₃) (all peaks split due to conformers) δ 140.5, 140.4, 139.2, 139.1, 138.6, 138.5, 137.3, 137.1, 134.4, 134.3, 132.27, 132.25, 131.0, 130.9, 129.9, 129.8, 129.44, 129.42, 128.6, 128.5, 128.3, 128.1, 128.0, 65.9, 65.8, 64.7, 64.6, 57.34, 57.32, 45.6, 45.5, 40.71, 40.67, 38.64, 38.56, 33.8, 33.7, 30.62, 30.60, 22.2, 22.1; LR-MS calcd. for C₂₁H₂₇ClN₃O₂S [M+H]⁺ 420.15, found 419.71.

### 3-Iodo-11-((3-methoxypropyl)amino)-6-methyl-6,11-dihydrodibenzo [c,f][1,2]thiazepine 5,5-dioxide (48)

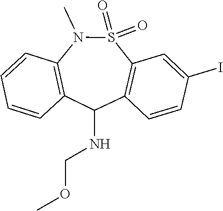 [SMILES: COCNC1c2ccccc2N(C)S(=O)(=O)c2cc(I)ccc21]

Method B. The product 48 was purified directly by column chromatography (hexanes:EtOAc—1:1, 2 column volumes→EtOAc, 2 column volumes) and obtained as a viscous pale-yellow oil (32.2 mg, 84%). ¹H NMR (400 MHz, CDCl₃) δ 8.26 (d, J=1.8 Hz, 1H), 7.81 (dd, J=8.1, 1.8 Hz, 1H), 7.43-7.32 (m, 3H), 7.31-7.27 (m, 1H), 7.24 (d, J=8.2 Hz, 1H), 5.02 (s, 1H), 3.46-3.39 (m, 2H), 3.37 (s, 3H), 3.29 (s, 3H), 2.65-2.52 (m, 2H), 2.40 (br s, 1H), 1.77 (p, J=6.3 Hz, 2H); ¹³C NMR (101 MHz, CDCl₃) δ 141.3, 140.6, 138.7, 136.9, 131.4, 130.0, 129.5, 128.3, 128.0, 93.0, 71.3, 66.1, 58.8, 45.9, 38.6, 30.0; LR-MS calcd. for C₁₈H₂₂IN₂O₃S [M+H]⁺473.04, found 472.79.

### Ethyl 7-((3-iodo-6-methyl-5, 5-dioxido-6,11-dihydrodibenzo[c,f][1,2]thiazepin-11-yl)amino)heptanoate (49)

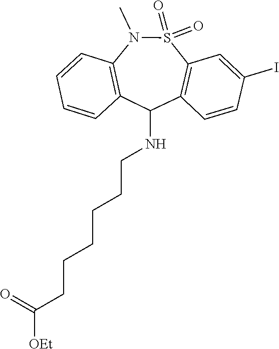 [SMILES: CCOC(=O)CCCCCCNC1c2ccccc2N(C)S(=O)(=O)c2cc(I)ccc21]

Method C. The product 49 was purified directly by column chromatography (CH₂Cl₂:Et₂O—20:1) and obtained as a viscous nearly-colorless oil (31.7 mg, 70%). ¹H NMR (400 MHz, CDCl₃) δ 8.26 (d, J=1.8 Hz, 1H), 7.81 (dd, J=8.1, 1.8 Hz, 1H), 7.41-7.32 (m, 3H), 7.31-7.26 (m, 1H), 7.21 (d, J=8.2 Hz, 1H), 4.98 (s, 1H), 4.11 (q, J=7.1 Hz, 2H), 3.36 (s, 3H), 2.46 (t, J=7.1 Hz, 2H), 2.26 (t, J=7.5 Hz, 2H), 2.05 (br s, 1H), 1.65-1.53 (m, 2H), 1.53-1.42 (m, 2H), 1.35-1.26 (m, 4H), 1.24 (t, J=7.1 Hz, 3H); ¹³C NMR (101 MHz, CDCl₃) δ 173.8, 141.3, 140.6, 138.7, 136.9, 131.5, 130.1, 129.5, 128.2, 128.1, 92.9, 66.3, 60.3, 48.2, 38.7, 34.4, 30.0, 29.1, 27.0, 25.0, 14.4; LR-MS calcd. for C₂₃H₃₀IN₂O₄S [M+H]⁺ 557.10, found 556.54.

### 7-Methoxy-11-((3-methoxypropyl)amino)-6-methyl-6,11-dihydrodibenzo [c,f][1,2]thiazepine 5,5-dioxide (50)

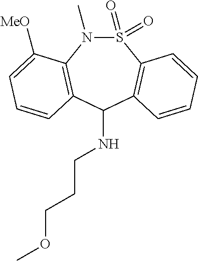 [SMILES: COCCCNC1c2ccccc2S(=O)(=O)N(C)c2c(OC)cccc21]

Method B. The product 50 was obtained as a viscous pale-yellow oil (36.6 mg, 97%). ¹H NMR (500 MHz, CDCl₃) (observed as a -55:45 ratio of 2 conformers resulting in partial integrals) δ 7.96 (q, J=8.0 Hz, 1H), 7.59 (d, J=7.7 Hz, 0.45H), 7.52-7.33 (m, 2.55H), 7.29-7.21 (m, 1H), 7.06-6.91 (m, 1H), 6.91-6.80 (m, 1H), 5.35 (s, 0.45H), 4.67 (s, 0.55H), 3.93 (s, 1.65H), 3.89 (s, 1.35H) 3.55-3.43 (m, 2.55H), 3.39-3.31 (m, 2.45H), 3.25 (s, 3H), 2.77-2.46 (m, 2H), 2.05 (br s, 1H), 1.91-1.76 (m, 1H), 1.76-1.64 (m, 1H); ¹³C NMR (126 MHz, CDCl₃) (additional peaks due to conformers) δ 158.2, 157.0, 142.5, 141.8, 138.5, 137.8, 132.2, 131.8, 131.7, 129.7, 129.3, 129.0, 128.9, 128.2, 127.8, 126.3, 122.5, 112.6, 111.2, 71.4, 71.3, 70.9, 58.8, 58.7, 56.3, 46.1, 45.9, 37.9, 36.4, 30.1; LR-MS calcd. for C₁₉H₂₅N₂O₄S [M+H]⁺ 377.15, found 377.13.

### Ethyl 7-((7-methoxy-6-methyl-5, 5-dioxido-6,11-dihydrodibenzo[c,f][1,2]thiazepin-11-yl)amino)heptanoate (51)

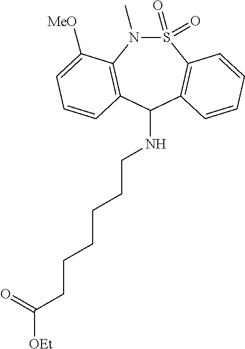 [SMILES: CCOC(=O)CCCCCCNC1c2ccccc2S(=O)(=O)N(C)c2c(OC)cccc21]

Method C. The product 51 was obtained as a viscous pale-yellow oil (44.6 mg, 97%). ¹H NMR (500 MHz, CDCl₃) (observed as a -55:45 ratio of 2 conformers resulting in partial integrals) δ 7.96 (dd, J=7.6, 1.6 Hz, 1H), 7.58 (d, J=7.6 Hz, 0.45H), 7.52-7.33 (m, 2.55H), 7.29-7.21 (m, 1H), 7.06-6.81 (m, 2H), 5.34 (s, 0.45H), 4.67 (s, 0.55H), 4.10 (p, J=7.2 Hz, 2H), 3.93 (s, 1.65H), 3.89 (s, 1.35H), 3.48 (s, 1.65H), 3.22 (s, 1.35H), 2.65-2.33 (m, 2H), 2.31-2.20 (m, 2H), 1.87 (br s, 1H), 1.66-1.50 (m, 3H), 1.46-1.29 (m, 3H), 1.28-1.18 (m, 5H); ¹³C NMR (126 MHz, CDCl₃) (additional peaks due to conformers) δ 173.8, 158.2, 156.9, 142.5, 141.8, 138.4, 137.9, 132.3, 131.7, 129.6, 129.3, 129.1, 128.9, 128.2, 127.9, 126.3, 122.5, 112.6, 111.3, 70.8, 60.3, 56.3, 48.5, 48.2, 38.0, 36.5, 34.3, 30.0, 29.8, 29.1, 27.1, 25.0, 24.9, 14.4; LR-MS calcd. for C₂₄H₃₃N₂O₅S [M+H]⁺ 461.21, found 460.98.

### 11-((7-(Aminooxy)-7-oxoheptyl)amino)-3-chloro-6-methyl-6,11-dihydrodibenzo[c,f][1,2]-thiazepine 5,5-dioxide (52)

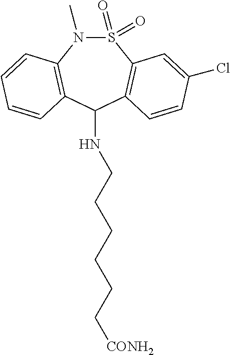 [SMILES: CN1c2ccccc2C(NCCCCCCC(N)=O)c2ccc(Cl)cc2S1(=O)=O]

Method A. The crude product was purified by column chromatography (hexanes:EtOAc—1:1->CH₂Cl₂+10% MeOH). The product 52 was obtained as a colorless oil (260 mg, 89%). ¹H NMR (400 MHz, Acetone-d₆) δ 7.82 (d, J=2.2 Hz, 1H), 7.73 (d, J=8.4 Hz, 1H), 7.63 (dd, J=8.3, 2.2 Hz, 1H), 7.52 (dd, J=7.6, 1.3 Hz, 1H), 7.48 (dd, J=7.8, 1.0 Hz, 1H), 7.41 (td, J=7.6, 1.6 Hz, 1H), 7.34 (td, J=7.5, 1.4 Hz, 1H), 6.61 (s, 1H), 5.99 (s, 1H), 5.20 (s, 1H), 3.41 (s, 3H), 2.50 (t, J=7.0 Hz, 2H), 2.12 (t, J=7.4 Hz, 2H), 1.63-1.44 (m, 4H), 1.38-1.21 (m, 4H). ¹³C NMR (101 MHz, Acetone-d₆) δ 175.2, 142.3, 140.6, 139.7, 138.6, 134.2, 133.0, 132.8, 131.0, 130.0, 129.4, 128.8, 128.2, 66.4, 48.7, 39.2, 36.0, 30.6, 27.8, 26.2; LR-MS calcd. for C₂₁H₂₇ClN₃O₃S [M+H]⁺ 436.15, found 435.70.

### 3-Bromo-11-((3-methoxypropyl)amino)-6-methyl-6,11-dihydrodibenzo[c,f][1,2]thiazepine 5,5-dioxide (53)

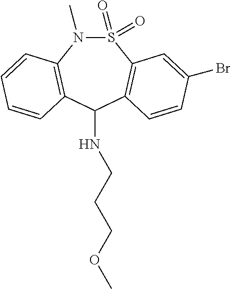 [SMILES: COCCCNC1c2ccccc2N(C)S(=O)(=O)c2cc(Br)ccc21]

Method A. The crude product was purified by column chromatography (hexanes:EtOAc—1:1). The product 53 was obtained as a colorless oil turning to a white solid (45 mg, 71%). ¹H NMR (400 MHz, Acetone-d₆) δ 7.96 (d, J=2.1 Hz, 1H), 7.77 (dd, J=8.3, 2.1 Hz, 1H), 7.65 (d, J=8.3 Hz, 1H), 7.51 (dd, J=7.6, 1.5 Hz, 1H), 7.49 (dd, J=7.9, 1.2 Hz, 1H), 7.41 (td, J=7.6, 1.6 Hz, 1H), 7.34 (td, J=7.4, 1.3 Hz, 1H), 5.18 (s, 1H), 3.42 (s, 3H), 3.39 (t, J=6.2 Hz, 2H), 3.22 (s, 3H), 2.58 (t, J=6.8 Hz, 2H), 2.54 (br s, 1H), 1.73 (p, J=6.6 Hz, 2H). ¹³C NMR (101 MHz, Acetone-d₆) δ 142.7, 140.8, 139.7, 139.1, 135.8, 133.1, 131.0, 130.8, 130.0, 129.4, 128.9, 121.9, 71.6, 66.4, 58.5, 46.2, 39.1, 30.8; LR-MS calcd. for C₁₈H₂₂BrN₂O₃S [M+H]⁺ 425.05, found 425.06.

### 11-((3-Methoxypropyl)amino)-6-methyl-3-(methylsulfonyl)-6,11-dihydrodibenzo[c,f][1,2]thiazepine 5,5-dioxide (54)

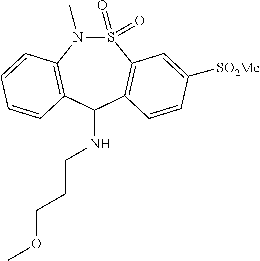 [SMILES: COCCCNC1c2ccccc2N(C)S(=O)(=O)c2cc(S(C)(=O)=O)ccc21]

Method B. The product 54 was purified directly by column chromatography (CH₂Cl₂:Et₂O—6:4) and obtained as a viscous colorless oil (38.8 mg, 91%). ¹H NMR (500 MHz, CDCl₃) δ 8.51 (d, J=1.9 Hz, 1H), 8.04 (dd, J=8.1, 1.9 Hz, 1H), 7.79 (d, J=8.2 Hz, 1H), 7.45-7.35 (m, 3H), 7.31 (td, J=7.4, 1.4 Hz, 1H), 5.24 (s, 1H), 3.45 (t, J=6.1 Hz, 2H), 3.43 (s, 3H), 3.31 (s, 3H), 3.06 (s, 3H), 2.71-2.58 (m, 2H), 2.27 (br s, 1H), 1.88-1.72 (m, 2H); ¹³C NMR (126 MHz, CDCl₃) δ 144.8, 140.8, 140.7, 139.2, 138.1, 130.8, 130.1, 129.7, 129.0, 128.6, 128.0, 127.9, 71.4, 64.9, 58.8, 46.2, 44.5, 38.2, 30.0; LR-MS calcd. for C₁₉H₂₅N₂O₅S₂ [M+H]⁺ 425.12, found 425.72.

### 11-((3-Methoxypropyl)amino)-6-methyl-3-(methylsulfinyl)-6,11-dihydrodibenzo[c,f][1,2]thiazepine 5,5-dioxide (55)

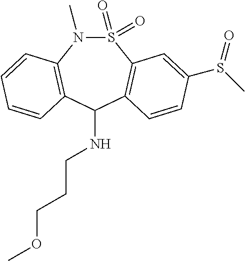 [SMILES: COCCCNC1c2ccccc2N(C)S(=O)(=O)c2cc(S(C)=O)ccc21]

Method B. The product 55 was purified directly by column chromatography (EtOAc:MeOH—20:1, 3 column volumes→EtOAc:MeOH—20:1+2% Et₃N, 3 column volumes) and obtained as a pale yellow-orange glass (35.5 mg, 87%, 1:1 mixture of diastereomers). ¹H NMR (500 MHz, CDCl₃) (partial integrals due to mixture of diastereomers) δ 8.10 (d, J=1.8 Hz, 0.5H), 8.08 (d, J=1.8 Hz, 0.5H), 7.88 (ddd, J=9.7, 8.1, 1.9 Hz, 1H), 7.74 (d, J=8.0 Hz, 1H), 7.43-7.34 (m, 3H), 7.29 (tt, J=7.5, 1.7 Hz, 1H), 5.15 (s, 1H) 3.46-3.39 (m, 5H), 3.29 (s, 1.5H), 3.29 (s, 1.5H), 2.73 (s, 1.5H), 2.73 (s, 1.5H), 2.67-2.56 (m, 2H), 2.24 (br s, 1H), 1.84-1.72 (m, 2H); ¹³C NMR (126 MHz, CDCl₃) (additional peaks due to mixture of diastereomers) δ 146.6, 146.5, 141.7, 140.4, 140.3, 139.5, 139.2, 138.3, 138.2, 130.6, 130.5, 129.6, 129.5, 129.3, 128.5, 128.4, 128.1, 128.0, 127.10, 127.07, 124.0, 123.9, 71.34, 71.30, 65.6, 58.8, 46.1, 46.0, 44.0, 43.9, 38.4, 38.3, 30.0. LR-MS calcd. for C₁₉H₂₅N₂O₄S₂[M+H]⁺ 409.13, found 409.31.

### 11-((3-Methoxypropyl)amino)-6-methyl-3-phenyl-6,11-dihydrodibenzo[c,f][1,2]thiazepine 5,5-dioxide (56)

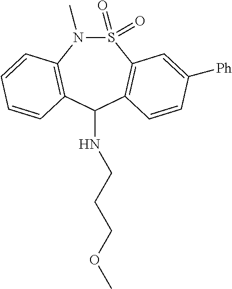 [SMILES: COCCCNC1c2ccccc2N(C)S(=O)(=O)c2cc(-c3ccccc3)ccc21]

Method A. The crude product was purified by column chromatography (hexanes:EtOAc—1:1). The product 56 was obtained as a colorless oil (75 mg, 89%). ¹H NMR (400 MHz, Acetone-d₆) δ 8.11 (d, J=1.9 Hz, 1H), 7.88 (dd, J=8.1, 1.9 Hz, 1H), 7.77 (d, J=8.1 Hz, 1H), 7.73-7.67 (m, 2H), 7.57-7.46 (m, 4H), 7.45-7.41 (m, 1H), 7.39 (dd, J=7.7, 1.6 Hz, 1H), 7.34 (td, J=7.5, 1.4 Hz, 1H), 5.24 (s, 1H), 3.41 (m, 5H), 3.23 (s, 3H), 2.62 (t, J=6.8 Hz, 2H), 2.57 (br s, 1H), 1.75 (p, J=6.6 Hz, 2H); ¹³C NMR (101 MHz, Acetone-d₆) δ 141.7, 141.1, 140.9, 140.1, 139.7, 138.7, 131.6, 131.1, 130.9, 130.0, 129.8, 129.1, 129.1, 128.6, 127.7, 126.8, 71.6, 66.6, 58.5, 46.2, 38.9, 30.9; LR-MS calcd. for C₂₄H₂₇N₂O₃S [M+H]⁺ 423.17, found 423.22.

Preparation of Compounds 57 and 58

Compounds 57 and 58 were prepared from tianeptine sodium according to the following procedure. To a solution of tianeptine sodium salt (91.8 mg, 0.200 mmol) and triphenylphosphine (157 mg, 0.600 mmol) in anhydrous CH₂Cl₂ (3.9 mL) was added ethanolamine (12.1 μL, 0.200 mmol) and diisopropylethylamine (105 μL, 0.600 mmol) and the solution was cooled to 0° C. Anhydrous carbon tetrachloride (96.5 μL, 1.00 mmol) was then added slowly over ˜75 min, and the reaction was allowed to warm to room temperature and stirred for 13 h. The reaction mixture was then purified directly by column chromatography (EtOAc, 6 column volumes→EtOAc+2% Et₃N, 3 column volumes→CH₂Cl₂:MeOH—10:1, 3 column volumes) to yield one fraction containing impure 58 and a second fraction containing 57 contaminated with a partial salt of Et₃N. The fraction containing 57 was dissolved in CH₂Cl₂ (10 mL) and washed with H₂O (5 mL) and 10% NH₄OH (5 mL). The organic layer was then dried over Na₂SO₄, concentrated, and dried thoroughly under high vacuum to yield the pure amide 57. The fraction containing 58 was purified on a second chromatography column (EtOAc, 8 column volumes→CH₂Cl₂:MeOH—10:1, 3 column volumes) to yield the pure oxazoline 58.

### 7-((3-Chloro-6-methyl-5, 5-dioxido-6,11-dihydrodibenzo [c,f][1,2]thiazepin-11-yl)amino)-N-(2-hydroxyethyl) heptanamide (57)

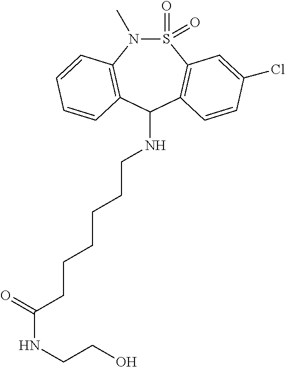 [SMILES: CN1c2ccccc2C(NCCCCCCC(=O)NCCO)c2ccc(Cl)cc2S1(=O)=O]

The product 57 was obtained as a viscous yellow oil (50.8 mg, 53%). ¹H NMR (400 MHz, CDCl₃) δ 7.94 (d, J=2.1 Hz, 1H), 7.49-7.26 (m, 6H), 6.17 (br s, 1H), 4.97 (s, 1H), 3.67-3.58 (m, 2H), 3.37-3.30 (m, 2H), 3.33 (s, 3H), 2.52 (br s, 2H), 2.49-2.37 (m, 2H), 2.14 (t, J=7.5 Hz, 2H), 1.64-1.51 (m, 2H), 1.51-1.38 (m, 2H), 1.33-1.18 (m, 4H); ¹³C NMR (101 MHz, CDCl₃) δ 174.5, 140.2, 138.7, 138.4, 136.9, 134.4, 132.4, 131.6, 130.5, 129.5, 128.5, 128.2, 128.0, 66.5, 62.3, 48.0, 42.5, 38.9, 36.5, 29.8, 29.0, 26.9, 25.6; LR-MS calcd. for C₂₃H₃₁ClN₃O₄S [M+H]⁺ 480.17, found 479.88.

### 3-Chloro-11-((6-(4, 5-dihydrooxazol-2-yl)hexyl)amino)-6-methyl-6,11-dihydrodibenzo[c,f][1,2]thiazepine 5,5-dioxide (58)

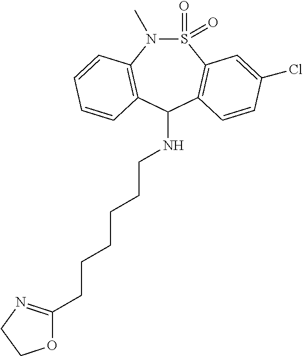 [SMILES: CN1c2ccccc2C(NCCCCCCC2=NCCO2)c2ccc(Cl)cc2S1(=O)=O]

The product 58 was obtained as a viscous pale-yellow oil (2.9 mg, 3.1%). ¹H NMR (400 MHz, CDCl₃) δ 7.96 (d, J=2.0 Hz, 1H), 7.55-7.28 (m, 6H), 5.06 (br s, 1H), 4.21 (t, J=9.4 Hz, 2H), 3.80 (t, J=9.4 Hz, 2H), 3.36 (s, 3H), 2.53-2.40 (m, 2H), 2.28-2.19 (m, 2H), 2.11 (br s, 1H), 1.60 (dt, J=14.6, 7.4 Hz, 2H), 1.50 (br s, 2H), 1.31 (dt, J=7.3, 3.7 Hz, 4H); ¹³C NMR (101 MHz, CDCl₃) δ 168.7, 140.5, 138.8, 134.5, 132.4, 131.4, 130.3, 129.6, 128.6, 128.3, 128.1, 67.3, 66.3, 54.5, 48.2, 38.8, 30.0, 29.2, 28.0, 27.0, 26.0; LR-MS calcd. for C₂₃H₂₉ClN₃O₃S [M+H]⁺ 462.16, found 462.10.

### 11-((3-Methoxypropyl)amino)-6-methyl-5, 5-dioxido-6,11-dihydrodibenzo[c,f][1,2]thiazepin-3-yl acetate (59)

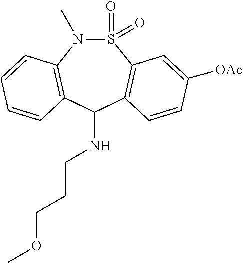 [SMILES: COCCCNC1c2ccccc2N(C)S(=O)(=O)c2cc(OC(C)=O)ccc21]

To a solution of alcohol 4m (65 mg, 0.223 mmol) in anhydrous CH₂Cl₂ (3.0 mL) was added 2M HCl in Et₂O (0.30 mL) and the resulting mixture was stirred for 4 h at room temperature and then concentrated in vacuo to provide the crude intermediate chloride as an orange solid (65 mg). This material was then aminated with 3-methoxypropylamine (0.10 mL) according to general Method A. The resulting crude product was dissolved in acetic acid (5 mL) and acetyl chloride (0.50 mL) was added at 5° C. The reaction was then allowed to warm to room temperature and stirred overnight. The mixture was then concentrated in vacuo and the residue was suspended/dissolved in H₂O and neutralized with saturated aqueous NaHCO₃. The aqueous mixture was then extracted with CH₂Cl₂ and the combined organics were dried over Na₂SO₄ and concentrated to provide the crude product. This material was purified by column chromatography (CH₂Cl₂+2% MeOH) to give pure product 59 as a yellowish oily solid (68 mg, 75% from alcohol 4m). ¹H NMR (400 MHz, Acetone-d₆) δ 7.71 (d, J=8.5 Hz, 1H), 7.62 (d, J=2.4 Hz, 1H), 7.51 (dd, J=7.6, 1.6 Hz, 1H), 7.48 (dd, J=7.8, 1.4 Hz, 1H), 7.43-7.30 (m, 3H), 5.19 (s, 1H), 3.43-3.36 (m, 2H), 3.41 (s, 3H), 3.22 (s, 3H), 2.82 (br s, 1H), 2.58 (t, J=6.8 Hz, 2H), 2.30 (s, 3H), 1.74 (p, J=6.5 Hz, 2H); ¹³C NMR (101 MHz, Acetone-d₆) δ 169.5, 151.2, 141.6, 141.0, 139.9, 137.1, 132.2, 130.8, 129.9, 129.3, 128.8, 126.3, 122.1, 71.6, 66.4, 58.5, 46.2, 38.9, 30.8, 20.9; LR-MS calcd. for C₂₀H₂₅N₂O₅S [M+H]⁺405.15, found 405.00.

### 2-Chloro-11-((3-methoxypropyl)amino)-6-methyl-6,11-dihydrodibenzo [c,f][1,2]thiazepine 5,5-dioxide (60)

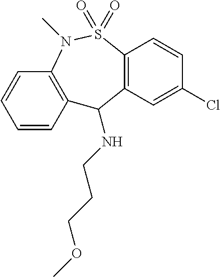 [SMILES: COCCCNC1c2ccccc2N(C)S(=O)(=O)c2ccc(Cl)cc21]

Method A. The product 60 was purified by column chromatography (hexanes:EtOAc—1:1) and obtained as a viscous colorless oil (72 mg, 95%). ¹H NMR (400 MHz, Acetone-d₆) δ 7.88 (d, J=8.5 Hz, 1H), 7.76 (d, J=2.2 Hz, 1H), 7.56-7.47 (m, 3H), 7.41 (td, J=7.6, 1.7 Hz, 1H), 7.35 (td, J=7.4, 1.5 Hz, 1H), 5.28 (s, 1H), 3.42 (t, J=6.1 Hz, 2H), 3.41 (s, 3H), 3.24 (s, 3H), 2.81 (br s, 1H), 2.63 (t, J=6.8 Hz, 2H), 1.77 (dc, J=12.2, 6.2 Hz, 2H); ¹³C NMR (101 MHz, Acetone-d₆) δ 142.4, 141.0, 139.7, 139.4, 138.3, 130.7, 130.0, 129.9, 129.8, 129.1, 128.92, 128.86, 71.5, 65.2, 58.6, 46.2, 38.4, 30.8; LR-MS calcd. for C₁₈H₂₂ClN₂OS [M+H]⁺ 381.10, found 381.46.

### Ethyl 7-((2-chloro-6-methyl-5, 5-dioxido-6,11-dihydrodibenzo[c,f][1,2]thiazepin-11-yl)amino)heptanoate (61)

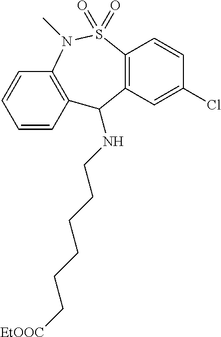 [SMILES: CCOC(=O)CCCCCCNC1c2ccccc2N(C)S(=O)(=O)c2ccc(Cl)cc21]

Method A. The product 61 was purified by column chromatography (hexanes:EtOAc—2:1) and obtained as a viscous colorless oil (120 mg, 81%). ¹H NMR (400 MHz, Acetone-d₆) δ 7.88 (d, J=8.5 Hz, 1H), 7.76 (d, J=2.2 Hz, 1H), 7.57-7.51 (m, 2H), 7.49 (dd, J=7.8, 1.4 Hz, 1H), 7.40 (td, J=7.6, 1.7 Hz, 1H), 7.34 (td, J=7.4, 1.5 Hz, 1H), 5.28 (s, 1H), 4.06 (q, J=7.1 Hz, 2H), 3.40 (s, 3H), 2.80 (br s, 1H), 2.55 (t, J=7.0 Hz, 2H), 2.25 (t, J=7.4 Hz, 2H), 1.62-1.49 (m, 4H), 1.41-1.26 (m, 4H), 1.19 (t, J=7.1 Hz, 3H); ¹³C NMR (101 MHz, Acetone-d₆) δ 173.60, 142.40, 140.87, 139.76, 139.28, 138.29, 130.68, 130.22, 129.95, 129.89, 129.11, 128.90, 128.81, 65.37, 60.40, 48.67, 38.57, 34.56, 30.58, 27.68, 25.61, 14.58; LR-MS calcd. for C₂₃H₃₀ClN₂O₄S [M+H]⁺ 465.16, found 465.68.

### 7-Hydroxy-11-((3-methoxypropyl)amino)-6-methyl-6,11-dihydrodibenzo[c,f][1,2]thiazepine 5,5-dioxide (62)

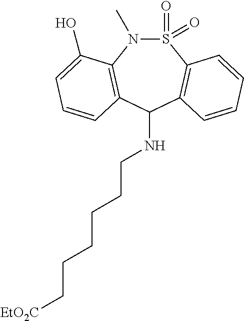 [SMILES: CCOC(=O)CCCCCCNC1c2ccccc2S(=O)(=O)N(C)c2c(O)cccc21]

Compound 62 was prepared according to the following procedure. To a solution of compound 51 (29.1 mg, 0.0631 mmol) in anhydrous CH₂Cl₂ (0.50 mL) at 0° C. was added aluminum chloride (50.5 mg, 0.379 mmol) followed by ethanethiol (84.4 μL, 1.14 mmol), and the resulting mixture was allowed to warm to room temperature and stirred for 1.5 h. The reaction was then quenched with water (5 mL) and extracted with CH₂Cl₂ (3×5 mL). The combined organics were washed with water (5 mL), dried over Na₂SO₄, and concentrated to give a white solid. This material was purified by column chromatography (CH₂Cl₂:Et₂O—6:1, 2 column volumes→7:3, 2 column volumes) to provide the pure product 62 as a white solid (23.3 mg, 83%). ¹H NMR (500 MHz, CDCl₃) (partial integrals due to conformers) δ 8.00-7.94 (m, 1H), 7.63-7.36 (m, 3H), 7.23-7.15 (m, 1H), 6.99 (d, J=8.0 Hz, 1H), 6.88 (dd, J=17.9, 7.7 Hz, 1H), 6.29 (br s, 1H), 5.24 (s, 0.5H), 4.74 (s, 0.5H), 4.11 (p, J=7.1 Hz, 2H), 3.56 (s, 1.5H), 3.17 (s, 1.5H), 2.67-2.57 (m, 0.5H), 2.51 (td, J=13.7, 6.8 Hz, 1H), 2.39 (dt, J=11.2, 7.3 Hz, 0.5H), 2.32-2.20 (m, 2H), 1.66-1.52 (m, 3H), 1.47-1.17 (m, 8H); ¹³C NMR (126 MHz, CDCl₃) (additional peaks due to conformers) δ 174.0, 173.90, 155.2, 153.8, 141.7, 140.9, 137.7, 132.8, 132.2, 131.8, 130.2, 129.7, 129.3, 128.7, 128.2, 125.0, 122.8, 117.4, 116.0, 71.2, 60.4, 48.7, 48.2, 39.7, 38.0, 34.4, 34.3, 30.0, 29.8, 29.12, 29.08, 27.10, 27.06, 25.0, 24.9, 14.4; LR-MS calcd. for C₂₃H₃₁N₂O₅S [M+H]⁺ 447.19, found 447.15.

### 3-(Isopropylthio)-11-((3-methoxypropyl)amino)-6-methyl-6,11-dihydrodibenzo[c,f][1,2]thiazepine 5,5-dioxide (63)

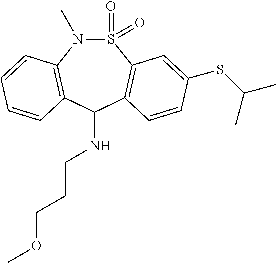 [SMILES: COCCCNC1c2ccccc2N(C)S(=O)(=O)c2cc(SC(C)C)ccc21]

Method B. The product 63 was purified by column chromatography (hexanes:EtOAc—1:1, 2 column volumes→EtOAc, 3 column volumes) and obtained as a viscous, pale-yellow oil (37.7 mg, 90%). ¹H NMR (400 MHz, CDCl₃) δ 7.93 (d, J=1.9 Hz, 1H), 7.45 (dd, J=8.0, 1.9 Hz, 1H), 7.42-7.31 (m, 4H), 7.27 (dt, J=7.2, 2.2 Hz, 1H), 4.99 (s, 1H), 3.50-3.38 (m, 3H), 3.36 (s, 3H), 3.29 (s, 3H), 2.58 (t, J=6.7 Hz, 2H), 2.32 (s, 1H), 1.75 (tt, J=7.9, 4.1 Hz, 2H), 1.31 (d, J=6.7 Hz, 6H); ¹³C NMR (101 MHz, CDCl₃) δ 139.4, 139.0, 138.9, 137.4, 136.2, 134.2, 130.3, 130.2, 130.0, 129.3, 128.1, 128.0, 71.3, 66.5, 58.8, 45.7, 38.6, 38.0, 30.1, 23.12, 23.10; LR-MS calcd. for C₂₁H₂₉N₂O₃S₂ [M+H]⁺ 421.16, found 421.50.

### 3-(Ethylthio)-11-((3-methoxypropyl)amino)-6-methyl-6,11-dihydrodibenzo[c,f][1,2]thiazepine 5,5-dioxide (64)

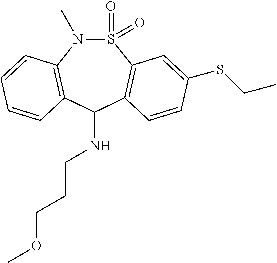 [SMILES: CCSc1ccc2c(c1)S(=O)(=O)N(C)c1ccccc1C2NCCCOC]

Method B. The product 64 was purified by column chromatography (hexanes:EtOAc—1:1, 2 column volumes→EtOAc, 3 column volumes) and obtained as a viscous, nearly colorless oil (36.6 mg, 90%). ¹H NMR (500 MHz, CDCl₃) δ 7.84 (s, 1H), 7.42-7.31 (m, 5H), 7.30-7.25 (m, 1H), 4.97 (s, 1H), 3.45-3.37 (m, 2H), 3.35 (s, 3H), 3.29 (s, 3H), 2.98 (q, J=7.4 Hz, 2H), 2.62-2.52 (m, 2H), 2.32 (br s, 1H), 1.81-1.69 (m, 2H), 1.32 (t, J=7.4 Hz, 3H); ¹³C NMR (126 MHz, CDCl₃) δ 139.4, 138.9, 138.5, 135.2, 131.6, 130.4, 130.3, 129.3, 128.1, 128.1, 127.2, 71.3, 66.6, 58.8, 45.7, 38.7, 30.1, 27.2, 14.2; LR-MS calcd. for C₂₀H₂₇N₂O₃S₂[M+H]⁺ 407.15, found 407.14.

### 6-Ethyl-11-((3-methoxypropyl)amino)-6,11-dihydrodibenzo[c,f][1,2]thiazepine 5,5-dioxide (65)

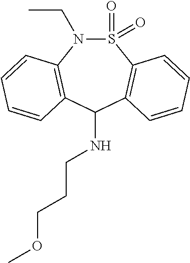 [SMILES: CCN1c2ccccc2C(NCCCOC)c2ccccc2S1(=O)=O]

Method B. The product 65 was purified by column chromatography (hexanes:EtOAc—1:1, 2 column volumes→EtOAc, 3 column volumes) and obtained as a viscous, colorless oil (30.6 mg, 85%). ¹H NMR (500 MHz, CDCl₃) δ 7.98-7.94 (m, 1H), 7.52-7.45 (m, 2H), 7.43-7.38 (m, 3H), 7.31 (dtd, J=19.3, 7.4, 1.5 Hz, 2H), 5.16 (s, 1H), 3.96-3.77 (m, 2H), 3.50-3.39 (m, 2H), 3.31 (s, 3H), 2.68-2.56 (m, 2H), 2.32 (br s, 1H), 1.83-1.73 (m, 2H), 1.18 (t, J=7.2 Hz, 3H); ¹³C NMR (126 MHz, CDCl₃) δ 140.7, 139.9, 138.7, 136.9, 132.2, 129.3, 129.1, 129.0, 128.7, 128.4, 128.3, 71.3, 65.2, 58.8, 46.3, 45.8, 30.1, 14.9; LR-MS calcd. for C₁₉H₂₅N₂O₃S [M+H]⁺ 361.16, found 361.06.

### Ethyl 7-((6-ethyl-5,5-dioxido-6,11-dihydrodibenzo[c,f][1,2]thiazepin-11-yl)amino)heptanoate (66)

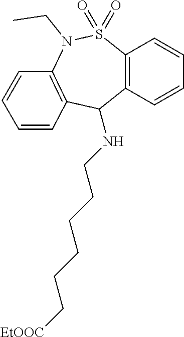 [SMILES: CCOC(=O)CCCCCCNC1c2ccccc2N(CC)S(=O)(=O)c2ccccc21]

Method C. The product 66 was purified by column chromatography (CH₂Cl₂:Et₂O—20:1, 4 column volumes→10:1, 2 column volumes) and obtained as a viscous, colorless oil (33.8 mg, 76%). ¹H NMR (500 MHz, CDCl₃) δ 7.96 (d, J=7.8 Hz, 1H), 7.50-7.46 (m, 2H), 7.43-7.38 (m, 3H), 7.35-7.27 (m, 2H), 5.13 (s, 1H), 4.11 (q, J=7.1 Hz, 2H), 3.95-3.77 (m, 2H), 2.55-2.43 (m, 2H), 2.26 (t, J=7.5 Hz, 2H), 2.09 (br s, 1H), 1.63-1.55 (m, 2H), 1.54-1.47 (m, 2H), 1.37-1.26 (m, 4H), 1.24 (t, J=7.1 Hz, 3H), 1.17 (t, J=7.2 Hz, 3H); ¹³C NMR (126 MHz, CDCl₃) δ 173.9, 139.9, 138.8, 136.9, 132.4, 129.5, 129.0, 129.0, 128.9, 128.5, 128.32, 128.26, 65.4, 60.3, 48.2, 46.4, 34.4, 30.0, 29.1, 27.1, 25.0, 14.8, 14.4; LR-MS calcd. for C₂₄H₃₃N₂O₄S [M+H]⁺ 445.22, found 445.15.

### 3-Chloro-11-((3-methoxybutyl)amino)-6-methyl-6,11-dihydrodibenzo [c,f][1,2]thiazepine 5,5-dioxide (67)

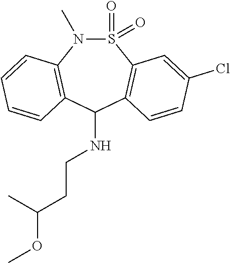 [SMILES: COC(C)CCNC1c2ccccc2N(C)S(=O)(=O)c2cc(Cl)ccc21]

Method B. The product 67 was purified by column chromatography (hexanes:EtOAc—1:1, 2 column volumes→EtOAc, 2 column volumes) and obtained as a viscous, colorless oil (30.4 mg, 77%, 1:1 mixture of diastereomers). ¹H NMR (400 MHz, CDCl₃) (partial integrals due to mixture of diastereomers) δ 7.95 (d, J=1.2 Hz, 1H), 7.49-7.32 (m, 5H), 7.32-7.27 (m, 1H), 5.04 (s, 0.5H), 5.02 (s, 0.5H), 3.43-3.34 (m, 1H), 3.38 (s, 1.5H), 3.37 (s, 1.5H), 3.27 (s, 1.5H), 3.26 (s, 1.5H), 2.64-2.51 (m, 2H), 2.36 (br s, 1H), 1.75-1.57 (m, 2H), 1.10 (t, J=6.0 Hz, 3H); ¹³C NMR (101 MHz, CDCl₃) (additional peaks due to mixture of diastereomers) δ 140.5, 140.4, 138.6, 137.0, 134.3, 132.3, 131.2, 130.0, 129.5, 128.5, 128.3, 128.1, 75.7, 75.4, 66.2, 65.9, 56.1, 45.2, 44.9, 38.7, 36.8, 36.7, 19.11, 19.08; LR-MS calcd. for C₁₉H₂₄ClN₂O₃S [M+H]⁺ 395.12, found 394.98.

### 3-Chloro-11-((3-methoxy-2-methylpropyl)amino)-6-methyl-6,11-dihydrodibenzo[c,f][1,2]thiazepine 5,5-dioxide (68)

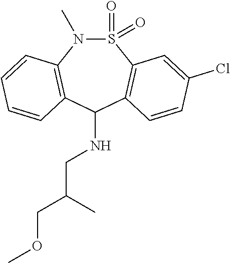 [SMILES: COCC(C)CNC1c2ccccc2N(C)S(=O)(=O)c2cc(Cl)ccc21]

Method B. The product 68 was purified by column chromatography (CH₂Cl₂:Et₂O—20:1, 2 column volumes→10:1, 2 column volumes) and obtained as a viscous, colorless oil (30.1 mg, 76%, 1:1 mixture of diastereomers). ¹H NMR (400 MHz, CDCl₃) (partial integrals due to mixture of diastereomers) δ 7.97-7.93 (m, 1H), 7.51-7.32 (m, 5H), 7.29 (td, J=7.2, 1.5 Hz, 1H), 5.04 (s, 0.5H), 5.02 (s, 0.5H), 3.38 (s, 1.5H), 3.37 (s, 1.5H), 3.32-3.22 (m, 2H), 3.31 (s, 1.5H), 3.29 (s, 1.5H), 2.59-2.31 (m, 2H), 2.43 (br s, 1H), 2.03-1.90 (m, 1H), 0.89 (dd, J=6.9, 4.8 Hz, 3H); ¹³C NMR (101 MHz, CDCl₃) (additional peaks due to mixture of diastereomers) δ 140.4, 140.3, 139.1, 138.59, 138.55, 137.2, 134.34, 134.26, 132.3, 131.1, 129.8, 129.43, 129.39, 128.49, 128.45, 128.3, 128.0, 77.32, 77.26, 66.1, 65.7, 59.0, 52.7, 52.5, 38.5, 34.0, 15.74, 15.67; LR-MS calcd. for C₁₉H₂₄ClN₂O₃S [M+H]⁺ 395.12, found 394.98.

### 8-Chloro-11-((3-methoxypropyl)amino)-6-methyl-6,11-dihydrodibenzo [c,f][1,2]thiazepine 5,5-dioxide (69)

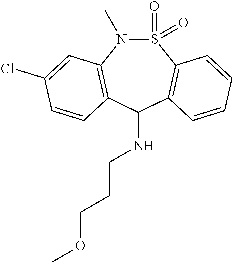 [SMILES: COCCCNC1c2ccc(Cl)cc2N(C)S(=O)(=O)c2ccccc21]

Method B. The product 69 was purified by column chromatography (hexanes:EtOAc—1:1, 2 column volumes→EtOAc, 3 column volumes) and obtained as a viscous, nearly colorless oil (33.8 mg, 89%). ¹H NMR (500 MHz, CDCl₃) δ 7.98 (dd, J=7.8, 0.9 Hz, 1H), 7.54 (td, J=7.6, 1.2 Hz, 1H), 7.52-7.41 (m, 3H), 7.33 (d, J=2.1 Hz, 1H), 7.25 (dd, J=8.6, 2.3 Hz, 1H), 5.09 (s, 1H), 3.45-3.38 (m, 2H), 3.28 (s, 3H), 3.28 (s, 3H), 3.01 (br s, 1H), 2.61 (dt, J=13.2, 6.7 Hz, 1H), 2.53 (dt, J=11.4, 6.7 Hz, 1H), 1.83-1.71 (m, 2H); ¹³C NMR (126 MHz, CDCl₃) δ 140.5, 138.3, 137.3, 136.2, 134.5, 132.8, 132.1, 130.3, 128.68, 128.65, 128.0, 127.5, 71.2, 66.4, 58.8, 45.7, 38.6, 29.8; LR-MS calcd. for C₁₈H₂₂ClN₂O₃S [M+H]⁺ 381.10, found 380.79.

### Ethyl 7-((8-chloro-6-methyl-5, 5-dioxido-6,11-dihydrodibenzo[c,f][1,2]thiazepin-11-yl)amino)heptanoate (70)

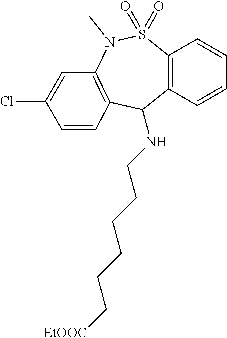 [SMILES: CCOC(=O)CCCCCCNC1c2ccc(Cl)cc2N(C)S(=O)(=O)c2ccccc21]

Method C. The product 70 was purified by column chromatography (CH₂Cl₂:Et₂O—20:1, 2 column volumes→10:1, 2 column volumes) and obtained as a viscous, nearly colorless oil (38.0 mg, 82%). ¹H NMR (500 MHz, CDCl₃) δ 7.98 (dd, J=8.0, 1.1 Hz, 1H), 7.56-7.51 (m, 1H), 7.48-7.43 (m, 2H), 7.38 (d, J=8.4 Hz, 1H), 7.33 (d, J=2.1 Hz, 1H), 7.24 (dd, J=8.3, 2.1 Hz, 1H), 5.00 (s, 1H), 4.10 (q, J=7.1 Hz, 2H), 3.28 (s, 3H), 2.49-2.38 (m, 2H), 2.25 (t, J=7.5 Hz, 2H), 2.16 (br s, 1H), 1.58 (p, J=7.4 Hz, 2H), 1.53-1.40 (m, 2H), 1.32-1.25 (m, 4H), 1.23 (t, J=7.1 Hz, 3H); ¹³C NMR (126 MHz, CDCl₃) δ 173.9, 140.4, 138.3, 137.8, 136.7, 134.3, 132.7, 132.1, 130.2, 128.7, 128.5, 128.0, 127.6, 66.6, 60.3, 48.0, 38.7, 34.4, 29.9, 29.1, 27.0, 24.9, 14.4; LR-MS calcd. for C₂₃H₃₀ClN₂O₄S [M+H]⁺ 465.16, found 464.58.

### 3-Bromo-11-((3-ethoxypropyl)amino)-6-methyl-6,11-dihydrodibenzo [c,f][1,2]thiazepine 5,5-dioxide (71)

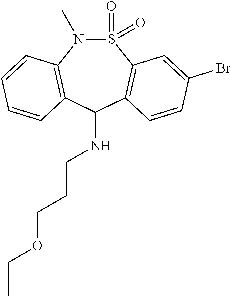 [SMILES: CCOCCCNC1c2ccccc2N(C)S(=O)(=O)c2cc(Br)ccc21]

Method B. The product 71 was purified by column chromatography (hexanes:EtOAc—1:1, 2 column volumes→EtOAc, 3 column volumes) and obtained as a viscous, colorless oil (37.5 mg, 85%). ¹H NMR (500 MHz, CDCl₃) δ 8.09 (d, J=2.0 Hz, 1H), 7.61 (dd, J=8.3, 2.0 Hz, 1H), 7.43-7.33 (m, 4H), 7.29 (td, J=7.4, 1.4 Hz, 1H), 5.05 (s, 1H), 3.53-3.40 (m, 4H), 3.38 (s, 3H), 2.64-2.53 (m, 2H), 2.31 (br s, 1H), 1.83-1.70 (m, 2H), 1.15 (t, J=7.0 Hz, 3H); ¹³C NMR (126 MHz, CDCl₃) δ 140.6, 138.6, 135.3, 131.3, 129.8, 129.4, 128.3, 128.1, 122.0, 69.1, 66.4, 65.7, 46.0, 38.6, 30.0, 15.3; LR-MS calcd. for C₁₉H₂₄BrN₂O₃S [M+H]⁺ 439.07, found 438.76.

### 7-Bromo-10-((3-methoxypropyl)amino)-4-methyl-4, 10-dihydrobenzo[f]thieno[3,2-c][1,2]thiazepine 5,5-dioxide (72)

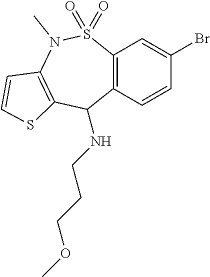 [SMILES: COCCCNC1c2ccc(Br)cc2S(=O)(=O)N(C)c2ccsc21]

Method B. The product 72 was purified by column chromatography (CH₂Cl₂:Et₂O—20:1, 3 column volumes→10:1, 3 column volumes→5:1, 4 column volumes) and obtained as a beige solid (22.1 mg, 51%). ¹H NMR (400 MHz, CDCl₃) δ 8.11 (d, J=2.0 Hz, 1H), 7.77 (dd, J=8.2, 2.0 Hz, 1H), 7.39 (d, J=8.1 Hz, 1H), 7.21 (d, J=5.5 Hz, 1H), 6.82 (d, J=5.5 Hz, 1H), 5.39 (s, 1H), 3.54-3.43 (m, 2H), 3.34 (s, 3H), 3.05 (s, 3H), 2.77 (t, J=6.4 Hz, 2H), 1.88-1.76 (m, 2H); ¹³C NMR (101 MHz, CDCl₃) δ 137.3, 136.7, 131.1, 130.8, 124.5, 124.3, 121.9, 71.2, 60.8, 58.8, 45.4, 39.6, 29.9; LR-MS calcd. for C₁₆H₂₀BrN₂O₃S₂ [M+H]⁺ 431.01, found 431.92.

### 7-Bromo-10-((3-ethoxypropyl)amino)-4-methyl-4, 10-dihydrobenzo[f]thieno[3,2-c][1,2]thiazepine 5,5-dioxide (73)

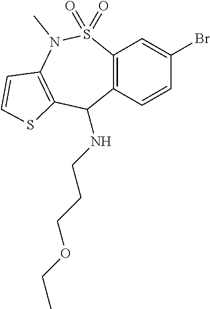 [SMILES: CCOCCCNC1c2ccc(Br)cc2S(=O)(=O)N(C)c2ccsc21]

Method B. The product 73 was purified by column chromatography (CH₂Cl₂:Et₂O—20:1, 3 column volumes→10:1, 3 column volumes→5:1, 4 column volumes) and obtained as a beige solid (27.5 mg, 62%). ¹H NMR (400 MHz, CDCl₃) δ 8.11 (d, J=2.1 Hz, 1H), 7.77 (dd, J=8.2, 2.1 Hz, 1H), 7.41 (d, J=8.1 Hz, 1H), 7.21 (d, J=5.5 Hz, 1H), 6.82 (d, J=5.5 Hz, 1H), 5.41 (s, 1H), 3.57-3.51 (m, 2H), 3.48 (q, J=7.0 Hz, 2H), 3.05 (s, 3H), 2.78 (t, J=6.4 Hz, 2H), 1.91-1.75 (m, 2H), 1.18 (t, J=7.0 Hz, 3H); ¹³C NMR (101 MHz, CDCl₃) δ 137.3, 136.7, 131.1, 130.8, 124.5, 124.3, 121.9, 69.0, 66.4, 60.6, 45.5, 39.6, 29.9, 15.4; LR-MS calcd. for C₁₇H₂₂BrN₂O₃S₂ [M+H]⁺ 445.02, found 446.00.

### Ethyl 7-((7-bromo-4-methyl-5, 5-dioxido-4, 10-dihydrobenzo[f]thieno[3,2-c][1,2]thiazepin-10-yl)amino)heptanoate (74)

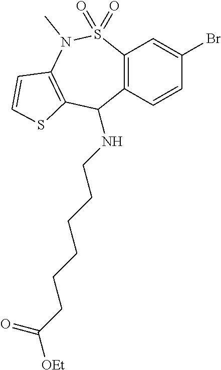 [SMILES: CCOC(=O)CCCCCCNC1c2ccc(Br)cc2S(=O)(=O)N(C)c2ccsc21]

Method C. The product 74 was purified by column chromatography (CH₂Cl₂:Et₂O—20:1, 4 column volumes→10:1, 4 column volumes) and obtained as a dark-brown solid (52.5 mg, 51%). ¹H NMR (400 MHz, CDCl₃) δ 8.11 (d, J=2.1 Hz, 1H), 7.76 (dd, J=8.2, 2.1 Hz, 1H), 7.30 (d, J=8.2 Hz, 1H), 7.19 (d, J=5.5 Hz, 1H), 6.81 (d, J=5.5 Hz, 1H), 5.31 (s, 1H), 4.11 (q, J=7.1 Hz, 2H), 3.03 (s, 3H), 2.62 (t, J=6.9 Hz, 2H), 2.27 (t, J=7.5 Hz, 2H), 1.66-1.55 (m, 2H), 1.55-1.45 (m, 2H), 1.40-1.27 (m, 4H), 1.24 (t, J=7.1 Hz, 3H); ¹³C NMR (101 MHz, CDCl₃) δ 173.9, 137.3, 136.5, 135.3, 135.0, 131.2, 130.8, 129.6, 124.5, 124.1, 121.7, 60.8, 60.3, 47.7, 39.6, 34.4, 29.9, 29.0, 26.9, 25.0, 14.4; LR-MS calcd. for C₂₁H₂₈BrN₂O₄S₂ [M+H]⁺ 515.07, found 516.24.

### 3-(Benzyloxy)-11-((3-methoxypropyl)amino)-6-methyl-6,11-dihydrodibenzo[c,f][1,2]thiazepine 5,5-dioxide (75)

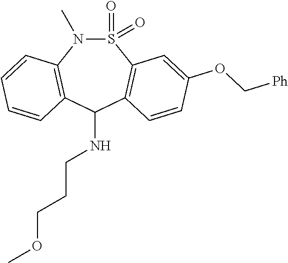 [SMILES: COCCCNC1c2ccccc2N(C)S(=O)(=O)c2cc(OCc3ccccc3)ccc21]

Method B. The product 75 was purified by column chromatography (hexanes:EtOAc—1:1, 3 column volumes→EtOAc, 4 column volumes) and obtained as a viscous, nearly colorless oil (31.9 mg, 70%). ¹H NMR (500 MHz, CDCl₃) δ 7.59 (d, J=2.7 Hz, 1H), 7.44-7.31 (m, 9H), 7.30-7.26 (m, 1H), 7.08 (dd, J=8.5, 2.7 Hz, 1H), 5.09 (s, 2H), 4.93 (s, 1H), 3.45-3.37 (m, 2H), 3.35 (s, 3H), 3.29 (s, 3H), 2.56 (t, J=6.8 Hz, 2H), 2.28 (br s, 1H), 1.80-1.68 (m, 2H); ¹³C NMR (126 MHz, CDCl₃) δ 158.4, 139.9, 139.3, 139.0, 136.2, 131.8, 130.7, 130.6, 129.2, 128.8, 128.4, 128.1, 128.0, 127.8, 119.3, 114.0, 71.3, 70.6, 66.9, 58.8, 45.6, 38.9, 30.2; LR-MS calcd. for C₂₅H₂₉N₂O₄S [M+H]⁺ 453.18, found 453.65.

### 11-((3-Ethoxypropyl)amino)-3-iodo-6-methyl-6,11-dihydrodibenzo [c,f][1,2]thiazepine 5,5-dioxide (76)

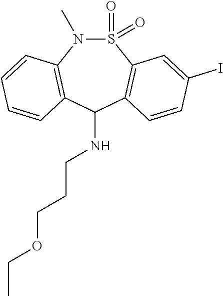 [SMILES: CCOCCCNC1c2ccccc2N(C)S(=O)(=O)c2cc(I)ccc21]

Method B. The product 76 was purified by column chromatography (hexanes:EtOAc—1:1, 2 column volumes→EtOAc, 3 column volumes) and obtained as a viscous, pale-yellow oil (43.0 mg, 88%). ¹H NMR (500 MHz, CDCl₃) δ 8.26 (d, J=1.6 Hz, 1H), 7.80 (dd, J=8.1, 1.7 Hz, 1H), 7.40-7.37 (m, 2H), 7.35 (td, J=7.5, 1.4 Hz, 1H), 7.28 (td, J=7.7, 1.2 Hz, 1H), 7.24 (d, J=8.2 Hz, 1H), 5.02 (s, 1H), 3.52-3.40 (m, 4H), 3.38 (s, 3H), 2.59 (t, J=6.6 Hz, 2H), 2.27 (br s, 1H), 1.83-1.68 (m, 2H), 1.15 (t, J=7.0 Hz, 3H); ¹³C NMR (126 MHz, CDCl₃) δ 141.2, 140.6, 139.3, 138.5, 138.4, 136.9, 131.1, 129.6, 129.4, 128.3, 128.1, 92.8, 69.1, 66.4, 65.7, 45.9, 38.6, 30.1, 15.3; LR-MS calcd. for C₁₉H₂₄IN₂O₃S [M+H]⁺ 487.05, found 487.69.

### 7-Chloro-10-((3-methoxypropyl)amino)-4-methyl-4, 10-dihydrobenzo[f]thieno[3,2-c][1,2]thiazepine 5,5-dioxide (77)

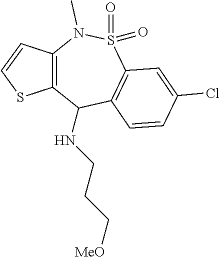 [SMILES: COCCCNC1c2ccc(Cl)cc2S(=O)(=O)N(C)c2ccsc21]

Method B. The product 77 was purified by column chromatography (20:1 CH₂Cl₂:Et₂O→10:1 CH₂Cl₂:Et₂O→5:1 CH₂Cl₂:Et₂O) and obtained as a dark-green solid (24.7 mg, 54%). ¹H NMR (400 MHz, CDCl₃) δ 7.97 (d, J=2.3 Hz, 1H), 7.61 (dd, J=8.2, 2.3 Hz, 1H), 7.42 (dd, J=8.2, 0.7 Hz, 1H), 7.19 (d, J=5.4 Hz, 1H), 6.82 (d, J=5.5 Hz, 1H), 5.37 (s, 1H), 3.50 (td, J=6.3, 2.0 Hz, 2H), 3.35 (s, 3H), 3.04 (s, 3H), 2.75 (d, J=6.5 Hz, 2H), 1.80 (q, J=6.0 Hz, 2H); ¹³C NMR (101 MHz, CDCl₃) δ 137.1, 134.7, 134.0, 133.6, 130.3, 130.1, 128.4, 124.5, 124.0, 110.1, 71.2, 60.8, 58.8, 45.3, 39.6, 30.2; LR-MS cald. for C₁₆H₂₀ClN₂O₃S₂ [M+H]⁺ 387.06, found 387.24.

### 7-Chloro-10-((3-ethoxypropyl)amino)-4-methyl-4, 10-dihydrobenzo[f]thieno[3,2-c][1,2]thiazepine 5,5-dioxide (78)

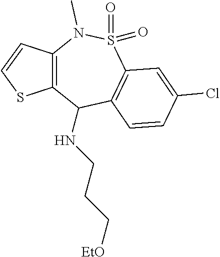 [SMILES: CCOCCCNC1c2ccc(Cl)cc2S(=O)(=O)N(C)c2ccsc21]

Method B. The product 78 was purified by column chromatography (20:1 CH₂Cl₂:Et₂O→10:1 CH₂Cl₂:Et₂O→5:1 CH₂Cl₂:Et₂O) and obtained as a dark-green solid (28.3 mg, 61%). ¹H NMR (400 MHz, CDCl₃) δ (d, J=2.3 Hz, 1H), 7.61 (dd, J=8.2, 2.3 Hz, 1H), 7.48-7.42 (m, 1H), 7.19 (d, J=5.5 Hz, 1H), 6.82 (d, J=5.5 Hz, 1H), 5.39 (s, 1H), 3.59-3.46 (m, 4H), 3.04 (s, 3H), 2.79-2.72 (m, 2H), 1.81 (q, J=6.3 Hz, 2H), 1.20 (t, J=7.0 Hz, 3H); ¹³C NMR (101 MHz, CDCl₃) δ 137.4, 135.4, 135.1, 134.3, 133.9, 130.6, 130.5, 128.7, 124.8, 124.3, 69.3, 66.7, 60.9, 45.7, 39.9, 30.6, 15.7; LR-MS cald. for C₁₇H₂₂ClN₂O₃S₂ [M+H]⁺ 401.08, found 401.23.

### Ethyl 7-((7-chloro-4-methyl-5, 5-dioxido-4, 10-dihydrobenzo[f]thieno[3,2-c][1,2]thiazepin-10-yl)amino)heptanoate (79)

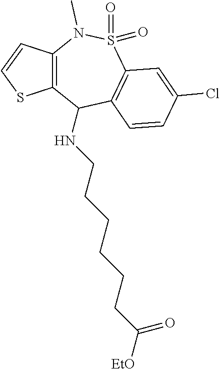 [SMILES: CCOC(=O)CCCCCCNC1c2ccc(Cl)cc2S(=O)(=O)N(C)c2ccsc21]

Method C. The product 79 was purified by column chromatography (20:1 CH₂Cl₂:Et₂O→10:1 CH₂Cl₂:Et₂O→5:1 CH₂Cl₂:Et₂O) and obtained as a dark reddish-brown solid (3.5 mg, 6%). ¹H NMR (400 MHz, CDCl₃) δ 7.98 (d, J=2.2 Hz, 1H), 7.61 (dd, J=8.2, 2.3 Hz, 1H), 7.37 (d, J=8.2 Hz, 1H), 7.20 (d, J=5.5 Hz, 1H), 6.82 (d, J=5.5 Hz, 1H), 5.33 (s, 1H), 4.12 (q, J=7.1 Hz, 2H), 3.04 (s, 3H), 2.63 (t, J=6.9 Hz, 2H), 2.28 (t, J=7.5 Hz, 2H), 1.67-1.58 (m, 2H), 1.53 (t, J=6.8 Hz, 3H), 1.35 (dq, J=10.0, 5.9 Hz, 4H), 1.25 (t, J=7.1 Hz, 3H); ¹³C NMR (126 MHz, CDCl₃) δ 180.3, 137.2, 135.3, 133.6, 133.5, 128.5, 124.5, 124.4, 124.1, 60.9, 60.3, 47.8, 39.6, 34.5, 30.0, 29.1, 27.0, 25.1; LR-MS cald. for C₂₁H₂₈ClN₂O₄S₂ [M+H]⁺ 471.12, found 471.42.

### 7-((6-Methyl-5, 5-dioxido-6,11-dihydrodibenzo[c,f][1,2]thiazepin-11-yl)amino)heptanoic acid hydrochloride salt (80)

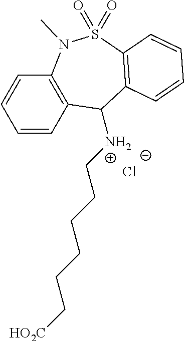 [SMILES: CN1c2ccccc2C([NH2+]CCCCCCC(=O)O)c2ccccc2S1(=O)=O ; [Cl-]]

Compound 9 (32.0 mg, 0.0743 mmol) was heated in aqueous HCl (0.5 M, 2.0 mL) at 70° C. for 2 h. The reaction mixture was then concentrated and dried thoroughly in vacuo to provide the pure HCl salt of the acid 80 as a colorless glass (32.6 mg, quantitative yield). ¹H NMR (400 MHz, Methanol-d₄) δ 8.15-8.10 (m, 1H), 7.94-7.89 (m, 1H), 7.89-7.81 (m, 2H), 7.75 (d, J=7.8 Hz, 1H), 7.67-7.61 (m, 1H), 7.56 (dd, J=8.1, 1.1 Hz, 1H), 7.48 (td, J=7.6, 1.3 Hz, 1H), 5.96 (s, 1H), 3.19 (s, 3H), 3.01-2.92 (m, 1H), 2.83-2.73 (m, 1H), 2.33-2.21 (m, 2H), 1.75-1.61 (m, 2H), 1.61-1.50 (m, 2H), 1.36-1.24 (m, 4H).

### Ethyl 7-((3-bromo-6-methyl-5, 5-dioxido-6,11-dihydrodibenzo[c,f][1,2]thiazepin-11-yl)amino)heptanoate (81)

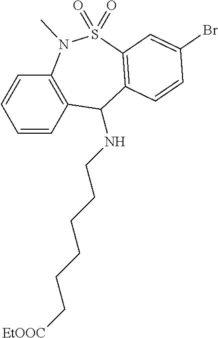 [SMILES: CCOC(=O)CCCCCCNC1c2ccccc2N(C)S(=O)(=O)c2cc(Br)ccc21]

Method C. The product 81 was purified by column chromatography (CH₂Cl₂:Et₂O—20:1, 4 column volumes→7:3, 2 column volumes) and obtained as a viscous, colorless oil (42.0 mg, 82%). ¹H NMR (500 MHz, CDCl₃) δ 8.09 (d, J=2.1 Hz, 1H), 7.61 (dd, J=8.2, 2.1 Hz, 1H), 7.40-7.33 (m, 4H), 7.31-7.27 (m, 1H), 4.98 (s, 1H), 4.11 (q, J=7.1 Hz, 2H), 3.37 (s, 3H), 2.45 (t, J=7.1 Hz, 2H), 2.26 (t, J=7.5 Hz, 2H), 2.03 (br s, 1H), 1.63-1.54 (m, 2H), 1.52-1.43 (m, 2H), 1.34-1.26 (m, 4H), 1.24 (t, J=7.1 Hz, 3H); ¹³C NMR (126 MHz, CDCl₃) δ 173.8, 140.6, 138.9, 138.6, 137.6, 135.3, 131.4, 131.3, 130.1, 129.4, 128.2, 128.1, 122.0, 66.3, 60.3, 48.2, 38.8, 34.4, 30.0, 29.1, 27.0, 25.0, 14.4; LR-MS cald. for C₂₃H₃₀BrN₂O₄S [M+H]⁺ 509.11, found 509.92.

### 7-((3-Bromo-6-methyl-5, 5-dioxido-6,11-dihydrodibenzo[c,f][1,2]thiazepin-11-yl)amino)heptanoic acid hydrochloride salt (82)

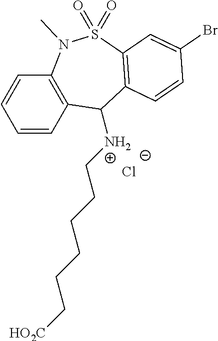 [SMILES: CN1c2ccccc2C([NH2+]CCCCCCC(=O)O)c2ccc(Br)cc2S1(=O)=O ; [Cl-]]

Compound 81 (20.0 mg, 0.0393 mmol) was heated in aqueous HCl (0.5 M, 1.5 mL) at 70° C. for 3 h. The reaction mixture was then concentrated and dried thoroughly in vacuo to provide the pure HCl salt of the acid 82 as a glassy, white foam (19.9 mg, 98%). ¹H NMR (500 MHz, Methanol-d₄) δ 8.22 (d, J=2.1 Hz, 1H), 8.02 (dd, J=8.2, 2.1 Hz, 1H), 7.86-7.81 (m, 1H), 7.74 (d, J=7.8 Hz, 1H), 7.66-7.62 (m, 1H), 7.57 (dd, J=8.1, 1.1 Hz, 1H), 7.49 (td, J=7.7, 1.3 Hz, 1H), 5.95 (s, 1H), 3.24 (s, 3H), 2.96 (ddd, J=12.1, 10.4, 5.5 Hz, 1H), 2.82 (ddd, J=12.2, 10.3, 5.9 Hz, 1H), 2.27 (t, J=7.3 Hz, 2H), 1.74-1.61 (m, 2H), 1.61-1.53 (m, 2H), 1.37-1.28 (m, 4H); ¹³C NMR (126 MHz, Methanol-d₄) δ 177.4, 142.5, 141.8, 138.3, 137.0, 134.7, 133.4, 132.2, 129.1, 128.7, 128.6, 127.4, 126.5, 67.6, 48.3, 39.7, 34.6, 29.5, 27.1, 26.8, 25.6; LR-MS cald. for C₂₁H₂₆BrN₂O₄S [M+H]⁺ 481.08, found 481.05.

### 3-Chloro-11-((3-hydroxypropyl)amino)-6-methyl-6,11-dihydrodibenzo[c,f][1,2]thiazepine 5,5-dioxide (83)

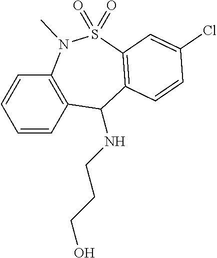 [SMILES: CN1c2ccccc2C(NCCCO)c2ccc(Cl)cc2S1(=O)=O]

Method B. The product 83 was purified by column chromatography (CH₂Cl₂:Et₂O—1:1, 3 column volumes→CH₂Cl₂:Et₂O—1:1+5% MeOH, 3 column volumes) and obtained as a cloudy, colorless glass (27.1 mg, 74%). ¹H NMR (500 MHz, CDCl₃) δ 7.98 (d, J=2.2 Hz, 1H), 7.50 (dd, J=8.2, 2.2 Hz, 1H), 7.45-7.40 (m, 2H), 7.38-7.31 (m, 2H), 7.31-7.27 (m, 1H), 5.01 (s, 1H), 3.82-3.74 (m, 2H), 3.25 (br s, 2H), 3.25 (s, 3H), 2.68-2.60 (m, 2H), 1.75-1.63 (m, 2H); ¹³C NMR (126 MHz, CDCl₃) δ 139.9, 139.2, 136.2, 135.6, 134.6, 132.5, 132.4, 131.5, 129.6, 128.8, 128.1, 127.6, 67.1, 63.7, 47.4, 39.1, 31.2; LR-MS cald. for C₁₇H₂₀ClN₂O₃S [M+H]⁺ 367.09, found 367.09.

### 7-((3-Iodo-6-methyl-5, 5-dioxido-6,11-dihydrodibenzo [c,f][1,2]thiazepin-11-yl)amino)heptanoic acid hydrochloride salt (84)

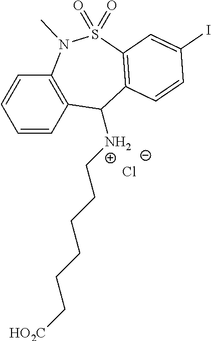 [SMILES: CN1c2ccccc2C([NH2+]CCCCCCC(=O)O)c2ccc(I)cc2S1(=O)=O ; [Cl-]]

Compound 49 (25 mg, 0.045 mmol) was heated in aqueous HCl (0.5 M, 1.5 mL) at 70° C. for 3 h. The reaction mixture was then concentrated and dried thoroughly in vacuo to provide the pure HCl salt of the acid 84 as a glassy off-white solid (25 mg, quantitative yield). ¹H NMR (400 MHz, Methanol-d₄) δ 8.38 (d, J=1.5 Hz, 1H), 8.22 (dd, J=7.5, 1.9 Hz, 1H), 7.78-7.72 (m, 1H), 7.65 (dd, J=15.3, 7.7 Hz, 2H), 7.57 (dd, J=8.1, 1.2 Hz, 1H), 7.48 (t, J=7.3 Hz, 1H), 5.95 (s, 1H), 3.22 (s, 3H), 3.01-2.92 (m, 1H), 2.86-2.78 (m, 1H), 2.26 (t, J=7.2 Hz, 2H), 1.74-1.62 (m, 2H), 1.61-1.53 (m, 2H), 1.33 (m, 4H); ¹¹C NMR (101 MHz, Methanol-d₄) δ 177.3, 144.5, 142.6, 141.4, 137.9, 136.9, 136.9, 133.5, 129.1, 128.7, 128.5, 126.3, 97.9, 67.9, 48.6, 39.8, 34.6, 29.4, 27.1, 26.7, 25.6; LR-MS calcd. for C₂₁H₂₆IN₂O₄S [M+H]⁺ 529.07, found 529.08.

### Example 1. Mu-Opioid Receptor Activity

Transfection. Human or mouse MOR cDNA was transfected alongside Gα_{oB} with RLuc8 inserted at position 91 (Gα_{oB}-RLuc8), Gβ₁ (β₁), and Gγ₂ fused to the full-length mVenus at its N terminus (mVenus γ2) into HEK-293T cells (5×10⁶ cells/plate) in 10-cm dishes using PEI (Polysciences Inc.; Warrington, PA) in a 1:1 ratio diluted in Opti-MEM (Life Technologies Corp.; Grand Island, NY) to assay for G protein activation as described previously (Rives, M.-L. et al. J. Biol. Chem. 2012, 287, 27050-4; Negri, A.; Rives, M.-L.; Caspers, M. J. et al. J. Chem. Inf. Model. 2013, 53, 521-526). Cells were maintained in the Dulbecco's Modified Eagle Medium (high glucose #11965; Life Technologies) supplemented with 10% FBS (Premium Select, Atlanta Biologicals; Atlanta, GA) and 100 U/mL penicillin and 100 μg/mL streptomycin (#15140, Life Technologies). After 24 hours the media was changed, and the experiment was performed 24 hours later (48 hours after transfection).

BRET. Transfected cells were dissociated and resuspended in phosphate-buffered saline (PBS). Approximately 200,000 cells/well were added to a black-framed, white well 96-well plate (#60050; Perkin Elmer; Waltham, MA). The microplate was centrifuged and the cells were resuspended in PBS. Then 5 μM of the luciferase substrate coelenterazine H was added to each well for 5 minutes. Following coelenterazine H addition, ligands were added and the BRET signal was measured at 5 minutes on a PHERAstar FS plate reader. Quantification of the BRET signal required calculating the ratio of the light emitted by the energy acceptor, mVenus (510-540 nm), over the light emitted by the energy donor, RLuc8 (485 nm). This drug-induced BRET signal was normalized using the Eₘₐₓ of DAMGO as the 100% maximal response for G protein activation. Dose response curves were fit using three-parameter logistics equation in GraphPad Prism 6.

The following compounds listed in Table 1 activated human and/or mouse MOR. Accordingly, the compounds listed in Table 1 are agonists of MOR.

TABLE 1
Data represents mean ± SEM of various independent trials
(n > 2); “—” indicates not tested.

| | | EC₅₀ | EC₅₀ |
|---|---|---|---|
| Compound | Structure | Human MOR | Mouse MOR |
| DAMGO | 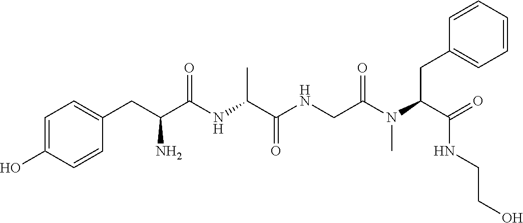 [SMILES: C[C@@H](NC(=O)[C@@H](N)Cc1ccc(O)cc1)C(=O)NCC(=O)N(C)[C@@H](Cc1ccccc1)C(=O)NCCO] | 2.07 ± 1.21 nM | 6.98 ± 2.10 nM |
| 6 | 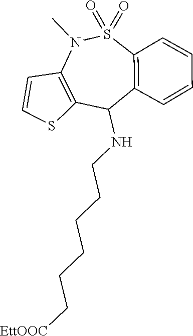 | <10 μM | — |
| 7 | 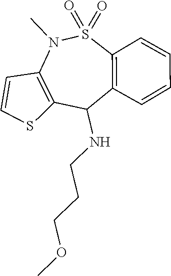 [SMILES: COCCCNC1c2ccccc2S(=O)(=O)N(C)c2ccsc21] | <10 μM | — |
| 8 | 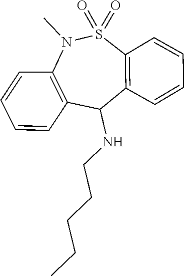 [SMILES: CCCCCNC1c2ccccc2N(C)S(=O)(=O)c2ccccc21] | <10 μM | <10 μM |
| 9 | 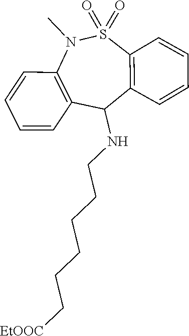 [SMILES: CCOC(=O)CCCCCCNC1c2ccccc2N(C)S(=O)(=O)c2ccccc21] | <10 μM | <10 μM |
| 10 | 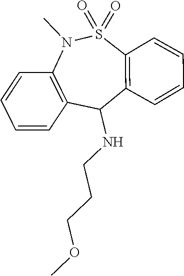 [SMILES: COCCCNC1c2ccccc2N(C)S(=O)(=O)c2ccccc21] | <10 μM | — |
| 11 | 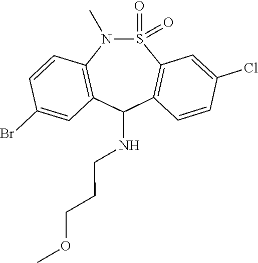 [SMILES: COCCCNC1c2cc(Br)ccc2N(C)S(=O)(=O)c2cc(Cl)ccc21] | <10 μM | — |
| 13 | 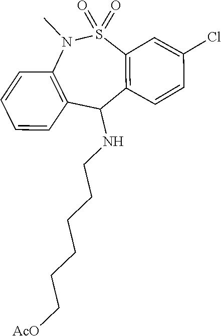 [SMILES: CC(=O)OCCCCCCNC1c2ccccc2N(C)S(=O)(=O)c2cc(Cl)ccc21] | <10 μM | — |
| 14 | 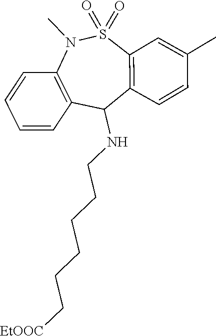 [SMILES: CCOC(=O)CCCCCCNC1c2ccccc2N(C)S(=O)(=O)c2cc(C)ccc21] | <10 μM | — |
| 15 | 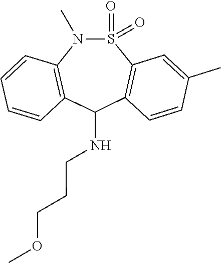 [SMILES: COCCCNC1c2ccccc2N(C)S(=O)(=O)c2cc(C)ccc21] | <10 μM | — |
| 16 | 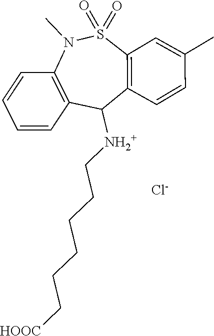 [SMILES: Cc1ccc2c(c1)S(=O)(=O)N(C)c1ccccc1C2[NH2+]CCCCCCC(=O)O ; [Cl-]] | <10 μM | — |
| 17 | 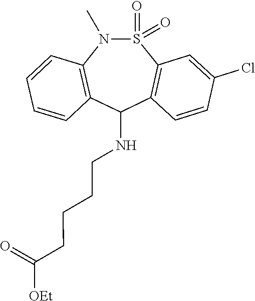 [SMILES: CCOC(=O)CCCCNC1c2ccccc2N(C)S(=O)(=O)c2cc(Cl)ccc21] | <10 μM | <10 μM |
| 18 | 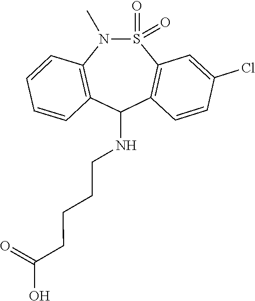 [SMILES: CN1c2ccccc2C(NCCCCC(=O)O)c2ccc(Cl)cc2S1(=O)=O] | <10 μM | <10 μM |
| 19 | 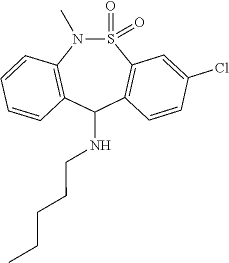 [SMILES: CCCCCNC1c2ccccc2N(C)S(=O)(=O)c2cc(Cl)ccc21] | <10 μM | <10 μM |
| 20 | 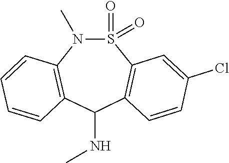 [SMILES: CNC1c2ccccc2N(C)S(=O)(=O)c2cc(Cl)ccc21] | <10 μM | <10 μM |
| 21 | 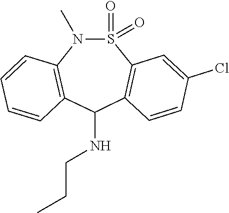 [SMILES: CCCNC1c2ccccc2N(C)S(=O)(=O)c2cc(Cl)ccc21] | <10 μM | <10 μM |
| 22 | 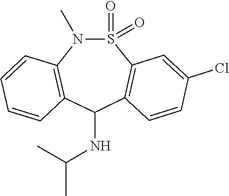 [SMILES: CC(C)NC1c2ccccc2N(C)S(=O)(=O)c2cc(Cl)ccc21] | <10 μM | <10 μM |
| 23 | 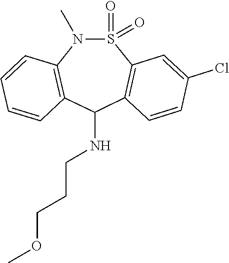 [SMILES: COCCCNC1c2ccccc2N(C)S(=O)(=O)c2cc(Cl)ccc21] | 264.5 ± 85.3 nM | 1.20 ± 0.03 μM |
| 24 | 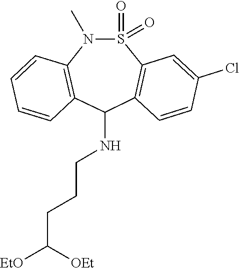 [SMILES: CCOC(CCCNC1c2ccccc2N(C)S(=O)(=O)c2cc(Cl)ccc21)OCC] | <10 μM | — |
| 25 | 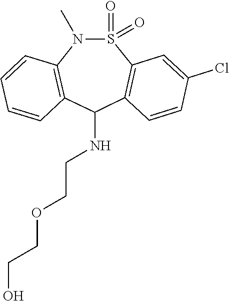 [SMILES: CN1c2ccccc2C(NCCOCCO)c2ccc(Cl)cc2S1(=O)=O] | 8.592 ± 0.5 μM | — |
| 26 | 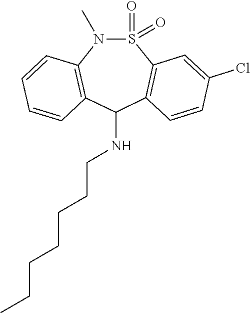 [SMILES: CCCCCCCNC1c2ccccc2N(C)S(=O)(=O)c2cc(Cl)ccc21] | <10 μM | — |
| 27 | 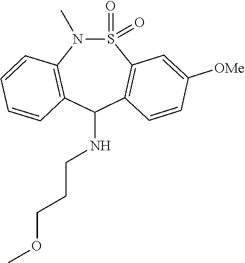 [SMILES: COCCCNC1c2ccccc2N(C)S(=O)(=O)c2cc(OC)ccc21] | <10 μM | — |
| 28 | 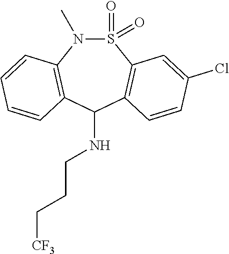 [SMILES: CN1c2ccccc2C(NCCCC(F)(F)F)c2ccc(Cl)cc2S1(=O)=O] | <10 μM | — |
| 29 | 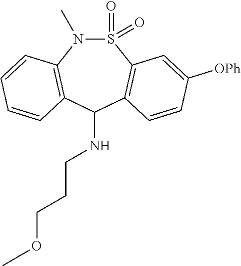 [SMILES: COCCCNC1c2ccccc2N(C)S(=O)(=O)c2cc(Oc3ccccc3)ccc21] | <10 μM | — |
| 31 | 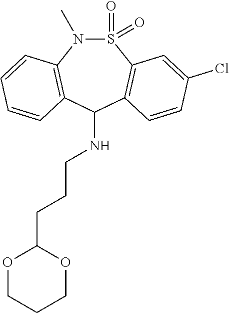 [SMILES: CN1c2ccccc2C(NCCCC2OCCCO2)c2ccc(Cl)cc2S1(=O)=O] | <10 μM | — |
| 32 | 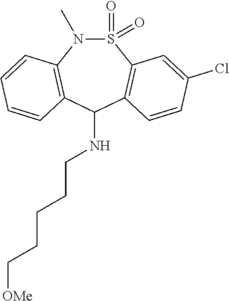 [SMILES: COCCCCCNC1c2ccccc2N(C)S(=O)(=O)c2cc(Cl)ccc21] | 563.1 ± 247 nM | — |
| 33 | 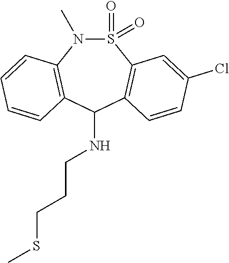 [SMILES: CSCCCNC1c2ccccc2N(C)S(=O)(=O)c2cc(Cl)ccc21] | <10 μM | — |
| 34 | 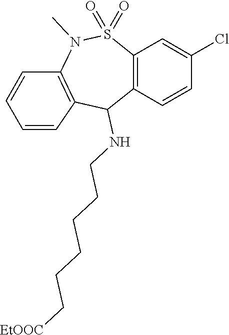 [SMILES: CCOC(=O)CCCCCCNC1c2ccccc2N(C)S(=O)(=O)c2cc(Cl)ccc21] | 657 ± 366 nM | — |
| 35 | 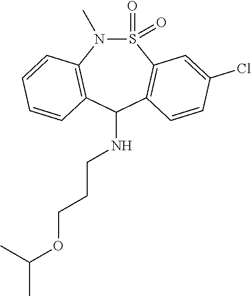 [SMILES: CC(C)OCCCNC1c2ccccc2N(C)S(=O)(=O)c2cc(Cl)ccc21] | <10 μM | — |
| 36 | 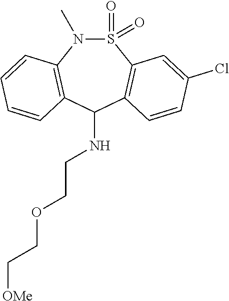 [SMILES: COCCOCCNC1c2ccccc2N(C)S(=O)(=O)c2cc(Cl)ccc21] | <10 μM | — |
| 37 | 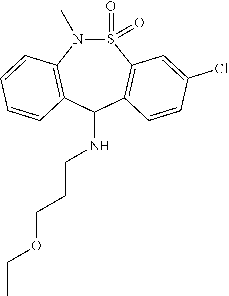 [SMILES: CCOCCCNC1c2ccccc2N(C)S(=O)(=O)c2cc(Cl)ccc21] | 893 ± 147 nM | — |
| 39 | 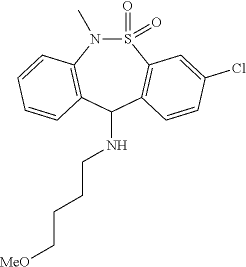 [SMILES: COCCCCNC1c2ccccc2N(C)S(=O)(=O)c2cc(Cl)ccc21] | <10 μM | — |
| 40 | 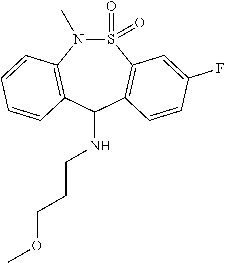 [SMILES: COCCCNC1c2ccccc2N(C)S(=O)(=O)c2cc(F)ccc21] | 5.53 ± 0.25 μM | — |
| 41 | 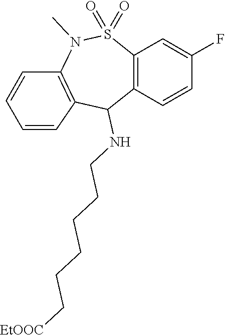 [SMILES: CCOC(=O)CCCCCCNC1c2ccccc2N(C)S(=O)(=O)c2cc(F)ccc21] | 2.04 ± 0.334 μM | — |
| 42 | 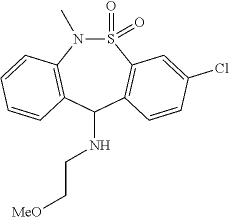 [SMILES: COCCNC1c2ccccc2N(C)S(=O)(=O)c2cc(Cl)ccc21] | <10 μM | — |
| 43 | 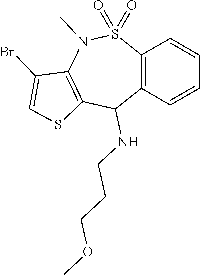 [SMILES: COCCCNC1c2ccccc2S(=O)(=O)N(C)c2c(Br)csc21] | <20 μM | — |
| 44 | 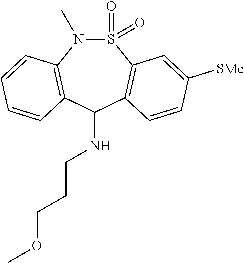 [SMILES: COCCCNC1c2ccccc2N(C)S(=O)(=O)c2cc(SC)ccc21] | <10 μM | <10 μM |
| 45 | 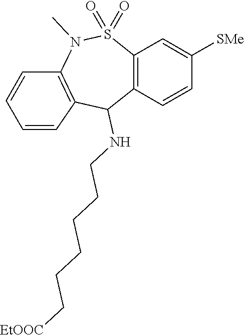 [SMILES: CCOC(=O)CCCCCCNC1c2ccccc2N(C)S(=O)(=O)c2cc(SC)ccc21] | <10 μM | <10 μM |
| 46 | 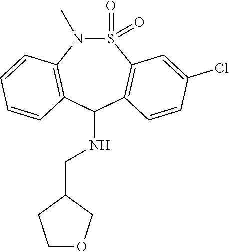 [SMILES: CN1c2ccccc2C(NCC2CCOC2)c2ccc(Cl)cc2S1(=O)=O] | <10 μM | — |
| 47 | 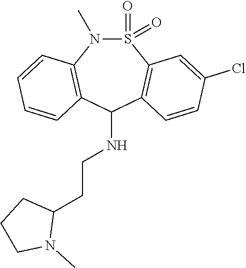 [SMILES: CN1CCCC1CCNC1c2ccccc2N(C)S(=O)(=O)c2cc(Cl)ccc21] | <20 μM | — |
| 48 | 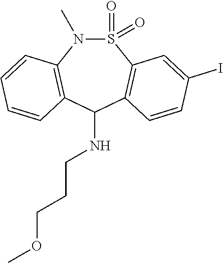 [SMILES: COCCCNC1c2ccccc2N(C)S(=O)(=O)c2cc(I)ccc21] | 15.2 ± 6.9 nM | |
| 49 | 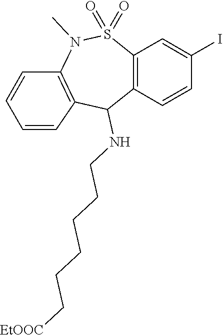 [SMILES: CCOC(=O)CCCCCCNC1c2ccccc2N(C)S(=O)(=O)c2cc(I)ccc21] | 137 ± 17 nM | — |
| 50 | 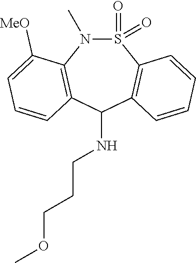 [SMILES: COCCCNC1c2ccccc2S(=O)(=O)N(C)c2c(OC)cccc21] | <20 μM | — |
| 51 | 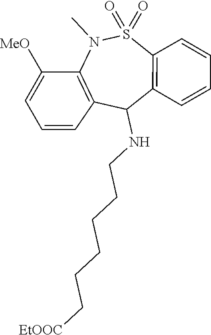 [SMILES: CCOC(=O)CCCCCCNC1c2ccccc2S(=O)(=O)N(C)c2c(OC)cccc21] | <10 μM | — |
| 52 | 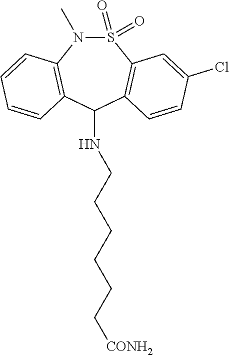 [SMILES: CN1c2ccccc2C(NCCCCCCC(N)=O)c2ccc(Cl)cc2S1(=O)=O] | <10 μM | — |
| 53 | 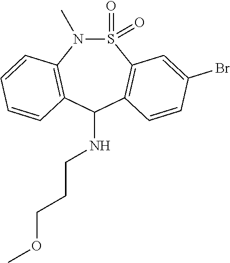 [SMILES: COCCCNC1c2ccccc2N(C)S(=O)(=O)c2cc(Br)ccc21] | 74.1 ± 17.9 nM | — |
| 55 | 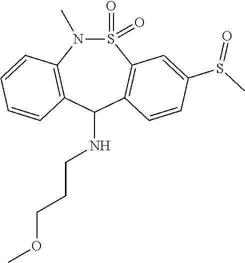 [SMILES: COCCCNC1c2ccccc2N(C)S(=O)(=O)c2cc(S(C)=O)ccc21] | <20 μM | — |
| 56 | 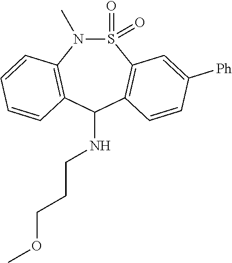 [SMILES: COCCCNC1c2ccccc2N(C)S(=O)(=O)c2cc(-c3ccccc3)ccc21] | <10 μM | — |
| 57 | 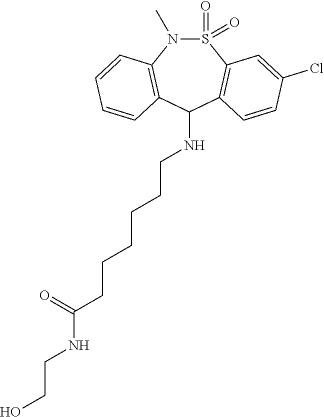 [SMILES: CN1c2ccccc2C(NCCCCCCC(=O)NCCO)c2ccc(Cl)cc2S1(=O)=O] | <10 μM | — |
| 58 | 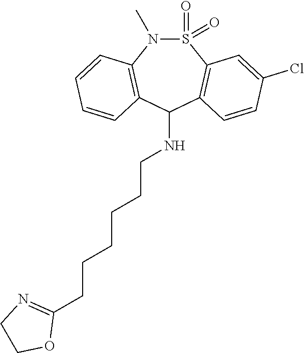 [SMILES: CN1c2ccccc2C(NCCCCCCC2=NCCO2)c2ccc(Cl)cc2S1(=O)=O] | <10 μM | — |
| 59 | 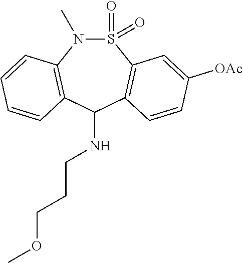 [SMILES: COCCCNC1c2ccccc2N(C)S(=O)(=O)c2cc(OC(C)=O)ccc21] | <10 μM | — |
| 60 | 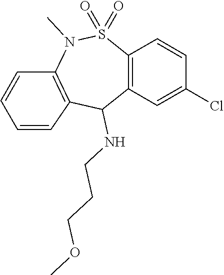 [SMILES: COCCCNC1c2ccccc2N(C)S(=O)(=O)c2ccc(Cl)cc21] | <20 μM | — |
| 61 | 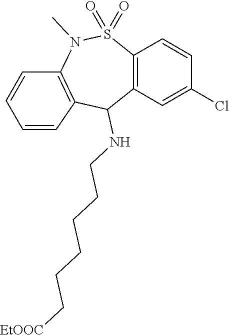 [SMILES: CCOC(=O)CCCCCCNC1c2ccccc2N(C)S(=O)(=O)c2ccc(Cl)cc21] | <10 μM | — |
| 62 | 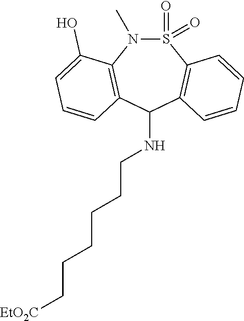 [SMILES: CCOC(=O)CCCCCCNC1c2ccccc2S(=O)(=O)N(C)c2c(O)cccc21] | >20 μM | — |
| 63 | 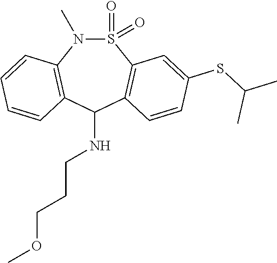 [SMILES: COCCCNC1c2ccccc2N(C)S(=O)(=O)c2cc(SC(C)C)ccc21] | <10 μM | — |
| 64 | 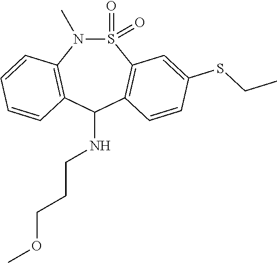 [SMILES: CCSc1ccc2c(c1)S(=O)(=O)N(C)c1ccccc1C2NCCCOC] | <10 μM | — |
| 65 | 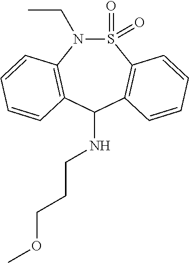 [SMILES: CCN1c2ccccc2C(NCCCOC)c2ccccc2S1(=O)=O] | >20 μM | — |
| 66 | 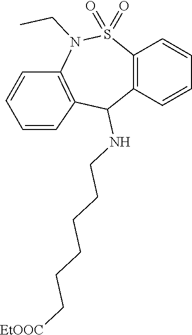 [SMILES: CCOC(=O)CCCCCCNC1c2ccccc2N(CC)S(=O)(=O)c2ccccc21] | <10 μM | — |
| 67 | 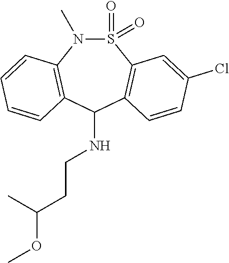 [SMILES: COC(C)CCNC1c2ccccc2N(C)S(=O)(=O)c2cc(Cl)ccc21] | <10 μM | — |
| 68 | 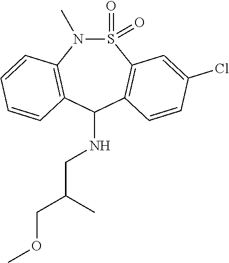 [SMILES: COCC(C)CNC1c2ccccc2N(C)S(=O)(=O)c2cc(Cl)ccc21] | <10 μM | — |
| 69 | 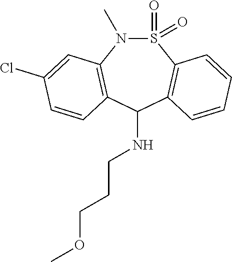 [SMILES: COCCCNC1c2ccc(Cl)cc2N(C)S(=O)(=O)c2ccccc21] | <20 μM | — |
| 70 | 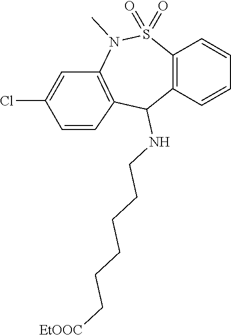 [SMILES: CCOC(=O)CCCCCCNC1c2ccc(Cl)cc2N(C)S(=O)(=O)c2ccccc21] | <10 μM | — |
| 71 | 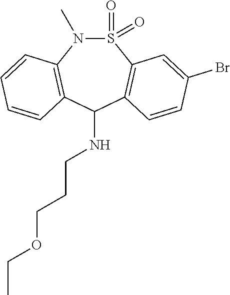 [SMILES: CCOCCCNC1c2ccccc2N(C)S(=O)(=O)c2cc(Br)ccc21] | 222 ± 78 nM | — |
| 72 | 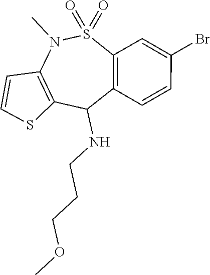 [SMILES: COCCCNC1c2ccc(Br)cc2S(=O)(=O)N(C)c2ccsc21] | <10 μM | — |
| 73 | 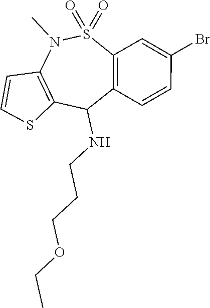 [SMILES: CCOCCCNC1c2ccc(Br)cc2S(=O)(=O)N(C)c2ccsc21] | <10 μM | — |
| 74 | 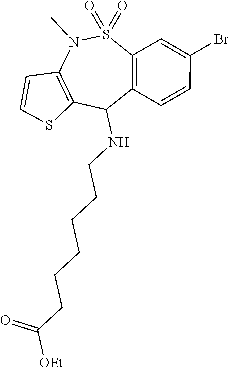 [SMILES: CCOC(=O)CCCCCCNC1c2ccc(Br)cc2S(=O)(=O)N(C)c2ccsc21] | <10 μM | — |
| 75 | 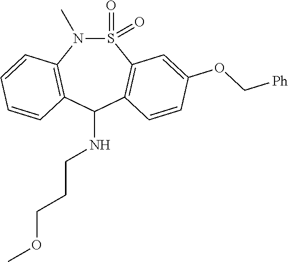 [SMILES: COCCCNC1c2ccccc2N(C)S(=O)(=O)c2cc(OCc3ccccc3)ccc21] | >20 μM | — |
| 76 | 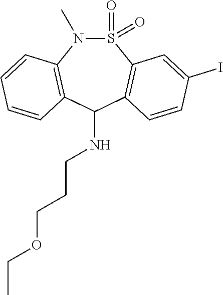 [SMILES: CCOCCCNC1c2ccccc2N(C)S(=O)(=O)c2cc(I)ccc21] | 145 nM | — |
| 77 | 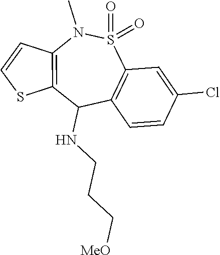 [SMILES: COCCCNC1c2ccc(Cl)cc2S(=O)(=O)N(C)c2ccsc21] | <10 μM | — |
| 78 | 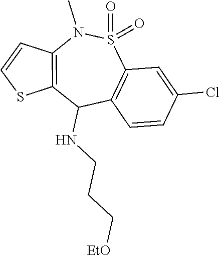 [SMILES: CCOCCCNC1c2ccc(Cl)cc2S(=O)(=O)N(C)c2ccsc21] | <10 μM | — |
| 79 | 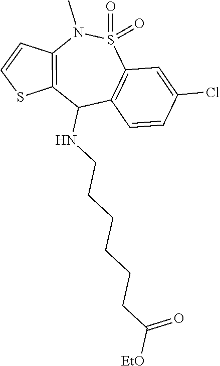 [SMILES: CCOC(=O)CCCCCCNC1c2ccc(Cl)cc2S(=O)(=O)N(C)c2ccsc21] | <10 μM | — |
| 80 | 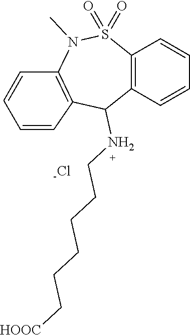 | <10 μM | <10 μM |
| 81 | 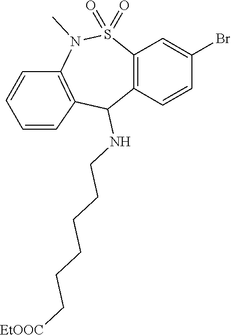 [SMILES: CCOC(=O)CCCCCCNC1c2ccccc2N(C)S(=O)(=O)c2cc(Br)ccc21] | 573 nM | — |
| 82 | 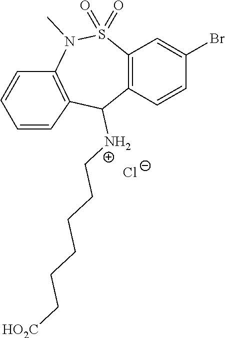 [SMILES: CN1c2ccccc2C([NH2+]CCCCCCC(=O)O)c2ccc(Br)cc2S1(=O)=O ; [Cl-]] | 122 nM | — |
| 83 | 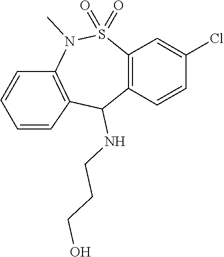 [SMILES: CN1c2ccccc2C(NCCCO)c2ccc(Cl)cc2S1(=O)=O] | 1.81 μM | — |
| 84 | 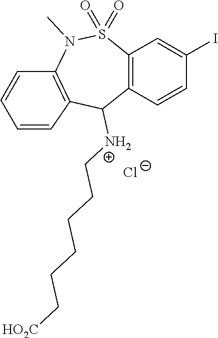 [SMILES: CN1c2ccccc2C([NH2+]CCCCCCC(=O)O)c2ccc(I)cc2S1(=O)=O ; [Cl-]] | 40.0 nM | — |
| tianeptine | 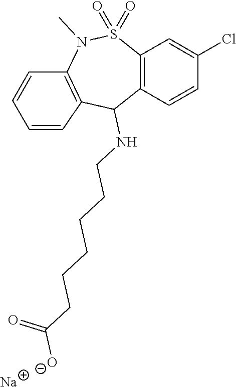 [SMILES: CN1c2ccccc2C(NCCCCCCC(=O)[O-])c2ccc(Cl)cc2S1(=O)=O ; [Na+]] | 194 ± 70 nM | 641 ± 120 nM |

### Example 2. Potency of Analogs Relative to Tianeptine

Potency of compounds showing improvement over tianeptine. Specifically, the data show that R₂ ether side chains are significantly more potent than analogous alcohol side chains (which were inactive or very low potency). Further, the data show the strong trend for increasing potency as the size of the halogen substituent is increased from fluoro through iodo, a trend that holds across several example subgenera. Accordingly, the compounds disclosed herein have increasing potency based on the following trend: R₅=I>R₅=Br>R₅=Cl>R₅=F. Compounds with activity similar to tianeptine have improved pharmacokinetic profiles.

TABLE 2
Data represent mean ± SEM of various independent trials. “X”
indicates not active (EC₅₀ > 50 μM); “—” indicates not tested.

| | | EC₅₀ | EC₅₀ |
|---|---|---|---|
| Compound | Structure | Human MOR | Mouse MOR |
| DAMGO | 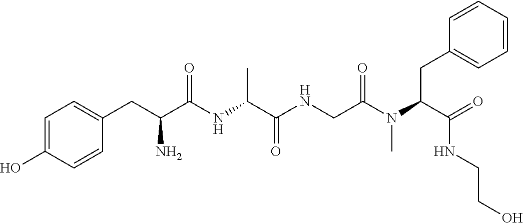 [SMILES: C[C@@H](NC(=O)[C@@H](N)Cc1ccc(O)cc1)C(=O)NCC(=O)N(C)[C@@H](Cc1ccccc1)C(=O)NCCO] | 2.07 ± 1.21 nM | 6.98 ± 2.10 nM |
| 23 | 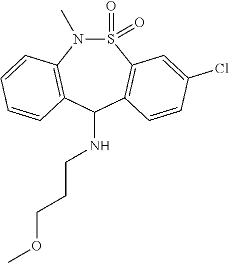 [SMILES: COCCCNC1c2ccccc2N(C)S(=O)(=O)c2cc(Cl)ccc21] | 264.5 ± 85.3 nM | 1.20 ± 0.03 μM |
| 83 | 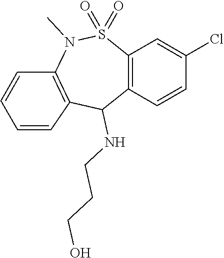 [SMILES: CN1c2ccccc2C(NCCCO)c2ccc(Cl)cc2S1(=O)=O] | 1.81 μM | — |
| 32 | 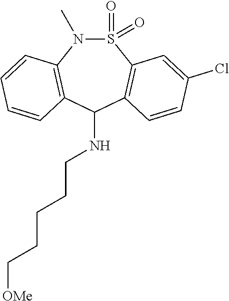 [SMILES: COCCCCCNC1c2ccccc2N(C)S(=O)(=O)c2cc(Cl)ccc21] | 563.1 ± 247 nM | — |
| 30 | 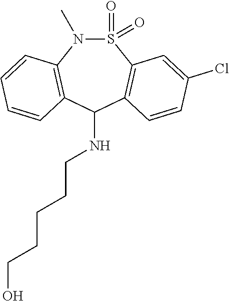 [SMILES: CN1c2ccccc2C(NCCCCCO)c2ccc(Cl)cc2S1(=O)=O] | X | — |
| 25 | 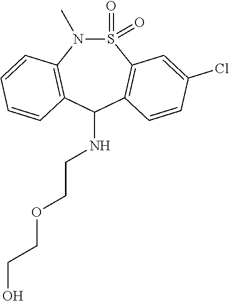 [SMILES: CN1c2ccccc2C(NCCOCCO)c2ccc(Cl)cc2S1(=O)=O] | 8.592 ± 0.5 μM | — |
| 40 | 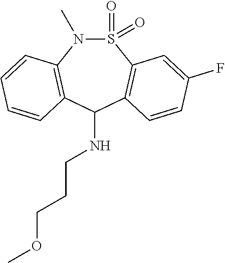 [SMILES: COCCCNC1c2ccccc2N(C)S(=O)(=O)c2cc(F)ccc21] | 5.53 ± 0.25 μM | — |
| 23 | 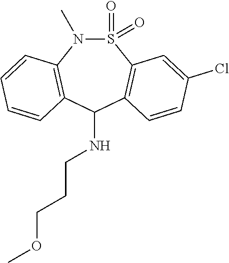 [SMILES: COCCCNC1c2ccccc2N(C)S(=O)(=O)c2cc(Cl)ccc21] | 264.5 ± 85.3 nM | 1.20 ± 0.03 μM |
| 53 | 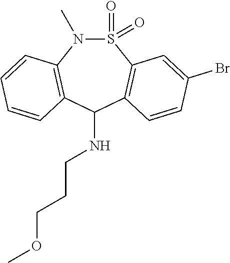 [SMILES: COCCCNC1c2ccccc2N(C)S(=O)(=O)c2cc(Br)ccc21] | 74.1 ± 17.9 nM | — |
| 48 | 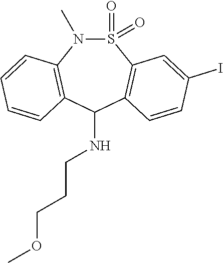 [SMILES: COCCCNC1c2ccccc2N(C)S(=O)(=O)c2cc(I)ccc21] | 15.2 ± 6.9 nM | |
| 37 | 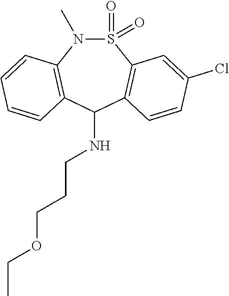 [SMILES: CCOCCCNC1c2ccccc2N(C)S(=O)(=O)c2cc(Cl)ccc21] | 893 ± 147 nM | — |
| 71 | 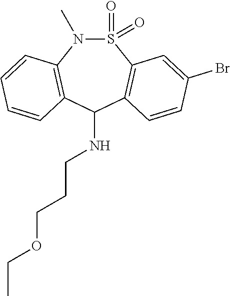 [SMILES: CCOCCCNC1c2ccccc2N(C)S(=O)(=O)c2cc(Br)ccc21] | 222 ± 78 nM | — |
| 76 | 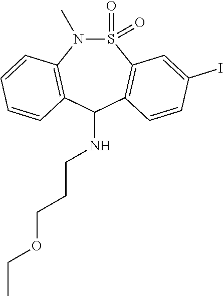 [SMILES: CCOCCCNC1c2ccccc2N(C)S(=O)(=O)c2cc(I)ccc21] | 145 nM | — |
| 41 | 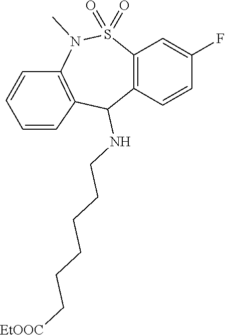 [SMILES: CCOC(=O)CCCCCCNC1c2ccccc2N(C)S(=O)(=O)c2cc(F)ccc21] | 2.04 ± 0.334 μM | — |
| 34 | 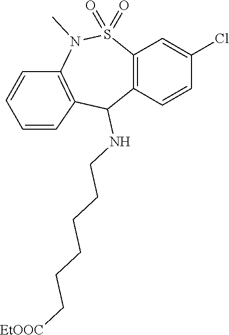 [SMILES: CCOC(=O)CCCCCCNC1c2ccccc2N(C)S(=O)(=O)c2cc(Cl)ccc21] | 657 ± 366 nM | — |
| 81 | 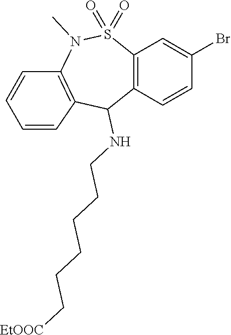 [SMILES: CCOC(=O)CCCCCCNC1c2ccccc2N(C)S(=O)(=O)c2cc(Br)ccc21] | 573 nM | — |
| 49 | 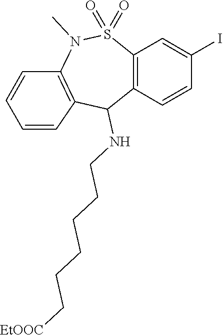 [SMILES: CCOC(=O)CCCCCCNC1c2ccccc2N(C)S(=O)(=O)c2cc(I)ccc21] | 137 ± 17 nM | — |
| tianeptine | 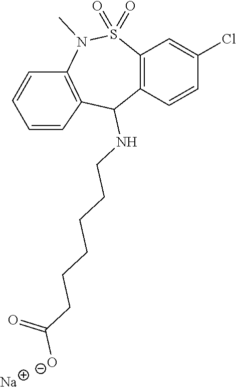 [SMILES: CN1c2ccccc2C(NCCCCCCC(=O)[O-])c2ccc(Cl)cc2S1(=O)=O ; [Na+]] | 194 ± 70 nM | 641 ± 120 nM |
| 82 | 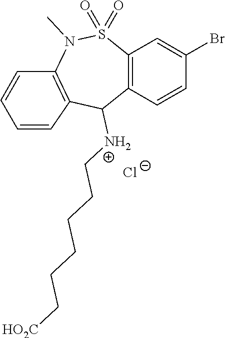 [SMILES: CN1c2ccccc2C([NH2+]CCCCCCC(=O)O)c2ccc(Br)cc2S1(=O)=O ; [Cl-]] | 122 nM | — |
| 84 | 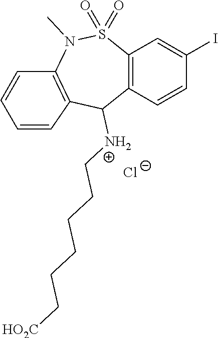 [SMILES: CN1c2ccccc2C([NH2+]CCCCCCC(=O)O)c2ccc(I)cc2S1(=O)=O ; [Cl-]] | 40.0 nM | — |

### Example 3. MOR Agonism of Additional 6-methyl-6,11-dihydrodibenzo[1,2]thiazepine 5,5-dioxide Compounds

An additional aspect of the present disclosure provides analogs of the compounds of Table 1 that are MOR agonists. Additional 6-methyl-6,11-dihydrodibenzo[1,2]thiazepine 5,5-dioxide compounds, which are analogs of those described in Table 1, are tested in in vitro human and mouse MOR assays. These analogs agonize MOR activity. Additional compounds disclosed herein which are analogs of those described in Table 1 or 2, are tested in in vitro human and mouse MOR assays. These analogs agonize MOR activity.

An additional aspect of the present disclosure provides analogs of the compounds of Table 2 that are MOR agonists. Additional 6-methyl-6,11-dihydrodibenzo[1,2]thiazepine 5,5-dioxide compounds, which are analogs of those described in Table 2, are tested in in vitro human and mouse MOR assays. These analogs agonize MOR activity. Compounds which are analogs of those described in Table 2 are tested in in vitro human and mouse MOR assays. These analogs agonize MOR activity.

### Example 4. Administration of MOR Agonists

An amount of any one of compounds 6-11, 13-29, 31-53 or 55-84 is administered to a subject afflicted with a neurological disorder as described herein. The amount of the compound is effective to treat the subject afflicted with the neurological disorder as described herein.

An amount of any one of compounds 6-11, 13-29, 31-53 or 55-84 is administered to a subject afflicted with Huntington's disease. The amount of the compound is effective to treat the subject afflicted with Huntington's disease.

An amount of any one of compounds 6-11, 13-29, 31-53 or 55-84 is administered to a subject afflicted with Rett syndrome. The amount of the compound is effective to treat the subject afflicted with Rett syndrome.

An amount of any one of compounds 6-11, 13-29, 31-53 or 55-84 is administered to a subject afflicted with CDKL5 disorder. The amount of the compound is effective to treat the subject afflicted with CDKL5 disorder.

An amount of any one of the compounds disclosed herein, which are analogs of those described in Table 1 or 2, is administered to a subject afflicted with a neurological disorder as described herein.

The amount of the compound is effective to treat the subject afflicted with a neurological disorder as described herein.

An amount of any one of the compounds disclosed herein, which are analogs of those described in Table 1 or 2, is administered to a subject afflicted with Huntington's disease. The amount of the compound is effective to treat the subject afflicted with Huntington's disease.

An amount of any one of the compounds disclosed herein, which are analogs of those described in Table 1 or 2, is administered to a subject afflicted with Rett syndrome. The amount of the compound is effective to treat the subject afflicted with Rett syndrome.

An amount of any one of the compounds disclosed herein, which are analogs of those described in Table 1 or 2, is administered to a subject afflicted with CDKL5 disorder. The amount of the compound is effective to treat the subject afflicted with CDKL5 disorder.

An amount of any one of compounds 59-84 is administered to a subject afflicted with a neurological disorder as described herein. The amount of the compound is effective to treat the subject afflicted with a neurological disorder as described herein.

An amount of any one of compounds 59-84 is administered to a subject afflicted with Huntington's disease. The amount of the compound is effective to treat the subject afflicted with Huntington's disease.

An amount of any one of compounds 59-84 is administered to a subject afflicted with Rett syndrome. The amount of the compound is effective to treat the subject afflicted with Rett syndrome.

An amount of any one of compounds 59-84 is administered to a subject afflicted with CDKL5 disorder. The amount of the compound is effective to treat the subject afflicted with CDKL5 disorder.

### Example 5. Metabolism and Pharmacokinetics

The pharmacokinetics and brain distribution of tianeptine and 23 were determined as follows in male C₅₇BL/6 mice following a single intraperitoneal dose administration. For each compound, a group of twenty-four male mice were administered with a solution formulation of the drug in normal saline intraperitoneally at a dose of 10 mg/kg. Blood samples (approximately 60 μL) were collected under light isoflurane anesthesia from the retro orbital plexus at 0.08, 0.25, 0.5, 1, 2, 4, 8 and 24 hr. Plasma samples were separated by centrifugation of whole blood and stored below −70° C. until bioanalysis. Immediately after collection of blood, brain samples were collected from each mouse at 0.08, 0.25, 0.5, 1, 2, 4, 8 and 24 hr. Brain samples were homogenized using ice-cold phosphate buffer saline (pH 7.4) and homogenates were stored below −70° C. until analysis. Total homogenate volume was three times the tissue weight. All samples were processed for analysis by protein precipitation using acetonitrile (ACN) and analyzed with fit-for-purpose LC/MS/MS method (LLOQ—1.01 ng/mL in plasma and brain). Pharmacokinetic parameters were calculated using the non-compartmental analysis tool of Phoenix WinNonlin® (Version 6.3).

Tianeptine is known to be metabolized primarily by 3-oxidation of the carboxylic acid side chain, in a manner similar to fatty acids (Grislain, L et al. 1990). Other carboxylic analogs known in the prior art are expected to be metabolized in a similar manner. Therefore, replacement of the carboxylic acid side chain with functionality that cannot be easily metabolized via R-oxidation results in compounds with slower metabolism, longer duration of action, and improved therapeutic profile. The compounds disclosed herein with modified side chains (e.g., ethers, esters, or alkyl groups) prevent β-oxidation. For example, the ether compound 23 has a much longer half-life and higher systemic exposure (AUC) than tianeptine, and is therefore expected to have an improved therapeutic profile (Tables 3 and 4).

Other compounds disclosed herein including compounds having an ether, polyether or ester at the R₂ group have an analogous half-life and systemic exposure to 23. Other compounds disclosed have pharmacokinetic profiles similar to compound 23. Further, these compounds have improved pharmacokinetics relative to tianeptine.

TABLE 3
Pharmacokinetic parameters of tianeptine in plasma and brain
following a single intraperitoneal administration in male C57BL/6 mice.

| | | Dose | Tₘₐₓ | Cₘₐₓ | AUCₗₐₛₜ | T_{1/2} | CL | MRTₗₐₛₜ |
|---|---|---|---|---|---|---|---|---|
| Route | Matrix | (mg/kg) | (hr) | (ng/mL) | (hr * ng/ml) | (hr) | (mL/min/kg) | (hr) |
| i.p. | Plasma | 10 | 0.08 | 4879.11 | 1110.23 | 0.12 | 149.65 | 0.19 |
| | Brain* | | 0.08 | 356.77 | 104.41 | 0.14 | 1580.88 | 0.23 |

*brain conc. and AUC expressed as ng/g and hr * ng/g respectively

TABLE 4
Pharmacokinetic parameters of 23 in plasma and brain
following a single intraperitoneal administration in male C57BL/6 mice.

| | | Dose | Tₘₐₓ | Cₘₐₓ | AUCₗₐₛₜ | T_{1/2} | CL | MRTₗₐₛₜ |
|---|---|---|---|---|---|---|---|---|
| Route | Matrix | (mg/kg) | (hr) | (ng/mL) | (hr * ng/ml) | (hr) | (mL/min/kg) | (hr) |
| i.p. | Plasma | 10 | 0.25 | 1535.74 | 3058.80 | 3.93 | 44.27 | 2.29 |
| | Brain* | | 0.08 | 377.63 | 335.03 | 5.12 | 260.44 | 1.27 |

*brain conc. and AUC expressed as ng/g and hr * ng/g respectively

### Example 6. Esters as Prodrugs

Carboxylate esters are well known as prodrugs for the corresponding carboxylic acids obtained by hydrolysis (Beaumont, et al. 2003). Such ester prodrugs show improved oral bioavailability, better brain penetration, or longer duration of action compared to their carboxylic acid counterparts. Accordingly, compounds of this application having an ester side chain, although biologically active on their own, also act as prodrugs for the corresponding carboxylic acids. Further, one skilled in the art will be able to apply the methods and knowledge of this application to prepare additional prodrugs. For example, the type of ester (e.g., methyl, ethyl, propyl, isopropyl, phenyl) or the length of the side chain may be varied to adjust the activity and pharmacokinetic properties of the prodrugs and their corresponding carboxylic acid hydrolysis products. Examples of additional prodrugs and their preparation are shown in Scheme 1.

Compounds 6, 9, 14, 17, 34, 41, 45, 49, 51, 61, 62, 66, 70, 74, 79, and 81 act as prodrugs to deliver the corresponding acid.

Scheme 1. Additional prodrugs are designed and synthesized as shown.

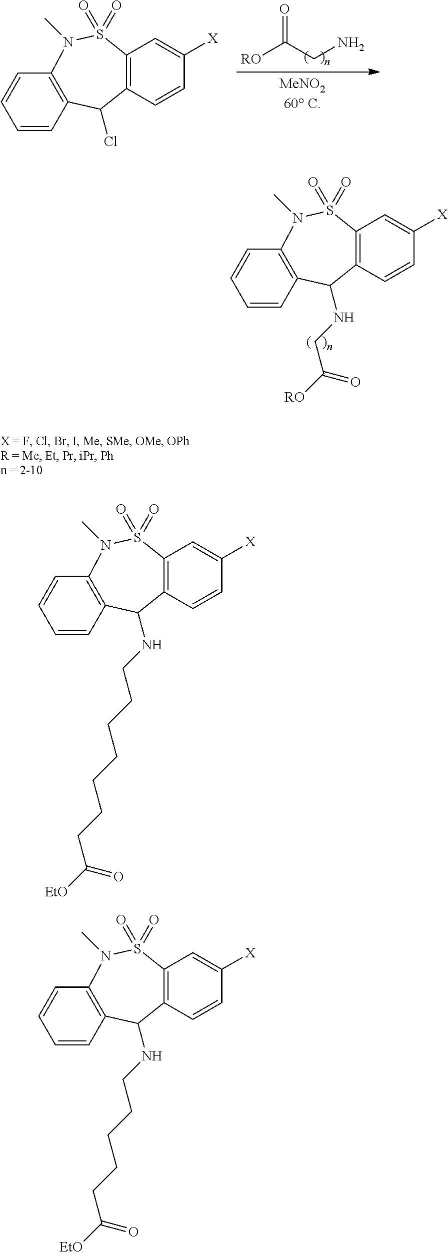

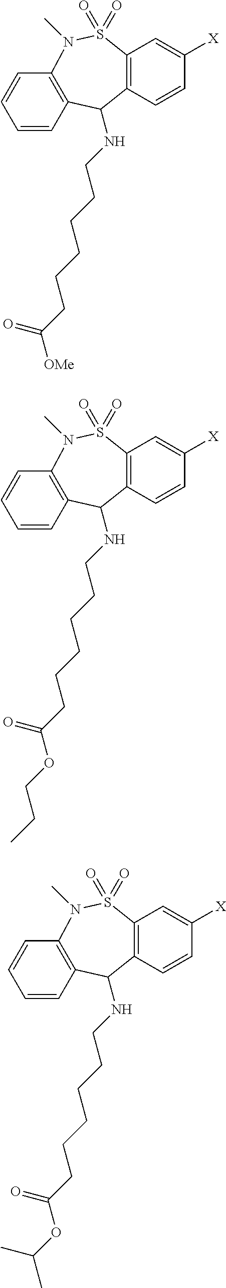

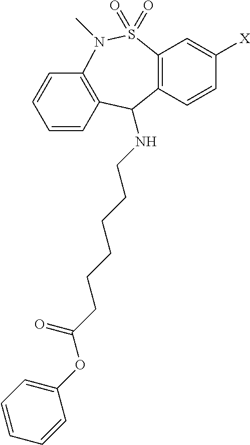

### Example 7. Combinations with NMDA Receptor Antagonists

Antagonists of the N-methyl-D-aspartate receptor (NMDAR) are known to potentiate the beneficial effects of opioid receptor agonists (Trujillo, K. A. et al. 1994; Mao, J. et al. 1996). Therefore, pharmaceutical compositions of tianeptine or the novel compounds disclosed herein, combined with NMDAR antagonists, may be useful in the treatment of neurological disorders, as described herein.

Alternatively, the opioid modulator and NMDAR antagonist may be dosed separately, as a novel method for treating neurological disorders.

Non-Limiting Examples of NMDA Receptor Antagonists:
Dextromorphinans—dextromethorphan, dextrorphan, dextrallorphan
Adamantanes—memantine, amantadine, rimantadine, nitromemantine (YQW-36); Aylcyclohexylamines: ketamine (and its analogs, e.g., tiletamine), phencyclidine (and its analogs, e.g., tenocyclidine, eticyclidine, rolicyclidine), methoxetamine (and its analogs), gacyclidine (GK-11); Miscellaneous: neramexane, lanicemine (AZD6765), diphenidine, dizocilpine (MK-801), 8a-phenyldecahydroquinoline (8A-PDHQ), remacemide, ifenprodil, traxoprodil (CP-101,606), eliprodil (SL-82.0715), etoxadrol (CL-1848C), dexoxadrol, WMS-2539, NEFA, delucemine (NPS-1506), aptiganel (Cerestat; CNS-1102), midafotel (CPPene; SDZ EAA 494), dexanabinol (HU-211 or ETS2101), selfotel (CGS-19755), 7-chlorokynurenic acid (7-CKA), 5,7-dichlorokynurenic acid (5,7-DCKA), L-683344, L-689560, L-701324, GV150526A, GV196771A, CERC-301 (formerly MK-0657), atomoxetine, LY-235959, CGP 61594, CGP 37849, CGP 40116 (active enantiomer of CG 37849), LY-233536, PEAQX (NVP-AAM077), ibogaine, noribogaine, Ro 25-6981, GW468816, EVT-101, indantadol, perzinfotel (EAA-090), SSR240600, 2-MDP (U-23807A), AP-7

### Example 8. Combinations with NMDA Receptor Partial Agonists

Weak partial agonists of NMDAR are also known (Moskal, J. R. et al. 2005), and may be expected to produce beneficial or synergistic effects similar to an antagonist when intrinsic glutamate signaling activity is high or over-activated. Therefore, pharmaceutical compositions of tianeptine or the novel compounds disclosed herein, combined with NMDAR partial agonists, may be useful in the treatment of neurological disorders. Alternatively, the opioid modulator and NMDAR partial agonist may be dosed separately, as a novel method for treating neurological disorders.

Non-Limiting Examples of NMDA Receptor Partial Agonists:
NRX-1074, rapastinel (GLYX-13)

### Example 9. Combinations with Neurokinin 1 Receptor Antagonists

Antagonists of the neurokin 1 receptor (NK-1) are known to modulate the effects of opioid agonists. More specifically, NK-1 antagonists attenuate opioid self-administration in animal models (Robinson, J. E. et al. 2012). Therefore, pharmaceutical compositions of tianeptine or the novel compounds disclosed herein, combined with NK-1 antagonists, may be useful in the treatment of neurological disorders with increased efficacy. Alternatively, the opioid modulator and NK-1 antagonist may be dosed separately, as a novel method for treating neurological disorders.

Non-Limiting Examples of Neurokinin 1 Receptor Antagonists:
aprepitant, fosaprepitant, casopitant, maropitant, vestipitant, vofopitant, lanepitant, orvepitant, ezlopitant, netupitant, rolapitant, L-733060, L-703606, L-759274, L-822429, L-760735, L-741671, L-742694, L-732138, CP-122721, RPR-100893, CP-96345, CP-99994, TAK-637, T-2328, CJ-11974, RP 67580, NKP608, VPD-737, GR 205171, LY686017, AV608, SR140333B, SSR240600C, FK 888, GR 82334

### Example 10. Combinations with Neurokinin 2 Receptor Antagonists

Antagonists of the neurokin 2 receptor (NK-2) are known to show antidepressant effects and to synergize with tricyclic antidepressants (Overstreet, D. H. et al. 2010). Therefore, pharmaceutical compositions of tianeptine or the novel compounds disclosed herein, combined with NK-2 antagonists, may be useful in the treatment of neurological disorders with increased efficacy. Alternatively, the opioid modulator and NK-2 antagonist may be dosed separately, as a novel method for treating neurological disorders.

Non-Limiting Examples of Neurokinin 2 Receptor Antagonists:
saredutant, ibodutant, nepadutant, GR-159897, MEN-10376

### Example 11. Combinations with Neurokinin 3 Receptor Antagonists

Antagonists of the neurokin 3 receptor (NK-3) are known to show a dependency on the opioid receptor system (Panocka, I. et al. 2001). Therefore, pharmaceutical compositions of tianeptine or the novel compounds disclosed herein, combined with NK-3 antagonists, may be useful in the treatment of neurological disorders with increased efficacy. Alternatively, the opioid modulator and NK-3 antagonist may be dosed separately, as a novel method for treating neurological disorders.

Non-Limiting Examples of Neurokinin 3 Receptor Antagonists:
osanetant, talnetant, SB-222200, SB-218795

### Example 12. Efficacy of Compounds in Treating Huntington's Disease (HD)

Overview—The compounds of the present invention were tested for their potential ability to reverse a Huntington's disease-like behavioral phenotype in mice using the SmartCube® (SC) system and associated data analysis methods. The tested compounds demonstrated activity likely to reverse the disease phenotype and therefore, are useful in treating Huntington's disease and other neurological disorders, as described herein.

Animals—All mouse husbandry and experimental procedures were conducted with the approval of the appropriate Animal Care and Use Committee. Q175 HD model heterozygous mice (PsychoGenics) were bred and aged. These mice were subjected to testing at 6 and 10 months of age in the SC system to establish a Huntington's Disease-like behavioral phenotype for comparison to drug-induced behavioral phenotypes, as described further below. For screening of the test compounds, wild-type male C₅₇BL/6 mice were used. All animals were examined, handled, and weighed prior to initiation of testing to assure adequate health and suitability and to minimize non-specific stress associated with manipulation. During the course of the study, mice were group-housed in OPTI mouse ventilated cages with 4 mice/cage. 12/12 light/dark cycles were maintained. The room temperature was maintained between 20 and 23° C. with a relative humidity maintained between 30 and 70%. Chow and water were provided ad libitum for the duration of the study. Animals were acclimated to the vivarium for at least one week prior to commencing testing and tested at 8-9 weeks of age. Body weight was measured prior to testing.

Treatment groups—12 mice were used in each of the following groups:
1. Vehicle
2. compound 84 1.5 mg/kg
3. compound 84 3.0 mg/kg
4. compound 84 6.0 mg/kg
5. compound 84 12 mg/kg
6. compound 7e 2.25 mg/kg
7. compound 7e 4.5 mg/kg
8. compound 7e 9.0 mg/kg
9. compound 7e 18 mg/kg
10. tianeptine, 10 mg/kg
11. tianeptine, 20 mg/kg

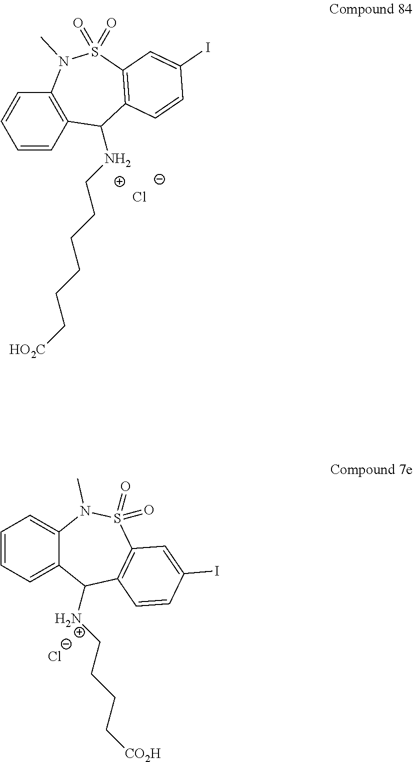 [SMILES: CN1c2ccccc2C([NH2+]CCCCCCC(=O)O)c2ccc(I)cc2S1(=O)=O ; [Cl-] ; CN1c2ccccc2C([NH2+]CCCCC(=O)O)c2ccc(I)cc2S1(=O)=O ; [Cl-]]

Administration of test compounds was by subcutaneous injection at an injection volume of 10 mL/kg, except for tianeptine, which was administered intraperitoneally at an injection volume of 10 mL/kg. Saline was used as the vehicle for all test compounds. All testing and data analysis was carried out blinded to treatment.

Behavior testing—The SC system (PsychoGenics) was utilized to measure numerous spontaneous behaviors and response to challenges in the same testing environment. The hardware included force sensors and a number of aversive stimuli to elicit behavior. Three high-resolution video cameras provided a constant 3D view of the mouse in the SC apparatus throughout the entire testing period. The mice were exposed to a sequence of challenges during the 45-minute test period. Mice were injected with vehicle or test compound and placed in the SC 15 minutes after administration. The cubes were cleaned between each run.

Computational modelling was performed for behavioral signatures, including tail flick, grooming/sniffing, ear twitch (orientation response), and orientation (walking, running, rearing, freezing).

Data analysis—Several analytical methods including Bayesian probabilistic density models were utilized alongside data mining algorithms (Alexandrov, V. et al. 2015). The algorithms can consider more than 2,000 measures including frequency and duration of behavioral states such as grooming, rearing, mobility, behavioral transitions and many other features obtained during the test session.

Two major types of analyses are routinely conducted: class and subclass. For class and subclass analysis, a reference data set was built from hundreds of drug doses in multiple drug classes plus a control group. Each reference drug was tested at multiple doses appropriate for that drug in mice. The best performing classifiers were chosen from evaluation tests and two separate types of classifiers were built that make independent predictions at drug class and subclass levels.

The class consists of drugs that are currently on the market or that have been clinically validated for that specific indication. The subclass consists of both marketed drugs and other compounds that have been mechanistically validated and is a larger set than the class. The reference databases have been continually expanded and new databases have been kept in development. Once validated, these new databases were incorporated into the analysis to provide additional information about each compound tested.

Data from the screening was processed using proprietary computer vision and data mining algorithms developed by PsychoGenics and the results were compared to signatures of the reference compounds in the database. Multiple analyses of the data were performed to quantitatively produce independent predictions of drug class, and drug subclass. The behavioral signatures of the test drugs were evaluated using these classifiers to confirm therapeutic utility. The results for the class and subclass analyses were presented as standardized bar charts with percentages that sum to 100 for each dose. The results of the classification at the drug level were presented as individual similarities.

Similarity analysis—The outcome from SC provides a large set of features (behavioral parameters) that has been used for various analyses. Many of these features have been correlated. Therefore, it has been useful to form statistically independent combinations of the original features (further referred to as de-correlated features). Each de-correlated feature extracted information from the whole cluster of original features, so the new feature space had lower dimensionality. Next, a feature ranking algorithm was applied to score each feature for its discrimination power (ability to separate the two groups, e.g., reference and test).

Ranking was used in the analyses because it weighs each feature change by its relevance. If there were to be a significant change in some irrelevant features measured for a particular phenotype, the low rank of these features will automatically reduce the effect of such change in the analyses. This means that a conventional “feature selection” approach did not need to be used and information was not lost among the less informative features. Ranking was applied to either the original or the de-correlated features.

It was also useful to examine the de-correlated ranked features as “clouds”. This refers to Gaussian distributions approximating the groups of mice (e.g., between test compound and vehicle or between test compound and a reference compound like tianeptine) in the ranked de-correlated features space. This was used to calculate a quantitative measure of separability (distinguishability) between the two groups. The two highest ranked de-correlated features were chosen to form the 2D coordinate plane for visualization purposes. For visualization purposes, it was possible to plot each cloud with its semi-axes equal to one standard deviation along the corresponding dimensions.

In “proximity-to-the-drug” experiments, the data are typically presented as three classes: reference, target, and test drug. Therefore, it was deemed instructive to consider and plot the third group, test drug, in the same coordinate system that best discriminates the other groups (reference and target). The drug treatment effect was then able to be represented as a combination of two components: one along the direction of the “proximity line” (the line connecting the centers of the target and reference clouds) and the component orthogonal to (“pointing away” from) that direction. The relative length of the “proximity” line with respect to the target-reference distance was then able to be interpreted as the “proximity to target”, whereas the relative length of the “other effect” line represents feature changes that move the target+treatment group away from the reference group. The summary of this analysis was effectively represented as a bar graph, which was referred to as the proximity signature.

Drug-induced behavior similarity analysis (DBSA)—Computational methods for in vivo drug discovery using large-scale datasets have been used to substantially reduce the high cost of drug screening by providing comprehensive and systematic means to discover new biological targets for existing drugs. One of the most successful computational methods for drug screening has been the correlative analysis, which uses a wide collection of genome-wide transcriptional expression data from cultured human cells or in vivo behavioral data following treatment with bioactive small molecules together with pattern-matching algorithms to discover functional connections between drugs and diseases.

The underlying hypothesis for correlative analysis is that a drug effective in the treatment of a disease (or reduction of side-effects of another compound) should induce an opposite behavioral profile to that seen in the un-treated disease state (or the other compound), whereas drugs with similar therapeutic indications should induce similar behavioral profiles. Based on this principle, an algorithm called Drug Behavioral Signature Analysis (DBSA; PsychoGenics) was employed. This has allowed drug-induced behavioral data to be used to predict functional connections among drugs or between drugs and diseases.

DBSA was used to compare the behavioral profile induced by a test drug in the SC to the behavioral profile of the disease model of interest in the SC and to compute a similarity score to identify the potential therapeutic benefit of the test drug. This analysis can thereby identify compounds with three different sets of profiles: a) ‘similar’ indicating a profile similar to that of the test compound or disease; b) ‘reverse’ indicating a profile opposite to that of the test compound or disease, thus suggesting a potential therapeutic effect on the disease; and c) ‘random’ indicating no similarity. The profile of any desired compound or disease can be queried against an existing database of compounds and disease states in order to identify compounds with similar or reverse profiles, depending on the underlying requirements.

For DBSA analysis in the present experiment, the SC behavioral profile induced by each test compound in wild-type mice was compared to the SC behavioral profile of Q175 heterozygous mice (a mouse model of Huntington's disease) at 6 and 10 months of age. Results of DBSA are presented as normalized enrichment scores (the sign +/− of which indicates similar/reverse signature), associated p-value (signifying random or non-random), and odds-ratio (level of enrichment).

Additional information on data analysis with the SC can be found in Alexandrov, V. et al. 2015, the contents of which are herein incorporated by reference.

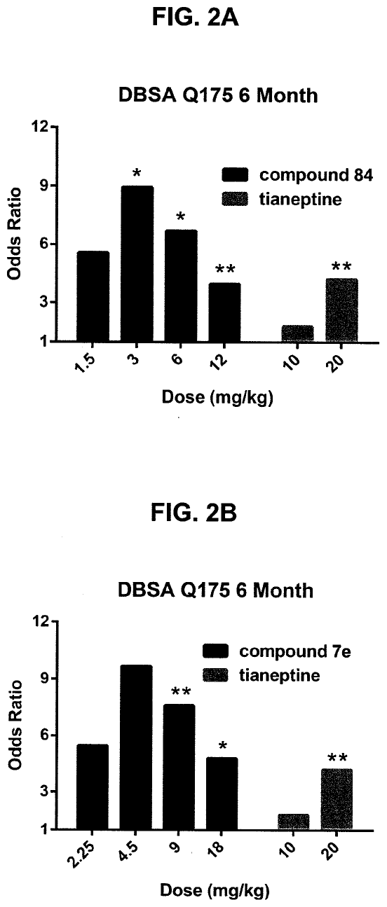

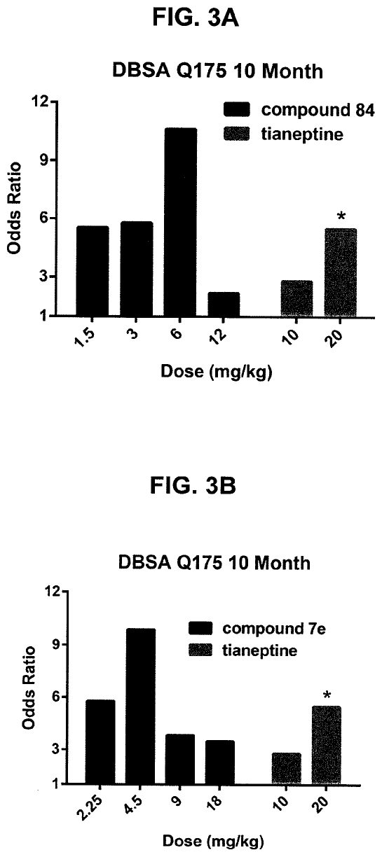

Results—Administration of the disclosed compounds to wild-type mice induced a behavioral phenotype opposed to the behavioral phenotype exhibited by Q175 mice at 6 and 10 months of age, as demonstrated by DBSA analysis (FIGS. 2A-3B), indicating that the compounds can be used to reverse the Huntington's disease-like behavioral deficits exhibited by Q175 mice. The compounds are therefore expected to be beneficial in the treatment of Huntington's disease and other neurological disorders as described herein. Comparison to tianeptine in the same experimental paradigm also revealed that the disclosed compounds are more potent and more efficacious than this commercially available pharmaceutical agent.

Discussion

The compounds disclosed herein activate MOR or dually activate MOR and DOR. This activity leads to modulation of the glutamatergic system and triggers corresponding effects. Accordingly, the compounds disclosed herein are useful as neurological therapeutics.

An additional aspect of the present disclosure provides synthetic methods and chemical intermediates that may be used to encompass chemical space around the 6-methyl-6,11-dihydrodibenzo[1,2]thiazepine 5,5-dioxide or 4-methyl-4,10-dihydrobenzo[f]thieno[3,2-c][1,2]thiazepine 5,5-dioxide core structure.

### REFERENCES

Alexandrov, V.; Brunner, D.; Hanania, T.; Leahy, E. European Journal of Pharmacology 2015, 753, 127-134.
Barbier, E.; Vendruscolo, L. F.; Schlosburg, J. E.; Edwards, S.; Juergens, N.; Park, P. E.; Misra, K. K.; Cheng, K.; Rice, K. C.; Schank, J.; Schulteis, G.; Koob, G. F.; Heilig, M. Neuropsychopharmacology 2013, 38, 976-984.
Beaumont, K.; Webster, R.; Gardner, I.; Dack, K. Curr. Drug Metab. 2003, 4, 461-485.
Berrocoso, E.; Sánchez-Blázquez, P.; Garzón, J.; Mico, J. A. Curr. Pharm. Des. 2009, 15, 1612-1622.
Besson, A.; Privat, A. M.; Eschalier, A.; Fialip, J. Psychopharmacology 1996, 123, 71-78.
Bodkin, J. A.; Zornberg, G. L.; Lukas, S. E.; Cole, J. O. J. Clin. Psychopharmacol. 1995, 15, 49-57.
Buchwald, S. L.; Huang, X.; Zim, D. Ligands for metals and improved metal-catalyzed processes based thereon. US2004/171833 A1, 2004.
Corbett, A. D.; Henderson, G.; McKnight, A. T.; Paterson, S. J.; Brit. J. Pharmacol. 2006, 147, S153-S162.
Dreher, S. D.; Lim, S.-E.; Sandrock, D. L.; Molander, G. A. J. Org. Chem. 2009, 74, 3626-3631.
Durand, P.; Richard, P.; Renaut, P. J. Org. Chem. 1998, 63, 9723-9727.
Gilleron, P.; Wlodarczyk, N.; Houssin, R.; Farce, A.; Laconde, G.; Gocssens, J.-F.; Lemoine, A.; Pommery, N.; Hénichart, J.-P.; Millet, R. Bioorg. Med. Chem. Lett. 2007, 17, 5465-5471.
Grislain, L.; Gele, P.; Bertrand, M.; Luijten, W.; Bromet, N.; Salvadori, C.; Kamoun, A. Drug Metab. Dispos. 1990, 18, 804-808.
Invernizzi, R.; Pozzi, L.; Garattini, S.; Samanin, R. Neuropharmacology 1992, 31, 221-227.
Jutkiewicz, E. M. Mol. Interv. 2006, 6, 162-169.
Kramer, M. S.; Winokur, A.; Kelsey, J.; Preskorn, S. H.; Rothschild, A. J.; Snavely, D.; Ghcsh, K.; Ball, W. A.; Reines, S. A.; Munjack, D.; Apter, J. T.; Cunningham, L.; Kling, M.; Bari, M.; Getson¬, A.; Lee, Y. Neuropsychopharmacology 2004, 29, 385-392.
Mac, J.; Price, D. D.; Caruso, F. S.; Mayer, D. J. Pain 1996, 67, 361-368.
McNeill, E.; Barder, T. E.; Buchwald, S. L. Org. Lett. 2007, 99, 3785-3788.
Moskal, J. R.; Kuo, A. G.; Weiss, C.; Wood, P. L.; Hanson, A. O.; Kelso, S.; Harris, R. B.; Disterhoft, J. F. Neuropharmacology 2005, 49, 1077-1087.
Murrough, J. W.; Iosifescu, D. V.; Chang, L. C.; Al Jurdi, R. K.; Green, C. E.; Perez, A. M.; Iqbal, S.; Pillemer, S.; Foulkes, A.; Shah, A.; Charney, D. S.; Mathew, S. J. Am. J. Psychiatry 2013, 170, 1134-1142.
Overstreet, D. H.; Naimoli, V. M.; Griebel, G. Pharmacol. Biochem. Behav. 2010, 96, 206-210.
Robinson, J. E.; Fish, E. W.; Krouse, M. C.; Thorsell, A.; Heilig, M.; Malanga, C. J. Psychopharmacology 2012, 220, 215-224.
Pan, J.; Wang, X.; Zhang, Y.; Buchwald, S. L. Org. Lett. 2011, 13, 4974-4976.
Panocka, I.; Massi, M.; Lapo, I.; Swiderski, T.; Kowalczyk, M.; Sadowski, B. Peptides 2001, 22, 1037-1042.
Paul, I. A.; Skolnick, P. Ann. N.Y. Acad. Sci. 2003, 1003, 250-272.
Salome, N.; Stemmelin, J.; Cohen, C.; Griebel, G. Pharmacol. Biochem. Behav. 2006, 83, 533-539.
Svoboda, K. R.; Adams, C. E.; Lupica, C. R.; J. Neurosci. 1999, 19, 85-95.
Trujillo, K. A.; Akil, H. Brain Res. 1994, 633, 178-188.
Uy, R.; Yang, L.; Zhou, H.; Price, S. C.; You, W. Macromolecules 2011, 44, 9146-9154.
Williams, J. T.; Ingram, S. L.; Henderson, G.; Chavkin, C.; von Zastrow, M.; Schultz, S.; Koch, T.; Evans, C. J.; MacDonald, J. C. Pharmacol. Rev. 2013, 65, 223-254.
Xie, C. W., Lewis D. V. J. Neurophysiol. 1997, 78: 759-766.
Zarate, C. A. Jr; Singh, J. B.; Carlson, P. J.; Brutsche, N. E.; Ameli, R.; Luckenbaugh, D. A.; Charney, D. S.; Manji, H. K. Arch. Gen. Psychiatry 2006, 63, 856-864.

Persons of ordinary skill can utilize the disclosures and teachings herein to produce other embodiments and variations without undue experimentation. All such embodiments and variations are considered to be part of this invention.

Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the invention is intended to encompass, within its scope, the modifications, substitutions, and variations to processes, manufactures, compositions of matter, compounds, means, methods, and/or steps disclosed herein.

The description herein may contain subject matter that falls outside of the scope of the claimed invention. This subject matter is included to aid understanding of the invention.

In this specification, where reference has been made to external sources of information, including patent specifications and other documents, this is generally for the purpose of providing a context for discussing the features of the present invention.

1. A compound having the structure: 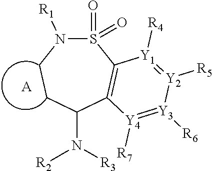
wherein
A is an aryl or heteroaryl, with or without substitution;
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkylaryl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
wherein when A is phenyl, R₁ is —CH₃, R₃, R₄, R₆, and R₇ are each —H, and R₅ is Cl, then R₂ is other than —(CH₂)₄C(O)NH₂, —(CH₂)₄CO₂H, —(CH₂)₅CO₂H, —(CH₂)₆CO₂H, —(CH₂)₇CO₂H, —(CH₂)₁₀CO₂H, —(CH₂)₆CO₂CH₂CH₃, —(CH₂)₆CH₃, —(CH₂)₂₀H, —(CH₂)₄₀H, —(CH₂)₇OH,
wherein when A is phenyl, R₁ is —CH₃, R₃, R₄, R₅, R₆, and R₇ are each —H, then R₂ is other than —(CH₂)CO₂CH₂CH₃, —(CH₂)₂CO₂CH₂CH₃, —(CH₂)CO₂H, —(CH₂)₃CO₂H, —(CH₂)₄CO₂H or —(CH₂)₆CO₂H,
wherein when R₁ is —CH₃, R₂ is —(CH₂)₅CO₂H, R₃ is —H, R₄ and R₇ are each H, R₅ is —Cl and R₆ is —H or R₅ and R₆ are each —H, then A is other than 2-chlorophenyl or 3-chlorophenyl,
wherein when R₁ is —CH₃, R₂ is —(CH₂)₅CO₂H, R₃ is —H or —CH₃, R₄ and R₇ are each —H, R₅ is Cl and R₆ is —H or R₆ is —Cl and R₅ is —H, then A is other than phenyl,
wherein when R₁ is —CH₃, R₂ is —(CH₂)₃CO₂H, R₃ is —CH₃, and R₄, R₅, R₆ and R₇ are each —H, then A is other than phenyl,
wherein when A is phenyl, R₁ is —CH₃, R₃, R₄, R₆, and R₇ are each —H, and R₅ is —SO₂CH₃, then R₂ is other than —(CH₂)₃OCH₃,
wherein when A is phenyl, R₁ is —CH₃, R₃, R₄, R₆, and R₇ are each —H, and R₅ is —F, then R₂ is other than —(CH₂)₆CO₂H,
or a pharmaceutically acceptable salt or ester thereof,
wherein the compound is for treating or preventing a neurological disorder in a subject, wherein the neurological disorder is selected from Huntington's disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; or Lennox-Gastaut syndrome.

2-5. (canceled)

6. The compound of claim 1, wherein
A is 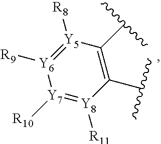
wherein R₈, R₉, R₁₀ and R₁₁ are each absent or present, and when present, are each independently —H, —Cl, —Br, —F, —I, —CN, —CF₃, —OCF₃, -(alkyl), -(aryl), -(heteroaryl)-(alkenyl), -(alkynyl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl);
Y₅, Y₆, Y₇ and Y₈ are each independently N or C,
wherein when Y₅ is N, then R₈ is absent, and when Y₅ is C, then R₈ is present; when Y₆ is N, then R₉ is absent, and when Y₆ is C, then R₉ is present; when Y₇ is N, then R₁₀ is absent, and when Y₇ is C, then R₁₀ is present; when Y₈ is N, then R₁₁ is absent, and when Y₈ is C, then R₁₁ is present.

7. A compound having the structure: 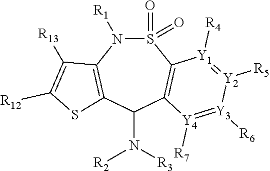
wherein
R₁ is —H or -(alkyl);
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-OCH₃, -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₃ is —H or -(alkyl);
R₄, R₅, R₆ and R₇ are each absent or present, and when present, are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl);
R₁₂ and R₁₃ are each independently —H, —Cl, —Br, —F, —I, —CN, —CF₃, —OCF₃, -(alkyl), -(aryl), -(heteroaryl)-(alkenyl), -(alkynyl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl), or —SO₂-(heteroaryl); and
Y₁, Y₂, Y₃ and Y₄ are each independently N or C,
wherein when Y₁ is N, then R₄ is absent, and when Y₁ is C, then R₄ is present; when Y₂ is N, then R₅ is absent, and when Y₂ is C, then R₅ is present; when Y₃ is N, then R₆ is absent, and when Y₃ is C, then R₆ is present; when Y₄ is N, then R₇ is absent, and when Y₄ is C, then R₇ is present,
or a pharmaceutically acceptable salt thereof,
wherein the compound is for treating or preventing a neurological disorder in a subject, wherein the neurological disorder is selected from Huntington's disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; or Lennox-Gastaut syndrome.

8. The compound of claim 1 having the structure: 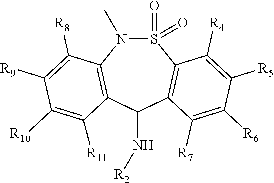
wherein
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₅ is —Br, or —I;
R₄, R₆ and R₇ are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl) or —SO₂-(heteroaryl),
or a pharmaceutically acceptable salt thereof,
wherein the compound is for treating or preventing a neurological disorder in a subject, wherein the neurological disorder is selected from Huntington's disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; or Lennox-Gastaut syndrome.

9. (canceled)

10. The compound of claim 7 having the structure: 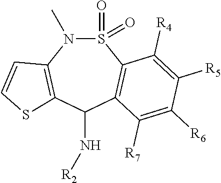
wherein
R₂ is -(alkyl), -(alkenyl), -(alkynyl), -(alkyl)-OH, -(alkyl)-CO₂H, -(alkyl)-CO₂-(alkyl), -(alkyl)-C(O)—NH₂, -(alkyl)-C(O)—NH(alkyl), -(alkyl)-C(O)—NH-(hydroxyalkyl), -(alkyl)-C(O)—N(alkyl)₂, -(alkyl)-C(O)—N(hydroxyalkyl)₂, -(alkyl)-O-(alkyl), -(alkyl)-S-(alkyl), -(alkyl)-CF₃, -(alkyl)-O-(hydroxyalkyl), -(alkyl)-O-(alkyl)-O-(alkyl), -(alkyl)-(CH)—(O-(alkyl))₂, -(alkyl)-(heterocyclyl), -(alkyl)-OAc, -(alkyl)-tetrahydrofuran, -(alkyl)-pyrrolidine, -(alkyl)-N-methylpyrrolidine, -(alkyl)-(1,3-dioxane) or -(alkyl)-(4,5-dihydrooxazole);
R₅ is —Cl, —Br, —F, or —I;
R₄, R₆ and R₇ are each independently —H, —Cl, —Br, —F, —I, —CN, —CF₃, —OCF₃, -(alkyl), -(alkenyl), -(alkynyl), -(aryl), —NH₂, —NH-(alkyl), —NH-(alkenyl), —NH-(alkynyl), —NH-(aryl), —NH-(heteroaryl), —OH, —OAc, —O—C(O)(alkyl), —O-(alkyl), —O-(alkenyl), —O-(alkynyl), —O-(aryl), —O-(heteroaryl), —S-(alkyl), —S-(alkenyl), —S-(alkynyl), —S-(aryl), —S-(heteroaryl), —S(O)-(alkyl), —S(O)-(aryl), —S(O)-(heteroaryl), —SO₂-(alkyl), —SO₂-(aryl) or —SO₂-(heteroaryl).

11. (canceled)

12. The compound of claim 1 having the structure: 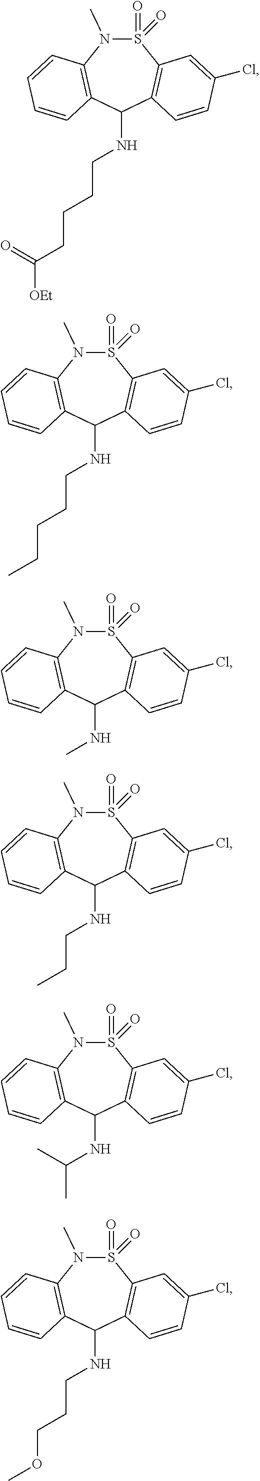 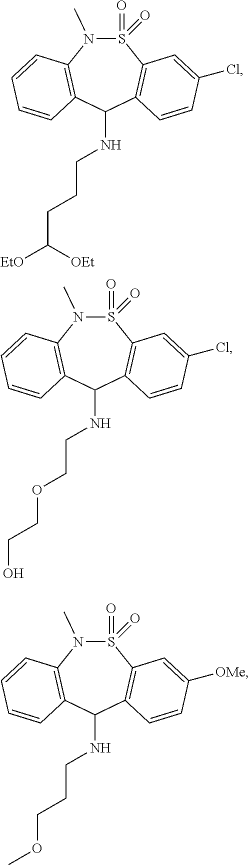 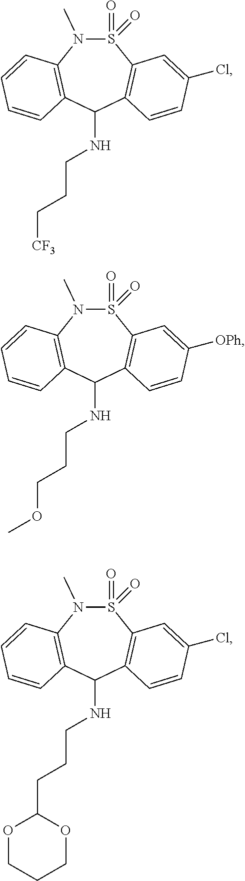 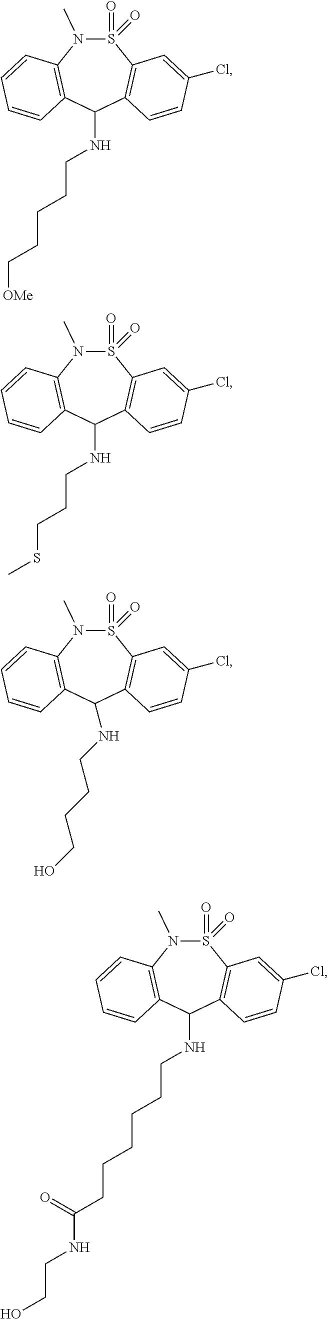 [SMILES: COCCCCCNC1c2ccccc2N(C)S(=O)(=O)c2cc(Cl)ccc21 ; CSCCCNC1c2ccccc2N(C)S(=O)(=O)c2cc(Cl)ccc21 ; CN1c2ccccc2C(NCCCCO)c2ccc(Cl)cc2S1(=O)=O ; CN1c2ccccc2C(NCCCCCCC(=O)NCCO)c2ccc(Cl)cc2S1(=O)=O] 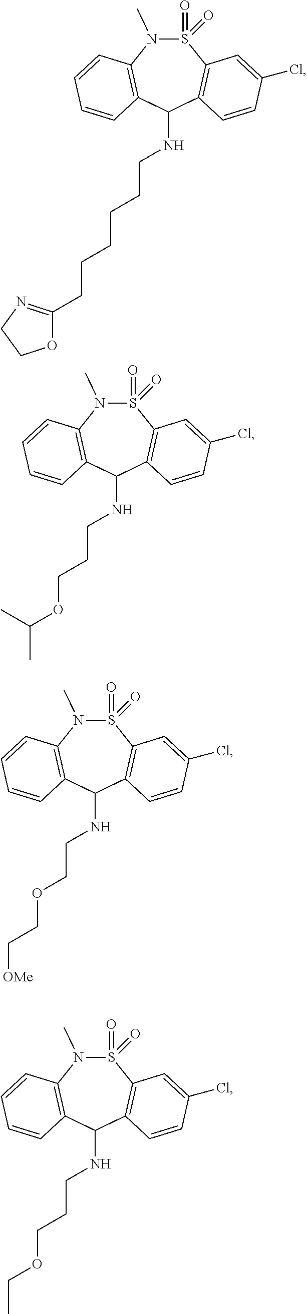 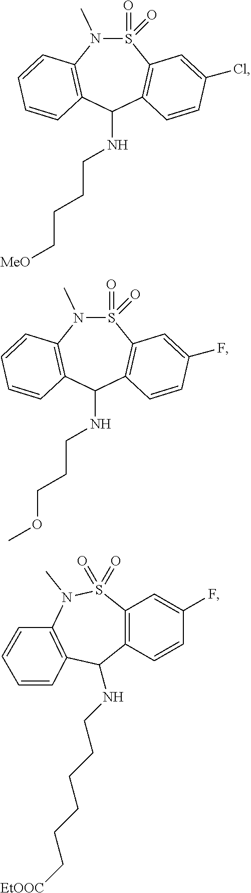 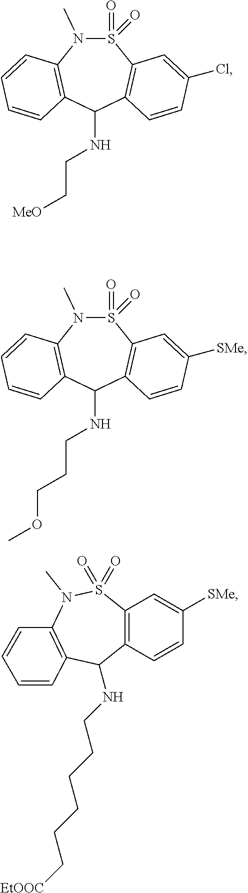 [SMILES: COCCNC1c2ccccc2N(C)S(=O)(=O)c2cc(Cl)ccc21 ; COCCCNC1c2ccccc2N(C)S(=O)(=O)c2cc(SC)ccc21 ; CCOC(=O)CCCCCCNC1c2ccccc2N(C)S(=O)(=O)c2cc(SC)ccc21] 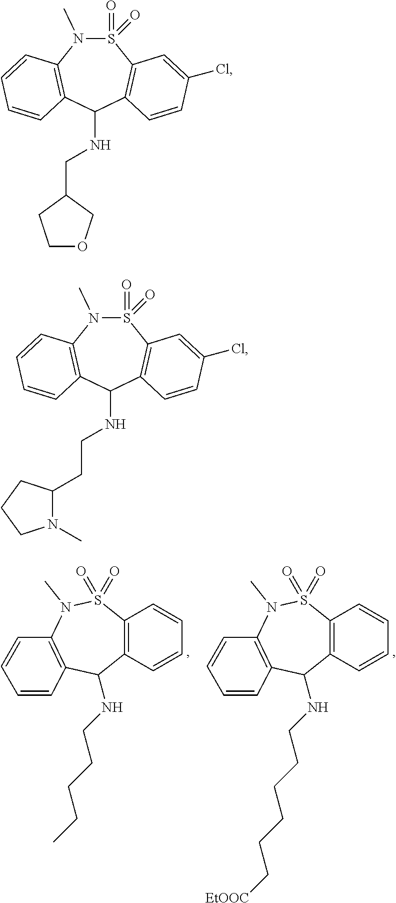 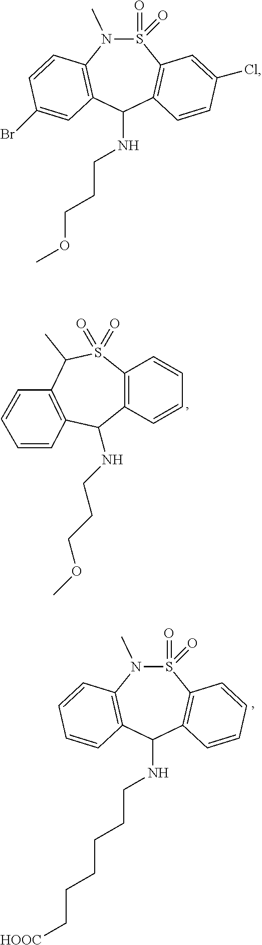 [SMILES: COCCCNC1c2cc(Br)ccc2N(C)S(=O)(=O)c2cc(Cl)ccc21 ; COCCCNC1c2ccccc2C(C)S(=O)(=O)c2ccccc21 ; CN1c2ccccc2C(NCCCCCCC(=O)O)c2ccccc2S1(=O)=O] 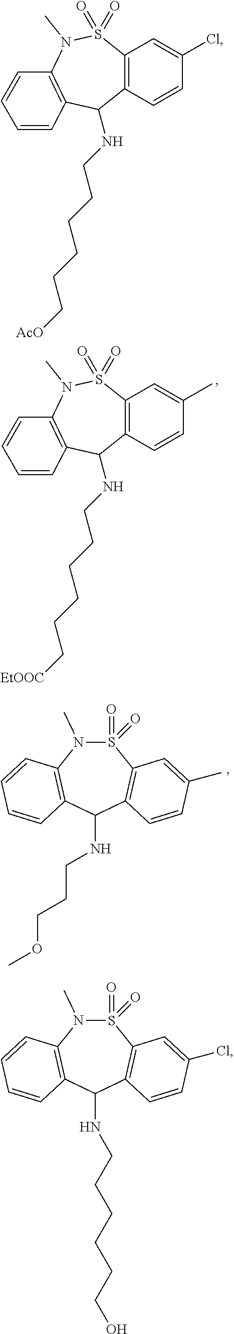 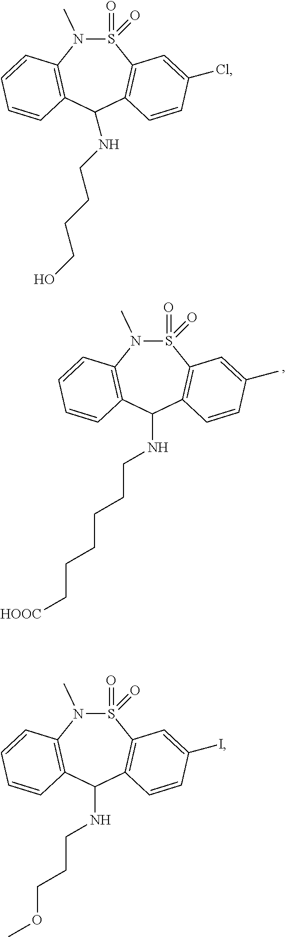 [SMILES: CN1c2ccccc2C(NCCCCO)c2ccc(Cl)cc2S1(=O)=O ; Cc1ccc2c(c1)S(=O)(=O)N(C)c1ccccc1C2NCCCCCCC(=O)O ; COCCCNC1c2ccccc2N(C)S(=O)(=O)c2cc(I)ccc21] 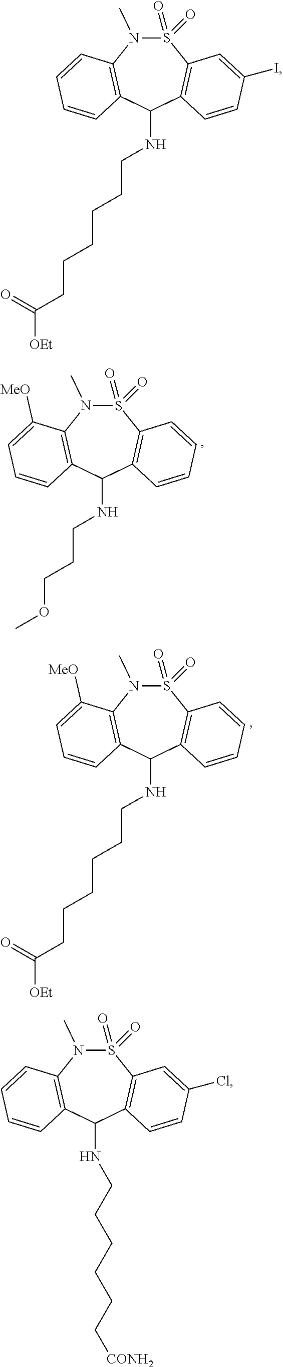 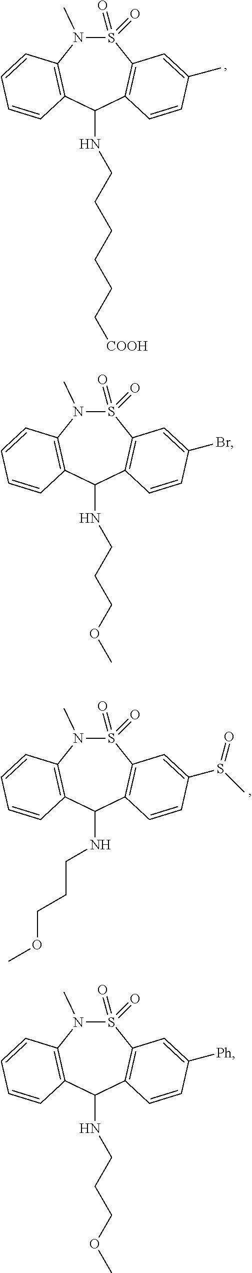 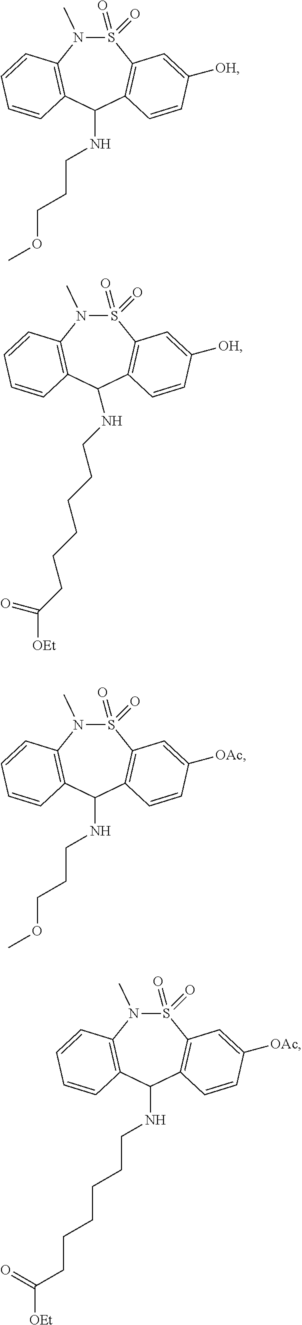 [SMILES: COCCCNC1c2ccccc2N(C)S(=O)(=O)c2cc(O)ccc21 ; CCOC(=O)CCCCCCNC1c2ccccc2N(C)S(=O)(=O)c2cc(O)ccc21 ; COCCCNC1c2ccccc2N(C)S(=O)(=O)c2cc(OC(C)=O)ccc21 ; CCOC(=O)CCCCCCNC1c2ccccc2N(C)S(=O)(=O)c2cc(OC(C)=O)ccc21] 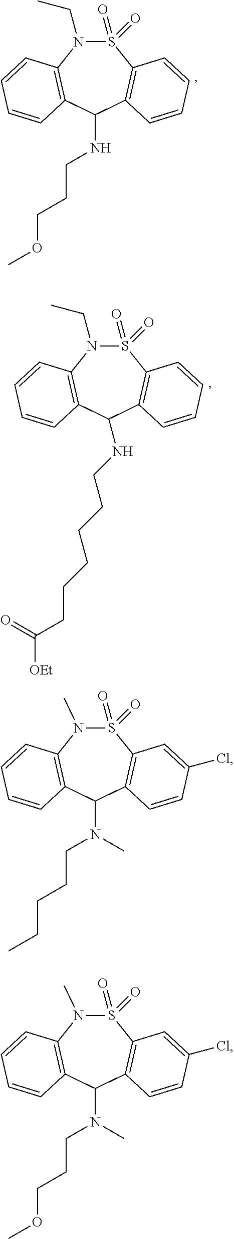 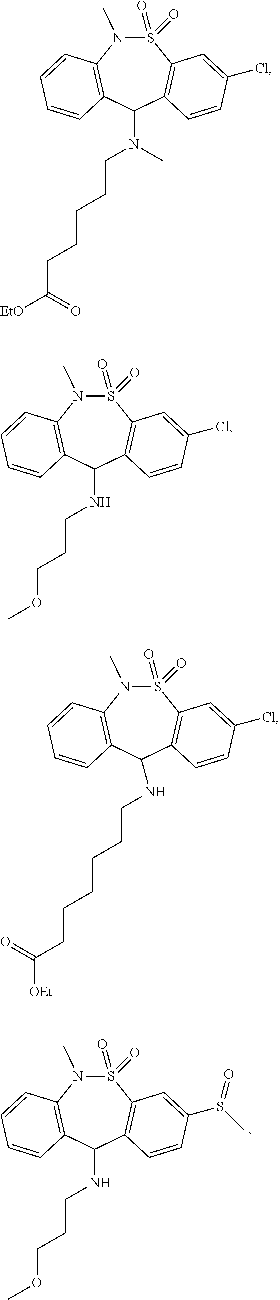 [SMILES: CCOC(=O)CCCCCN(C)C1c2ccccc2N(C)S(=O)(=O)c2cc(Cl)ccc21 ; COCCCNC1c2ccccc2N(C)S(=O)(=O)c2cc(Cl)ccc21 ; CCOC(=O)CCCCCCNC1c2ccccc2N(C)S(=O)(=O)c2cc(Cl)ccc21 ; COCCCNC1c2ccccc2N(C)S(=O)(=O)c2cc(S(C)=O)ccc21] 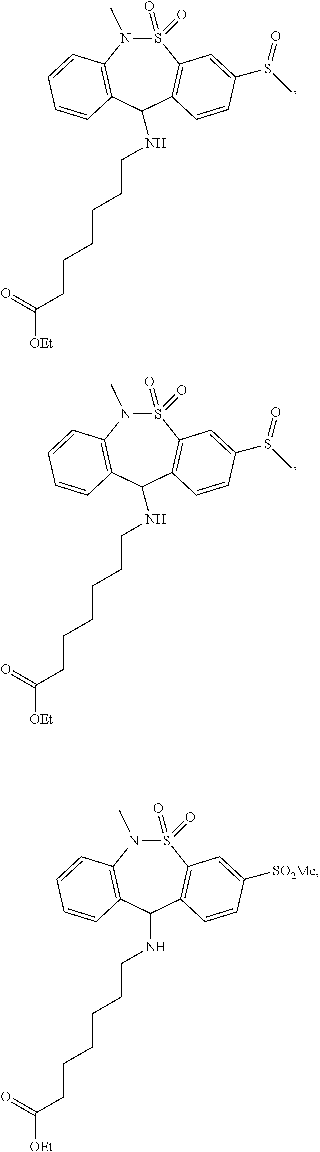 [SMILES: CCOC(=O)CCCCCCNC1c2ccccc2N(C)S(=O)(=O)c2cc(S(C)=O)ccc21 ; CCOC(=O)CCCCCCNC1c2ccccc2N(C)S(=O)(=O)c2cc(S(C)=O)ccc21 ; CCOC(=O)CCCCCCNC1c2ccccc2N(C)S(=O)(=O)c2cc(S(C)(=O)=O)ccc21] 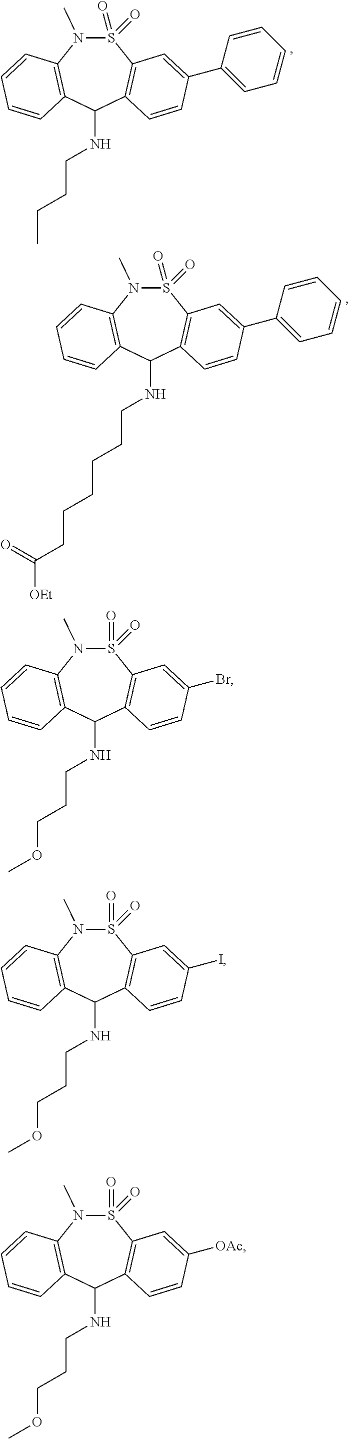 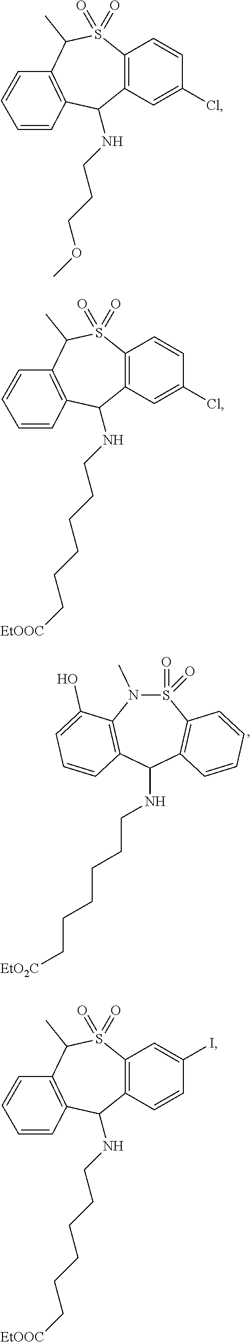 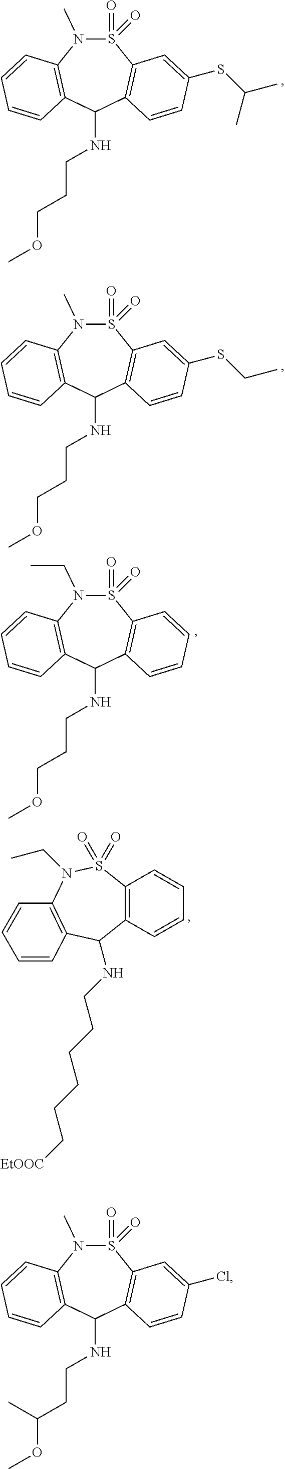 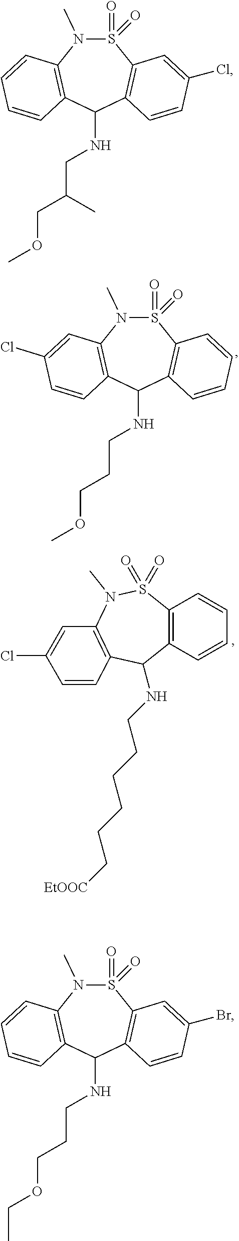 [SMILES: COCC(C)CNC1c2ccccc2N(C)S(=O)(=O)c2cc(Cl)ccc21 ; COCCCNC1c2ccc(Cl)cc2N(C)S(=O)(=O)c2ccccc21 ; CCOC(=O)CCCCCCNC1c2ccc(Cl)cc2N(C)S(=O)(=O)c2ccccc21 ; CCOCCCNC1c2ccccc2N(C)S(=O)(=O)c2cc(Br)ccc21] 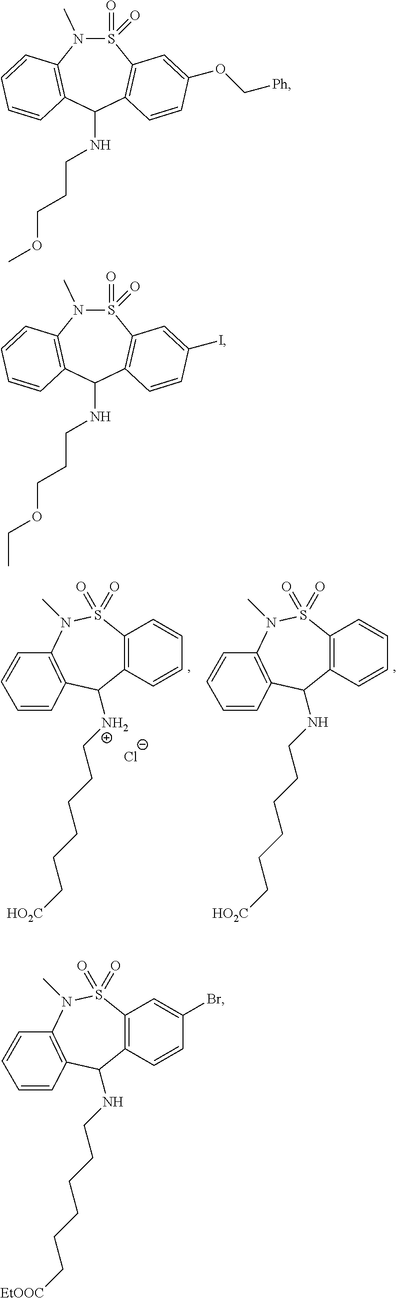 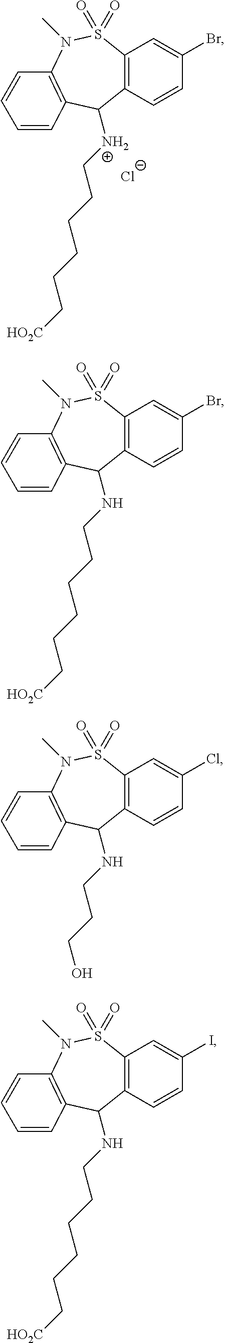 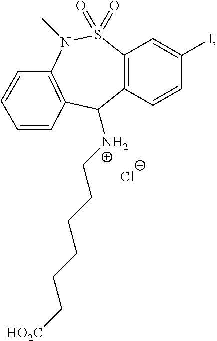 [SMILES: CN1c2ccccc2C([NH2+]CCCCCCC(=O)O)c2ccc(I)cc2S1(=O)=O ; [Cl-]]
or a pharmaceutically acceptable salt thereof.

13-14. (canceled)

15. The compound of claim 7 having the structure: 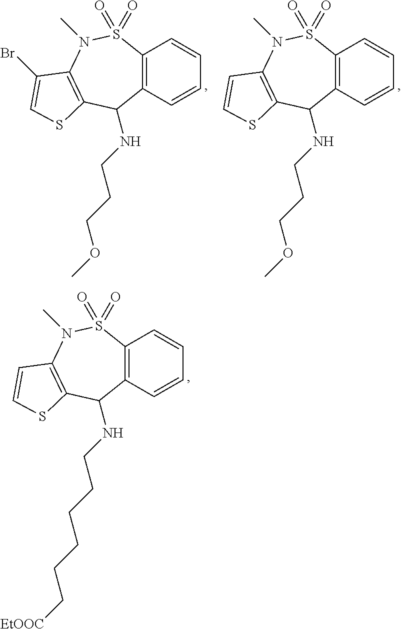 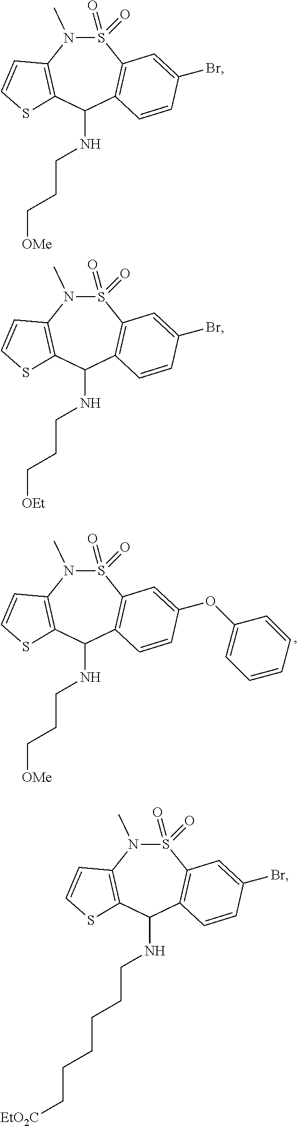 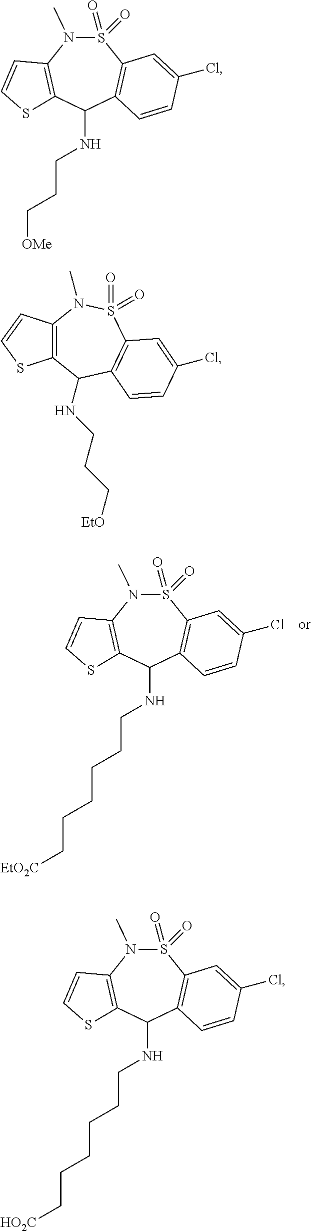
or a pharmaceutically acceptable salt thereof.

16-18. (canceled)

19. A pharmaceutical composition comprising the compound of claim 1 and a pharmaceutically acceptable carrier for use in treating or preventing the neurological disorder selected from Huntington's disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; or Lennox-Gastaut syndrome.

20. (canceled)

21. A method of treating a neurological disorder in a subject, wherein the neurological disorder is selected from Huntington's disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; or Lennox-Gastaut syndrome, comprising administering an effective amount of the compound of claim 1 to the subject so as to treat the neurological disorder.

22-34. (canceled)

35. The compound of claim 1 having the structure: 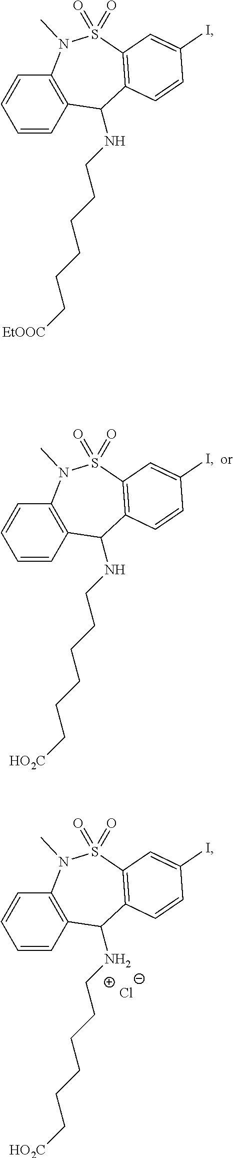 [SMILES: CCOC(=O)CCCCCCNC1c2ccccc2N(C)S(=O)(=O)c2cc(I)ccc21 ; CN1c2ccccc2C(NCCCCCCC(=O)O)c2ccc(I)cc2S1(=O)=O ; CN1c2ccccc2C(NCCCCCCC(=O)O)c2ccc(I)cc2S1(=O)=O ; Cl]
or a pharmaceutically acceptable salt thereof; wherein the compound is for treating or preventing a neurological disorder in a subject, wherein the neurological disorder is selected from Huntington's disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; or Lennox-Gastaut syndrome.

36. The compound of claim 10 having the structure: 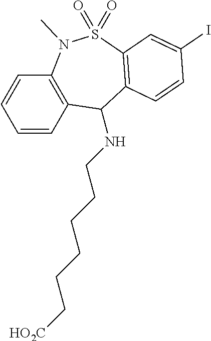 [SMILES: CN1c2ccccc2C(NCCCCCCC(=O)O)c2ccc(I)cc2S1(=O)=O]
or a pharmaceutically acceptable salt thereof; wherein the compound is for treating or preventing a neurological disorder in a subject, wherein the neurological disorder is selected from Huntington's disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; or Lennox-Gastaut syndrome.

37. The compound of claim 1 having the structure: 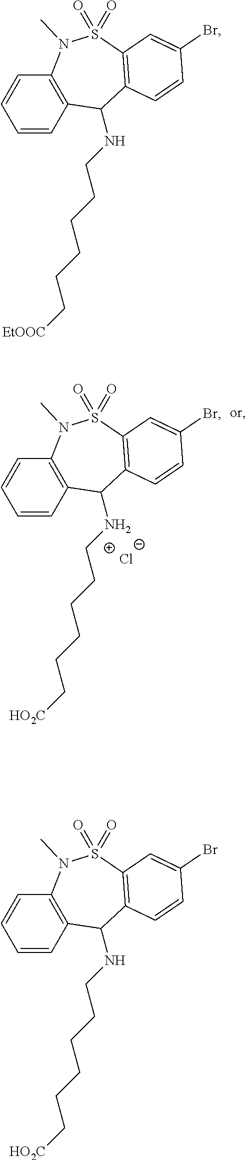 [SMILES: CCOC(=O)CCCCCCNC1c2ccccc2N(C)S(=O)(=O)c2cc(Br)ccc21 ; CN1c2ccccc2C(NCCCCCCC(=O)O)c2ccc(Br)cc2S1(=O)=O ; Cl ; CN1c2ccccc2C(NCCCCCCC(=O)O)c2ccc(Br)cc2S1(=O)=O]
or a pharmaceutically acceptable salt thereof; wherein the compound is for treating or preventing a neurological disorder in a subject, wherein the neurological disorder is selected from Huntington's disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; or Lennox-Gastaut syndrome.

38. The compound of claim 12 having the structure: 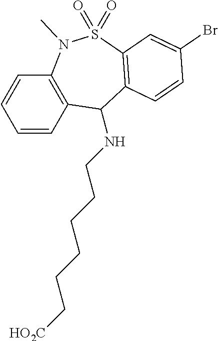 [SMILES: CN1c2ccccc2C(NCCCCCCC(=O)O)c2ccc(Br)cc2S1(=O)=O]
or a pharmaceutically acceptable salt thereof; wherein the compound is for treating or preventing a neurological disorder in a subject, wherein the neurological disorder is selected from Huntington's disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; or Lennox-Gastaut syndrome.

39. A method of treating a neurological disorder in a subject, wherein the neurological disorder is selected from Huntington's disease; Rett syndrome; a Rett syndrome variant; Angelman syndrome; Prader-Willi syndrome; neonatal onset encephalopathy; X-linked recessive mental retardation; fetal alcohol spectrum disorder; Hirschsprung disease; CDKL5 disorder; West syndrome; FOXG1 syndrome; or Lennox-Gastaut syndrome, comprising administering an effective amount of the compound of claim 3 to the subject so as to treat the neurological disorder.
